(19) United States
(12) Patent Application Publication
(10) Pub. No.: US 2023/0115534 A1
(43) Pub. Date: Apr. 13, 2023

(54) PROGRANULIN MODULATORS AND METHODS OF USING THE SAME

(71) Applicant: ARKUDA THERAPEUTICS, Watertown, MA (US)

(72) Inventors: Duane A. Burnett, Watertown, MA (US); James C. Lanter, Watertown, MA (US); Johannes Paulus Gerardus Seerden, Groningen (NL); Wim Zonneveld, Nijmegen (NL); Roel Peter Liesbeth Bell, Nijmegen (NL)

(21) Appl. No.: 17/786,250

(22) PCT Filed: Dec. 18, 2020
(86) PCT No.: PCT/US20/65787

§ 371 Date: Jun. 16, 2022

Related U.S. Application Data
(60) Provisional application No. 62/951,324, filed on Dec. 20, 2019.

(51) Int. Cl. C07D 217/14; C07D 401/12

(52) U.S. Cl. CPC C07D 217/14; C07D 401/12

(57) ABSTRACT

Provided herein are compounds that modulate progranulin and methods of using the compounds in progranulin-associated disorders, such as Frontotemperal lobe dementia (FTLD).

### BACKGROUND

Provided herein are compounds that modulate progranulin levels and can be useful as therapeutics for granulin (GRN)- and/or progranulin (PGRN)-associated disorders. Mutations in the GRN gene cause Frontotemporal lobar degeneration (FTLD) (see, e.g., Cruts et al., Granulin Mutations Associated with Frontotemporal Lobar Degeneration and Related Disorders: An Update, Hu Mutation, 2008 and Baker et al., Nature, 2006.) FTLD-associated mutations in GRN result in a reduction of progranulin protein expression, which suggests that haploinsufficiency of progranulin is the critical pathogenic factor in FTLD-GRN. Plasma and CSF progranulin levels are reduced by up to 70% in pathogenic GRN mutation carriers (Ghidoni, et al., Neurodegen Dis, 2012). More than 60 non-sense mutations in the GRN gene have been described. Plasma can be easily monitored for PGRN (see e.g., Meeter, Nature Neurology, volume 13, 2017). Thus, granulin- and/or progranulin-associated disorders can be modulated by compounds which increase progranulin secretion and/or activity.

All known FTLD-GRN-associated mutations cause haploinsufficiency of progranulin, suggesting that restoration of proper progranulin levels or progranulin protein function will be therapeutically beneficial for FTLD-GRN patients. Several studies have shown that even subtle reductions in progranulin levels by genetic modifiers (e.g., TMEM106B, SLPI, Rs5848) have significant effects on the age-of-onset of FTLD, increase the risk of developing FTLD, or worsen the course of autoimmune diseases such as osteoarthritis (see, e.g., Nicholson et al., J Neurochem, 2013; Cruchaga et al., Arch Neurol, 2012; and Wei et al, Plos One, 2014). Polymorphisms that affect progranulin levels have also been identified as genetic modifiers of several other neurodegenerative diseases, such as Alzheimer's disease and C9orf72-linked FTLD (see, e.g., Sheng et al., Gene, 2014 and van Blitterswijk et al., Mol Neurodegen, 2014). As such, it is contemplated herein that progranulin-targeted therapeutics are effective across multiple neurodegenerative and autoimmune disorders.

Granulins are a family of secreted and glycosylated proteins. They are cleaved from a common precursor protein called progranulin (PGRN). Progranulin is a secreted glycoprotein and is expressed in neurons, neuroglia, chondrocytes, epithelial cells and leukocytes (Toh H et al. J Mol Neurosci 2011 November; 45(3):538-48). It is a precursor protein with an N-terminal signal peptide and seven granulin motifs. Each of these granulin motifs contains 12 cysteines, which are responsible for 6 disulfide bridges in every granulin (Bateman A et al. Bioessays 2009:1245-54). Progranulin is coded by the GRN gene. Mutations in the GRN gene have been implicated in up to 25% of frontotemporal lobar degeneration, inherited in an autosomal dominant fashion with high penetrance (see, e.g., Mackenzie, Acta Neuropathologica, 114(1): 49-54 (2007)). Thus, modulation of progranulin activity is an attractive target for treating disorders associated with GRN activity or GRN-gene mutations.

### SUMMARY

Provided herein are compounds and methods for modulating progranulin, e.g., increasing the level of progranulin or granulin in a subject. More particularly, provided are modulators of progranulin and the uses of such modulators in treating progranulin-associated disorders, e.g., Alzheimer's disease (AD), Parkinson's disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington's disease (HD), Spinocerebellar ataxia (SCA), Spinal muscular atrophy (SMA), lysosomal storage diseases, diseases associated with inclusions and/or misfunction of C9orf72, TDP-43, FUS, UBQLN2, VCP, CHMP28, and/or MAPT, acute neurological disorders, glioblastoma, or neuroblastoma.

In one aspect, the disclosure provides compounds of Formula (I):

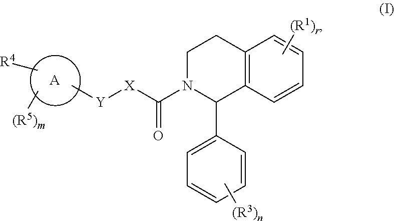

wherein X is O, NR², or CR^{A}R^{B}, Y is C₀₋₆alkylene; ring A is a 3- to 8-membered carbocycle; R^{A} is hydrogen, OH, or O—C₁₋₃alkyl; R^{B} is hydrogen; or R^{A} and R^{B} together are oxo; R¹ is hydrogen, C₁₋₆alkyl, C₀₋₃ haloalkyl, O—C₁₋₃ haloalkyl, C₀₋₃ alkylene-CN, C₀₋₃ alkylene-NR^{N}₂, C₀₋₆ alkylene-OR^{N}, C₀₋₆ alkylene-C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2; R² is hydrogen, C₁₋₆alkyl, C₃₋₆alkynyl, or C₁₋₆alkylene-SO₂—C₁₋₃alkyl; each R³ is independently halo; R⁴ is hydrogen, OH, halo, or C₀₋₆alkylene-NR⁶R⁷; each R⁵ is independently hydrogen, C₁₋₃alkyl, C₀₋₃hydroxyalkyl, or halo; R⁶ is hydrogen or C₁₋₃alkyl; R⁷ is hydrogen, C₁₋₃alkyl, C₁₋₃haloalkyl, C₁₋₃hydroxyalkyl, C₂₋₆alkynyl, C₁₋₃alkylene-O—C₂₋₆alkynyl, C₀₋₃alkylene-SO₂—C₁₋₃alkyl, C₀₋₃alkylene-SO₂—C₁₋₃ haloalkyl, C(O)C₁₋₆alkyl, or C(O)C₁₋₆haloalkyl; or R⁶ and R⁷ together with the nitrogen to which they are attached form a 4- to 6-membered heterocycle having 0-1 additional ring heteroatoms selected from N, O, and S and optionally substituted with 1, 2, or 3 groups independently selected from halo and oxo; m is 1 or 2; n is 0-3; and r is 1 or 2. In some embodiments, X is O, NR², or CR^{A}R^{B}, Y is C₀₋₆alkylene; ring A is a 3- to 8-membered carbocycle; R^{A} is hydrogen, OH, or O—C₁₋₃alkyl; R^{B} is hydrogen; or R^{A} and R^{B} together are oxo; R¹ is hydrogen, C₁₋₆alkyl, C₀₋₃ haloalkyl, O—C₁₋₃ haloalkyl, C₀₋₃ alkylene-CN, C₀₋₃ alkylene-NR^{N}₂, C₀₋₆ alkylene-OR^{N}, C₀₋₆ alkylene-C(O)OR^{N}, C₀₋₆ alkylene-C(O)N(R^{N})₂, or C₀₋₆ alkylene-SOₚR^{N} each R^{N} is independently hydrogen or C₁₋₆alkyl, and p is 0-2; R² is hydrogen, C₁₋₆alkyl, C₃₋₆alkynyl, or C₁₋₆alkylene-SO₂—C₁₋₃alkyl; each R³ is independently halo; R⁴ is hydrogen, OH, halo, or C₀₋₆alkylene-NR⁶R⁷; each R⁵ is independently hydrogen, C₁₋₃alkyl, or halo; R⁶ is hydrogen or C₁₋₃alkyl; R⁷ is hydrogen, C₁₋₃alkyl, C₁₋₃haloalkyl, C₂₋₆alkynyl, C₁₋₃alkylene-O—C₂₋₆alkynyl, C₀₋₃alkylene-SO₂—C₁₋₃alkyl, C₀₋₃alkylene-SO₂—C₁₋₃haloalkyl, C(O)C₁₋₆alkyl, or C(O)C₁₋₆haloalkyl; or R⁶ and R⁷ together with the nitrogen to which they are attached form a 4- to 6-membered heterocycle having 0-1 additional ring heteroatoms selected from N, O, and S and optionally substituted with 1, 2, or 3 groups independently selected from halo and oxo; m is 1 or 2; n is 0-3; and r is 1 or 2.

In some cases, ring A is

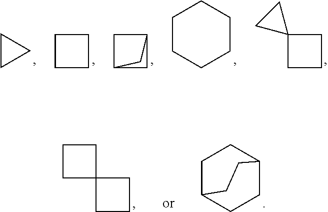

Further provided are methods of synthesizing compounds of Formula (I). In some cases, the method comprises heating an acyl azide compound to undergo a Curtius rearrangement to form an isocyanate compound; and admixing the isocyanate compound with an amine compound to form a urea derivative.

Further provided are methods of modulating progranulin in a subject. In some embodiments, provided are methods of treating a progranulin-associated disorder in a subject.

Other aspects of the disclosure include a compound as disclosed herein for use in the preparation of a medicament for the modulation of progranulin, and the use of a compound as disclosed herein in a method of treating or preventing a progranulin-associated disorder in a subject.

### DETAILED DESCRIPTION

### Compounds as Progranulin Modulators

Provided herein are compounds that can modulate progranulin production and/or secretion. In some cases, the compounds can increase the the level of progranulin or granulin in a subject.

The disclosure provides compounds of Formula (I):

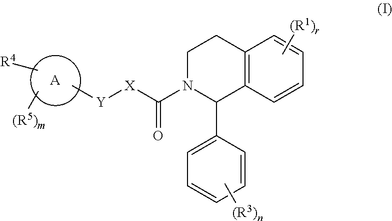

wherein
X is O, NR², or CR^{A}R^{B};
Y is C₀₋₆alkylene;
ring A is a 3- to 8-membered carbocycle;
R^{A} is hydrogen, OH, or O—C₁₋₃alkyl;
R^{B} is hydrogen; or
R^{A} and R^{B} together are oxo;
R¹ is hydrogen, C₁₋₆alkyl, C₀₋₃ haloalkyl, O—C₁₋₃ haloalkyl, C₀₋₃ alkylene-CN, C₀₋₃ alkylene-NR^{N}₂, C₀₋₆ alkylene-OR^{N}, C₀₋₆ alkylene-C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2;
R² is hydrogen, C₁₋₆alkyl, C₃₋₆alkynyl, or C₁₋₆alkylene-SO₂—C₁₋₃alkyl;
each R³ is independently halo;
R⁴ is hydrogen, OH, halo, or C₀₋₆alkylene-NR⁶R⁷;
each R⁵ is independently hydrogen, C₁₋₃alkyl, C₀₋₃hydroxyalkyl, or halo;
R⁶ is hydrogen or C₁₋₃alkyl;
R⁷ is hydrogen, C₁₋₃alkyl, C₁₋₃haloalkyl, C₁₋₃hydroxyalkyl, C₂₋₆alkynyl, C₁₋₃alkylene-O—C₂₋₆alkynyl, C₀₋₃alkylene-SO₂—C₁₋₃alkyl, C₀₋₃alkylene-SO₂—C₁₋₃haloalkyl, C(O)C₁₋₆alkyl, or C(O)C₁₋₆haloalkyl; or
R⁶ and R⁷ together with the nitrogen to which they are attached form a 4- to 6-membered heterocycle having 0-1 additional ring heteroatoms selected from N, O, and S and optionally substituted with 1, 2, or 3 groups independently selected from halo and oxo;
m is 1 or 2;
n is 0-3; and
r is 1 or 2.

In some cases, X is O, NR², or CR^{A}R^{B};
Y is C₀₋₆alkylene;
ring A is a 3- to 8-membered carbocycle;
R^{A} is hydrogen, OH, or O—C₁₋₃alkyl;
R^{B} is hydrogen; or
R^{A} and R^{B} together are oxo;
R¹ is hydrogen, C₁₋₆alkyl, C₀₋₃haloalkyl, O—C₁₋₃ haloalkyl, C₀₋₃ alkylene-CN, C₀₋₃ alkylene-NR^{N}₂, C₀₋₆ alkylene-OR^{N}, C₀₋₆ alkylene-C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2;
R² is hydrogen, C₁₋₆alkyl, C₃₋₆alkynyl, or C₁₋₆alkylene-SO₂—C₁₋₃alkyl;
each R³ is independently halo;
R⁴ is hydrogen, OH, halo, or C₀₋₆alkylene-NR⁶R⁷;
each R⁵ is independently hydrogen, C₁₋₃alkyl, or halo;
R⁶ is hydrogen or C₁₋₃alkyl;
R⁷ is hydrogen, C₁₋₃alkyl, C₁₋₃haloalkyl, C₂₋₆alkynyl, C₁₋₃alkylene-O—C₂₋₆alkynyl, C₀₋₃alkylene-SO₂—C₁₋₃alkyl, C₀₋₃alkylene-SO₂—C₁₋₃haloalkyl, C(O)C₁₋₆alkyl, or C(O)C₁₋₆haloalkyl; or
R⁶ and R⁷ together with the nitrogen to which they are attached form a 4- to 6-membered heterocycle having 0-1 additional ring heteroatoms selected from N, O, and S and optionally substituted with 1, 2, or 3 groups independently selected from halo and oxo;
m is 1 or 2;
n is 0-3; and
r is 1 or 2.

In some cases, X is O. In some cases, X is CR^{A}R^{B}. In some cases, X is CH₂. In some cases, X is NR².

In some cases, R^{A} is OH. In some cases, R^{A} and R^{B} together are oxo.

In some cases, Y is C₀alkylene. In some cases, Y is C₁₋₆alkylene. In some cases, Y is C₀alkylene. In some cases, Y is C₂alkylene. In some cases, Y is C₀alkylene.

In some cases, ring A is a 3- to 5-membered carbocycle. In some cases, ring A is a 6- to 8-membered carbocycle. In some cases, ring A is a 3-membered carbocycle. In some cases, ring A is a 4-membered carbocycle. In some cases, ring A is a 5-membered carbocycle. In some cases, ring A is a 6-membered carbocycle. In some cases, ring A is a 7-membered carbocycle. In some cases, ring A is a 8-membered carbocycle. In some cases, ring A is

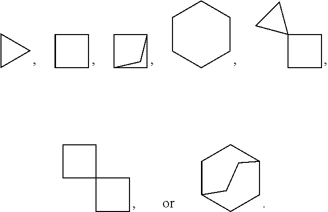

In some cases, ring A is [symbol] .

In some cases, R¹ is hydrogen. In some cases, R¹ is C₁₋₆alkyl, C₀₋₃haloalkyl, O—C₁₋₃ haloalkyl, C₀₋₃ alkylene-CN, C₀₋₃ alkylene-NR^{N}₂, C₀₋₆ alkylene-OR^{N}, C₀₋₆ alkylene-C(O)OR^{N}, C₀₋₆ alkylene-C(O)N(R^{N})₂, or C₀₋₆ alkylene-SOₚR^{N}. In some cases, R¹ is F, Cl, OH, OMe, OiPr, OBn, O-cyclopropyl, CF₃, OCF₃, CN, SO₂Me, SO₂-cyclopropyl, SO₂iPr, oxo, imidazolyl, triazolyl, pyrrolidinyl, pyrrolidinonyl, thiadiazolyl, methyl-thiadiazolyl, trifluoromethyl-thiadiazolyl, oxadiazolyl, methyl-oxadiazolyl, trifluoromethyl-oxadiazolyl, or phenyl. In some cases, R¹ is F or Cl. In some cases, R¹ is F. In some cases, R¹ is hydrogen, C₁₋₆alkyl, C₀₋₃ haloalkyl, O—C₁₋₃ haloalkyl, C₀₋₃ alkylene-CN, C₀₋₃ alkylene-NR^{N}₂, C₀₋₆ alkylene-OR^{N}, C₀₋₆ alkylene-C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2.

In some cases, at least one R^{N} is hydrogen. In some cases, each R^{N} is hydrogen.

In some cases, at least one R^{N} is C₁₋₆alkyl. In some cases, each R^{N} is C₁₋₆akyl.

In some cases, R² is hydrogen. In some cases, R² is C₁₋₆alkyl, C₃₋₆alkynyl, or C₁₋₆alkylene-SO₂—C₁₋₃alkyl. In some cases, R² is CH₃.

In some cases, R³ is fluoro.

In some cases, R⁴ is hydrogen. In some cases, R⁴ is OH. In some cases, R⁴ is halo. In some cases, R⁴ is F. In some cases, R⁴ is C₀₋₆alkylene-NR⁶R⁷. In some cases, R⁴ is —CH₂NH₂, —CH₂NHCH₃, —CH₂CH₂NH₂, —CH₂CH(CH₃)NH₂, —CH₂C(CH₃)₂NH₂, or —CH₂CH₂N(CH₃)₂. In some cases, R⁴ is NR⁶R⁷. In some cases, R⁴ is C₁alkylene-NR⁶R⁷. In some cases, R⁴ is —NH₂, —NHCH₃, —N(CH₃)₂, —NHCH₂CH₃, —NHCH₂CF₃, —NH-propargyl, —NHC(O)CH₃, —NHC(O)CF₃, —NHSO₂CH₃, —NHSO₂CF₃, or —NHCH₂CH₂SO₂CH₃. In some cases, R⁴ is —NH₂.

In some cases, R⁵ is hydrogen, C₁₋₃alkyl, or halo.

In some cases, R⁶ is hydrogen. In some cases, R⁶ is C₁₋₃alkyl. In some cases, R⁶ is methyl.

In some cases, R⁷ is hydrogen. In some cases, R⁷ is C₁₋₃alkyl. In some cases, R⁷ is methyl. In some cases, R⁷ is ethyl. In some cases, R⁷ is C₁₋₃haloalkyl. In some cases, R⁷ is trifluoroethyl. In some cases, R⁷ is C₂₋₆alkynyl. In some cases, R⁷ is propargyl. In some cases, R⁷ is C₀₋₃alkylene-SO₂—C₁₋₃alkyl or C₀₋₃alkylene-SO₂—C₁₋₃haloalkyl. In some cases, R⁷ is SO₂-methyl or SO₂CF₃. In some cases, R⁷ is C₂alkylene-SO₂-methyl. In some cases, wherein R⁷ is C(O)C₁₋₆alkyl or C(O)C₁₋₆haloalkyl. In some cases, R⁷ is C(O)CH₃ or C(O)CF₃. In some cases, R⁷ is hydrogen, C₁₋₃alkyl, C₁₋₃haloalkyl, C₂₋₆alkynyl, C₁₋₃alkylene-O—C₂₋₆alkynyl, C₀₋₃alkylene-SO₂—C₁₋₃alkyl, C₀₋₃alkylene-SO₂—C₁₋₃haloalkyl, C(O)C₁₋₆alkyl, or C(O)C₁₋₆haloalkyl.

In some cases, R⁶ and R⁷ together with the nitrogen to which they are attached form a 4- to 6-membered heterocycle. In some cases, R⁶ and R⁷ together with the nitrogen to which they are attached form a heterocycle selected from

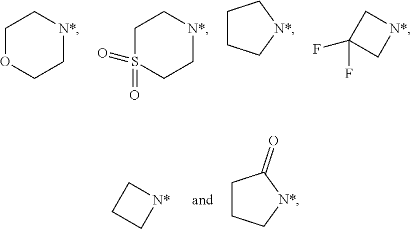

wherein N* indicates the nitrogen to which R⁶ and R⁷ are attached.

In some cases, at least one R⁵ is H. In some cases, each R⁵ is hydrogen. In some cases, at least one R⁵ is C₁₋₃alkyl. In some cases, at least one R⁵ is methyl. In some cases, each R⁵ is methyl. In some cases, at least one R⁵ is halo. In some cases, at least one R⁵ is fluoro. In some cases, each R⁵ is fluoro.

In some cases, R^{N} is hydrogen. In some cases, R^{N} is C₁₋₆alkyl. In some cases, R^{N} is methyl.

In some cases, m is 1. In some cases, m is 2.

In some cases, n is 0. In some cases, n is 1. In some cases, n is 2. In some cases, n is 3.

In some cases, p is 0. In some cases, p is 1. In some cases, p is 2.

In some cases, r is 1. In some cases, r is 2.

Specific compounds contemplated include those listed in Table A, or a pharmaceutically acceptable salt thereof:

TABLE A

| | Cpd |
|---|---|
| Structure | No. |
| 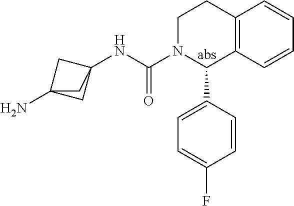 [SMILES: NC12CC(NC(=O)N3CCc4ccccc4[C@@H]3c3ccc(F)cc3)(C1)C2] | 4002 |
| 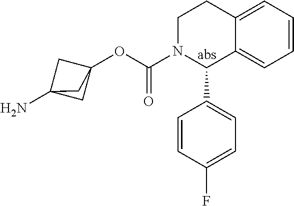 [SMILES: NC12CC(OC(=O)N3CCc4ccccc4[C@@H]3c3ccc(F)cc3)(C1)C2] | 4001 |
| 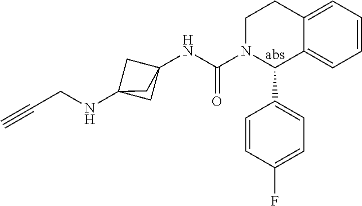 [SMILES: C#CCNC12CC(NC(=O)N3CCc4ccccc4[C@@H]3c3ccc(F)cc3)(C1)C2] | 4005 |
| 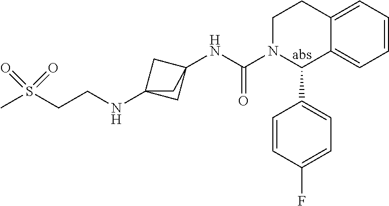 [SMILES: CS(=O)(=O)CCNC12CC(NC(=O)N3CCc4ccccc4[C@@H]3c3ccc(F)cc3)(C1)C2] | 4007 |
| 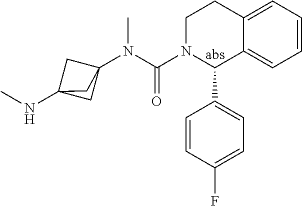 [SMILES: CNC12CC(N(C)C(=O)N3CCc4ccccc4[C@@H]3c3ccc(F)cc3)(C1)C2] | 4009 |
| 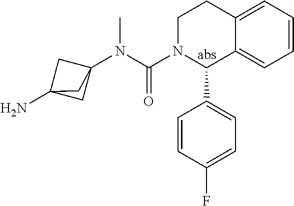 [SMILES: CN(C(=O)N1CCc2ccccc2[C@@H]1c1ccc(F)cc1)C12CC(N)(C1)C2] | 4010 |
| 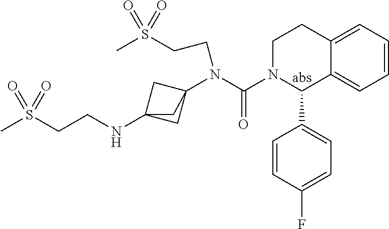 [SMILES: CS(=O)(=O)CCNC12CC(N(CCS(C)(=O)=O)C(=O)N3CCc4ccccc4[C@@H]3c3ccc(F)cc3)(C1)C2] | 4008 |
| 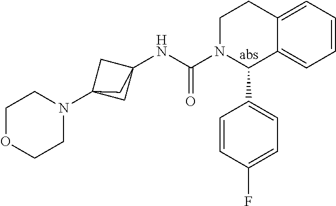 [SMILES: O=C(NC12CC(N3CCOCC3)(C1)C2)N1CCc2ccccc2[C@@H]1c1ccc(F)cc1] | 4013 |
| 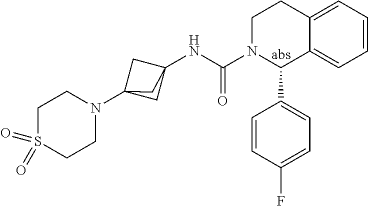 [SMILES: O=C(NC12CC(N3CCS(=O)(=O)CC3)(C1)C2)N1CCc2ccccc2[C@@H]1c1ccc(F)cc1] | 4021 |
| 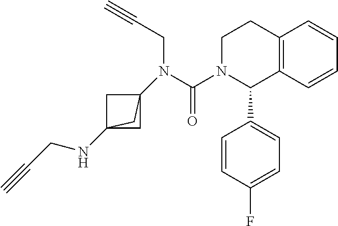 [SMILES: C#CCNC12CC(N(CC#C)C(=O)N3CCc4ccccc4[C@@H]3c3ccc(F)cc3)(C1)C2] | 4006 |
| 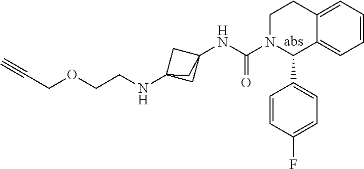 [SMILES: C#CCOCCNC12CC(NC(=O)N3CCc4ccccc4[C@@H]3c3ccc(F)cc3)(C1)C2] | 4011 |
| 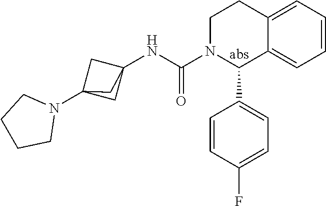 [SMILES: O=C(NC12CC(N3CCCC3)(C1)C2)N1CCc2ccccc2[C@@H]1c1ccc(F)cc1] | 4012 |
| 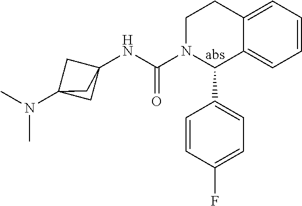 [SMILES: CN(C)C12CC(NC(=O)N3CCc4ccccc4[C@@H]3c3ccc(F)cc3)(C1)C2] | 4022 |
| 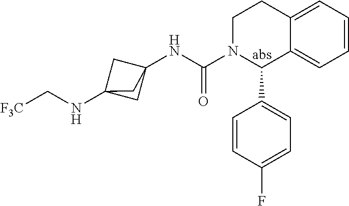 [SMILES: O=C(NC12CC(NCC(F)(F)F)(C1)C2)N1CCc2ccccc2[C@@H]1c1ccc(F)cc1] | 4023 |
| 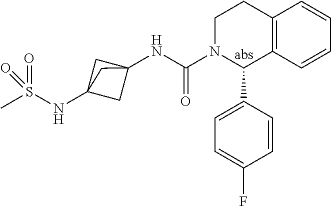 [SMILES: CS(=O)(=O)NC12CC(NC(=O)N3CCc4ccccc4[C@@H]3c3ccc(F)cc3)(C1)C2] | 4017 |
| 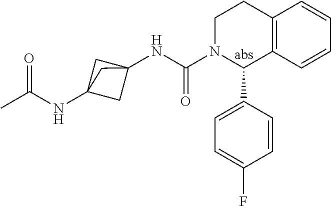 [SMILES: CC(=O)NC12CC(NC(=O)N3CCc4ccccc4[C@@H]3c3ccc(F)cc3)(C1)C2] | 4016 |
| 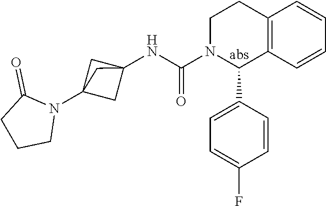 [SMILES: O=C(NC12CC(N3CCCC3=O)(C1)C2)N1CCc2ccccc2[C@@H]1c1ccc(F)cc1] | 4024 |
| 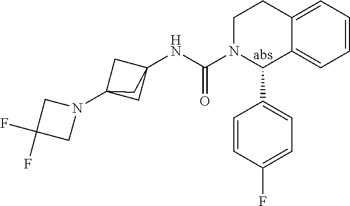 [SMILES: O=C(NC12CC(N3CC(F)(F)C3)(C1)C2)N1CCc2ccccc2[C@@H]1c1ccc(F)cc1] | 4052 |
| 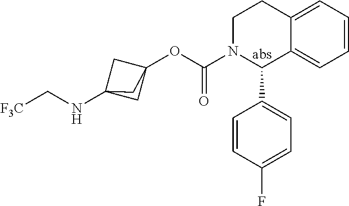 [SMILES: O=C(OC12CC(NCC(F)(F)F)(C1)C2)N1CCc2ccccc2[C@@H]1c1ccc(F)cc1] | 4014 |
| 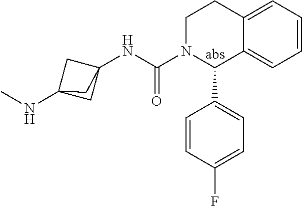 [SMILES: CNC12CC(NC(=O)N3CCc4ccccc4[C@@H]3c3ccc(F)cc3)(C1)C2] | 4004 |
| 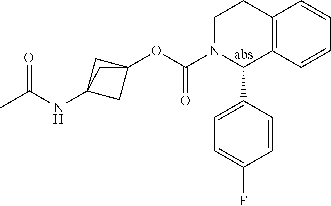 [SMILES: CC(=O)NC12CC(OC(=O)N3CCc4ccccc4[C@@H]3c3ccc(F)cc3)(C1)C2] | 4015 |
| 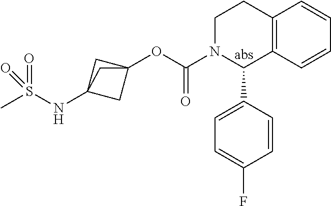 [SMILES: CS(=O)(=O)NC12CC(OC(=O)N3CCc4ccccc4[C@@H]3c3ccc(F)cc3)(C1)C2] | 4018 |
| 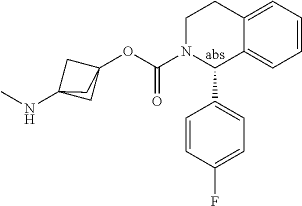 [SMILES: CNC12CC(OC(=O)N3CCc4ccccc4[C@@H]3c3ccc(F)cc3)(C1)C2] | 4026 |
| 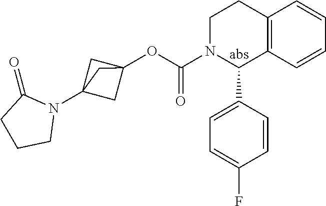 [SMILES: O=C(OC12CC(N3CCCC3=O)(C1)C2)N1CCc2ccccc2[C@@H]1c1ccc(F)cc1] | 4025 |
| 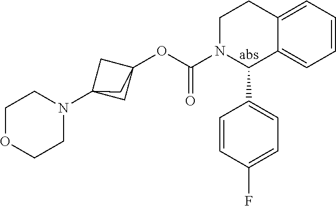 [SMILES: O=C(OC12CC(N3CCOCC3)(C1)C2)N1CCc2ccccc2[C@@H]1c1ccc(F)cc1] | 4049 |
| 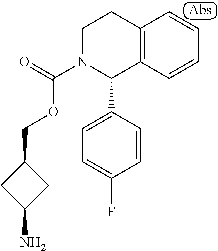 [SMILES: N[C@H]1C[C@@H](COC(=O)N2CCc3ccccc3[C@@H]2c2ccc(F)cc2)C1] | 4046 |
| 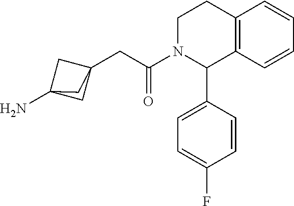 [SMILES: NC12CC(CC(=O)N3CCc4ccccc4C3c3ccc(F)cc3)(C1)C2] | 4056 |
| 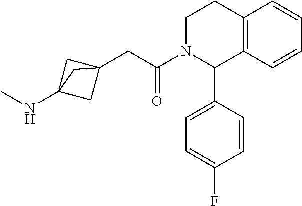 [SMILES: CNC12CC(CC(=O)N3CCc4ccccc4C3c3ccc(F)cc3)(C1)C2] | 4057 |
| 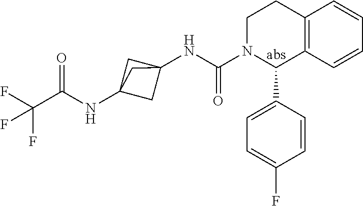 [SMILES: O=C(NC12CC(NC(=O)C(F)(F)F)(C1)C2)N1CCc2ccccc2[C@@H]1c1ccc(F)cc1] | 4019 |
| 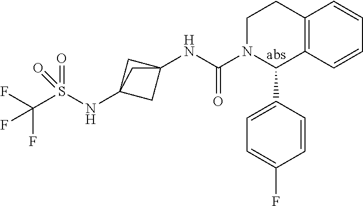 [SMILES: O=C(NC12CC(NS(=O)(=O)C(F)(F)F)(C1)C2)N1CCc2ccccc2[C@@H]1c1ccc(F)cc1] | 4020 |
| 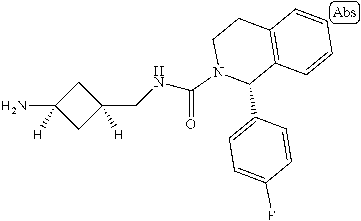 [SMILES: N[C@H]1C[C@@H](CNC(=O)N2CCc3ccccc3[C@@H]2c2ccc(F)cc2)C1] | 4028 |
| 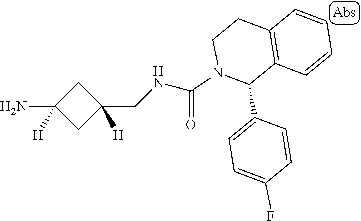 [SMILES: N[C@H]1C[C@H](CNC(=O)N2CCc3ccccc3[C@@H]2c2ccc(F)cc2)C1] | 4029 |
| 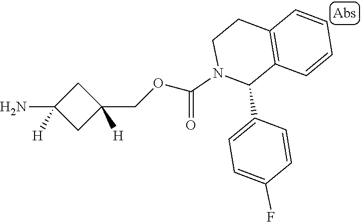 [SMILES: N[C@H]1C[C@H](COC(=O)N2CCc3ccccc3[C@@H]2c2ccc(F)cc2)C1] | 4047 |
| 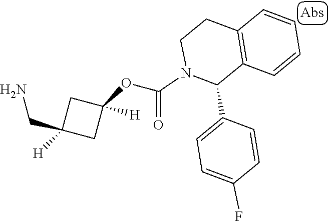 [SMILES: NC[C@H]1C[C@@H](OC(=O)N2CCc3ccccc3[C@@H]2c2ccc(F)cc2)C1] | 4030 |
| 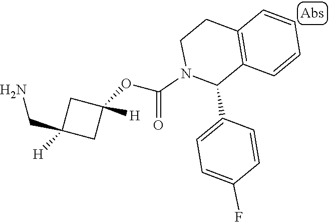 [SMILES: NC[C@H]1C[C@H](OC(=O)N2CCc3ccccc3[C@@H]2c2ccc(F)cc2)C1] | 4031 |
| 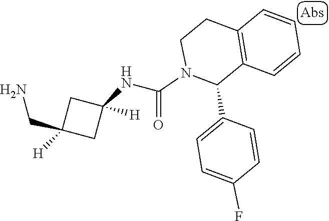 [SMILES: NC[C@H]1C[C@@H](NC(=O)N2CCc3ccccc3[C@@H]2c2ccc(F)cc2)C1] | 4032 |
| 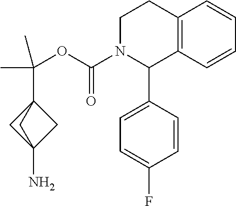 [SMILES: CC(C)(OC(=O)N1CCc2ccccc2C1c1ccc(F)cc1)C12CC(N)(C1)C2] | 4048 |
| 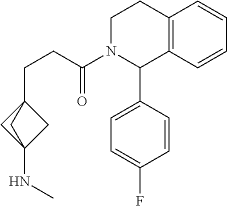 [SMILES: CNC12CC(CCC(=O)N3CCc4ccccc4C3c3ccc(F)cc3)(C1)C2] | 4058 |
| 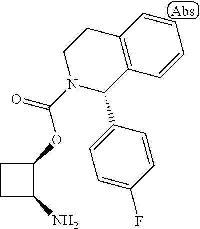 [SMILES: N[C@H]1CC[C@H]1OC(=O)N1CCc2ccccc2[C@@H]1c1ccc(F)cc1] | 4037 |
| 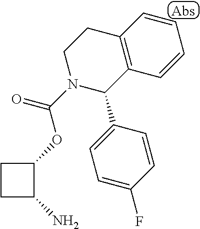 [SMILES: N[C@@H]1CC[C@@H]1OC(=O)N1CCc2ccccc2[C@@H]1c1ccc(F)cc1] | 4038 |
| 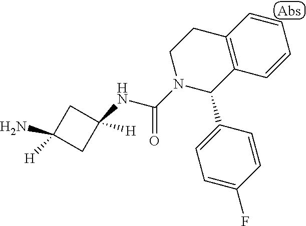 [SMILES: N[C@H]1C[C@@H](NC(=O)N2CCc3ccccc3[C@@H]2c2ccc(F)cc2)C1] | 4034 |
| 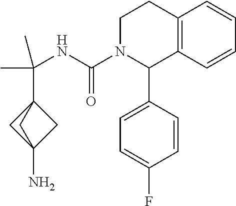 [SMILES: CC(C)(NC(=O)N1CCc2ccccc2C1c1ccc(F)cc1)C12CC(N)(C1)C2] | 4050 |
| 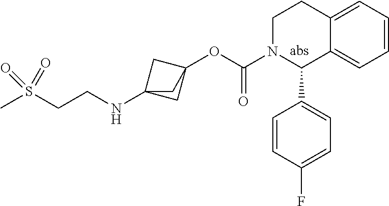 [SMILES: CS(=O)(=O)CCNC12CC(OC(=O)N3CCc4ccccc4[C@@H]3c3ccc(F)cc3)(C1)C2] | 4027 |
| 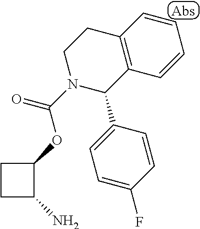 [SMILES: N[C@@H]1CC[C@H]1OC(=O)N1CCc2ccccc2[C@@H]1c1ccc(F)cc1] | 4039 |
| 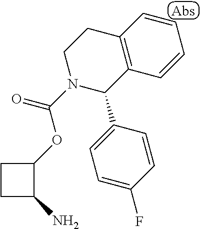 [SMILES: N[C@H]1CCC1OC(=O)N1CCc2ccccc2[C@@H]1c1ccc(F)cc1] | 4040 |
| 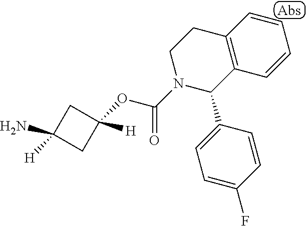 [SMILES: N[C@H]1C[C@H](OC(=O)N2CCc3ccccc3[C@@H]2c2ccc(F)cc2)C1] | 4036 |
| 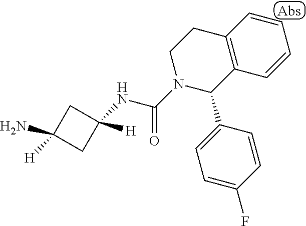 [SMILES: N[C@H]1C[C@H](NC(=O)N2CCc3ccccc3[C@@H]2c2ccc(F)cc2)C1] | 4035 |
| 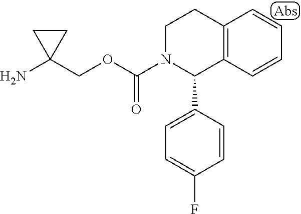 [SMILES: NC1(COC(=O)N2CCc3ccccc3[C@@H]2c2ccc(F)cc2)CC1] | 4033 |
| 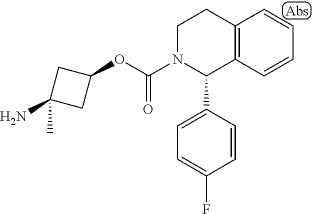 [SMILES: C[C@]1(N)C[C@@H](OC(=O)N2CCc3ccccc3[C@@H]2c2ccc(F)cc2)C1] | 4041 |
| 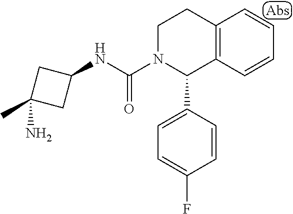 [SMILES: C[C@]1(N)C[C@H](NC(=O)N2CCc3ccccc3[C@@H]2c2ccc(F)cc2)C1] | 4042 |
| 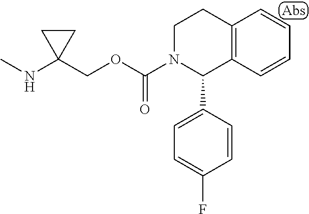 [SMILES: CNC1(COC(=O)N2CCc3ccccc3[C@@H]2c2ccc(F)cc2)CC1] | 4051 |
| 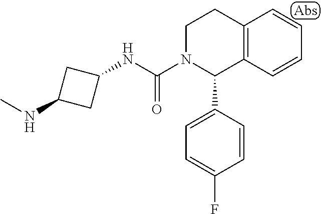 [SMILES: CN[C@H]1C[C@H](NC(=O)N2CCc3ccccc3[C@@H]2c2ccc(F)cc2)C1] | 4043 |
| 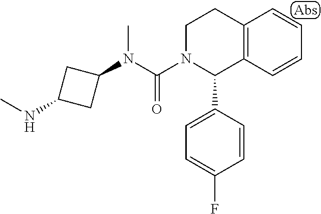 [SMILES: CN[C@H]1C[C@H](N(C)C(=O)N2CCc3ccccc3[C@@H]2c2ccc(F)cc2)C1] | 4044 |
| 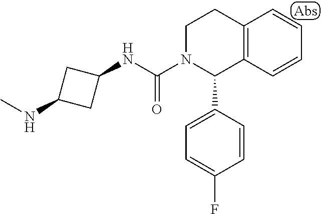 [SMILES: CN[C@H]1C[C@@H](NC(=O)N2CCc3ccccc3[C@@H]2c2ccc(F)cc2)C1] | 4045 |
| 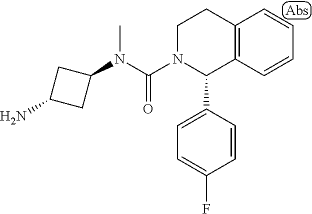 [SMILES: CN(C(=O)N1CCc2ccccc2[C@@H]1c1ccc(F)cc1)[C@H]1C[C@H](N)C1] | 4055 |
| 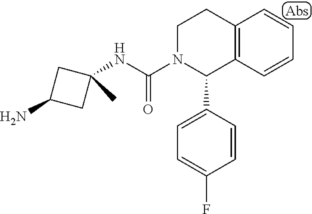 [SMILES: C[C@]1(NC(=O)N2CCc3ccccc3[C@@H]2c2ccc(F)cc2)C[C@H](N)C1] | 4053 |
| 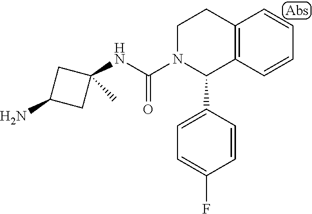 [SMILES: C[C@]1(NC(=O)N2CCc3ccccc3[C@@H]2c2ccc(F)cc2)C[C@@H](N)C1] | 4054 |
| 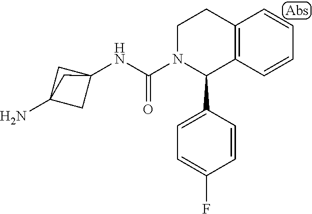 [SMILES: NC12CC(NC(=O)N3CCc4ccccc4[C@H]3c3ccc(F)cc3)(C1)C2] | 4003 |
| 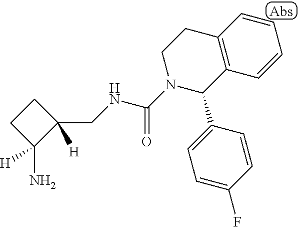 [SMILES: N[C@H]1CC[C@@H]1CNC(=O)N1CCc2ccccc2[C@@H]1c1ccc(F)cc1] | 4060 |
| 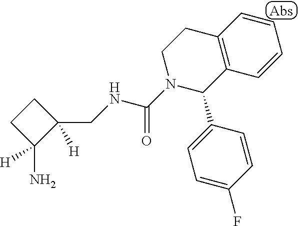 [SMILES: N[C@H]1CC[C@H]1CNC(=O)N1CCc2ccccc2[C@@H]1c1ccc(F)cc1] | 4061 |
| 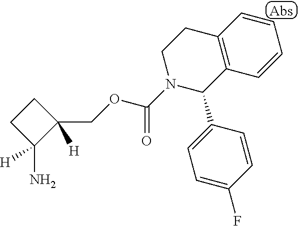 [SMILES: N[C@H]1CC[C@@H]1COC(=O)N1CCc2ccccc2[C@@H]1c1ccc(F)cc1] | 4062 |
| 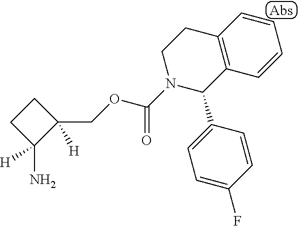 [SMILES: N[C@H]1CC[C@H]1COC(=O)N1CCc2ccccc2[C@@H]1c1ccc(F)cc1] | 4063 |
| 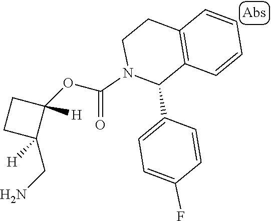 [SMILES: NC[C@H]1CC[C@@H]1OC(=O)N1CCc2ccccc2[C@@H]1c1ccc(F)cc1] | 4064 |
| 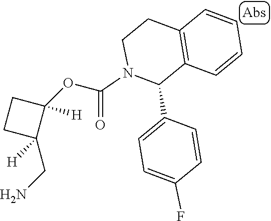 [SMILES: NC[C@H]1CC[C@H]1OC(=O)N1CCc2ccccc2[C@@H]1c1ccc(F)cc1] | 4065 |
| 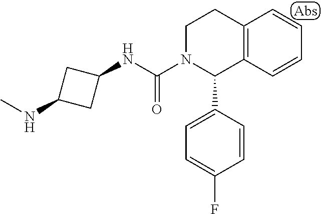 [SMILES: CN[C@H]1C[C@@H](NC(=O)N2CCc3ccccc3[C@@H]2c2ccc(F)cc2)C1] | 4066 |
| 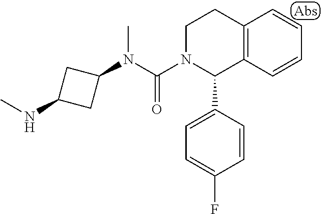 [SMILES: CN[C@H]1C[C@@H](N(C)C(=O)N2CCc3ccccc3[C@@H]2c2ccc(F)cc2)C1] | 4067 |
| 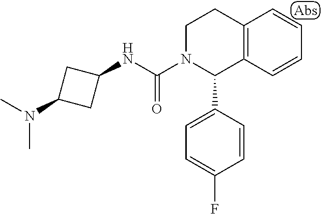 [SMILES: CN(C)[C@H]1C[C@@H](NC(=O)N2CCc3ccccc3[C@@H]2c2ccc(F)cc2)C1] | 4068 |
| 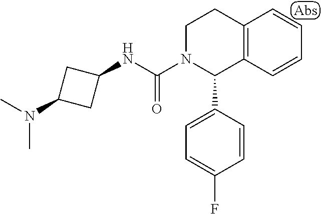 [SMILES: CN(C)[C@H]1C[C@@H](NC(=O)N2CCc3ccccc3[C@@H]2c2ccc(F)cc2)C1] | 4069 |
| 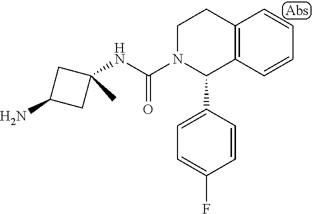 [SMILES: C[C@]1(NC(=O)N2CCc3ccccc3[C@@H]2c2ccc(F)cc2)C[C@H](N)C1] | 4070 |
| 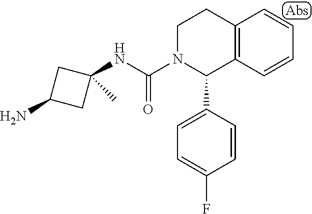 [SMILES: C[C@]1(NC(=O)N2CCc3ccccc3[C@@H]2c2ccc(F)cc2)C[C@@H](N)C1] | 4071 |
| 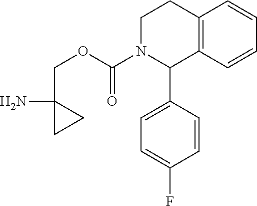 [SMILES: NC1(COC(=O)N2CCc3ccccc3C2c2ccc(F)cc2)CC1] | 4072 |
| 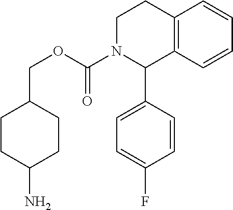 [SMILES: NC1CCC(COC(=O)N2CCc3ccccc3C2c2ccc(F)cc2)CC1] | 4073 |
| 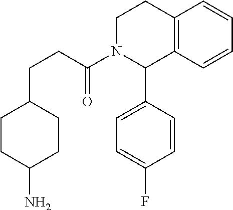 [SMILES: NC1CCC(CCC(=O)N2CCc3ccccc3C2c2ccc(F)cc2)CC1] | 4074 |
| 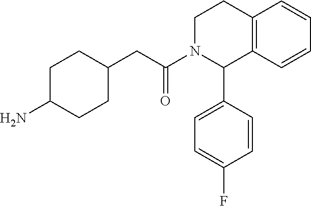 [SMILES: NC1CCC(CC(=O)N2CCc3ccccc3C2c2ccc(F)cc2)CC1] | 4075 |
| 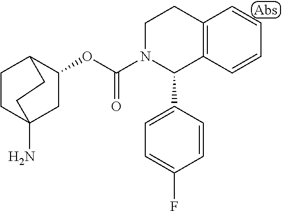 [SMILES: NC12CCC(CC1)[C@H](OC(=O)N1CCc3ccccc3[C@@H]1c1ccc(F)cc1)C2] | 4076 |
| 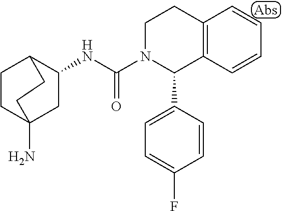 [SMILES: NC12CCC(CC1)[C@H](NC(=O)N1CCc3ccccc3[C@@H]1c1ccc(F)cc1)C2] | 4077 |
| 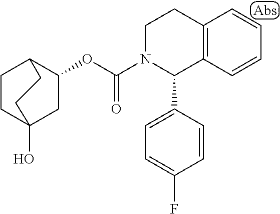 [SMILES: O=C(O[C@@H]1CC2(O)CCC1CC2)N1CCc2ccccc2[C@@H]1c1ccc(F)cc1] | 4078 |
| 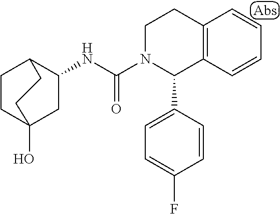 [SMILES: O=C(N[C@@H]1CC2(O)CCC1CC2)N1CCc2ccccc2[C@@H]1c1ccc(F)cc1] | 4079 |
| 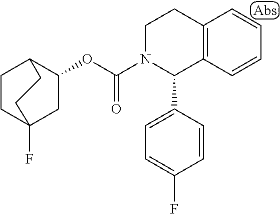 [SMILES: O=C(O[C@@H]1CC2(F)CCC1CC2)N1CCc2ccccc2[C@@H]1c1ccc(F)cc1] | 4080 |
| 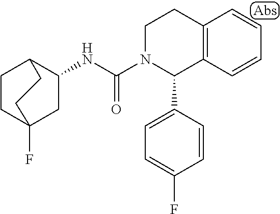 [SMILES: O=C(N[C@@H]1CC2(F)CCC1CC2)N1CCc2ccccc2[C@@H]1c1ccc(F)cc1] | 4081 |
| 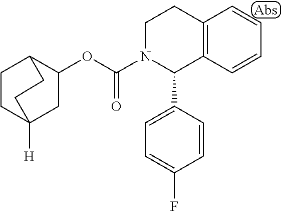 [SMILES: O=C(OC1CC2CCC1CC2)N1CCc2ccccc2[C@@H]1c1ccc(F)cc1] | 4082 |
| 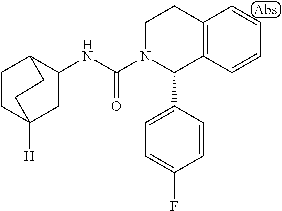 [SMILES: O=C(NC1CC2CCC1CC2)N1CCc2ccccc2[C@@H]1c1ccc(F)cc1] | 4083 |
| 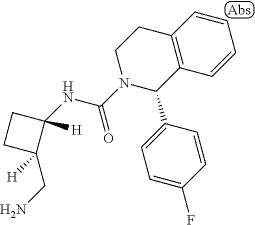 [SMILES: NC[C@H]1CC[C@@H]1NC(=O)N1CCc2ccccc2[C@@H]1c1ccc(F)cc1] | 4084 |
| 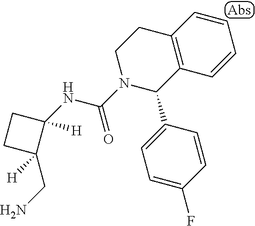 [SMILES: NC[C@H]1CC[C@H]1NC(=O)N1CCc2ccccc2[C@@H]1c1ccc(F)cc1] | 4085 |
| 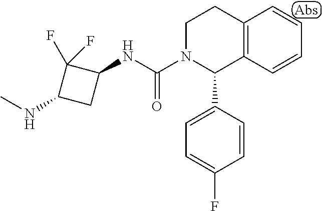 [SMILES: CN[C@H]1C[C@H](NC(=O)N2CCc3ccccc3[C@@H]2c2ccc(F)cc2)C1(F)F] | 4086 |
| 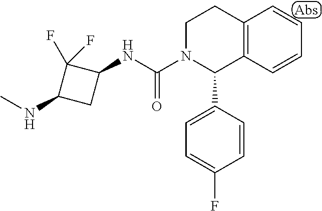 [SMILES: CN[C@@H]1C[C@H](NC(=O)N2CCc3ccccc3[C@@H]2c2ccc(F)cc2)C1(F)F] | 4087 |
| 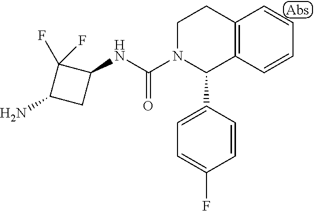 [SMILES: N[C@H]1C[C@H](NC(=O)N2CCc3ccccc3[C@@H]2c2ccc(F)cc2)C1(F)F] | 4088 |
| 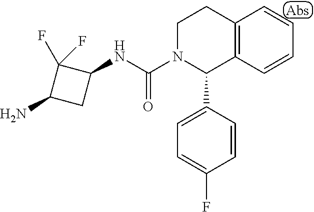 [SMILES: N[C@@H]1C[C@H](NC(=O)N2CCc3ccccc3[C@@H]2c2ccc(F)cc2)C1(F)F] | 4089 |
| 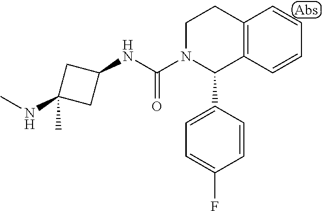 [SMILES: CN[C@]1(C)C[C@H](NC(=O)N2CCc3ccccc3[C@@H]2c2ccc(F)cc2)C1] | 4090 |
| 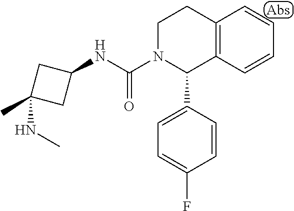 [SMILES: CN[C@]1(C)C[C@@H](NC(=O)N2CCc3ccccc3[C@@H]2c2ccc(F)cc2)C1] | 4091 |
| 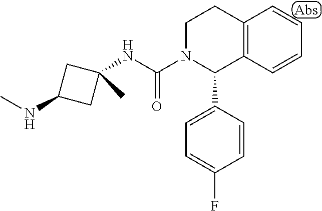 [SMILES: CN[C@H]1C[C@](C)(NC(=O)N2CCc3ccccc3[C@@H]2c2ccc(F)cc2)C1] | 4092 |
| 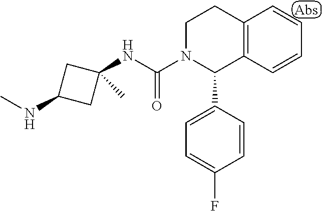 [SMILES: CN[C@H]1C[C@@](C)(NC(=O)N2CCc3ccccc3[C@@H]2c2ccc(F)cc2)C1] | 4093 |
| 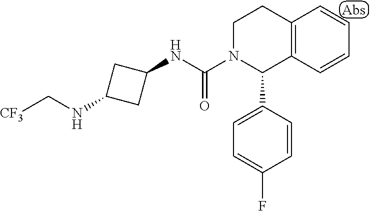 [SMILES: O=C(N[C@H]1C[C@H](NCC(F)(F)F)C1)N1CCc2ccccc2[C@@H]1c1ccc(F)cc1] | 4094 |
| 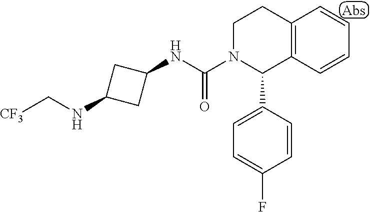 [SMILES: O=C(N[C@H]1C[C@@H](NCC(F)(F)F)C1)N1CCc2ccccc2[C@@H]1c1ccc(F)cc1] | 4095 |
| 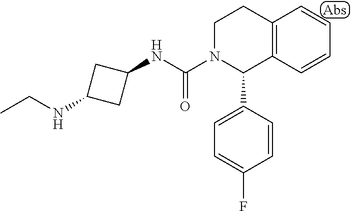 [SMILES: CCN[C@H]1C[C@H](NC(=O)N2CCc3ccccc3[C@@H]2c2ccc(F)cc2)C1] | 4096 |
| 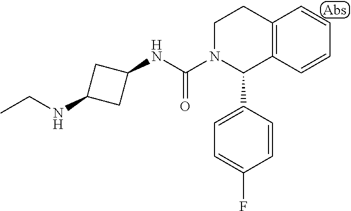 [SMILES: CCN[C@H]1C[C@@H](NC(=O)N2CCc3ccccc3[C@@H]2c2ccc(F)cc2)C1] | 4097 |
| 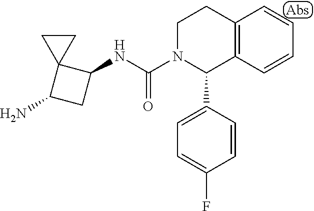 [SMILES: N[C@H]1C[C@H](NC(=O)N2CCc3ccccc3[C@@H]2c2ccc(F)cc2)C12CC2] | 4098 |
| 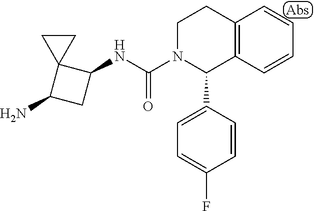 [SMILES: N[C@@H]1C[C@H](NC(=O)N2CCc3ccccc3[C@@H]2c2ccc(F)cc2)C12CC2] | 4099 |
| 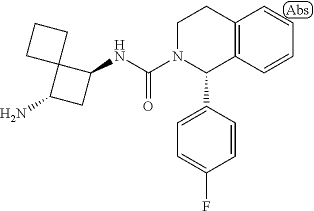 [SMILES: N[C@H]1C[C@H](NC(=O)N2CCc3ccccc3[C@@H]2c2ccc(F)cc2)C12CCC2] | 4100 |
| 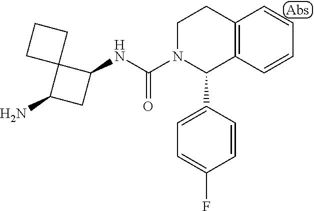 [SMILES: N[C@@H]1C[C@H](NC(=O)N2CCc3ccccc3[C@@H]2c2ccc(F)cc2)C12CCC2] | 4101 |
| 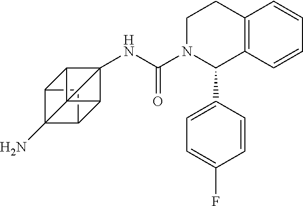 [SMILES: NC12C3CC1C1C4(NC(=O)N5CCc6ccccc6[C@@H]5c5ccc(F)cc5)C3C124] | 4102 |
| 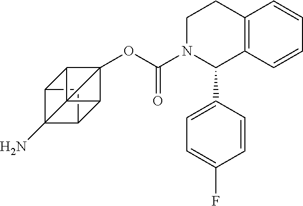 [SMILES: NC12C3CC1C1C4(OC(=O)N5CCc6ccccc6[C@@H]5c5ccc(F)cc5)C3C124] | 4103 |
| 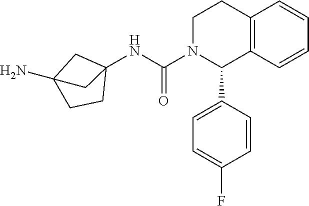 [SMILES: NC12CCC(NC(=O)N3CCc4ccccc4[C@@H]3c3ccc(F)cc3)(C1)C2] | 4104 |
| 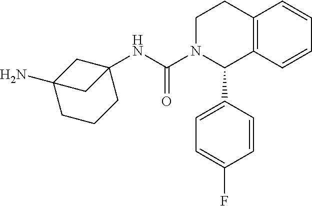 [SMILES: NC12CCCC(NC(=O)N3CCc4ccccc4[C@@H]3c3ccc(F)cc3)(C1)C2] | 4105 |
| 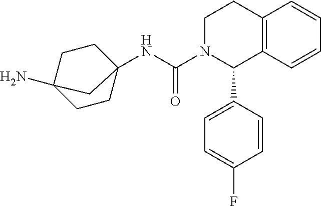 [SMILES: NC12CCC(NC(=O)N3CCc4ccccc4[C@@H]3c3ccc(F)cc3)(CC1)C2] | 4106 |
| 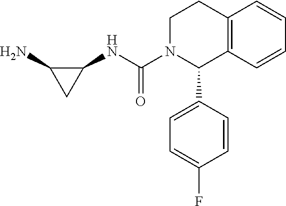 [SMILES: N[C@@H]1C[C@@H]1NC(=O)N1CCc2ccccc2[C@@H]1c1ccc(F)cc1] | 4107 |
| 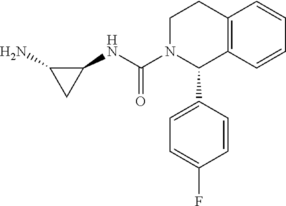 [SMILES: N[C@H]1C[C@@H]1NC(=O)N1CCc2ccccc2[C@@H]1c1ccc(F)cc1] | 4108 |
| 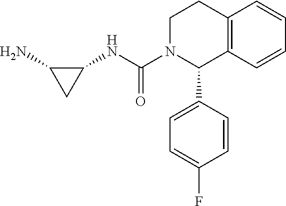 [SMILES: N[C@H]1C[C@H]1NC(=O)N1CCc2ccccc2[C@@H]1c1ccc(F)cc1] | 4109 |
| 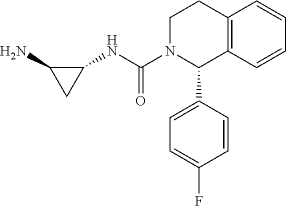 [SMILES: N[C@@H]1C[C@H]1NC(=O)N1CCc2ccccc2[C@@H]1c1ccc(F)cc1] | 4110 |
| 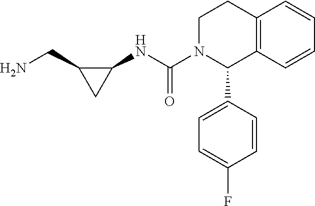 [SMILES: NC[C@@H]1C[C@@H]1NC(=O)N1CCc2ccccc2[C@@H]1c1ccc(F)cc1] | 4111 |
| 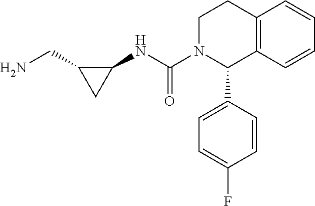 [SMILES: NC[C@H]1C[C@@H]1NC(=O)N1CCc2ccccc2[C@@H]1c1ccc(F)cc1] | 4112 |
| 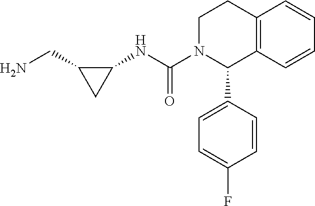 [SMILES: NC[C@H]1C[C@H]1NC(=O)N1CCc2ccccc2[C@@H]1c1ccc(F)cc1] | 4113 |
| 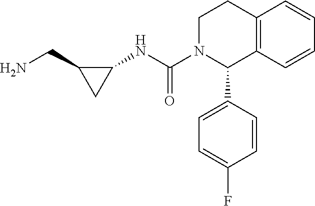 [SMILES: NC[C@@H]1C[C@H]1NC(=O)N1CCc2ccccc2[C@@H]1c1ccc(F)cc1] | 4114 |
| 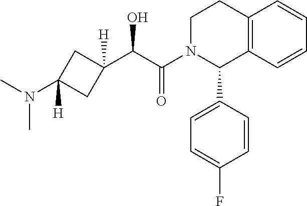 [SMILES: CN(C)[C@H]1C[C@H]([C@@H](O)C(=O)N2CCc3ccccc3[C@@H]2c2ccc(F)cc2)C1] | 4115 |
| 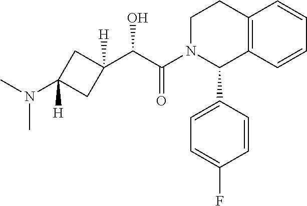 [SMILES: CN(C)[C@H]1C[C@H]([C@H](O)C(=O)N2CCc3ccccc3[C@@H]2c2ccc(F)cc2)C1] | 4116 |
| 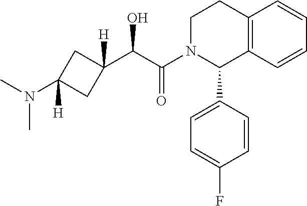 [SMILES: CN(C)[C@H]1C[C@@H]([C@@H](O)C(=O)N2CCc3ccccc3[C@@H]2c2ccc(F)cc2)C1] | 4117 |
| 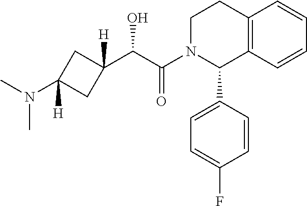 [SMILES: CN(C)[C@H]1C[C@@H]([C@H](O)C(=O)N2CCc3ccccc3[C@@H]2c2ccc(F)cc2)C1] | 4118 |
| 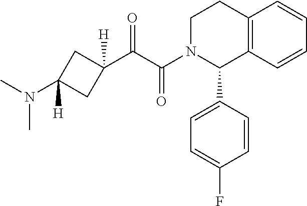 [SMILES: CN(C)[C@H]1C[C@H](C(=O)C(=O)N2CCc3ccccc3[C@@H]2c2ccc(F)cc2)C1] | 4119 |
| 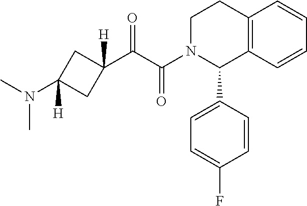 [SMILES: CN(C)[C@H]1C[C@@H](C(=O)C(=O)N2CCc3ccccc3[C@@H]2c2ccc(F)cc2)C1] | 4120 |
| 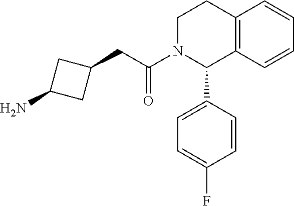 [SMILES: N[C@H]1C[C@@H](CC(=O)N2CCc3ccccc3[C@@H]2c2ccc(F)cc2)C1] | 4121 |
| 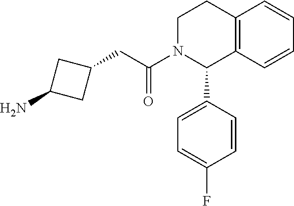 [SMILES: N[C@H]1C[C@H](CC(=O)N2CCc3ccccc3[C@@H]2c2ccc(F)cc2)C1] | 4122 |
| 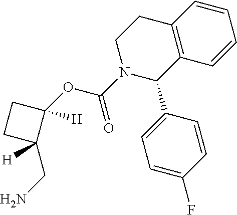 [SMILES: NC[C@@H]1CC[C@H]1OC(=O)N1CCc2ccccc2[C@@H]1c1ccc(F)cc1] | 4123 |
| 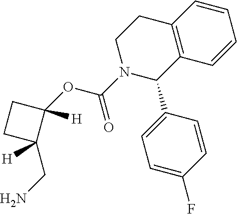 [SMILES: NC[C@@H]1CC[C@@H]1OC(=O)N1CCc2ccccc2[C@@H]1c1ccc(F)cc1] | 4124 |
| 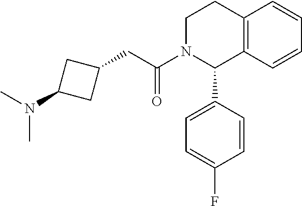 [SMILES: CN(C)[C@H]1C[C@H](CC(=O)N2CCc3ccccc3[C@@H]2c2ccc(F)cc2)C1] | 4125 |
| 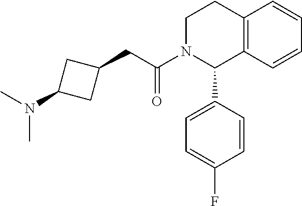 [SMILES: CN(C)[C@H]1C[C@@H](CC(=O)N2CCc3ccccc3[C@@H]2c2ccc(F)cc2)C1] | 4126 |
| 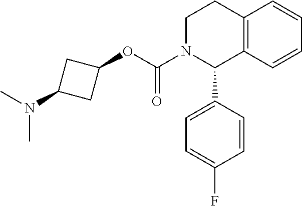 [SMILES: CN(C)[C@H]1C[C@@H](OC(=O)N2CCc3ccccc3[C@@H]2c2ccc(F)cc2)C1] | 4127 |
| 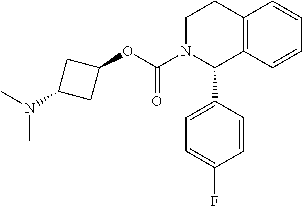 [SMILES: CN(C)[C@H]1C[C@H](OC(=O)N2CCc3ccccc3[C@@H]2c2ccc(F)cc2)C1] | 4128 |
| 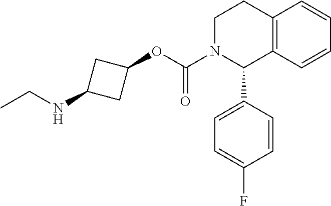 [SMILES: CCN[C@H]1C[C@@H](OC(=O)N2CCc3ccccc3[C@@H]2c2ccc(F)cc2)C1] | 4129 |
| 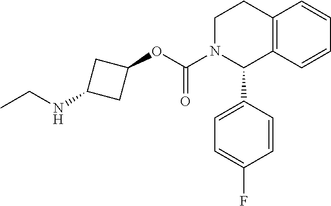 [SMILES: CCN[C@H]1C[C@H](OC(=O)N2CCc3ccccc3[C@@H]2c2ccc(F)cc2)C1] | 4130 |
| 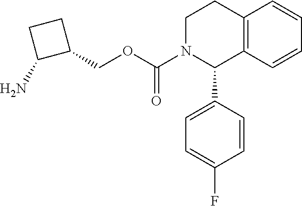 [SMILES: N[C@@H]1CC[C@@H]1COC(=O)N1CCc2ccccc2[C@@H]1c1ccc(F)cc1] | 4132 |
| 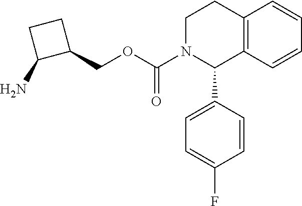 [SMILES: N[C@H]1CC[C@H]1COC(=O)N1CCc2ccccc2[C@@H]1c1ccc(F)cc1] | 4133 |
| 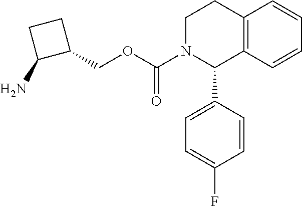 [SMILES: N[C@H]1CC[C@@H]1COC(=O)N1CCc2ccccc2[C@@H]1c1ccc(F)cc1] | 4134 |
| 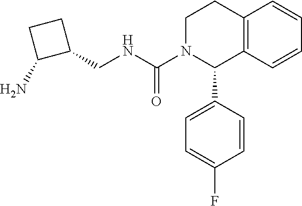 [SMILES: N[C@@H]1CC[C@@H]1CNC(=O)N1CCc2ccccc2[C@@H]1c1ccc(F)cc1] | 4131 |
| 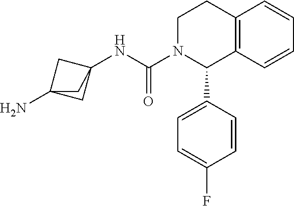 [SMILES: NC12CC(NC(=O)N3CCc4ccccc4[C@@H]3c3ccc(F)cc3)(C1)C2] | 4135 |
| 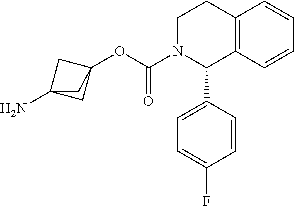 [SMILES: NC12CC(OC(=O)N3CCc4ccccc4[C@@H]3c3ccc(F)cc3)(C1)C2] | 4136 |
| 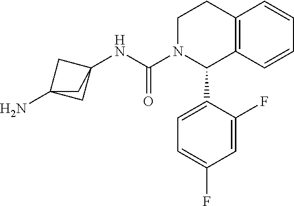 [SMILES: NC12CC(NC(=O)N3CCc4ccccc4[C@@H]3c3ccc(F)cc3F)(C1)C2] | 4137 |
| 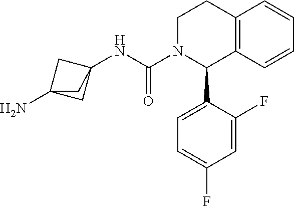 [SMILES: NC12CC(NC(=O)N3CCc4ccccc4[C@H]3c3ccc(F)cc3F)(C1)C2] | 4138 |
| 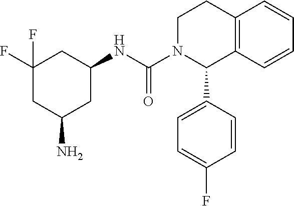 [SMILES: N[C@@H]1C[C@H](NC(=O)N2CCc3ccccc3[C@@H]2c2ccc(F)cc2)CC(F)(F)C1] | 4141 |
| 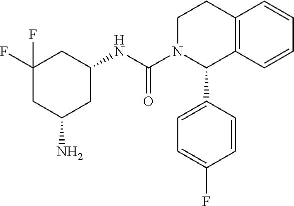 [SMILES: N[C@H]1C[C@@H](NC(=O)N2CCc3ccccc3[C@@H]2c2ccc(F)cc2)CC(F)(F)C1] | 4142 |
| 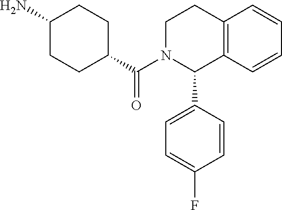 [SMILES: N[C@H]1CC[C@@H](C(=O)N2CCc3ccccc3[C@@H]2c2ccc(F)cc2)CC1] | 4143 |
| 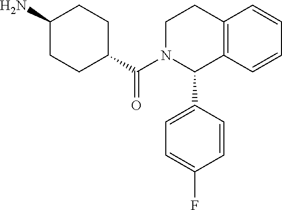 [SMILES: N[C@H]1CC[C@H](C(=O)N2CCc3ccccc3[C@@H]2c2ccc(F)cc2)CC1] | 4144 |
| 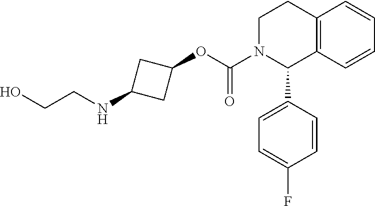 [SMILES: O=C(O[C@H]1C[C@@H](NCCO)C1)N1CCc2ccccc2[C@@H]1c1ccc(F)cc1] | 4145 |
| 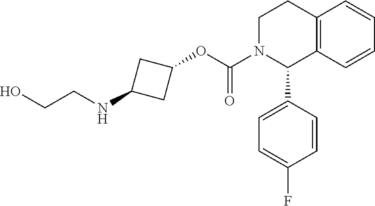 [SMILES: O=C(O[C@H]1C[C@H](NCCO)C1)N1CCc2ccccc2[C@@H]1c1ccc(F)cc1] | 4146 |
| 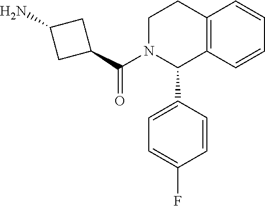 [SMILES: N[C@H]1C[C@H](C(=O)N2CCc3ccccc3[C@@H]2c2ccc(F)cc2)C1] | 4147 |
| 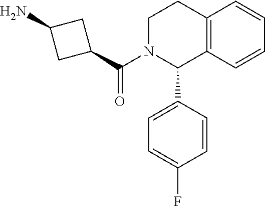 [SMILES: N[C@H]1C[C@@H](C(=O)N2CCc3ccccc3[C@@H]2c2ccc(F)cc2)C1] | 4148 |
| 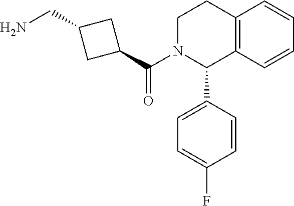 [SMILES: NC[C@H]1C[C@H](C(=O)N2CCc3ccccc3[C@@H]2c2ccc(F)cc2)C1] | 4149 |
| 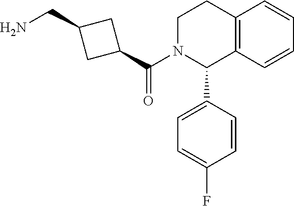 [SMILES: NC[C@H]1C[C@@H](C(=O)N2CCc3ccccc3[C@@H]2c2ccc(F)cc2)C1] | 4150 |
| 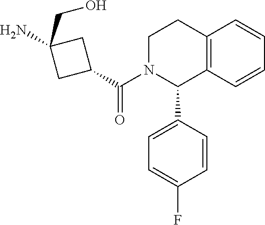 [SMILES: N[C@]1(CO)C[C@H](C(=O)N2CCc3ccccc3[C@@H]2c2ccc(F)cc2)C1] | 4151 |
| 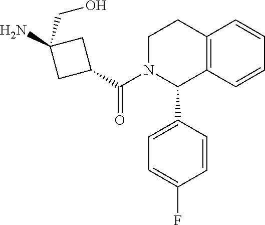 [SMILES: N[C@]1(CO)C[C@@H](C(=O)N2CCc3ccccc3[C@@H]2c2ccc(F)cc2)C1] | 4152 |
| 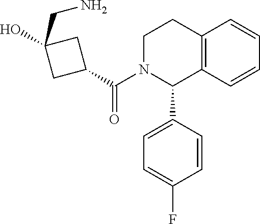 [SMILES: NC[C@]1(O)C[C@@H](C(=O)N2CCc3ccccc3[C@@H]2c2ccc(F)cc2)C1] | 4153 |
| 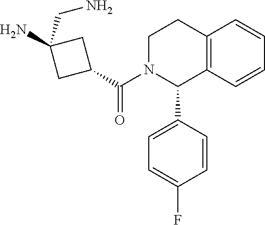 [SMILES: NC[C@]1(N)C[C@H](C(=O)N2CCc3ccccc3[C@@H]2c2ccc(F)cc2)C1] | 4154 |
| | 4155 |

As used herein, the term “alkyl” refers to straight chained and branched saturated hydrocarbon groups containing one to six carbon atoms. The term Cₙ means the alkyl group has “n” carbon atoms. For example, C alkyl refers to an alkyl group that has 6 carbon atoms. C₁-C₆ alkyl refers to an alkyl group having a number of carbon atoms encompassing the entire range (e.g., 1 to 6 carbon atoms), as well as all subgroups (e.g., 1-6, 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), t-butyl (1,1-dimethylethyl), and 3-methylpentyl. Unless otherwise indicated, an alkyl group can be an unsubstituted alkyl group or a substituted alkyl group.

The term “alkylene” used herein refers to an alkyl group having a substituent. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. A C₀ alkylene indicates a direct bond. Unless otherwise indicated, an alkylene group can be an unsubstituted alkylene group or a substituted alkylene group. Particular substitutions on the alkylene group can be specified, e.g., alkylene-CN, or the like.

The term “alkyne” or “alkynyl” used herein refers to an unsaturated aliphatic group analogous in length and possible substitution to an alkyl group described above, but that contains at least one triple bond. For example, the term “alkynyl” includes straight chain alkynyl groups (e.g., ethynyl, propynyl, butynyl, pentynyl, hexynyl), and branched alkynyl groups. For example, a straight chain or branched alkynyl group can have six or fewer carbon atoms in its backbone (e.g., C₂-C₆ for straight chain, C₃-C₆ for branched chain). The term “C₂-C₆” includes chains having a number of carbon atoms encompassing the entire range (e.g., 2 to 6 carbon atoms), as well as all subgroups (e.g., 2-6, 2-5, 2-4, 3-6, 2, 3, 4, 5, and 6 carbon atoms). The term “C₃-C₆” includes chains having a number of carbon atoms encompassing the entire range (e.g., 3 to 6 carbon atoms), as well as all subgroups (e.g., 3-6, 3-5, 3-4, 3, 4, 5, and 6 carbon atoms). Unless otherwise indicated, an alkynyl group can be an unsubstituted alkynyl group or a substituted alkynyl group.

As used herein, the term “haloalkyl” refers to an alkyl group substituted with one or more halogen substituents. For example, C₁-C₆haloalkyl refers to a C₁-C₆ alkyl group substituted with one or more halogen atoms, e.g., 1, 2, 3, 4, 5, or 6 halogen atoms. A C₀haloalkyl refers simply to a halo substituent. Non-limiting examples of haloalkyl groups include fluoromethyl, difluoromethyl, trifluoromethyl, chloromethyl, dichloromethyl, and trichloromethyl groups.

As used herein, the term “halo” or “halogen” refers to fluorine, chlorine, bromine, or iodine.

As used herein, the term “hydroxyalkyl” refers to an alkyl group substituted with one or more hydroxyl substituents. For example, C₁-C₃hydroxyalkyl refers to a C₁-C₆ alkyl group substituted with one or more hydroxyl groups, e.g., 1, 2, 3, 4, 5, or 6 hydroxyl groups. A C₀hydroxyalkyl refers to a hydroxyl group. Non-limiting examples of hydroxyalkyl groups include hydroxymethyl and 2-hydroxyethyl groups.

As used herein, the term “oxo” refers to ═O substituent, e.g., a carbon can be substituted with an oxo to form a carbonyl (C═O) group.

As used herein, the term “carbocycle” or “carbocyclyl” refers to a cyclic hydrocarbon group containing three to eight carbon atoms (e.g., 3, 4, 5, 6, 7, or 8 carbon atoms). The term “n-membered carbocycle” means the carbocycle group has “n” carbon atoms. For example, 5-membered carbocycle refers to a carbocycle group that has 5 carbon atoms in the ring. 6- to 8-membered carbocycle refers to carbocycle groups having a number of carbon atoms encompassing the entire range (e.g., 6 to 8 carbon atoms), as well as all subgroups (e.g., 6-7, 6-8, 7-8, 6, 7, and 8 carbon atoms). Nonlimiting examples of carbocycle groups include cyclopropyl, cyclobutyl, cyclopentyl, cyclohexyl, cycloheptyl, and cyclooctyl. Unless otherwise indicated, a carbocycle group can be an unsubstituted carbocycle group or a substituted carbocycle group. The carbocycle groups described herein can be isolated or fused to another carbocycle group. In particular, the carbocycles described herein can have a fused, bridged, or spiro structure. When a carbocycle group is fused to another carbocycle group, then each of the carbocycle groups can contain three to eight carbon atoms unless specified otherwise.

As used herein, the term “heterocycle” is defined similarly as carbocycle, except the ring contains one to four heteroatoms independently selected from oxygen, nitrogen, and sulfur. In particular, the term “heterocycle” refers to a ring containing a total of three to eight atoms (e.g., three to five, or five to eight), of which 1, 2, 3 or 4 of those atoms are heteroatoms independently selected from the group consisting of oxygen, nitrogen, and sulfur, and the remaining atoms in the ring are carbon atoms. Nonlimiting examples of heterocycle groups include azetidine, piperdine, piperazine, pyrazolidine, tetrahydrofuran, tetrahydropyran, dihydrofuran, morpholine, quinuclidine, and the like. Heterocycle groups can be saturated or partially unsaturated ring systems optionally substituted with, for example, one to three groups, such as halo, C₁₋₆alkyl, C₁₋₆haloalkyl, C₁₋₆alkoxy, C₁₋₆haloalkoxy, CN, and SO₂C₁₋₃alkyl. The heterocycle groups described herein can be isolated or fused to another heterocycle group and/or a carbocycle group. When a heterocycle group is fused to another heterocycle group, then each of the heterocycle groups can contain three to ten total ring atoms, and one to four heteroatoms.

As used herein, the term “substituted,” when used to modify a chemical functional group, refers to the replacement of at least one hydrogen radical on the functional group with a substituent. Unless otherwise specified for a particular moiety, substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

Compounds of the present disclosure can exist in particular geometric or stereoisomeric forms having one or more asymmetric carbon atoms. The present disclosure contemplates such forms, including cis- and trans-isomers, R- and S-enantiomers, diastereomers, racemic mixtures thereof, and other mixtures thereof, as falling within the scope of the disclosed compounds. Additional asymmetric carbon atoms may be present in substituents such as alkyl groups. All such isomers, as well as mixtures thereof, are intended for inclusion herein.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

The compounds disclosed herein can be as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N*(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Pharmaceutical Formulations, Dosing, and Routes of Administration

Further provided are pharmaceutical formulations comprising a compound as described herein or pharmaceutically acceptable salt thereof, and a pharmaceutically acceptable excipient.

The compounds described herein can be administered to a subject in a therapeutically effective amount, alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to affect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, for example, from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by physico-chemical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods of the invention may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, Pa., pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus, the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds disclosed herein (e.g., compounds of Formula I and as shown in Table A) can increase the amount of progranulin or granulin in a subject. In some cases, the compounds increase the amount of progranulin in a subject. In some cases, the compounds increase the amount of granulin in a subject. In some cases, the compounds affect cells to increase secretion of progranulin. As such, the compounds disclosed herein, ((e.g., compounds of Formula I and as shown in Table A) can be useful in treating disorders associated with aberrant (e.g., reduced) progranulin secretion or activity.

Specifically contemplated are methods of using a therapeutically effective amount of a compound disclosed herein to modulate progranulin (e.g., to increase secretion of progranulin), for use as a therapeutic in a subject. As used herein, the term “therapeutically effective amount” means an amount of a compound or combination of therapeutically active compounds (e.g., a progranulin modulator or combination of modulators) that ameliorates, attenuates or eliminates one or more symptoms of a particular disease or condition (e.g., progranulin- or granulin-associated disorders), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

Contemplated disorders associated with aberrant progranulin activity include Alzheimer's disease (AD), Parkinson's disease (PD) and PD-related disorders, Amytrophic lateral sclerosis (ALS), Frontotemperal lobe dementia (FTLD), Lewy body dementia (LBD), Prion disease, Motor neurone diseases (MND), Huntington's disease (HD), Spinocerebellar ataxia (SCA), Spinal muscular atrophy (SMA) and other neurodegenerative diseases. Other disorders contemplated include lysosomal dys- or misfunction disorders, such lysosomal storage diseases (e.g., Paget's disease, Gaucher's disease, Nieman's Pick disease, Tay-Sachs Disease, Fabry Disease, Pompes disease, and Naso-Hakula disease). Other diseases contemplated include those associated with inclusions and/or misfunction of C9orf72, TDP-43, FUS, UBQLN2, VCP, CHMP28, and/or MAPT. Other diseases include acute neurological disorders such as stroke, cerebral hemorrhage, traumatic brain injury and other head traumas as well as diseases of the brain such as glioblastoma and neuroblastomas.

In some cases, the progranulin-associated disorder is Alzheimer's disease (AD), Parkinson's disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington's disease (HD), Spinocerebellar ataxia (SCA), Spinal muscular atrophy (SMA), a lysosomal storage disease, nephropathy, a disease associated with inclusions and/or misfunction of C9orf72, TDP-43, FUS, UBQLN2, VCP, CHMP28, and/or MAPT, an acute neurological disorder, glioblastoma, or neuroblastoma. In some cases, the lysosomal storage disease is Paget's disease, Gaucher's disease, Nieman's Pick disease, Tay-Sachs Disease, Fabry Disease, Pompes disease, or Naso-Hakula disease. In some cases, the acute neurological disorder is stroke, cerebral hemorrhage, traumatic brain injury or head trauma. In some cases, the progranulin-associated disorder is Frontotemporal dementia (FTD). In some cases, the progranulin-associated disorder is Frontotemporal dementia-Granulin subtype (FTD-GRN).

### Synthesis of Compounds Disclosed Herein

Compounds can be synthesized using typical synthetic chemistry techniques using commercially available starting materials, compounds known in the literature, or from readily prepared intermediates, by employing standard synthetic methods and procedures either known to those of skill in the art, or in light of the teachings herein. Generally, the synthesis of the disclosed compounds can be achieved following similar syntheses as detailed in Schemes A and B below and in the Examples.

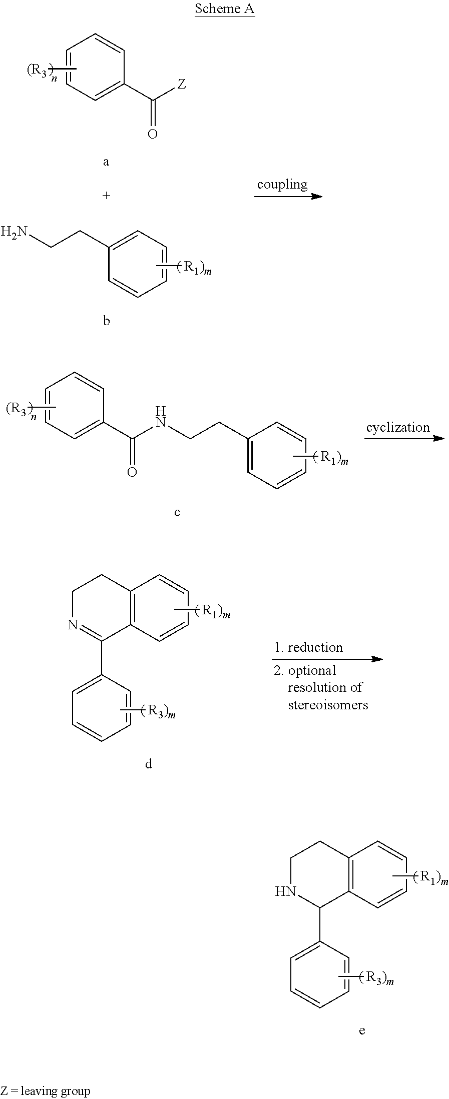

Compounds having structure e can be synthesized using the procedure shown in Scheme A. For example, reaction of an optionally substituted benzoic acid derivative having structure a with an optionally substituted 2-phenylethan-1-amine b produces optionally substituted N-phenethylbenzamide compounds having structure c. Cyclization under appropriate conditions gives optionally substituted 1-phenyl-3,4-dihydroisoquinoline compounds having structure d. Subsequent reduction followed by optional isolation of single stereoisomers by resolution or chromatographic means gives substituted tetrahydroquinoline compounds having structure e.

The coupling of compounds a and b can be catalyzed by appropriate reagents selected based on the precise nature of compounds a and b. For example, when compound a is an acid chloride compound (i.e., when Z is Cl), the coupling of compounds a and b can be catalyzed by e.g., triethylamine. Compounds a and b can be purchased commercially or prepared by a variety of methods from commercially-available starting materials.

Cyclization of compound c can be effected with the use of various reactions known in the art. For example, the cyclization can involve an acid-catalyzed electrophilic aromatic substitution reaction, e.g., cyclization under Bischler-Napieralski reaction conditions. For example, c can be cyclized by treatment with triflic anhydride in the presence of e.g., chloropyridine in a solvent, e.g., dichloromethane. Alternately, compound c can be cyclized by treatment with polyphosphoric acid (PPA).

Compound d can be reduced to form compound e with or without asymmetric induction of a stereocenter. For example, compound d can be treated with a reducing agent, e.g., sodium borohydride, in a solvent, e.g., methanol. Reduction of compound d can be followed by the formation of a desired stereoisomer, e.g., by crystallization in the presence of D-tartaric acid. Alternately, compound d can be reduced via asymmetric hydrogenation to directly produce substituted tetrahydroquinoline compound e as the desired stereoisomer. For example, compound d can be reduced with H₂ gas in the presence of an iridium catalyst, such as [{Ir(H)[(S,S)-(f)-binaphane]}₂(μ-I)₃]⁺|⁻.

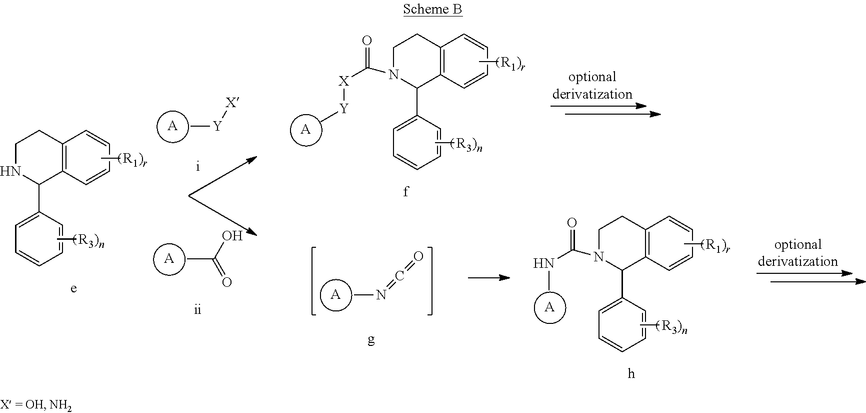

Compounds described herein, e.g., compounds of Formula I, can be synthesized from compounds of structure e using the procedures shown in Scheme B. For example, reaction of a compound having structure e with an alcohol or amine compound having structure i produces a substituted tetrahydroquinolinyl compound having structure f. The coupling can be facilitated by an appropriate reagent, such as bis-(p-nitrophenyl carbonate). Subsequent optional derivatization gives compounds as disclosed herein, e.g., compounds of Formula (I).

Alternately, reaction of a compound having structure e with a carboxylic acid compound having structure ii produces a substituted tetrahydroquinolinyl urea compound having structure h. Carboxylic acid compound ii is reacted in situ with an azide, e.g., diphenyl phosphoryl azide (DPPA) under heating. The compound can undergo e.g., a Curtius rearrangement, to form a reactive isocyanate intermediate g in situ. The isocyanate g reacts with the THIQ amine to form substituted tetrahydroquinolinyl urea h, which can be optionally further derivatized to give compounds as disclosed herein, e.g., compounds of Formula (I).

### EXAMPLES

### General Methods

### Analytical Methods:

LCMS: Apparatus: Agilent 1260 Bin. Pump: G1312B, degasser; autosampler, ColCom, DAD: Agilent G1315D, 220-320 nm, MSD: Agilent LC/MSD G6130B ESI, pos/neg 100-1000, ELSD Alltech 3300 gas flow 1.5 ml/min, gas temp: 40° C., Eluent A: 0.1% formic acid in acetonitrile, Eluent B: 0.1% formic acid in water).

Method A: column: Waters XSelect™ 018, 30×2.1 mm, 3.5μ, Temp: 35° C., Flow: 1 mL/min, Gradient: t₀=5% A, t_{1.6 min}=98% A, t_{3 min}=98% A, Posttime: 1.3 min.

Method C: column: Waters XSelect™ C18, 50×2.1 mm, 3.5μ, Temp: 35° C., Flow: 0.8 mL/min, Gradient: t₀=5% A, t_{3.5 min}=98% A, t_{6 min}=98% A, Posttime: 2 min.

LCMS: Apparatus: Agilent 1260 Bin. Pump: G1312B, degasser; autosampler, ColCom, DAD: Agilent G1315C, 220-320 nm, MSD: Agilent LC/MSD G6130B ESI, pos/neg 100-1000, Eluent A: acetonitrile, Eluent B: 10 mM ammoniumbicarbonate in water (pH=9.5).

Method B: column: Waters XSelect™ CSH C18, 30×2.1 mm, 3.5μ, Temp: 25° C., Flow: 1 mL/min, Gradient: t₀=5% A, t_{1.6 min}=98% A, t_{3 min}=98% A, Posttime: 1.3 min.

Method D: column: Waters XSelect™ CSH C18, 50×2.1 mm, 3.5μ, Temp: 25° C., Flow: 0.8 mL/min, Gradient: t₀=5% A, t_{3.5 min}=98% A, t_{6 min}=98% A, Posttime: 2 min.

LCMS: Apparatus: Agilent 1290 series with UV detector (220 nm, 270 nm (band width 100 nm)), and HP 6130 MSD mass detector (API-ES positive and negative).

Method E: column: Waters XBridge BEH XP (2.1×50 mm; 2.5 μm; 1034 bar), Temp: 35° C., Flow: 0.6 mL/min, t₀=80% A, t_{1.5 min}=0% A, t_{3 min}=0% A. Eluent A: 100% water, Eluent B: 100% methanol/acetonitrile 1:1.

Method O: column: Waters XBridge BEH XP (2.1×50 mm; 2.5 μm; 1034 bar), Temp: 35° C., Flow: 0.6 mL/min, t₀=100% A, t_{1.5 min}=50% A, t_{2 min}=20% A. Eluent A: 0.05% trifluoroacetic acid in water, Eluent B: 100% acetonitrile.

Method K: column: Waters XBridge BEH XP (2.1×50 mm; 2.5 μm; 1034 bar), Temp: 35° C., Flow: 0.6 mL/min, t₀=80% A, t_{1.5 min}=0% A, t_{4 min}=0% A. Eluent A: ammonium acetate (10 mM); water/methanol/acetonitrile (90:6:4), Eluent B: ammonium acetate (10 mM); water/methanol/acetonitrile (10:54:36).

LCMS: Apparatus: Agilent Infinty II; Bin. Pump: G7120A, Multisampler, VTC, DAD: Agilent G7117B, 220 and 220-320 nm, PDA: 210-320 nm, MSD: Agilent G6135B ESI, pos/neg 100-1000, ELSD G7102A: Evap 40° C., Neb 40° C., gasflow 1.6 ml/min.

Method P: column: Waters XSelect CSH C18, 50×2.1 mm, 2.5 μm, Temp: 40° C., Flow: 0.6 mL/min, Gradient: t₀=5% A, t_{2 min}=98% A, t_{2.7 min}=98% A, Posttime: 0.3 min, Eluent A: 0.1% formic acid in acetonitrile, Eluent B: 0.1% formic acid in water.

LCMS: Apparatus: Waters Acquity UPLC H-Class with PDA detector and SQD mass detector (API-ES positive and negative.

Method R: column: Waters XBridge BEH C18 (2.1×50 mm; 2.5 μm), Temp: 30° C., Flow: 0.6 mL/min, t₀=80% A, t_{1.5 min}=5% A, t_{2.5 min}=5% A, Posttime: 0.5 min, Eluent A: 10 mM ammonium acetate in water with 5% acetonitrile, Eluent B: acetonitrile.

Chiral LC:

Apparatus: Agilent 1260 Quat. Pump: G1311C, degasser; autosampler, ColCom, DAD: Agilent G1315D (210 nm, 220 nm, 220-320 nm).

Method AB: column: Chiralpak AD-H (250×4.6 mm, 5 μm); Column temp: 25° C., flow: 0.8 mL/min, Isocratic gradient of 0.1% diethylamine in heptane/ethanol 30/70.

Chiral SFC: Apparatus: Waters Acquity UPC²: Waters ACQ-ccBSM Binary Pump; Waters ACQ-CCM Convergence Manager; Waters ACQ-SM Sample Manager—Fixed Loop; Waters ACQ-CM Column Manager-30S; Waters ACQ-PDA Photodiode Array Detector (210-400 nm); Waters ACQ-ISM Make Up Pump, Waters Acquity QDa MS Detector (pos 100-650).

Method G: column: Phenomenex Amylose-1 (100×4.6 mm, 5 μm), Temp: 35° C., BPR: 170 bar, Flow: 2.5 mL/min, Gradient: t₀=5% B, t_{2.5 min}=50% B, t_{10 min}=50% B, Posttime: 0.5 min; Eluent A: CO₂, Eluent B: 20 mM ammonia in methanol

Method W: column: Phenomenex Amylose-1 (100×4.6 mm, 5 μm), Temp: 35° C., BPR: 170 bar, Flow: 2.5 mL/min, Gradient: t₀=5% B, t_{5 min}=50% B, t_{6 min}=50% B, Posttime: 0.5 min; Eluent A: CO₂, Eluent B: 20 mM ammonia in methanol

Method X: column: Phenomenex Amylose-1 (100×4.6 mm, 5 μm), Temp: 35° C., BPR: 170 bar, Flow: 2.5 mL/min, Gradient: t₀=5% B, t_{5 min}=30% B, t_{6 min}=30% B, Posttime: 0.5 min; Eluent A: CO₂, Eluent B: 20 mM ammonia in methanol

Method AA: column: Phenomenex Lux Cellulose 4 (3.0×150 mm, 3 μm); Temp: 40° C., BPR: 138 bar; Flow: 2.5 mL/min, Gradient: t₀=2% B, t_{4 min}=27% B, Posttime: 1 min; Eluent A: CO₂, Eluent B: methanol

### Preparative Methods

Basic reversed phase MPLC: Instrument type: Reveleris™ prep MPLC; Eluent A: 99% acetonitrile+1% 10 mM ammoniumbicarbonate in water (pH=9.0); Eluent B: 10 mM ammoniumbicarbonate in water (pH=9.0). Unless otherwise stated, column: Waters XSelect CSH C18 (145×25 mm, 10p); Flow: 40 mL/min; Column temp: room temperature, was used. Otherwise Column: Phenomenex Gemini C18 (185×25 mm, 10p); Flow: 40 mL/min; Column temp: room temperature.

Acidic reversed phase MPLC: Instrument type: Reveleris™ prep MPLC; Column: Phenomenex LUNA C18(3) (150×25 mm, 10p); Flow: 40 mL/min; Column temp: room temperature; Eluent A: 0.1% (v/v) Formic acid in acetonitrile, Eluent B: 0.1% (v/v) Formic acid in water.

Preparative Chiral SFC: Apparatus: Waters Prep 100 SFC UV/MS directed system; Waters 2998 Photodiode Array (PDA) Detector; Waters Acquity QDa MS detector; Waters 2767 Sample Manager. Eluent A: CO₂, Eluent B: 20 mM ammonia in methanol.

Method Y: Column: Phenomenex Lux Amylose-1 (250×21 mm, 5 μm); Column temp: 35° C.; flow: 70 mL/min; ABPR: 120 bar; Linear gradient: t=0 min 10% B, t=6 min 40% B; t=7.5 min 40% B; Detection: PDA (210-400 nm)/TIC.

Method Z: Column: Phenomenex Lux Amylose-1 (250×21 mm, 5 μm); Column temp: 35° C.; flow: 70 mL/min; ABPR: 120 bar; Linear gradient: t=0 min 10% B, t=6 min 50% B; t=7.5 min 50% B; Detection: PDA (210-400 nm)/TIC.

¹H-NMR:

400 MHz ¹H-NMR spectra were recorded on a BrukerAvance-400 ultrashield NMR spectrometer, using CDCl₃ or DMSO-d6 as solvent and are reported in ppm using TMS (0.00 ppm) as an internal standard.

300 MHz ¹H-NMR spectra were recorded on a Varian VNMRS: 7.05 Tesla magnet from Oxford Instruments. Chemical shifts are denoted in 5 (ppm) and are referenced to the residual protic solvent.

### Example 1. Synthesis of 3-Aminobicyclo[1.1.1]pentan-1-yl (S)-1-(4-fluorophenyl)-3,4-dihydroisoquinoline-2(1H)-carboxylate, Compound 4001

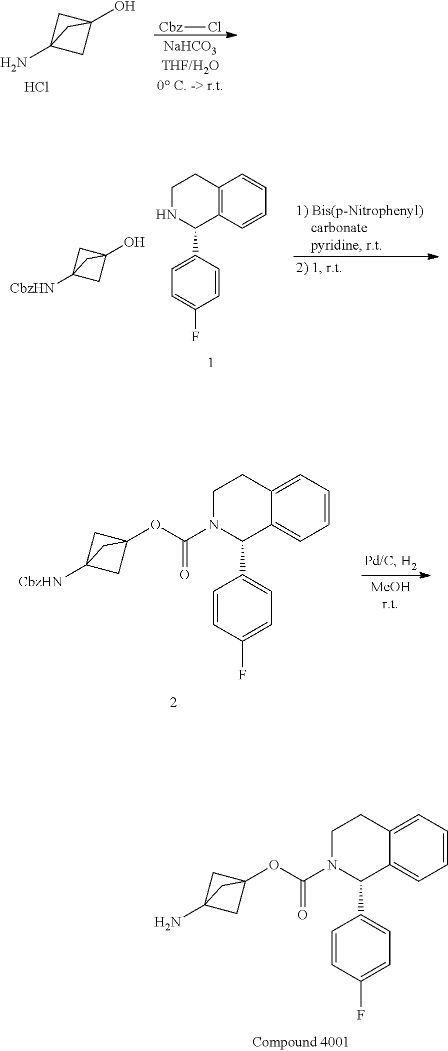

### Step 1. Synthesis of benzyl (3-hydroxybicyclo[1.1.1]pentan-1-yl)carbamate

Sodium bicarbonate (891 mg, 10.60 mmol) was added to a turbid solution of 3-aminobicyclo[1.1.1]pentan-1-ol hydrochloride (494.0 mg, 3.53 mmol) in tetrahydrofuran (5.4 mL) and water (2.7 mL). The reaction mixture was cooled to 0° C. and stirred for 5 minutes before benzyl chloroformate (0.577 mL, 3.92 mmol) was added dropwise. The reaction mixture was stirred for 10 minutes before being allowed to warm to room temperature and being stirred for further 3 hours. Next, the reaction mixture was diluted with ethyl acetate (30 mL) and washed with brine (10 mL). The aqueous layer was extracted with ethyl acetate (15 mL) and the combined organics were dried on Na₂SO₄ and evaporated under reduced pressure. The residue was purified by flash column chromatography (silica, 0 to 7% methanol in dichloromethane) to yield benzyl (3-hydroxybicyclo[1.1.1]pentan-1-yl)carbamate (797 mg) as a white solid. LCMS: 96%, RT=1.66 min., (M+H)⁺=234 (method B). ¹H NMR (400 MHz, chloroform-d) δ 7.40-7.28 (m, 5H), 5.24-4.91 (m, 3H), 2.37 (s, 1H), 2.21 (s, 6H).

### Step 2. Synthesis of 3-(((benzyloxy)carbonyl)amino)bicyclo[1.1.1]pentan-1-yl (S)-1-(4-fluorophenyl)-3,4-dihydroisoquinoline-2(1H)-carboxylate

Bis(p-nitrophenyl) carbonate (1012 mg, 3.29 mmol) was added to a solution of benzyl (3-hydroxybicyclo[1.1.1]pentan-1-yl)carbamate (792 mg, 3.29 mmol) in pyridine (dry, 33 mL). The mixture was stirred under argon atmosphere at room temperature for 17 hours before (S)-1-(4-fluorophenyl)-1,2,3,4-tetrahydroisoquinoline 1 (749 mg, 3.29 mmol) was added. Stirring was continued for another 23 hours after which the reaction mixture was concentrated to dryness under reduced pressure. The residue was purified by flash column chromatography (silica, 1 to 30% ethyl acetate in heptane) and flash column chromatography (silica, 100% chloroform) to give 3-(((benzyloxy)carbonyl)amino)bicyclo[1.1.1]pentan-1-yl (S)-1-(4-fluorophenyl)-3,4-dihydroisoquinoline-2(1H)-carboxylate, 2 (1.17 g) as a white foam. LCMS: 99%, RT=2.29 min., (M+H)⁺=487 (method B). ¹H NMR (400 MHz, chloroform-d) mixture of rotamers δ 7.40-7.29 (m, 5H), 7.24-7.08 (m, 5H), 7.05-6.92 (m, 3H), 6.45-6.13 (m, 1H), 5.18 (br s, 1H), 5.10 (s, 2H), 4.17-3.86 (m, 1H), 3.29-3.08 (m, 1H), 3.07-2.89 (m, 1H), 2.81-2.70 (m, 1H), 2.45 (s, 6H).

### Step 3. Synthesis of 3-aminobicyclo[1.1.1]pentan-1-yl (S)-1-(4-fluorophenyl)-3,4-dihydroisoquinoline-2(1H)-carboxylate, Compound 4001

Palladium (10% on activated carbon, 50% wet, 262 mg, 0.123 mmol) was added to a solution of 3-(((benzyloxy)carbonyl)amino)bicyclo[1.1.1]pentan-1-yl (S)-1-(4-fluorophenyl)-3,4-dihydroisoquinoline-2(1H)-carboxylate, 2 (600.0 mg, 1.233 mmol) in methanol (10 mL). Then, hydrogen gas was bubbled through the reaction mixture at room temperature for 1 hour. Water (100 μL) was added and the reaction mixture was filtered over Celite®. The filtrate was concentrated to dryness under reduced pressure. The residue was purified by flash column chromatography (silica, 0 to 2.5% (7M ammonia in methanol) in chloroform) and basic preparative MPLC (Linear Gradient: t=0 min 15% A, t=1 min 15% A; t=2 min 25% A; t=16 min 65% A; t=17 min 100% A; t=22 min 100% A; detection: 210 nm). The product containing fractions were pooled and lyophilised to yield 3-aminobicyclo[1.1.1]pentan-1-yl (S)-1-(4-fluorophenyl)-3,4-dihydroisoquinoline-2(1H)-carboxylate, compound 4001. LCMS: 99%, RT=2.89 min., (M+H)⁺=353 (method C). ¹H NMR (400 MHz, chloroform-d) mixture of rotamers δ 7.25-7.09 (m, 5H), 7.05-6.90 (m, 3H), 6.46-6.14 (m, 1H), 4.19-3.85 (m, 1H), 3.27-3.07 (m, 1H), 3.07-2.89 (m, 1H), 2.75 (d, J=16.0 Hz, 1H), 2.21 (s, 6H).

### Example 2. (S)—N-(3-aminobicyclo[1.1.1]pentan-1-yl)-1-(4-fluorophenyl)-3,4-dihydroisoquinoline-2(1H)-carboxamide, Compound 4002

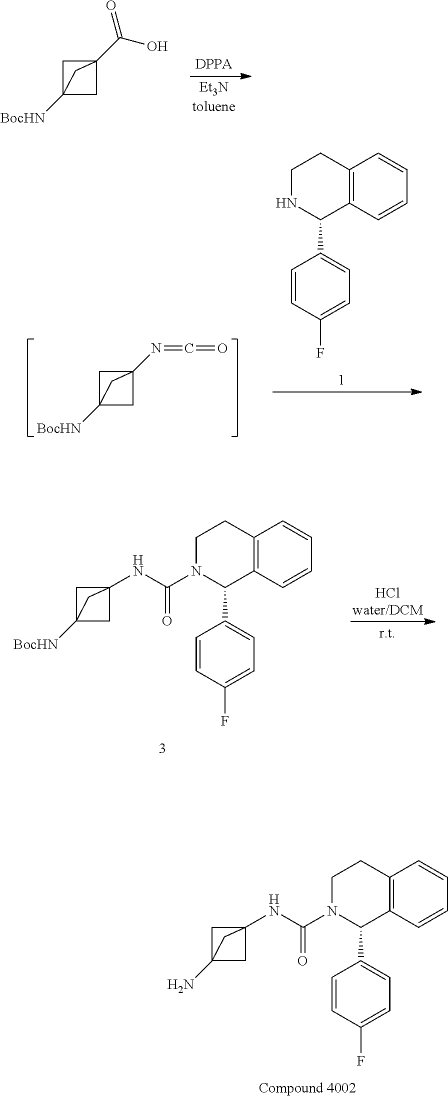

### Step 1. Synthesis of tert-butyl (S)-(3-(1-(4-fluorophenyl)-1,2,3,4-tetrahydroisoquinoline-2-carboxamido)bicyclo[1.1.1]pentan-1-yl)carbamate, 3

In a 2 L three-necked round bottomed flask under a nitrogen atmosphere, a magnetically stirred solution of 3-((tert-butoxycarbonyl)amino)bicyclo[1.1.1]pentane-1-carboxylic acid (50 g, 0.22 mol) and triethylamine (40 mL, 0.28 mol) in toluene (0.5 L) was warmed to 60° C. Diphenyl phosphoryl azide (52 mL, 0.24 mol) was added dropwise over 10 minutes (gas evolution). The solution was warmed to reflux for 2 hours (after which time no more gas evolution took place and a pale yellow solution was obtained). (S)-1-(4-fluorophenyl)-1,2,3,4-tetrahydroisoquinoline (50 g, 0.22 mol) was added in portions over 15 minutes (a small exotherm was observed). The reaction mixture was heated to reflux for 1 hour and then cooled to below 50° C. Water (0.4 L) and ethyl acetate (0.4 L) were added, then the mixture was stirred for 5 minutes and transferred to a 6 L phase separator to separate the layers. The organic layer was washed twice with water (0.2 L and 0.1 L). The combined water layers were extracted with a mixture of toluene and ethyl acetate (1:2, 300 mL). The combined organic layers were washed with water (2×100 mL) and brine (2×100 mL), dried on Na₂SO₄, and evaporated under reduced pressure. The resulting beige solid residue (99 g) was dissolved in refluxing dichloromethane (0.12 L), diluted with pentane (0.36 L), and the resulting suspension was left to cool to room temperature overnight. The formed solid was filtered, washed with pentane (0.1 L), and dried under reduced pressure at 50° C. to give tert-butyl (S)-(3-(1-(4-fluorophenyl)-1,2,3,4-tetrahydroisoquinoline-2-carboxamido) bicyclo[1.1.1]pentan-1-yl)carbamate, 3 (93 g) as a white solid. LCMS: 98%, RT=2.21 min., (M+H)⁺=452 (Method K). Chiral SFC: 99.2%, RT=3.19 min, (M+H)⁺=452 (Method AA). ¹H NMR (300 MHz, chloroform-d) 5 (ppm): 7.27-7.11 (m, 6H), 7.01-6.88 (m, 2H), 6.39 (s, 1H), 5.0 (s, 1H), 4.97 (s, 1H), 3.50 (t, J=6.3 Hz, 2H), 2.98-2.83 (m, 1H), 2.80-2.70 (m, 1H), 2.29 (s, 6H), 1.44 (s, 9H).

### Step 2. Synthesis of (S)—N-(3-aminobicyclo[1.1.1]pentan-1-yl)-1-(4-fluorophenyl)-3,4-dihydroisoquinoline-2(1H)-carboxamide, Compound 4002

In a 1 L round bottomed flask under nitrogen atmosphere, a solution of tert-butyl (S)-(3-(1-(4-fluorophenyl)-1,2,3,4-tetrahydroisoquinoline-2-carboxamido)bicyclo[1.1.1]pentan-1-yl)carbamate, 3, (93 g, 0.20 mol) in dichloromethane (0.5 L) was vigorously stirred and cooled in an ice-water bath to an internal temperature of 5° C. Aqueous HCl (37%, 67 mL, 0.81 mol) was added over a period of 5 minutes. The cooling bath was removed and the mixture was vigorously stirred at room temperature (gas evolution took place). After stirring for 3 hours, additional aqueous HCl (37%, 10 mL, 0.12 mol) was added and the mixture was stirred for another 2 hours. The reaction mixture was cooled in an ice-water bath to an internal temperature of 8° C. and diluted with water (0.5 L). Aqueous NaOH (30%, 100 mL, 1.0 mol) was added over a period of 5 minutes. The temperature of the mixture increased to room temperature and the pH of the mixture was 10 to 11 according to pH indicator paper. The layers were separated. The basic water layer was extracted with dichloromethane (2×100 mL). The combined organic layers were washed with water (2×50 mL), brine (50 mL), dried on Na₂SO₄, and evaporated under reduced pressure to afford (S)—N-(3-aminobicyclo[1.1.1]pentan-1-yl)-1-(4-fluorophenyl)-3,4-dihydroisoquinoline-2(1H) carboxamide, Compound 4002. Three batches of compound 4002, prepared as described above, were combined and dried under reduced pressure to obtain a single batch of Compound 4002. LCMS: 97.2%, RT=1.34 min., (M+H)⁺=352 (Method E). The batch was further purified by gravitational column chromatography (silica (2.5 kg), eluting with 1 to 5% ammonia in methanol (7 M) in ethyl acetate). Two product-containing fractions of 122 g and 15 g, respectively, were collected. According to ¹H NMR analysis, the fraction of 122 g comprised compound 4002 and the fraction of 15 g contained Compound 4002 and acetamide (formed on the column from a reaction between ethyl acetate and ammonia). To remove the acetamide, the 15 g batch was dissolved in ethyl acetate (100 mL) and washed with water (2×50 mL), brine (50 mL), dried on Na₂SO₄, and evaporated under reduced pressure to give 11.3 g of a white solid. This material was combined with the main fraction of 122 g and dried on a rotavap at 10 mbar/70° C. for 4 hours to a constant weight of 127.6 g of white solid. LCMS: 98.2%, RT=1.04 min., (M+H)+=352 (Method P). Chiral SFC: 99.2%, RT=3.50 min, (M+H)⁺=352 (Method G). ¹H NMR (400 MHz, chloroform-d) δ 7.26-7.10 (m, 6H), 6.94 (t, J=8.6 Hz, 2H), 6.39 (s, 1H), 4.96 (s, 1H), 3.49 (td, J=6.1, 1.6 Hz, 2H), 2.89 (dt, J=15.8, 6.5 Hz, 1H), 2.75 (dt, J=15.8, 6.0 Hz, 1H), 2.09 (s, 6H).

### Example 3. (R)—N-(3-aminobicyclo[1.1.1]pentan-1-yl)-1-(4-fluorophenyl)-3,4-dihydroisoquinoline-2(1H)-carboxamide, Compound 4003

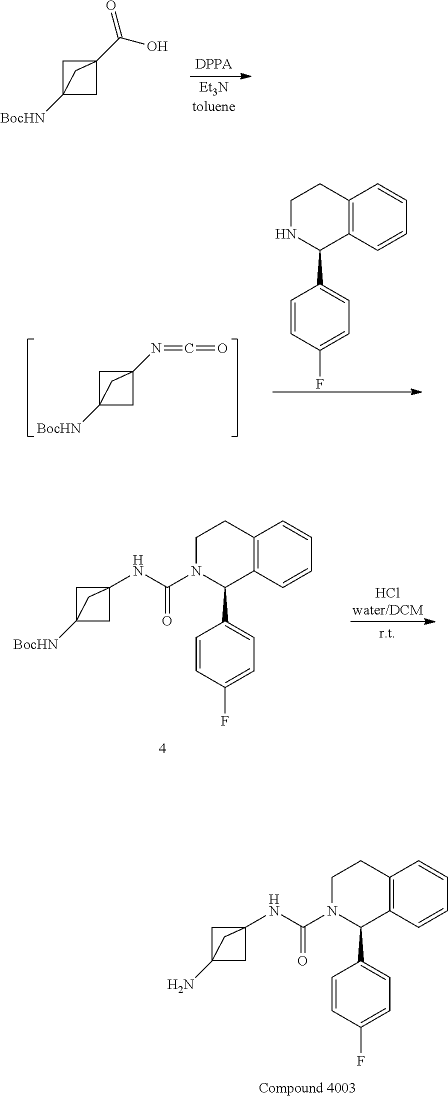

### Step 1. Synthesis of tert-butyl (R)-(3-(1-(4-fluorophenyl)-1,2,3,4-tetrahydroisoquinoline-2-carboxamido)bicyclo[1.1.1]pentan-1-yl)carbamate, 4

Starting from tert-butyl (3-isocyanatobicyclo[1.1.1]pentan-1-yl)carbamate (˜6 mmol) and (R)-1-(4-fluorophenyl)-1,2,3,4-tetrahydroisoquinoline (1.21 g, 5.3 mol), tert-butyl (R)-(3-(1-(4-fluorophenyl)-1,2,3,4-tetrahydroisoquinoline-2-carboxamido)bicyclo[1.1.1]pentan-1-yl)carbamate, intermediate 4 was prepared analogously to the procedure described for tert-butyl (S)-(3-(1-(4-fluorophenyl)-1,2,3,4-tetrahydroisoquinoline-2-carboxamido)bicyclo[1.1.1]pentan-1-yl)carbamate, 3 (see Example 2). White solid (1.9 g). LCMS: 98%, RT=1.34 min., (M+H)⁺=452 (Method R). Chiral SFC: 98.8%, RT=3.45 min, (M+H)⁺=452 (Method AA). ¹H NMR (300 MHz, chloroform-d) δ 7.27-7.12 (m, 6H), 6.96-6.91 (m, 2H), 6.39 (s, 1H), 5.0 (s, 1H), 4.97 (s, 1H), 3.51 (t, J=6.3 Hz, 2H), 2.95-2.85 (m, 1H), 2.80-2.70 (m, 1H), 2.29 (s, 6H), 1.44 (s, 9H).

### Step 2. Synthesis of (R)—N-(3-aminobicyclo[1.1.1]pentan-1-yl)-1-(4-fluorophenyl)-3,4-dihydroisoquinoline-2(1H)-carboxamide, Compound 4003

Starting from tert-butyl (R)-(3-(1-(4-fluorophenyl)-1,2,3,4-tetrahydroisoquinoline-2-carboxamido)bicyclo[1.1.1]pentan-1-yl)carbamate, 4 (1.0 g, 2.2 mmol), (R)—N-(3-aminobicyclo[1.1.1]pentan-1-yl)-1-(4-fluorophenyl)-3,4-dihydroisoquinoline-2(1H) carboxamide, compound 4003 (0.72 g) was prepared according to the procedure described for (S)—N-(3-aminobicyclo[1.1.1]pentan-1-yl)-1-(4-fluorophenyl)-3,4-dihydroisoquinoline-2(1H) carboxamide, compound 4002. White solid foam. LCMS: 98.9%, RT=1.04 min., (M+H)+=352 (Method P). Chiral LC: 98.1%, RT=7.88 min (Method AB). ¹H NMR (400 MHz, chloroform-d) δ 7.26-7.13 (m, 6H), 6.94 (t, J=8.7 Hz, 2H), 6.39 (s, 1H), 4.92 (s, 1H), 3.49 (t, J=6.3 Hz, 2H), 2.90 (dt, J=15.9, 6.5 Hz, 1H), 2.76 (dt, J=15.6, 5.9 Hz, 1H), 2.09 (s, 6H).

### Example 4. (S)-1-(4-fluorophenyl)-N-(3-(methylamino)bicyclo[1.1.1]pentan-1-yl)-3,4-dihydroisoquinoline-2(1H)-carboxamide, Compound 4004

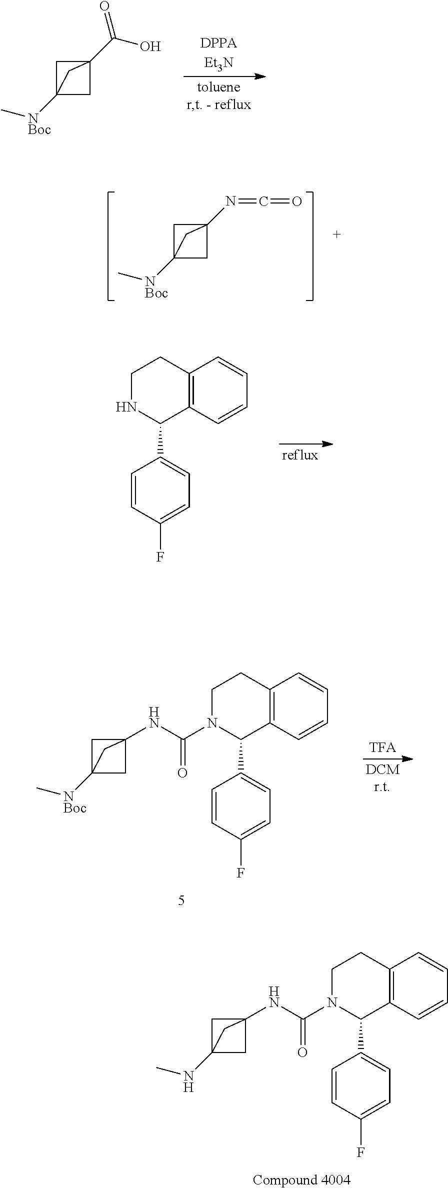

### Step 1. Synthesis of tert-butyl (S)-(3-(1-(4-fluorophenyl)-1,2,3,4-tetrahydroisoquinoline-2-carboxamido)bicyclo[1.1.1]-pentan-1-yl)(methyl)carbamate, 5

Starting from 3-((tert-butoxycarbonyl)(methyl)amino)bicyclo[1.1.1]pentane-1-carboxylic acid (192 mg, 0.796 mmol) and (S)-1-(4-fluorophenyl)-1,2,3,4-tetrahydroisoquinoline (181 mg, 0.796 mg), tert-butyl (S)-(3-(1-(4-fluorophenyl)-1,2,3,4-tetrahydroisoquinoline-2-carboxamido)bicyclo[1.1.1]pentan-1-yl)(methyl)carbamate, 5 (275 mg) was prepared according to the procedure described for was prepared according to the procedure described for tert-butyl (S)-(3-(1-(4-fluorophenyl)-1,2,3,4-tetrahydroisoquinoline-2-carboxamido)bicyclo[1.1.1]pentan-1-yl)carbamate, 3 (see Example 2). White solid. LCMS: 100%, RT=2.35 min., (M+H)⁺=466 (method K). ¹H NMR (300 MHz, chloroform-d) δ 7.27-7.10 (m, 6H), 6.95 (t, J=8.7 Hz, 2H), 6.39 (s, 1H), 4.94 (s, 1H), 3.51 (t, J=6.3 Hz, 2H), 2.99-2.83 (m, 1H), 2.81 (s, 3H), 2.81-2.69 (m, 1H), 2.31 (s, 6H), 1.46 (s, 9H).

### Step 2. Synthesis of (S)-1-(4-fluorophenyl)-N-(3-(methylamino)bicyclo[1.1.1]pentan-1-yl)-3,4-dihydroisoquinoline-2(1H)-carboxamide, Compound 4004

Starting from tert-butyl (S)-(3-(1-(4-fluorophenyl)-1,2,3,4-tetrahydroisoquinoline-2-carboxamido)bicyclo[1.1.1]pentan-1-yl)(methyl)carbamate, 5 (275 mg, 0.591 mmol), (S)-1-(4-fluorophenyl)-N-(3-(methylamino)bicyclo[1.1.1]pentan-1-yl)-3,4-dihydroisoquinoline-2(1H)-carboxamide, compound 4004 (210 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, compound 4005 (see Scheme 5) after basic workup (saturated aqueous NaHCO₃/dichloromethane extraction). White solid. LCMS: 97%, RT=1.376 min., (M+H)⁺=366 (method E). ¹H NMR (300 MHz, chloroform-d) 5 (ppm) 7.23-7.11 (m, 6H), 7.00-6.88 (m, 2H), 6.39 (s, 1H), 4.95 (s, 1H), 3.50 (t, J=6.2 Hz, 2H), 2.96-2.83 (m, 1H), 2.83-2.68 (m, 1H), 2.38 (s, 3H), 2.06 (s, 6H).

### Example 5. (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, Compound 4005, and (S)-1-(4-fluorophenyl)-N-(prop-2-yn-1-yl)-N-(3-(prop-2-yn-1-ylamino)bicyclo[1.1.1]pentan-1-yl)-3,4-dihydroisoquinoline-2(1H)-carboxamide, Compound 4006

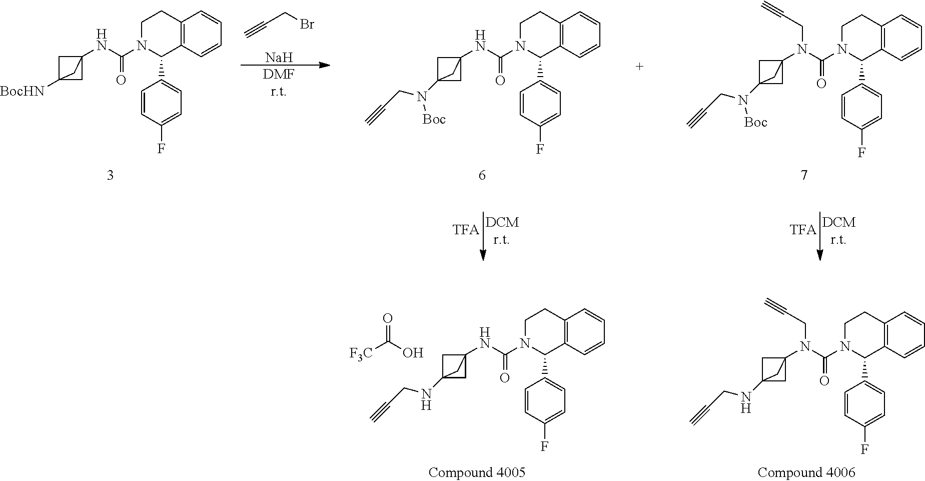

### Step 1. Synthesis of tert-butyl (S)-(3-(1-(4-fluorophenyl)-1,2,3,4-tetrahydroisoquinoline-2-carboxamido)bicyclo[1.1.1]pentan-1-yl)(prop-2-yn-1-yl)carbamate, 6, and tert-butyl (S)-(3-(1-(4-fluorophenyl)-N-(prop-2-yn-1-yl)-1,2,3,4-tetrahydroisoquinoline-2-carboxamido)bicyclo[1.1.1]pentan-1-yl)(prop-2-yn-1-yl)carbamate, 7

Under a nitrogen atmosphere, sodium hydride (60% dispersion in oil, 18 mg, 0.40 mmol) was added to a solution of tert-butyl (S)-(3-(1-(4-fluorophenyl)-1,2,3,4-tetrahydroisoquinoline-2-carboxamido)bicyclo[1.1.1]pentan-1-yl)carbamate, 3 (100 mg, 0.221 mmol) in N,N-dimethylformamide (dry, 0.2 mL) while stirring. After 10 minutes, propargyl bromide (80% in toluene, 50 μL, 0.464 mmol) was added and stirring was continued for 30 minutes. Water (5 mL) was added and stirring was continued overnight. The reaction mixture was extracted with ethyl acetate (2×25 mL). The combined organic layers were dried on Na₂SO₄ and evaporated under reduced pressure. The residue was purified by flash column chromatography (silica, eluting with 0 to 15% ethyl acetate in heptane) to afford tert-butyl (S)-(3-(1-(4-fluorophenyl)-1,2,3,4-tetrahydroisoquinoline-2-carboxamido)bicyclo[1.1.1]pentan-1-yl)(prop-2-yn-1-yl)carbamate, 6 (36 mg) as an oil (LCMS: 95%, RT=1.50 min., (M+H)⁺=490 (method R)) and tert-butyl (S)-(3-(1-(4-fluorophenyl)-N-(prop-2-yn-1-yl)-1,2,3,4-tetrahydroisoquinoline-2-carboxamido)bicyclo[1.1.1]pentan-1-yl)(prop-2-yn-1-yl)carbamate, 7 (62 mg) as an oil (LCMS: 70%, RT=1.67 min., (M+H)⁺=528 (method R)).

### Step 2. Synthesis of (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, Compound 4005

Trifluoroacetic acid (0.1 mL) was added to a solution of tert-butyl (S)-(3-(1-(4-fluorophenyl)-1,2,3,4-tetrahydroisoquinoline-2-carboxamido)bicyclo[1.1.1]pentan-1-yl)(prop-2-yn-1-yl)carbamate, 6 (18 mg, 0.037 mmol) in dichloromethane (2.0 mL). After stirring at room temperature for 4.5 hours, the reaction mixture was evaporated under reduced pressure to give (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, compound 4005 (6.8 mg) as an oil. LCMS: 95.2%, RT=1.855 min., (M+H)⁺=390 (method 0). ¹H NMR (300 MHz, chloroform-d) δ 8.88 (br s, 2H), 7.26-7.03 (m, 6H), 6.94 (td, J=8.6, 2.5 Hz, 2H), 6.33 (s, 1H), 5.68 (br s, 1H), 3.70 (s, 2H), 3.60-3.41 (m, 2H), 2.96-2.81 (m, 1H), 2.81-2.66 (m, 1H), 2.49 (s, 1H), 2.48-2.34 (m, 6H).

### Step 2′. Synthesis of (S)-1-(4-fluorophenyl)-N-(prop-2-yn-1-yl)-N-(3-(prop-2-yn-1-ylamino)bicyclo[1.1.1]pentan-1-yl)-3,4-dihydroisoquinoline-2(1H)-carboxamide, Compound 4006

Starting from tert-butyl (S)-(3-(1-(4-fluorophenyl)-N-(prop-2-yn-1-yl)-1,2,3,4-tetrahydroisoquinoline-2-carboxamido)bicyclo[1.1.1]pentan-1-yl)(prop-2-yn-1-yl)carbamate, 7 (62 mg, 0.12 mmol), (S)-1-(4-fluorophenyl)-N-(prop-2-yn-1-yl)-N-(3-(prop-2-yn-1-ylamino)bicyclo[1.1.1]pentan-1-yl)-3,4-dihydroisoquinoline-2(1H)-carboxamide, compound 4006 (8.5 mg) was prepared according to the procedure described for Step 2, above. The material was purified by flash column chromatography (silica, eluting with 0 to 5% methanol in dichloromethane) after basic workup (aqueous NaOH (15%)/dichloromethane extraction). Oil. LCMS: 99.2%, RT=1.64 min., (M+H)⁺=428 (method E). ¹H NMR (300 MHz, chloroform-d) δ 7.29-7.07 (m, 5H), 7.02-6.87 (m, 3H), 6.36 (s, 1H), 4.04-3.87 (m, 2H), 3.71 (dt, J=17.5, 2.1 Hz, 1H), 3.35 (t, J=2.0 Hz, 2H), 3.26-3.10 (m, 1H), 3.07-2.89 (m, 1H), 2.87-2.74 (m, 1H), 2.25 (q, J=2.2 Hz, 1H), 2.15-2.12 (m, 2H), 2.08 (s, 6H).

### Example 6. (S)-1-(4-fluorophenyl)-N-(3-((2-(methylsulfonyl)ethyl)amino)bicyclo[1.1.1]pentan-1-yl)-3,4-dihydroisoquinoline-2(1H)-carboxamide 2,2,2-trifluoroacetate, Compound 4007 and (S)-1-(4-fluorophenyl)-N-(2-(methylsulfonyl)ethyl)-N-(3-((2-(methylsulfonyl)ethyl)amino)bicyclo[1.1.1]pentan-1-yl)-3,4-dihydroisoquinoline-2(1H)-carboxamide, Compound 4008

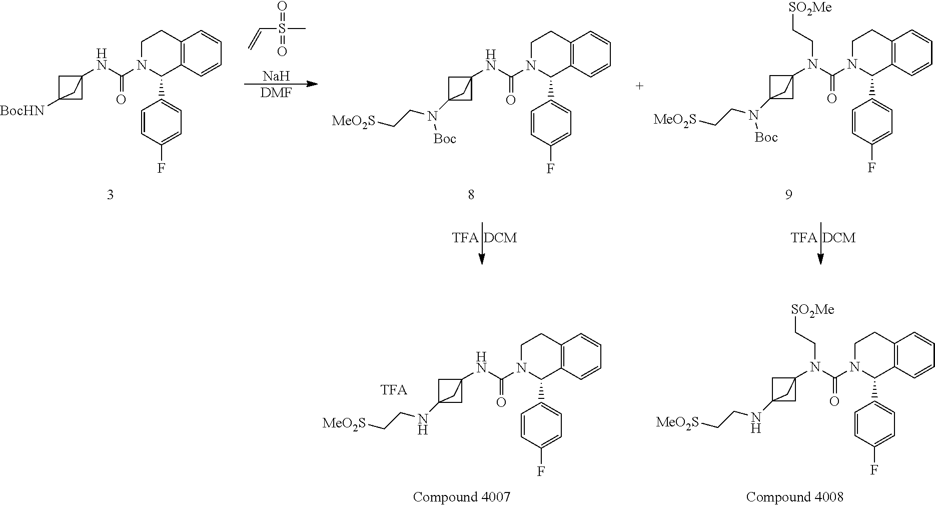

### Step 1. Synthesis of tert-butyl (S)-(3-(1-(4-fluorophenyl)-1,2,3,4-tetrahydroisoquinoline-2-carboxamido)bicyclo[1.1.1]pentan-1-yl)(2-(methylsulfonyl)ethyl)carbamate, 8, and tert-butyl (S)-(3-(1-(4-fluorophenyl)-N-(2-(methylsulfonyl)ethyl)-1,2,3,4-tetrahydroisoquinoline-2-carboxamido)bicyclo[1.1.1]pentan-1-yl)(2-(methylsulfonyl)ethyl)carbamate, 9

Starting from tert-butyl (S)-(3-(1-(4-fluorophenyl)-1,2,3,4-tetrahydroisoquinoline-2-carboxamido)bicyclo[1.1.1]pentan-1-yl)carbamate, 3 (100 mg, 0.221 mmol) and methyl vinyl sulfone (51 mg, 0.48 mmol), tert-butyl (S)-(3-(1-(4-fluorophenyl)-1,2,3,4-tetrahydroisoquinoline-2-carboxamido)bicyclo[1.1.1]pentan-1-yl)(2-(methylsulfonyl)ethyl)carbamate, 8 (57 mg) and tert-butyl (S)-(3-(1-(4-fluorophenyl)-N-(2-(methylsulfonyl)ethyl)-1,2,3,4-tetrahydroisoquinoline-2-carboxamido)bicyclo[1.1.1]pentan-1-yl)(2-(methylsulfonyl)ethyl)carbamate, 9 was isolated (10 mg) were prepared according to the procedure described for tert-butyl (S)-(3-(1-(4-fluorophenyl)-1,2,3,4-tetrahydroisoquinoline-2-carboxamido)bicyclo[1.1.1]pentan-1-yl)(prop-2-yn-1-yl)carbamate, intermediate 6 (see Example 5).

Tert-butyl (S)-(3-(1-(4-Fluorophenyl)-1,2,3,4-tetrahydroisoquinoline-2-carboxamido)bicyclo[1.1.1]pentan-1-yl)(2-(methylsulfonyl)ethyl)carbamate, 8: LCMS: 100%, RT=2.24 min., (M+H)⁺=558 (Method K). ¹H NMR (300 MHz, chloroform-d) δ 7.25-7.07 (m, 6H), 7.01-6.85 (m, 2H), 6.38 (s, 1H), 5.00 (s, 1H), 3.74-3.57 (m, 2H), 3.50 (dd, J=7.0, 5.5 Hz, 2H), 3.24 (dd, J=8.9, 5.9 Hz, 2H), 2.92 (s, 3H), 2.90-2.76 (m, 2H), 2.35 (m, 6H), 1.47 (s, 9H).

Tert-butyl (S)-(3-(1-(4-fluorophenyl)-N-(2-(methylsulfonyl)ethyl)-1,2,3,4-tetrahydroisoquinoline-2-carboxamido)bicyclo[1.1.1]pentan-1-yl)(2-(methylsulfonyl)ethyl)carbamate, 9: LCMS: 87%, RT=2.23 min., (M+H)⁺=664 (method K). ¹H NMR (300 MHz, chloroform-d): δ 7.24-7.08 (m, 6H), 7.01-6.92 (m, 2H), 6.43 (s, 1H), 4.02 (dd, J=13.2, 5.4 Hz, 1H), 3.77-3.60 (m, 2H), 3.55 (dd, J=8.6, 6.0 Hz, 2H), 3.41-3.04 (m, 7H), 2.89 (s, 3H), 2.87 (s, 3H), 2.14 (s, 6H), 1.40 (s, 9H).

### Step 2. Synthesis of (S)-1-(4-fluorophenyl)-N-(3-((2-(methylsulfonyl)ethyl)amino)bicyclo[1.1.1]pentan-1-yl)-3,4-dihydroisoquinoline-2(1H)-carboxamide 2,2,2-trifluoroacetate, Compound 4007

Starting from tert-butyl (S)-(3-(1-(4-fluorophenyl)-1,2,3,4-tetrahydroisoquinoline-2-carboxamido)bicyclo[1.1.1]pentan-1-yl)(2-(methylsulfonyl)ethyl)carbamate, 8 (56 mg, 0.10 mmol), (S)-1-(4-fluorophenyl)-N-(3-((2-(methylsulfonyl)ethyl)amino)bicyclo[1.1.1]pentan-1-yl)-3,4-dihydroisoquinoline-2(1H)-carboxamide 2,2,2-trifluoroacetate, compound 4007 (57 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, compound 4005. LCMS: 99%, RT=1.824 min., (M+H)⁺=458 (method E). ¹H NMR (300 MHz, chloroform-d) δ 7.30-7.07 (m, 6H), 7.01-6.87 (m, 2H), 6.70-6.00 (br s, 2H), 6.33 (s, 1H), 5.67 (br s, 1H), 3.58-3.46 (m, 4H), 3.51-3.36 (m, 2H), 3.00 (s, 3H), 3.01-2.82 (m, 1H), 2.75 (dt, J=16.0, 5.9 Hz, 1H), 2.35 (s, 6H).

### Step 2′. Synthesis of (S)-1-(4-fluorophenyl)-N-(2-(methylsulfonyl)ethyl)-N-(3-((2-(methylsulfonyl)ethyl)amino)bicyclo[1.1.1]pentan-1-yl)-3,4-dihydroisoquinoline-2(1H)-carboxamide, Compound 4008

Starting from tert-butyl (S)-(3-(1-(4-fluorophenyl)-N-(2-(methylsulfonyl)ethyl)-1,2,3,4-tetrahydroisoquinoline-2-carboxamido)bicyclo[1.1.1]pentan-1-yl)(2-(methylsulfonyl)ethyl)carbamate, 9 (10 mg, 0.015 mmol), (S)-1-(4-fluorophenyl)-N-(2-(methylsulfonyl)ethyl)-N-(3-((2-(methylsulfonyl)ethyl)amino)bicyclo[1.1.1]pentan-1-yl)-3,4-dihydroisoquinoline-2(1H)-carboxamide, compound 4008 (5.1 mg) was prepared according to the procedure described above for compound 4007 and purified by flash column chromatography (silica, eluting with 0 to 10% methanol in dichloromethane) after basic workup (aqueous NaOH (15%)/dichloromethane extraction). Oil. LCMS: 97.6%, RT=1.371 min. (M+H)⁺=564 (method E). ¹H NMR (300 MHz, chloroform-d) δ 7.27-7.07 (m, 5H), 7.04-6.89 (m, 3H), 6.44 (s, 1H), 4.03 (dd, J=13.0, 5.4 Hz, 1H), 3.73-3.60 (m, 1H), 3.40-2.98 (m, 9H), 2.97-2.75 (m, 2H), 2.93 (s, 3H), 2.88 (s, 3H), 1.88 (s, 6H).

### Example 7. (S)-1-(4-fluorophenyl)-N-methyl-N-(3-(methylamino)bicyclo[1.1.1]pentan-1-yl)-3,4-dihydroisoquinoline-2(1H)-carboxamide, Compound 4009

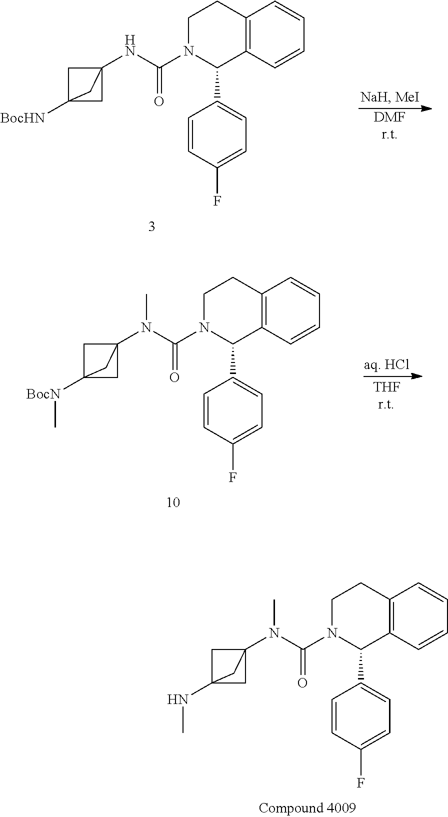 [SMILES: CC(C)(C)OC(=O)NC12CC(NC(=O)N3CCc4ccccc4[C@@H]3c3ccc(F)cc3)(C1)C2 ; "DMFr.t." ; CN(C(=O)OC(C)(C)C)C12CC(N(C)C(=O)N3CCc4ccccc4[C@@H]3c3ccc(F)cc3)(C1)C2 ; CNC12CC(N(C)C(=O)N3CCc4ccccc4[C@@H]3c3ccc(F)cc3)(C1)C2]

### Step 1. Synthesis of tert-butyl (S)-(3-(1-(4-fluorophenyl)-N-methyl-1,2,3,4-tetrahydroisoquinoline-2-carboxamido)bicyclo[1.1.1]pentan-1-yl)(methyl)carbamate, 10

Sodium hydride (60% dispersion in oil, 16 mg, 0.40 mmol) was added to a solution of tert-butyl (S)-(3-(1-(4-fluorophenyl)-1,2,3,4-tetrahydroisoquinoline-2-carboxamido)bicyclo[1.1.1]-pentan-1-yl)carbamate, 3 (91 mg, 0.20 mmol) in N,N-dimethylformamide (dry, 0.2 mL). After 10 minutes, a solution of iodomethane (38 μL, 0.61 mmol) in N,N-dimethylformamide (dry, 0.3 mL) was added. After stirring the reaction mixture for 4 hours, water (5 mL) was added, followed by ethyl acetate (20 mL). The mixture was dried on Na₂SO₄ and evaporated under reduced pressure. The residue was purified by flash column chromatography (silica, 0 to 50% ethyl acetate in heptane) to afford tert-butyl (S)-(3-(1-(4-fluorophenyl)-N-methyl-1,2,3,4-tetrahydroisoquinoline-2-carboxamido)bicyclo[1.1.1]pentan-1-yl)(methyl)carbamate, 10 (50 mg). LCMS: 100%, RT=1.70 min., (M+H)⁺=480 (Method R). ¹H NMR (300 MHz, chloroform-d) δ 7.27-7.03 (m, 5H), 7.01-6.87 (m, 3H), 6.23 (s, 1H), 3.86-3.72 (m, 1H), 3.19 (ddd, J=13.3, 11.3, 4.3 Hz, 1H), 2.97 (ddd, J=17.1, 11.3, 5.8 Hz, 1H), 2.83-2.80 (m, 1H), 2.78 (s, 3H), 2.74 (s, 3H), 2.33-2.12 (m, 6H), 1.43 (s, 9H).

### Step 2. Synthesis of (S)-1-(4-fluorophenyl)-N-methyl-N-(3-(methylamino)bicyclo[1.1.1]pentan-1-yl)-3,4-dihydroisoquinoline-2(1H)-carboxamide, Compound 4009

Aqueous HCl (concentrated, 1.0 mL, 12 mmol) was added to a solution of tert-butyl (S)-(3-(1-(4-fluorophenyl)-N-methyl-1,2,3,4-tetrahydroisoquinoline-2-carboxamido)bicyclo[1.1.1]pentan-1-yl)(methyl)carbamate (50 mg, 0.10 mmol) in tetrahydrofuran (5 mL). The reaction mixture was stirred for 20 hours and then evaporated to dryness (at 65° C.). The residue was triturated with diethyl ether (20 mL) and a mixture of acetone and diethyl ether (1:4, 25 mL), dissolved in methanol (3 mL) and brought onto an SCX-2 column (1.2 g) and eluted with methanol until neutral. Next, the column was eluted with ammonia in methanol (3.5 M, 10 mL). The basic fraction was concentrated to dryness under reduced pressure to give (S)-1-(4-fluorophenyl)-N-methyl-N-(3-(methylamino)bicyclo[1.1.1]pentan-1-yl)-3,4-dihydroisoquinoline-2(1H)-carboxamide, compound 4009 (17.6 mg) as an oil. LCMS: 97.5%, RT=1.547 min. (M+H)⁺=390 (Method E). ¹H NMR (300 MHz, chloroform-d) δ 7.26-7.03 (m, 5H), 7.01-6.87 (m, 3H), 6.42 (s, 1H), 3.87-3.74 (m, 1H), 3.17 (ddd, J=13.2, 11.3, 4.3 Hz, 1H), 3.02-2.88 (m, 1H), 2.87-2.77 (m, 1H), 2.74 (s, 3H), 2.34 (s, 3H), 2.02-1.87 (m, 6H).

### Example 8. (S)—N-(3-aminobicyclo[1.1.1]pentan-1-yl)-1-(4-fluorophenyl)-N-methyl-3,4-dihydroisoquinoline-2(1H)-carboxamide, Compound 4010

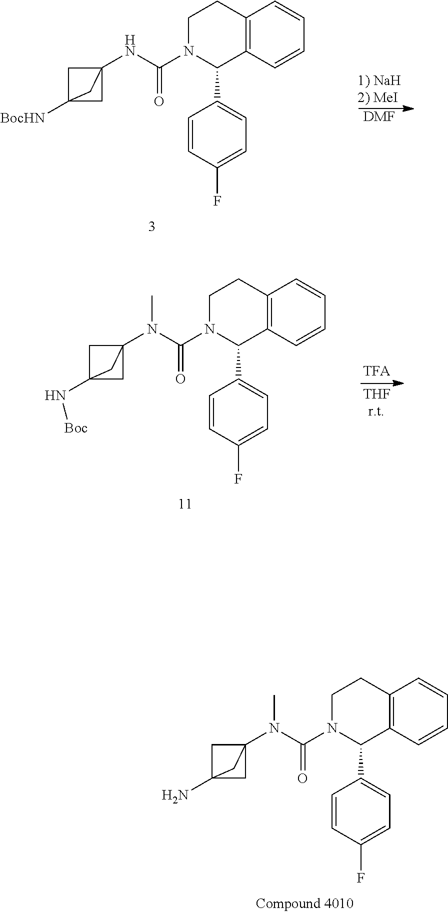

### Step 1. Synthesis of tert-butyl (S)-(3-(1-(4-fluorophenyl)-1,2,3,4-tetrahydroisoquinoline-2-carboxamido)bicyclo[1.1.1]pentan-1-yl)(methyl)carbamate, 5 and tert-butyl (S)-(3-(1-(4-fluorophenyl)-N-methyl-1,2,3,4-tetrahydroisoquinoline-2-carboxamido)bicyclo[1.1.1]pentan-1-yl)carbamate, 11

Starting from tert-butyl (S)-(3-(1-(4-fluorophenyl)-1,2,3,4-tetrahydroisoquinoline-2-carboxamido)bicyclo[1.1.1]pentan-1-yl)carbamate, 3 (55 mg, 0.12 mmol) and iodomethane (35 mg, 0.60 mmol), a mixture of tert-butyl (S)-(3-(1-(4-fluorophenyl)-1,2,3,4-tetrahydroisoquinoline-2-carboxamido)bicyclo[1.1.1]pentan-1-yl)(methyl)carbamate, 5 and tert-butyl (S)-(3-(1-(4-fluorophenyl)-N-methyl-1,2,3,4-tetrahydroisoquinoline-2-carboxamido)bicyclo[1.1.1]pentan-1-yl)carbamate, 11 (10 mg) was prepared according to the procedure described in Scheme 5, Step 1. Used as such in the next step. LCMS: 66%, RT=1.47 min., (M+H)⁺=466 (method R).

### Step 2. Synthesis of (S)—N-(3-aminobicyclo[1.1.1]pentan-1-yl)-1-(4-fluorophenyl)-N-methyl-3,4-dihydroisoquinoline-2(1H)-carboxamide

Trifluoroacetic acid (0.2 mL) was added to a solution of a mixture of tert-butyl (S)-(3-(1-(4-fluorophenyl)-1,2,3,4-tetrahydroisoquinoline-2-carboxamido)bicyclo[1.1.1]pentan-1-yl)(methyl)carbamate, 5 and tert-butyl (S)-(3-(1-(4-fluorophenyl)-N-methyl-1,2,3,4-tetrahydroisoquinoline-2-carboxamido)bicyclo[1.1.1]pentan-1-yl)carbamate, 11 (10 mg, ˜0.021 mmol) in tetrahydrofuran (3 mL) at room temperature. The reaction mixture was stirred at room temperature overnight. The reaction mixture was evaporated under reduced pressure. The residue was taken into dichloromethane (15 mL) and saturated aqueous NaHCO₃ (2 mL) and aqueous NaOH (30%, 2 mL) were added. The mixture was thoroughly mixed and poured out on excess Na₂SO₄. The organic layer was separated and evaporated under reduced pressure. The residue was purified by flash column chromatography (silica, eluting with 0 to 15% methanol in dichloromethane) to give (S)—N-(3-aminobicyclo[1.1.1]pentan-1-yl)-1-(4-fluorophenyl)-N-methyl-3,4-dihydroisoquinoline-2(1H)-carboxamide, compound 4010 (0.3 mg) as an oil. LCMS: 96%, RT=1.48 min., (M+H)⁺=366 (method E). ¹H NMR (300 MHz, chloroform-d) δ 7.25-6.80 (m, 8H), 6.23 (s, 1H), 3.91-3.66 (m, 1H), 3.25-2.84 (m, 2H), 2.84-2.57 (m, 1H), 2.70 (s, 3H), 2.05-1.82 (m, 6H), 1.25 (br s, 2H).

### Example 9. (S)-1-(4-fluorophenyl)-N-(3-((2-(prop-2-yn-1-yloxy)ethyl)amino)bicyclo[1.1.1]pentan-1-yl)-3,4-dihydroisoquinoline-2(1H)-carboxamide, Compound 4011

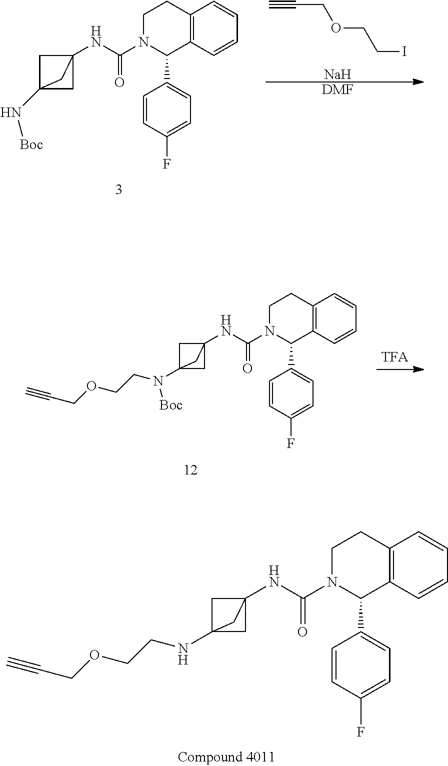

### Step 1. Synthesis of tert-butyl (S)-(3-(1-(4-fluorophenyl)-1,2,3,4-tetrahydroisoquinoline-2-carboxamido)bicyclo[1.1.1]pentan-1-yl)(2-(prop-2-yn-1-yloxy)ethyl)carbamate, 12

Starting from (S)-(3-(1-(4-fluorophenyl)-1,2,3,4-tetrahydroisoquinoline-2-carboxamido)bicyclo[1.1.1]pentan-1-yl)carbamate, 3 (110 mg, 0.24 mmol) and 3-(2-iodoethoxy)prop-1-yne (53.5 mg, 0.255 mmol), tert-butyl (S)-(3-(1-(4-fluorophenyl)-1,2,3,4-tetrahydroisoquinoline-2-carboxamido)bicyclo[1.1.1]pentan-1-yl)(2-(prop-2-yn-1-yloxy)ethyl)carbamate, 12 (76 mg) was prepared according to the procedure described for tert-butyl (S)-(3-(1-(4-fluorophenyl)-1,2,3,4-tetrahydroisoquinoline-2-carboxamido)bicyclo[1.1.1]pentan-1-yl)(prop-2-yn-1-yl)carbamate, 6 (see Scheme 5, step 1). White solid. LCMS: 56%, RT=2.24 min., (M+H)⁺=534 (method K).

### Step 2. Synthesis of (S)-1-(4-fluorophenyl)-N-(3-((2-(prop-2-yn-1-yloxy)ethyl)amino)bicyclo[1.1.1]pentan-1-yl)-3,4-dihydroisoquinoline-2(1H)-carboxamide, Compound 4011

Starting from tert-butyl (S)-(3-(1-(4-fluorophenyl)-1,2,3,4-tetrahydroisoquinoline-2-carboxamido)bicyclo[1.1.1]pentan-1-yl)(2-(prop-2-yn-1-yloxy)ethyl)carbamate, 12 (76 mg, 0.134 mmol), (S)-1-(4-fluorophenyl)-N-(3-((2-(prop-2-yn-1-yloxy)ethyl)amino)bicyclo[1.1.1]pentan-1-yl)-3,4-dihydroisoquinoline-2(1H)-carboxamide, compound 4011 (29.2 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, compound 4005 and purified by flash column chromatography (silica, eluting with 0 to 4% methanol in dichloromethane) after basic workup (aqueous NaOH (15%)/dichloromethane extraction). Oil. LCMS: 98.5%, RT=1.515 min., (M+H)⁺=434 (method R). ¹H NMR (300 MHz, chloroform-d) δ 7.23-7.02 (m, 6H), 6.94 (t, J=8.6 Hz, 2H), 6.39 (s, 1H), 4.94 (s, 1H), 4.16 (d, J=1.6 Hz, 2H), 3.64 (t, J=5.2 Hz, 2H), 3.60-3.38 (m, 1H), 3.50 (t, J=6.2 Hz, 2H) 2.96-2.70 (m, 4H), 2.43 (t, J=1.9 Hz, 1H), 2.08 (s, 6H).

### Example 10. (S)-1-(4-fluorophenyl)-N-(3-(pyrrolidin-1-yl)bicyclo[1.1.1]pentan-1-yl)-3,4-dihydroisoquinoline-2(1H)-carboxamide, Compound 4012

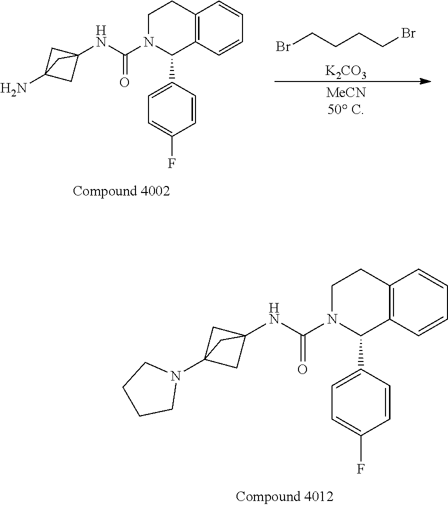

### Synthesis of (S)-1-(4-fluorophenyl)-N-(3-(pyrrolidin-1-yl)bicyclo[1.1.1]pentan-1-yl)-3,4-dihydroisoquinoline-2(1H)-carboxamide, Compound 4012

In a microwave vial 1,4-dibromobutane (20.4 μL, 0.171 mmol) was added to a stirred mixture of (S)—N-(3-aminobicyclo[1.1.1]pentan-1-yl)-1-(4-fluorophenyl)-3,4-dihydroisoquinoline-2(1H)-carboxamide, compound 4002 (50.0 mg, 0.142 mmol) and potassium carbonate (39.3 mg, 0.285 mmol) in acetonitrile (1 mL). The vial was capped and heated overnight at 50° C. After cooling to room temperature, the solids were removed by filtration and the filtrate was concentrated under reduced pressure. The residue was purified by flash column chromatography (silica, eluting with 0 to 15% methanol in dichloromethane) and acidic preparative MPLC (Linear Gradient: t=0 min 5% A, t=1 min 5% A, t=16 min 40% A; t=17 min 100% A; t=22 min 100%; detection: 215/263 nm). The product fractions were combined and lyophilised to afford (S)-1-(4-fluorophenyl)-N-(3-(pyrrolidin-1-yl)bicyclo[1.1.1]pentan-1-yl)-3,4-dihydroisoquinoline-2(1H)-carboxamide, compound 4012 (7 mg) as a white solid. LCMS: 99.7%, RT=2.766 min., (M+H)⁺=406 (method C). ¹H NMR (400 MHz, chloroform-d) δ 7.25-7.12 (m, 6H), 6.95 (t, J=8.7 Hz, 2H), 6.40 (s, 1H), 4.97 (s, 1H), 3.50 (t, J=6.2 Hz, 2H), 2.91 (dt, J=13.6, 6.6 Hz, 1H), 2.77 (dt, J=15.8, 5.9 Hz, 1H), 2.59 (br s, 4H), 2.08 (s, 6H), 1.81 (br s, 4H).

### Example 11. (S)-1-(4-fluorophenyl)-N-(3-morpholinobicyclo[1.1.1]pentan-1-yl)-3,4-dihydroisoquinoline-2(1H)-carboxamide, Compound 4013

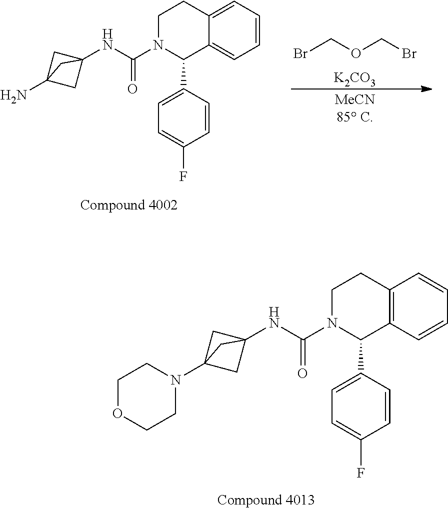

### Synthesis of (S)-1-(4-fluorophenyl)-N-(3-morpholinobicyclo[1.1.1]pentan-1-yl)-3,4-dihydroisoquinoline-2(1H)-carboxamide, Compound 4013

Starting from (S)—N-(3-aminobicyclo[1.1.1]pentan-1-yl)-1-(4-fluorophenyl)-3,4-dihydroisoquinoline-2(1H)-carboxamide, compound 4002 (50.0 mg, 0.142 mmol) and 1-bromo-2-(2-bromoethoxy)ethane (35.8 μL, 0.285 mmol), (S)-1-(4-fluorophenyl)-N-(3-morpholinobicyclo[1.1.1]pentan-1-yl)-3,4-dihydroisoquinoline-2(1H)-carboxamide, compound 4013 (27.6 mg) was prepared according to the above procedure (reaction temperature: 85° C.) described for (S)-1-(4-fluorophenyl)-N-(3-(pyrrolidin-1-yl)bicyclo[1.1.1]pentan-1-yl)-3,4-dihydroisoquinoline-2(1H)-carboxamide, compound 4012. White solid. LCMS: 97%, RT=1.49 min., (M+H)⁺=422 (method E). ¹H NMR (300 MHz, chloroform-d) δ 7.27-7.10 (m, 6H), 7.02-6.89 (m, 2H), 6.39 (s, 1H), 4.97 (s, 1H), 3.74 (t, J=4.5 Hz, 4H), 3.51 (t, J=6.2 Hz, 2H), 2.91 (dt, J=15.9, 6.5 Hz, 1H), 2.77 (dt, J=15.8, 5.9 Hz, 1H), 2.47 (t, J=4.8 Hz, 4H), 2.07 (s, 6H).

### Example 12. 3-((2,2,2-trifluoroethyl)amino)bicyclo[1.1.1]pentan-1-yl (S)-1-(4-fluorophenyl)-3,4-dihydroisoquinoline-2(1H)-carboxylate, Compound 4014

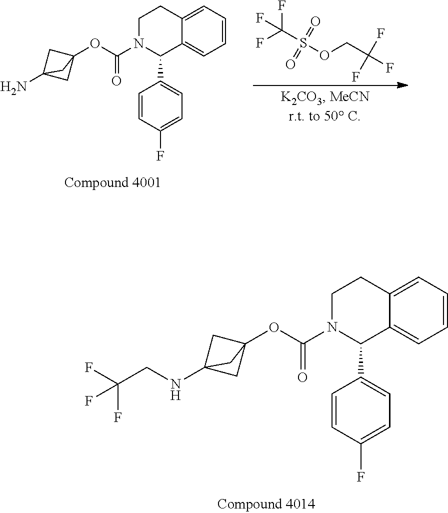

### Synthesis of 3-((2,2,2-trifluoroethyl)amino)bicyclo[1.1.1]pentan-1-yl (S)-1-(4-fluorophenyl)-3,4-dihydroisoquinoline-2(1H)-carboxylate, Compound 4014

Starting from 3-aminobicyclo[1.1.1]pentan-1-yl (S)-1-(4-fluorophenyl)-3,4-dihydroisoquinoline-2(1H)-carboxylate, compound 4001 (26 mg, 0.074 mmol) and 2,2,2-trifluoroethyl trifluoromethanesulfonate (13 μL, 0.089 mmol), 3-((2,2,2-trifluoroethyl)amino)bicyclo[1.1.1]pentan-1-yl (S)-1-(4-fluorophenyl)-3,4-dihydroisoquinoline-2(1H)-carboxylate, compound 4014 (17 mg) was prepared according to the procedure described for (S)-1-(4-fluorophenyl)-N-(3-(pyrrolidin-1-yl)bicyclo[1.1.1]pentan-1-yl)-3,4-dihydroisoquinoline-2(1H)-carboxamide, compound 4010 and purified by acidic preparative MPLC (Linear Gradient: t=0 min 5% A; t=1 min 5% A; t=2 min 30% A; t=17 min 70% A; t=18 min 100% A; t=23 min 100% A; detection: 220/260/277 nm). White solid. LCMS: 99%, RT=4.55 min., (M+H)⁺=435 (method D). ¹H NMR (400 MHz, chloroform-d) mixture of rotamers δ 7.24-7.07 (m, 5H), 7.05-6.91 (m, 3H), 6.46-6.12 (m, 1H), 4.20-3.83 (m, 1H), 3.29-3.07 (m, 3H), 3.07-2.89 (m, 1H), 2.84-2.68 (m, 1H), 2.23 (s, 6H), 1.87 (br s, 1H).

### Example 13. 3-Acetamidobicyclo[1.1.1]pentan-1-yl (S)-1-(4-fluorophenyl)-3,4-dihydroisoquinoline-2(1H)-carboxylate, Compound 4015

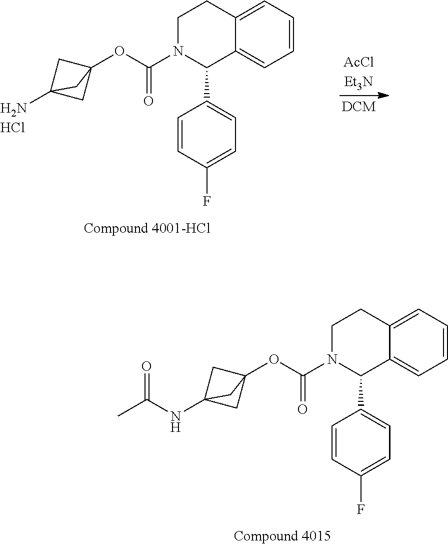 [SMILES: Cl ; NC12CC(OC(=O)N3CCc4ccccc4[C@@H]3c3ccc(F)cc3)(C1)C2 ; CC(=O)NC12CC(OC(=O)N3CCc4ccccc4[C@@H]3c3ccc(F)cc3)(C1)C2]

### Synthesis of 3-acetamidobicyclo[1.1.1]pentan-1-yl (S)-1-(4-fluorophenyl)-3,4-dihydroisoquinoline-2(1H)-carboxylate, Compound 4015

Acetyl chloride (0.34 M in dichloromethane, 100 μL, 0.034 mmol) was added to a solution of 3-aminobicyclo[1.1.1]pentan-1-yl (S)-1-(4-fluorophenyl)-3,4-dihydroisoquinoline-2(1H)-carboxylate hydrochloride, compound 4001-HCl (12.1 mg, 0.031 mmol) and triethylamine (13 μL, 0.093 mmol) in dichloromethane (0.50 mL). After 45 minutes, the reaction mixture was diluted with dichloromethane (5 mL) and washed with aqueous HCl (1 M, 1 mL) and saturated aqueous NaHCO₃ (1 mL). The organic layer was passed through a phase-separator and the filtrate was evaporated under reduced pressure. The residue was purified by flash column chromatography (silica, eluting with 25% to 100% ethyl acetate in heptane) to give 3-acetamidobicyclo[1.1.1]pentan-1-yl (S)-1-(4-fluorophenyl)-3,4-dihydroisoquinoline-2(1H)-carboxylate, compound 4015 (10.1 mg) as a white solid after lyophilization from a mixture of acetonitrile and water (1:1, 4 mL). LCMS: 99.4%, RT=3.355 min., (M+H)⁺=395 (method C). ¹H NMR (400 MHz, chloroform-d) δ 7.24-7.08 (m, 5H), 7.06-6.91 (m, 3H), 6.45-6.14 (m, 1H), 5.88-5.74 (m, 1H), 4.18-3.86 (m, 1H), 3.29-2.88 (m, 2H), 2.83-2.69 (m, 1H), 2.49 (s, 6H), 1.97 (s, 3H).

### Example 14. (S)—N-(3-acetamidobicyclo[1.1.1]pentan-1-yl)-1-(4-fluorophenyl)-3,4-dihydroisoquinoline-2(1H)-carboxamide, Compound 4016

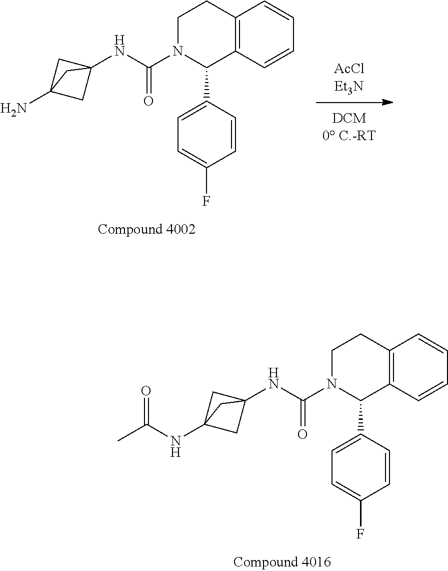 [SMILES: NC12CC(NC(=O)N3CCc4ccccc4[C@@H]3c3ccc(F)cc3)(C1)C2 ; CC(=O)NC12CC(NC(=O)N3CCc4ccccc4[C@@H]3c3ccc(F)cc3)(C1)C2]

### Synthesis of (S)—N-(3-acetamidobicyclo[1.1.1]pentan-1-yl)-1-(4-fluorophenyl)-3,4-dihydroisoquinoline-2(1H)-carboxamide, Compound 4016

Starting from (S)—N-(3-aminobicyclo[1.1.1]pentan-1-yl)-1-(4-fluorophenyl)-3,4-dihydroisoquinoline-2(1H)-carboxamide, compound 4002 (53.7 mg, 0.153 mmol) and acetyl chloride (16.4 μL, 0.229 mmol), (S)—N-(3-acetamidobicyclo[1.1.1]pentan-1-yl)-1-(4-fluorophenyl)-3,4-dihydroisoquinoline-2(1H)-carboxamide, compound 4016 (20 mg) was prepared according to the procedure described for 3-acetamidobicyclo[1.1.1]pentan-1-yl (S)-1-(4-fluorophenyl)-3,4-dihydroisoquinoline-2(1H)-carboxylate, compound 4015 and additionally purified by acidic preparative MPLC (Linear Gradient: t=0 min 5% A, t=1 min 5% A, t=2 min 20% A, t=17 min 60% A; t=18 min 100% A; t=26 min 100%; detection: 220/263 nm). White solid. LCMS: 98.8%, RT=3.022 min., (M+H)⁺=394 (method C). ¹H NMR (400 MHz, chloroform-d) δ 7.25-7.12 (m, 6H), 6.98-6.89 (m, 2H), 6.38 (s, 1H), 5.83 (s, 1H), 4.99 (s, 1H), 3.57-3.43 (m, 2H), 2.97-2.82 (m, 1H), 2.81-2.71 (m, 1H), 2.37 (s, 6H), 1.95 (s, 3H).

### Example 15. (S)-1-(4-fluorophenyl)-N-(3-(methylsulfonamido)bicyclo[1.1.1]pentan-1-yl)-3,4-dihydroisoquinoline-2(1H)-carboxamide, Compound 4017

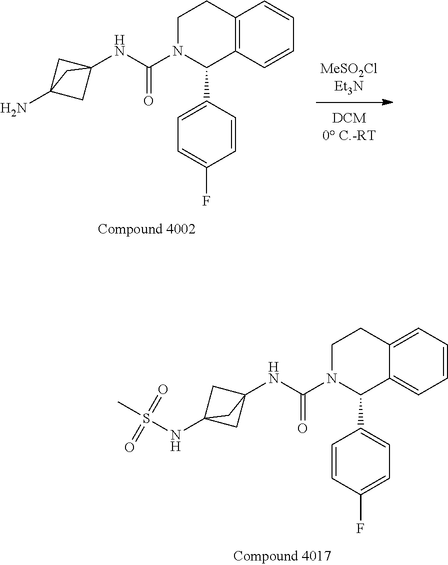 [SMILES: NC12CC(NC(=O)N3CCc4ccccc4[C@@H]3c3ccc(F)cc3)(C1)C2 ; CS(=O)(=O)NC12CC(NC(=O)N3CCc4ccccc4[C@@H]3c3ccc(F)cc3)(C1)C2]

### Synthesis of (S)-1-(4-fluorophenyl)-N-(3-(methylsulfonamido)bicyclo[1.1.1]pentan-1-yl)-3,4-dihydroisoquinoline-2(1H)-carboxamide, Compound 4017

Starting from (S)—N-(3-aminobicyclo[1.1.1]pentan-1-yl)-1-(4-fluorophenyl)-3,4-dihydroisoquinoline-2(1H)-carboxamide, compound 4002 (48.9 mg, 0.139 mmol) and methanesulfonyl chloride (16.3 μL, 0.209 mmol), (S)-1-(4-fluorophenyl)-N-(3-(methylsulfonamido)bicyclo[1.1.1]pentan-1-yl)-3,4-dihydroisoquinoline-2(1H)-carboxamide, compound 4017 (18 mg) was prepared according to the procedure described for 3-acetamidobicyclo[1.1.1]pentan-1-yl (S)-1-(4-fluorophenyl)-3,4-dihydroisoquinoline-2(1H)-carboxylate, compound 4015 and additionally purified by acidic preparative MPLC (Linear Gradient: t=0 min 5% A, t=1 min 5% A, t=2 min 20% A, t=17 min 60% A; t=18 min 100% A; t=26 min 100%; detection: 220/263 nm). White solid. LCMS: 100%, RT=3.127 min., (M+H)⁺=430 (method C). ¹H NMR (400 MHz, chloroform-d) δ 7.25-7.22 (m, 2H), 7.21-7.12 (m, 4H), 7.00-6.88 (m, 2H), 6.38 (s, 1H), 4.99 (s, 1H), 4.88 (s, 1H), 3.56-3.44 (m, 2H), 2.99 (s, 3H), 2.91 (dt, J=15.6, 6.5 Hz, 1H), 2.78 (dt, J=15.9, 6.0 Hz, 1H), 2.37 (s, 6H).

### Example 16. 3-(Methylsulfonamido)bicyclo[1.1.1]pentan-1-yl (S)-1-(4-fluorophenyl)-3,4-dihydroisoquinoline-2(1H)-carboxylate, Compound 4018

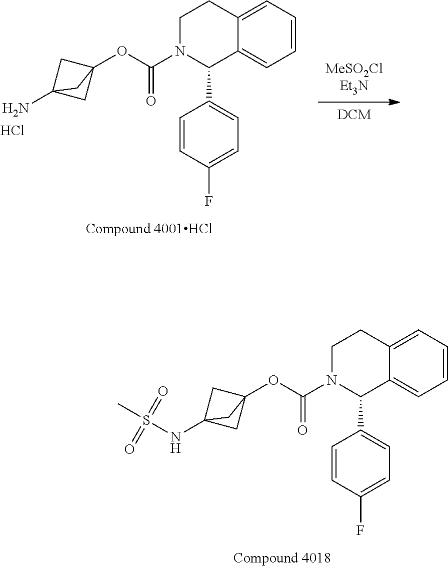

### Synthesis of 3-(methylsulfonamido)bicyclo[1.1.1]pentan-1-yl (S)-1-(4-fluorophenyl)-3,4-dihydroisoquinoline-2(1H)-carboxylate, Compound 4018

Starting from 3-aminobicyclo[1.1.1]pentan-1-yl (S)-1-(4-fluorophenyl)-3,4-dihydroisoquinoline-2(1H)-carboxylate hydrochloride, compound 4001.HCl (12.1 mg, 0.031 mmol 3-(methylsulfonamido)bicyclo[1.1.1]pentan-1-yl (S)-1-(4-fluorophenyl)-3,4-dihydroisoquinoline-2(1H)-carboxylate, compound 4018 (10.2 mg) was prepared according to the procedure described for 3-acetamidobicyclo[1.1.1]pentan-1-yl (S)-1-(4-fluorophenyl)-3,4-dihydroisoquinoline-2(1H)-carboxylate, compound 4015. White solid. LCMS: 99.2%, RT=3.940 min., (M+H)⁺=431 (method C). ¹H NMR (400 MHz, chloroform-d) δ 7.25-7.07 (m, 5H), 7.06-6.91 (m, 3H), 6.46-6.10 (m, 1H), 4.84 (s, 1H), 4.17-3.85 (m, 1H), 3.29-3.09 (m, 1H), 3.07-2.91 (m, 1H), 2.99 (s, 3H), 2.83-2.71 (m, 1H), 2.48 (s, 6H).

### Example 17. (S)—N-(3-acetamidobicyclo[1.1.1]pentan-1-yl)-1-(4-fluorophenyl)-3,4-dihydroisoquinoline-2(1H)-carboxamide, Compound 4019

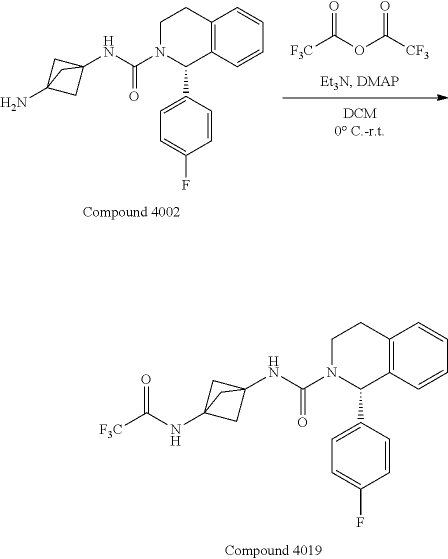 [SMILES: NC12CC(NC(=O)N3CCc4ccccc4[C@@H]3c3ccc(F)cc3)(C1)C2 ; O=C(OC(=O)C(F)(F)F)C(F)(F)F ; O=C(NC12CC(NC(=O)C(F)(F)F)(C1)C2)N1CCc2ccccc2[C@@H]1c1ccc(F)cc1]

### Synthesis of (S)—N-(3-acetamidobicyclo[1.1.1]pentan-1-yl)-1-(4-fluorophenyl)-3,4-dihydroisoquinoline-2(1H)-carboxamide, Compound 4019

Starting from (S)—N-(3-aminobicyclo[1.1.1]pentan-1-yl)-1-(4-fluorophenyl)-3,4-dihydroisoquinoline-2(1H)-carboxamide, compound 4002 (75 mg, 0.21 mmol), 2,2,2-trifluoroacetic anhydride (30 μL, 0.21 mmol), and 4-dimethylaminopyridine (2.6 mg, 0.021 mmol), (S)—N-(3-acetamidobicyclo[1.1.1]pentan-1-yl)-1-(4-fluorophenyl)-3,4-dihydroisoquinoline-2(1H)-carboxamide, compound 4019 (11.8 mg) was prepared according to the procedure described for 3-acetamidobicyclo[1.1.1]pentan-1-yl (S)-1-(4-fluorophenyl)-3,4-dihydroisoquinoline-2(1H)-carboxylate, compound 4015. White solid. LCMS: 98.5%, RT=1.562 min., (M+H)⁺=448 (method E). ¹H NMR (300 MHz, methanol-d₄) δ 7.31-7.09 (m, 6H), 7.06-6.92 (m, 2H), 6.41 (s, 1H), 4.57 (br s, 2H), 3.73-3.59 (m, 1H), 3.42-3.30 (m, 1H), 2.91 (ddd, J=14.8, 8.9, 5.8 Hz, 1H), 2.72 (dt, J=16.2, 5.2 Hz, 1H), 2.38 (s, 6H).

### Example 18. (S)-1-(4-fluorophenyl)-N-(3-((trifluoromethyl)sulfonamido)bicyclo[1.1.1]pentan-1-yl)-3,4-dihydroisoquinoline-2(1H)-carboxamide, Compound 4020

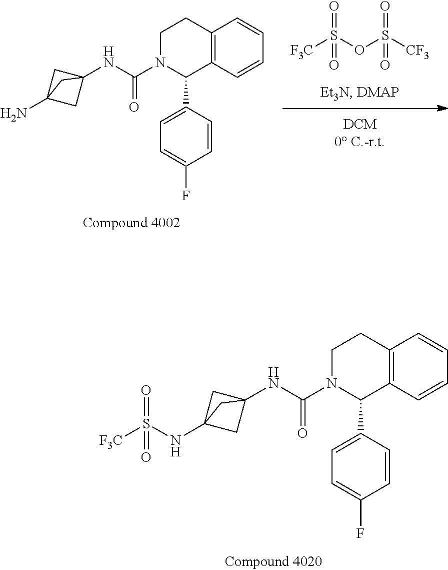 [SMILES: NC12CC(NC(=O)N3CCc4ccccc4[C@@H]3c3ccc(F)cc3)(C1)C2 ; O=S(=O)(OS(=O)(=O)C(F)(F)F)C(F)(F)F ; O=C(NC12CC(NS(=O)(=O)C(F)(F)F)(C1)C2)N1CCc2ccccc2[C@@H]1c1ccc(F)cc1]

### Synthesis of (S)-1-(4-fluorophenyl)-N-(3-((trifluoromethyl)sulfonamido)bicyclo[1.1.1]pentan-1-yl)-3,4-dihydroisoquinoline-2(1H)-carboxamide, Compound 4020

Starting from (S)—N-(3-aminobicyclo[1.1.1]pentan-1-yl)-1-(4-fluorophenyl)-3,4-dihydroisoquinoline-2(1H)-carboxamide, compound 4002 (75 mg, 0.21 mmol), trifluoromethanesulfonic anhydride (36 μL, 0.21 mmol), and 4-dimethylaminopyridine (2.6 mg, 0.021 mmol), (S)-1-(4-fluorophenyl)-N-(3-((trifluoromethyl)sulfonamido)bicyclo[1.1.1]pentan-1-yl)-3,4-dihydroisoquinoline-2(1H)-carboxamide, compound 4020 (11.4 mg) was prepared according to the procedure described for 3-acetamidobicyclo[1.1.1]pentan-1-yl (S)-1-(4-fluorophenyl)-3,4-dihydroisoquinoline-2(1H)-carboxylate, compound 4015. White solid. LCMS: 97.3%, RT=1.531 min., (M+H)⁺=484 (method E). ¹H NMR (300 MHz, chloroform-d) δ 7.29-7.10 (m, 6H), 7.02-6.88 (m, 2H), 6.35 (s, 1H), 4.96 (s, 1H), 3.50 (t, J=6.3 Hz, 2H), 2.98-2.84 (m, 1H), 2.77 (dt, J=15.8, 6.0 Hz, 1H), 2.40 (s, 6H).

### Example 19. (S)—N-(3-(1,1-dioxidothiomorpholino)bicyclo[1.1.1]pentan-1-yl)-1-(4-fluorophenyl)-3,4-dihydroisoquinoline-2(1H)-carboxamide, Compound 4021

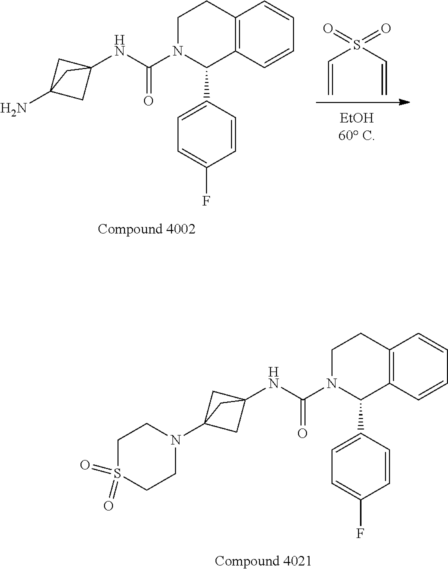

### Synthesis of (S)—N-(3-(1,1-dioxidothiomorpholino)bicyclo[1.1.1]pentan-1-yl)-1-(4-fluorophenyl)-3,4-dihydroisoquinoline-2(1H)-carboxamide, Compound 4021

In a microwave vial (vinylsulfonyl)ethene (28.6 μL, 0.285 mmol) was added to a stirred solution of (S)—N-(3-aminobicyclo[1.1.1]pentan-1-yl)-1-(4-fluorophenyl)-3,4-dihydroisoquinoline-2(1H)-carboxamide, compound 4002 (50.0 mg, 0.142 mmol) in ethanol (absolute, 1 mL). The vial was capped and heated for 2.5 hours at 60° C. The solvent was removed under reduced pressure. The residue was purified by flash column chromatography (silica, eluing with 0 to 5% methanol in dichloromethane) to obtain (S)-1-(4-fluorophenyl)-N-(3-morpholinobicyclo[1.1.1]pentan-1-yl)-3,4-dihydroisoquinoline-2(1H)-carboxamide, compound 4021 (36.9 mg) as a white solid. LCMS: 99.0%, RT=1.44 min., (M+H)⁺=470 (method E). ¹H NMR (300 MHz, chloroform-d) δ 7.28-7.10 (m, 6H), 7.01-6.89 (m, 2H), 6.37 (s, 1H), 4.96 (s, 1H), 3.50 (t, J=6.3 Hz, 2H), 3.12-2.96 (m, 8H), 2.91 (dt, J=15.8, 5.9 Hz, 1H), 2.77 (dt, J=15.8, 5.9 Hz, 1H), 2.10 (s, 6H).

### Example 20. (S)—N-(3-(dimethylamino)bicyclo[1.1.1]pentan-1-yl)-1-(4-fluorophenyl)-3,4-dihydroisoquinoline-2(1H)-carboxamide, Compound 4022

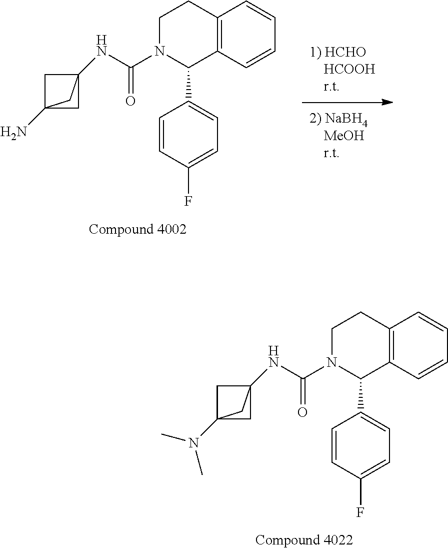 [SMILES: NC12CC(NC(=O)N3CCc4ccccc4[C@@H]3c3ccc(F)cc3)(C1)C2 ; CN(C)C12CC(NC(=O)N3CCc4ccccc4[C@@H]3c3ccc(F)cc3)(C1)C2]

### Synthesis of (S)—N-(3-(dimethylamino)bicyclo[1.1.1]pentan-1-yl)-1-(4-fluorophenyl)-3,4-dihydroisoquinoline-2(1H)-carboxamide, Compound 4022

A mixture of (S)—N-(3-aminobicyclo[1.1.1]pentan-1-yl)-1-(4-fluorophenyl)-3,4-dihydroisoquinoline-2(1H)-carboxamide, compound 4002 (100 mg, 0.285 mmol), formaldehyde (37% aqueous; 7 mL) and formic acid (7 mL) was stirred at room temperature for 30 minutes. The reaction mixture was evaporated under reduced pressure at 65° C. Sodium borohydride (818 mg, 21.6 mmol), followed by methanol (20 mL) was added to the residue while stirring. After 5 minutes stirring, the resulting slurry was diluted with methanol (20 mL) and evaporated under reduced pressure. The residue was diluted with water (20 mL) and ethyl acetate (20 mL). After vigorous mixing the organic layer was separated, dried on Na₂SO₄ and evaporated under reduced pressure. The residue was purified by flash column chromatography (silica, eluting with 0 to 10% methanol in dichloromethane) followed by repeated reversed phase chromatography on a 12 g RP-Sepracor column (first run eluting with a water:acetonitrile gradient, up to 100% acetonitrile; second run eluting with water:acetonitrile gradient, up to 55% acetonitrile) gave (S)—N-(3-(dimethylamino)bicyclo[1.1.1]pentan-1-yl)-1-(4-fluorophenyl)-3,4-dihydroisoquinoline-2(1H)-carboxamide, compound 4022 (4.9 mg) as solid. LCMS: 99.0%, RT=1.54 min., (M+H)⁺=380 (Method E). ¹H NMR (300 MHz, chloroform-d) δ 7.29-7.10 (m, 6H), 6.94 (td, J=8.6, 1.4 Hz, 2H), 6.40 (s, 1H), 4.95 (s, 1H), 3.51 (td, J=6.2, 5.5, 1.6 Hz, 2H), 2.91 (dt, J=16.1, 6.5 Hz, 1H), 2.76 (dt, J=15.9, 6.0 Hz, 1H), 2.24 (s, 6H), 2.04 (s, 6H).

### Example 21. (S)-1-(4-fluorophenyl)-N-(3-((2,2,2-trifluoroethyl)amino)bicyclo[1.1.1]pentan-1-yl)-3,4-dihydroisoquinoline-2(1H)-carboxamide, Compound 4023

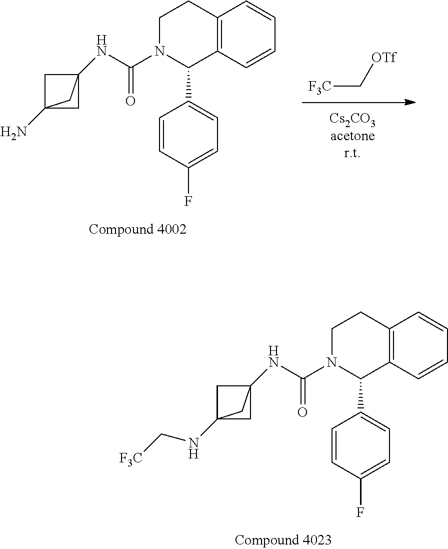 [SMILES: NC12CC(NC(=O)N3CCc4ccccc4[C@@H]3c3ccc(F)cc3)(C1)C2 ; O=S(=O)(OCC(F)(F)F)C(F)(F)F ; O=C(NC12CC(NCC(F)(F)F)(C1)C2)N1CCc2ccccc2[C@@H]1c1ccc(F)cc1]

### Synthesis of (S)-1-(4-fluorophenyl)-N-(3-((2,2,2-trifluoroethyl)amino)bicyclo[1.1.1]pentan-1-yl)-3,4-dihydroisoquinoline-2(1H)-carboxamide, Compound 4023

Cesium carbonate (890 mg, 2.73 mmol) and 2,2,2-trifluoroethyl trifluoromethanesulfonate (447 mg, 1.93 mmol) were added to a solution of (S)—N-(3-aminobicyclo[1.1.1]pentan-1-yl)-1-(4-fluorophenyl)-3,4-dihydroisoquinoline-2(1H)-carboxamide, compound 4002 (200 mg, 0.569 mmol) in acetone (4 mL) at room temperature. The mixture was stirred for 3 days and then filtered. The filtrate was evaporated under reduced pressure. The residue was purified by flash column chromatography (silica, eluting with 0 to 100% ethyl acetate in heptane) to give (S)-1-(4-fluorophenyl)-N-(3-((2,2,2-trifluoroethyl)amino)bicyclo[1.1.1]pentan-1-yl)-3,4-dihydroisoquinoline-2(1H)-carboxamide, compound 4023 (45 mg) as an oil. LCMS: 98.5%, RT=1.58 min., (M+H)⁺=434 (Method E). ¹H NMR (300 MHz, chloroform-d) δ 7.27-7.06 (m, 6H), 7.03-6.80 (m, 2H), 6.38 (s, 1H), 5.00 (s, 1H), 3.59-3.37 (m, 2H), 3.16 (q, J=9.4 Hz, 2H), 3.00-2.64 (m, 2H), 2.11 (s, 6H), 1.88 (br s, 1H).

### Example 22. (S)-1-(4-fluorophenyl)-N-(3-(2-oxopyrrolidin-1-yl)bicyclo[1.1.1]pentan-1-yl)-3,4-dihydroisoquinoline-2(1H)-carboxamide, Compound 4024

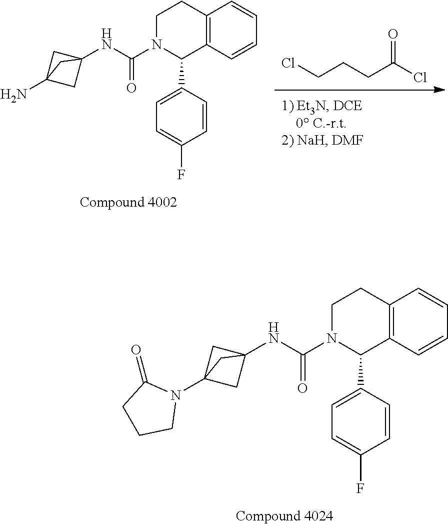 [SMILES: NC12CC(NC(=O)N3CCc4ccccc4[C@@H]3c3ccc(F)cc3)(C1)C2 ; O=C(Cl)CCCCl ; O=C(NC12CC(N3CCCC3=O)(C1)C2)N1CCc2ccccc2[C@@H]1c1ccc(F)cc1]

### Synthesis of (S)-1-(4-fluorophenyl)-N-(3-(2-oxopyrrolidin-1-yl)bicyclo[1.1.1]pentan-1-yl)-3,4-dihydroisoquinoline-2(1H)-carboxamide, Compound 4024

4-Chlorobutanoyl chloride (16.6 μL, 0.148 mmol) was added to a stirred solution of (S)—N-(3-aminobicyclo[1.1.1]pentan-1-yl)-1-(4-fluorophenyl)-3,4-dihydroisoquinoline-2(1H)-carboxamide, compound 4002 (43.2 mg, 0.123 mmol) and triethylamine (26 μL, 0.184 mmol) in 1,2-dichloroethane (2 mL). After 1.5 hours, the reaction mixture was diluted with dichloromethane (5 mL) and washed with aqueous HCl (1 M, 2×1 mL). The organic layer was dried on Na₂SO₄ and evaporated under reduced pressure. The residue was dissolved in N,N-dimethylformamide (2 mL) and NaH (60% in oil, 7.4 mg, 0.184 mmol). was added. After stirring for 2 hours and 15 minutes, the reaction mixture was diluted with dichloromethane (10 mL) and water (5 mL) and acidified with aqueous HCl (1 M, 2 drops). The layers were separated and the aqueous phase was extracted with dichloromethane (2×5 mL). The combined organic layers were washed with brine (3 mL), dried on Na₂SO₄, and evaporated under reduced pressure. The residue was purified by flash column chromatography (silica, eluting with 0 to 5% methanol in dichloromethane) and acidic preparative MPLC (Linear Gradient: t=0 min 5% A, t=1 min 5% A, t=2 min 20% A, t=17 min 60% A; t=18 min 100% A; t=26 min 100%; detection: 220/263 nm). The product fractions were combined and lyophilised to afford (S)-1-(4-fluorophenyl)-N-(3-(2-oxopyrrolidin-1-yl)bicyclo[1.1.1]pentan-1-yl)-3,4-dihydroisoquinoline-2(1H)-carboxamide, compound 4024 (8 mg) as an off-white solid. LCMS: 98.3%, RT=3.533 min., (M+H)⁺=420 (method D). ¹H NMR (400 MHz, chloroform-d) δ 7.25-7.20 (m, 2H), 7.20-7.11 (m, 4H), 6.99-6.90 (m, 2H), 6.39 (s, 1H), 5.00 (s, 1H), 3.58-3.45 (m, 2H), 3.45-3.30 (m, 2H), 2.96-2.86 (m, 1H), 2.82-2.72 (m, 1H), 2.41 (s, 6H), 2.39-2.33 (m, 2H), 2.04-1.94 (m, 2H).

### Example 23. 3-(2-Oxopyrrolidin-1-yl)bicyclo[1.1.1]pentan-1-yl (S)-1-(4-fluorophenyl)-3,4-dihydroisoquinoline-2(1H)-carboxylate, Compound 4025

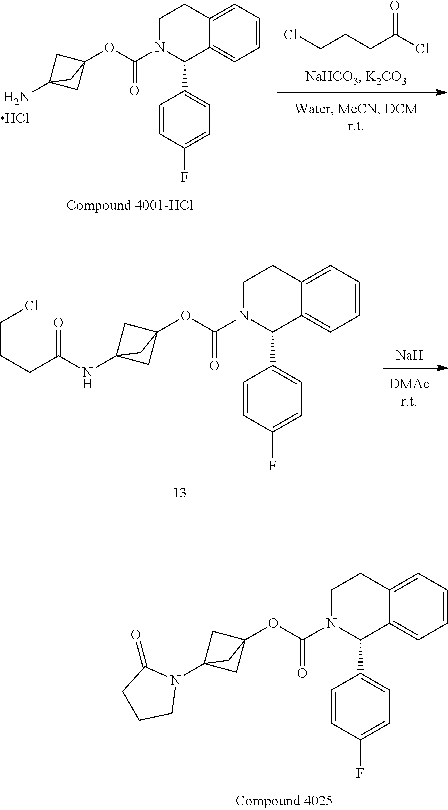

### Step 1. Synthesis of 3-(4-chlorobutanamido)bicyclo[1.1.1]pentan-1-yl (S)-1-(4-fluorophenyl)-3,4-dihydroisoquinoline-2(1H)-carboxylate, 13

3-Aminobicyclo[1.1.1]pentan-1-yl (S)-1-(4-fluorophenyl)-3,4-dihydroisoquinoline-2(1H)-carboxylate hydrochloride, compound 4001-HCl (9.6 mg, 0.025 mmol) was dissolved in water (0.5 mL) and acetonitrile (0.5 mL). NaHCO₃ (11.6 mg, 0.138 mmol) was added, followed by 4-chlorobutyryl chloride (5.53 μL, 0.049 mmol), and the reaction mixture was stirred at room temperature for 2 hours. Then, dichloromethane (1 mL) was added, followed by 4-chlorobutyl chloride (5 drops (excess)), water (1 mL), and saturated aqueous K₂CO₃ (0.3 mL). The biphasic mixture was stirred overnight after which the layers were separated using a phase separator. The organic layer was evaporated under reduced pressure. The residue was purified by flash column chromatography (silica, eluting with 5 to 50% ethyl acetate in heptane) to give 3-(4-chlorobutanamido)bicyclo[1.1.1]pentan-1-yl (S)-1-(4-fluorophenyl)-3,4-dihydroisoquinoline-2(1H)-carboxylate, 13 (10.7 mg) as an oil. LCMS: 100%, RT=2.15 min., (M+H)⁺=457/459, chloro pattern (method A). ¹H NMR (400 MHz, chloroform-d) mixture of rotamers δ 7.26-7.08 (m, 5H), 7.07-6.91 (m, 3H), 6.46-6.15 (m, 1H), 5.88 (s, 1H), 4.21-3.86 (m, 1H), 3.61 (t, J=6.1 Hz, 2H), 3.32-2.89 (m, 2H), 2.83-2.68 (m, 1H), 2.49 (s, 6H), 2.35 (t, J=7.1 Hz, 2H), 2.11 (p, J=6.6 Hz, 2H).

### Step 2. Synthesis of 3-(2-oxopyrrolidin-1-yl)bicyclo[1.1.1]pentan-1-yl (S)-1-(4-fluorophenyl)-3,4-dihydroisoquinoline-2(1H)-carboxylate, Compound 4025

Sodium hydride (60% dispersion in oil, 0.94 mg, 0.023 mmol) was added to a solution of 3-(4-chlorobutanamido)bicyclo[1.1.1]pentan-1-yl (S)-1-(4-fluorophenyl)-3,4-dihydroisoquinoline-2(1H)-carboxylate, 13 (10.7 mg, 0.023 mmol) in N,N-dimethylacetamide (dry, 0.2 mL). The reaction mixture was stirred at room temperature and after 5 hours, the reaction mixture was dropped in a mixture of ice (1 mL) and water (1 mL). The mixture was extracted with ethyl acetate (4×2 mL) and the combined organics were evaporated under reduced pressure. The residue was dissolved in a mixture of acetonitrile and water (2:1, 1 mL), filtered through a PFTE filter (0.45 μm) and purified by basic preparative MPLC (Linear Gradient: t=0 min 5% A, t=1 min 5% A; t=1 min 20% A; t=15 min 60% A; t=1 min 100% A; t=5 min 100% A, detection: 210/263 nm) and acidic preparative MPLC (Linear Gradient: t=0 min 30% A, t=2 min 30% A; t=15 min 70% A; t=1 min 100%; t=25 min 100% A; detection: 210/263 nm). The product containing fractions were combined and evaporated under reduced pressure. The residue was lyophilized from a mixture of acetonitrile and water (1:1, 4 mL) to give 3-(2-oxopyrrolidin-1-yl)bicyclo[1.1.1]pentan-1-yl (S)-1-(4-fluorophenyl)-3,4-dihydroisoquinoline-2(1H)-carboxylate, compound 4025 (6 mg) as a slightly brown solid. LCMS: 100%, RT=4.14 min., (M+H)⁺=421 (method C). ¹H NMR (400 MHz, chloroform-d) mixture of rotamers δ 7.27-7.07 (m, 5H), 7.07-6.87 (m, 3H), 6.55-6.06 (m, 1H), 4.23-3.82 (m, 1H), 3.37 (t, J=7.1 Hz, 2H), 3.29-3.08 (m, 1H), 3.08-2.90 (m, 1H), 2.85-2.69 (m, 1H), 2.53 (s, 6H), 2.37 (t, J=8.1 Hz, 2H), 2.01 (p, J=7.7 Hz, 2H).

### Example 24. 3-(Methylamino)bicyclo[1.1.1]pentan-1-yl (S)-1-(4-fluorophenyl)-3,4-dihydroisoquinoline-2(1H)-carboxylate hydrochloride, Compound 4026

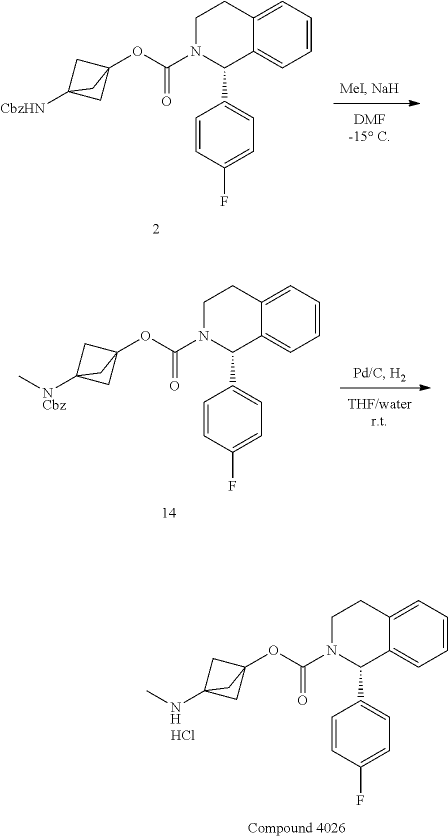

### Step 1. Synthesis of 3-(((benzyloxy)carbonyl)(methyl)amino)bicyclo[1.1.1]pentan-1-yl (S)-1-(4-fluorophenyl)-3,4-dihydroisoquinoline-2(1H)-carboxylate

A solution of 3-(((benzyloxy)carbonyl)amino)bicyclo[1.1.1]pentan-1-yl (S)-1-(4-fluorophenyl)-3,4-dihydroisoquinoline-2(1H)-carboxylate, 2 (50 mg, 0.103 mmol) in N,N-dmethylformamide (dry, 2 mL) was cooled to −15° C. after which methyl iodide (32 μL, 0.514 mmol) was added, followed by sodium hydride (60% dispersion in oil, 4.52 mg, 0.113 mmol). The mixture was stirred at −15° C. under nitrogen atmosphere for 3 hours. The reaction mixture was quenched with saturated aqueous NH₄Cl and extracted with dichloromethane (3 times). The combined organic layers were dried on Na₂SO₄ and evaporated under reduced pressure. The residue was purified by acidic preparative MPLC (Linear Gradient: t=0 min 10% A, t=1 min 10% A; t=2 min 40%, t=17 min 80% A; t=18 min 100%; t=23 min 100% A; detection: 220/254/280 nm) to give 3-(((benzyloxy)carbonyl)(methyl)amino)bicyclo[1.1.1]pentan-1-yl (S)-1-(4-fluorophenyl)-3,4-dihydroisoquinoline-2(1H)-carboxylate, 14 (30 mg) as a white solid after lyophilization of the product fractions. LCMS: 99%, RT=2.383 min., (M+H)⁺=501 (method B).

### Step 2. Synthesis of 3-(methylamino)bicyclo[1.1.1]pentan-1-yl (S)-1-(4-fluorophenyl)-3,4-dihydroisoquinoline-2(1H)-carboxylate hydrochloride, Compound 4026

A solution of 3-(((benzyloxy)carbonyl)(methyl)amino)bicyclo[1.1.1]pentan-1-yl (S)-1-(4-fluorophenyl)-3,4-dihydroisoquinoline-2(1H)-carboxylate, 14 (30 mg, 0.060 mmol) in tetrahydrofuran (dry, 3 mL) and water (2 mL) was hydrogenated at atmospheric pressure in the presence of palladium (10% on activated carbon, 50% wet, catalytic) at room temperature for 1 hour. The reaction mixture was filtered over a 0.45 μm nylon filter and the filtrate was concentrated under reduced pressure. The residue was diluted with acetonitrile and purified by acidic preparative MPLC (Linear Gradient: t=0 min 5% A, t=1 min 5% A; t=2 min 10%, t=17 min 50% A; t=18 min 100%; t=23 min 100% A; detection: 220/254/280 nm). The product containing fractions were diluted with aqueous HCl (2 M, 2 mL) and lyophilized to give 3-(methylamino)bicyclo[1.1.1]pentan-1-yl (S)-1-(4-fluorophenyl)-3,4-dihydroisoquinoline-2(1H)-carboxylate hydrochloride, compound 4026 (12 mg) as a colorless glass. LCMS: 99%, RT=4.052 min., (M+H)⁺=367 (method D). ¹H NMR (400 MHz, chloroform-d) mixture of rotamers δ 10.47 (s, 2H), 7.25-7.06 (m, 5H), 7.05-6.89 (m, 3H), 6.43-6.09 (m, 1H), 4.19-3.83 (m, 1H), 3.31-3.09 (m, 1H), 3.09-2.88 (m, 1H), 2.85-2.71 (m, 1H), 2.67 (s, 3H), 2.59 (s, 6H).

### Example 25. 3-((2-(Methylsulfonyl)ethyl)amino)bicyclo[1.1.1]pentan-1-yl (S)-1-(4-fluorophenyl)-3,4-dihydroisoquinoline-2(1H)-carboxylate hydrochloride, Compound 4027

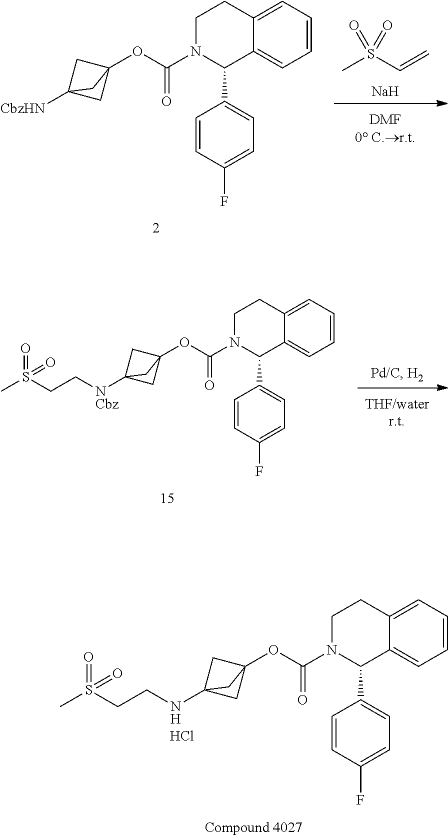

### Step 1. Synthesis of 3-(((benzyloxy)carbonyl)(2-(methylsulfonyl)ethyl)amino)bicyclo[1.1.1]pentan-1-yl (S)-1-(4-fluorophenyl)-3,4-dihydroisoquinoline-2(1H)-carboxylate, 15

Starting from 3-(((benzyloxy)carbonyl)amino)bicyclo[1.1.1]pentan-1-yl (S)-1-(4-fluorophenyl)-3,4-dihydroisoquinoline-2(1H)-carboxylate, 2 (75 mg, 0.15 mmol) and (methylsulfonyl)ethene (0.040 ml, 0.46 mmol), 3-(((benzyloxy)carbonyl)(2-(methylsulfonyl)ethyl)amino)bicyclo[1.1.1]pentan-1-yl (S)-1-(4-fluorophenyl)-3,4-dihydroisoquinoline-2(1H)-carboxylate, 15 (50 mg) was prepared according to the procedure described for 3-(((benzyloxy)carbonyl)(methyl)amino)bicyclo[1.1.1]pentan-1-yl (S)-1-(4-fluorophenyl)-3,4-dihydroisoquinoline-2(1H)-carboxylate, 14 (see Scheme 24, Step 1) and purified by flash column chromatography (silica, eluting with 10% to 70% ethyl acetate in heptane). Syrup. LCMS: 98%, RT=2.27 min., (M+H)⁺=593, (Method A). ¹H NMR (400 MHz, chloroform-d) mixture of rotamers δ 7.46-7.29 (m, 5H), 7.24-7.06 (m, 5H), 7.06-6.90 (m, 3H), 6.38-6.18 (m, 1H), 5.15 (s, 2H), 4.18-3.86 (m, 1H), 3.78-3.67 (m, 2H), 3.32-3.09 (m, 3H), 3.04-2.68 (m, 5H), 2.49 (s, 6H).

### Step 2. Synthesis of 3-((2-(methylsulfonyl)ethyl)amino)bicyclo[1.1.1]pentan-1-yl (S)-1-(4-fluorophenyl)-3,4-dihydroisoquinoline-2(1H)-carboxylate hydrochloride, Compound 4027

Under an argon atmosphere, palladium on carbon (10% (w/w), spatula tip) was added to a solution of 3-(((benzyloxy)carbonyl)(2-(methylsulfonyl)ethyl)amino)bicyclo[1.1.1]pentan-1-yl (S)-1-(4-fluorophenyl)-3,4-dihydroisoquinoline-2(1H)-carboxylate, 15 (50 mg, 0.084 mmol) in tetrahydrofuran (1 mL) and water (0.5 mL). Hydrogen was applied (balloon) and the mixture was stirred at room temperature for 2 hours. The mixture was filtered over Celite and the filtrate was partially concentrated under reduced pressure. aqueous HCl (2 M, 0.1 mL) was added and the mixture was further concentrated. The residue was purified by acidic preparative MPLC (Linear Gradient: t=0 min 5% A, t=1 min 10% A; t=16 min 50% A; t=17 min 100%; t=22 min 100% A; detection: 220/264 nm). The product fractions were combined and concentrated under reduced pressure. The residue was lyophilized from a mixture of acetonitrile and water (1:1, 4 mL) to give 3-((2-(methylsulfonyl)ethyl)amino)bicyclo[1.1.1]pentan-1-yl (S)-1-(4-fluorophenyl)-3,4-dihydroisoquinoline-2(1H)-carboxylate hydrochloride, compound 4027 (28 mg) as a white amorphous solid. LCMS: 93%, RT=3.20 min., (M+H)⁺=459, (method C). ¹H NMR (400 MHz, chloroform-d) mixture of rotamers δ 7.25-7.07 (m, 5H), 7.06-6.88 (m, 3H), 6.44-6.08 (m, 1H), 4.20-3.85 (m, 1H), 3.76 (t, J=7.2 Hz, 2H), 3.42 (t, J=7.2 Hz, 2H), 3.32-3.15 (m, 1H), 3.11 (s, 3H), 3.06-2.90 (m, 1H), 2.84-2.73 (m, 1H), 2.57 (s, 6H).

### Example 26. Cis-(S)—N-((3-aminocyclobutyl)methyl)-1-(4-fluorophenyl)-3,4-dihydroisoquinoline-2(1H)-carboxamide, Compound 4028

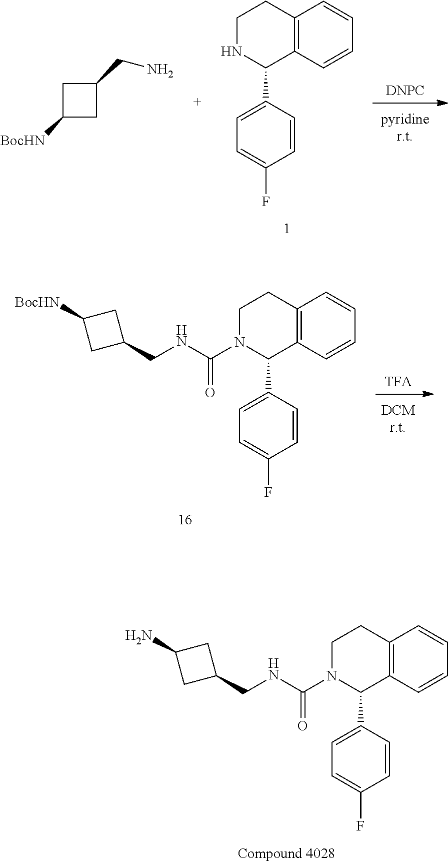

### Step 1. Synthesis of cis-tert-butyl (S)-(3-((1-(4-fluorophenyl)-1,2,3,4-tetrahydroisoquinoline-2-carboxamido)methyl)cyclobutyl)carbamate, 16

Starting from cis-tert-butyl (3-(aminomethyl)cyclobutyl)carbamate (100 mg, 0.499 mmol) and (S)-1-(4-fluorophenyl)-1,2,3,4-tetrahydroisoquinoline, 1 (113 mg, 0.499 mmol), cis-tert-butyl (S)-(3-((1-(4-fluorophenyl)-1,2,3,4-tetrahydroisoquinoline-2-carboxamido)methyl)cyclobutyl)carbamate, 16 (167 mg) was prepared according to the procedure described for 3-(((benzyloxy)carbonyl)amino)bicyclo[1.1.1]pentan-1-yl (S)-1-(4-fluorophenyl)-3,4-dihydroisoquinoline-2(1H)-carboxylate, compound 4001 (see Scheme 1, Step 2). White solid. ¹H NMR (300 MHz, chloroform-d) δ 7.27-7.10 (m, 6H), 7.01-6.90 (m, 2H), 6.36 (s, 1H), 4.58 (br s, 2H), 4.00-3.80 (m, 1H), 3.66-3.46 (m, 2H), 3.27 (dq, J=14.6, 7.6 Hz, 2H), 2.99-2.85 (m, 1H), 2.85-2.71 (m, 1H), 2.46-2.31 (m, 2H), 2.19-2.01 (m, 1H), 1.66-1.44 (m, 2H), 1.43 (s, 9H).

### Step 2. Synthesis of cis-(S)—N-((3-aminocyclobutyl)methyl)-1-(4-fluorophenyl)-3,4-dihydroisoquinoline-2(1H)-carboxamide, Compound 4028

Starting from cis-tert-butyl (S)-(3-((1-(4-fluorophenyl)-1,2,3,4-tetrahydroisoquinoline-2-carboxamido)methyl)cyclobutyl)carbamate, 16 (167 mg, 0.368 mmol), cis-(S)—N-((3-aminocyclobutyl)methyl)-1-(4-fluorophenyl)-3,4-dihydroisoquinoline-2(1H)-carboxamide, compound 4028 (28 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (compound 4005) and purified by flash column chromatography (silica, eluting with 0 to 20% methanol in dichloromethane) after basic workup (aqueous NaOH (2 M)/dichloromethane extraction).

LCMS: 96.5%, RT=1.23 min., (M+H)⁺=354 (method E). ¹H NMR (300 MHz, chloroform-d) δ 7.27-7.09 (m, 6H), 7.01-6.88 (m, 2H), 6.37 (s, 1H), 4.79 (t, J=5.2 Hz, 1H), 3.67-3.44 (m, 2H), 3.37-3.15 (m, 3H), 2.97-2.70 (m, 2H), 2.48-2.29 (m, 2H), 2.17-1.96 (m, 1H), 1.40-1.22 (m, 2H).

### Example 27. Trans-(S)—N-((3-aminocyclobutyl)methyl)-1-(4-fluorophenyl)-3,4-dihydroisoquinoline-2(1H)-carboxamide, Compound 4029

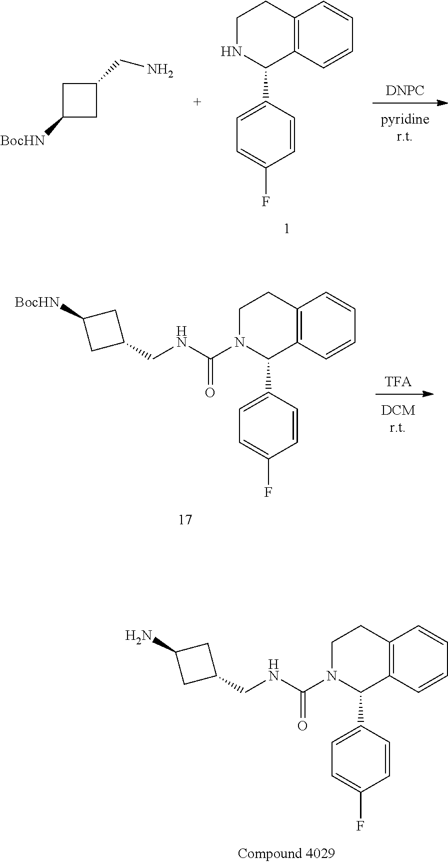

### Step 1. Synthesis of trans-tert-butyl (S)-(3-((1-(4-fluorophenyl)-1,2,3,4-tetrahydroisoquinoline-2-carboxamido)methyl)cyclobutyl)carbamate, 17

Starting from trans-tert-butyl (3-(aminomethyl)cyclobutyl)carbamate (100 mg, 0.499 mmol) and (S)-1-(4-fluorophenyl)-1,2,3,4-tetrahydroisoquinoline, 1 (113 mg, 0.499 mmol), trans-tert-butyl (S)-(3-((1-(4-fluorophenyl)-1,2,3,4-tetrahydroisoquinoline-2-carboxamido)methyl)cyclobutyl)carbamate, 17 (160 mg) was prepared according to the procedure described for 3-(((benzyloxy)carbonyl)amino)bicyclo[1.1.1]pentan-1-yl (S)-1-(4-fluorophenyl)-3,4-dihydroisoquinoline-2(1H)-carboxylate, compound 4001 (see Scheme 1, Step 2). Light yellow solid. ¹H NMR (300 MHz, chloroform-d) δ 7.27-7.10 (m, 6H), 7.03-6.89 (m, 2H), 6.35 (s, 1H), 4.76-4.57 (m, 1H), 4.56-4.45 (m, 1H), 4.25-4.05 (m, 1H), 3.54 (t, J=6.2 Hz, 2H), 3.44-3.22 (m, 2H), 2.98-2.71 (m, 2H), 2.44-2.28 (m, 1H), 2.15-2.01 (m, 2H), 2.00-1.86 (m, 2H), 1.43 (s, 9H).

### Step 2. Synthesis of trans-(S)—N-((3-aminocyclobutyl)methyl)-1-(4-fluorophenyl)-3,4-dihydroisoquinoline-2(1H)-carboxamide, Compound 4029

Starting from trans-tert-butyl (S)-(3-((1-(4-fluorophenyl)-1,2,3,4-tetrahydroisoquinoline-2-carboxamido)methyl)cyclobutyl)carbamate, 17 (160 mg, 0.353 mmol), trans-(S)—N-((3-aminocyclobutyl)methyl)-1-(4-fluorophenyl)-3,4-dihydroisoquinoline-2(1H)-carboxamide, compound 4029 (39.6 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (compound 4005) and purified by flash column chromatography (silica, eluting with 0 to 20% methanol in dichloromethane) after basic workup (aqueous NaOH (2 M)/dichloromethane extraction). White solid. LCMS: 96.5%, RT=1.22 min., (M+H)⁺=354 (method E). ¹H NMR (300 MHz, chloroform-d) δ 7.27-7.10 (m, 6H), 7.01-6.89 (m, 2H), 6.36 (s, 1H), 4.51-4.41 (m, 1H), 3.60-3.45 (m, 3H), 3.42-3.24 (m, 2H), 2.96-2.72 (m, 2H), 2.38-2.22 (m, 1H), 2.10-1.95 (m, 2H), 1.86-1.71 (m, 2H).

### Example 28. Cis-3-(aminomethyl)cyclobutyl (S)-1-(4-fluorophenyl)-3,4-dihydroisoquinoline-2(1H)-carboxylate, Compound 4030

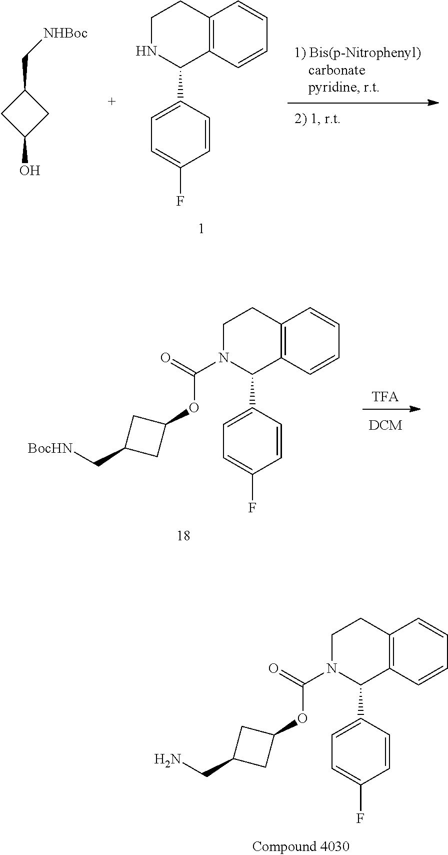

### Step 1. Synthesis of cis-3-(((tert-butoxycarbonyl)amino)methyl)cyclobutyl (S)-1-(4-fluorophenyl)-3,4-dihydroisoquinoline-2(1H)-carboxylate, 18

Starting from tert-butyl ((cis-3-hydroxycyclobutyl)methyl)carbamate (100 mg, 0.497 mmol) and (S)-1-(4-fluorophenyl)-1,2,3,4-tetrahydroisoquinoline, 1 (113 mg, 0.497 mmol), cis-3-(((tert-butoxycarbonyl)amino)methyl)cyclobutyl (S)-1-(4-fluorophenyl)-3,4-dihydroisoquinoline-2(1H)-carboxylate, 18 (152 mg) as a 5:3 mixture with 4-nitrophenyl (S)-1-(4-fluorophenyl)-3,4-dihydroisoquinoline-2(1H)-carboxylate was prepared according to the procedure described for 3-(((benzyloxy)carbonyl)amino)bicyclo[1.1.1]pentan-1-yl (S)-1-(4-fluorophenyl)-3,4-dihydroisoquinoline-2(1H)-carboxylate (see Scheme 1) and purified by acidic preparative MPLC (Linear Gradient: t=0 min 5% A, t=1 min 5% A; t=2 min 30%, t=17 min 70% A; t=18 min 100%; t=23 min 100% A; detection: 210/220 nm). Solid. LCMS: RT=2.264 min., (M+Na)⁺=477 (Method A).

### Step 2. Synthesis of cis-3-(aminomethyl)cyclobutyl (S)-1-(4-fluorophenyl)-3,4-dihydroisoquinoline-2(1H)-carboxylate, Compound 4030

Starting from a 5:3 mixture of cis-3-(((tert-butoxycarbonyl)amino)methyl)cyclobutyl (S)-1-(4-fluorophenyl)-3,4-dihydroisoquinoline-2(1H)-carboxylate, 18 (152 mg, 60% (w/w), 0.20 mmol) and 4-nitrophenyl (S)-1-(4-fluorophenyl)-3,4-dihydroisoquinoline-2(1H)-carboxylate, cis-3-(aminomethyl)cyclobutyl (S)-1-(4-fluorophenyl)-3,4-dihydroisoquinoline-2(1H)-carboxylate, compound 4030 (37 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by basic preparative MPLC (Linear Gradient: t=0 min 5% A, t=1 min 5% A; t=2 min 20%, t=17 min 60% A; t=18 min 100%; t=23 min 100% A; detection: 210/220/285 nm) after basic workup (saturated aqueous NaHCO₃/dichloromethane extraction). Sticky solid. LCMS: 100%, RT=2,783 min., (M+H)⁺=355 (Method D). ¹H NMR (400 MHz, chloroform-d) mixture of rotamers δ 7.25-7.11 (m, 5H), 7.01 (d, J=7.5 Hz, 1H), 6.96 (t, J=8.7 Hz, 2H), 6.49-6.14 (m, 1H), 5.00-4.87 (m, 1H), 4.24-3.87 (m, 1H), 3.27-3.09 (m, 1H), 3.07-2.89 (m, 1H), 2.83-2.67 (m, 3H), 2.59-2.43 (m, 2H), 2.04-1.87 (m, 1H), 1.82-1.63 (m, 2H).

### Example 29. Trans-3-(aminomethyl)cyclobutyl (S)-1-(4-fluorophenyl)-3,4-dihydroisoquinoline-2(1H)-carboxylate, Compound 4031

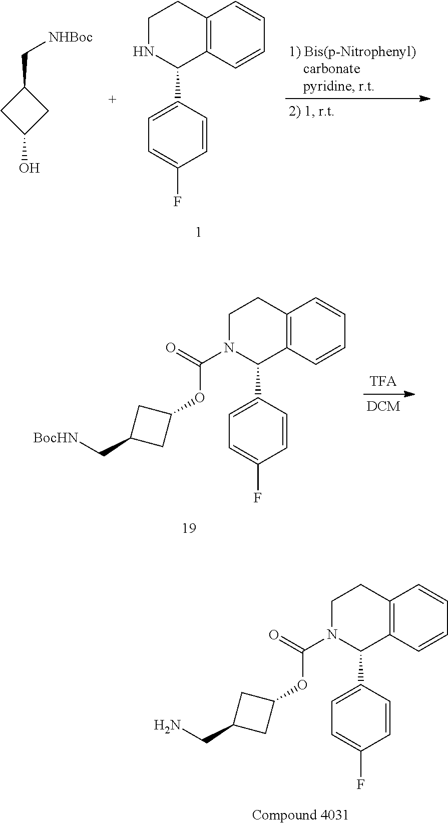 [SMILES: CC(C)(C)OC(=O)NC[C@H]1C[C@H](O)C1 ; Fc1ccc([C@@H]2NCCc3ccccc32)cc1 ; CC(C)(C)OC(=O)NC[C@H]1C[C@H](OC(=O)N2CCc3ccccc3[C@@H]2c2ccc(F)cc2)C1 ; NC[C@H]1C[C@H](OC(=O)N2CCc3ccccc3[C@@H]2c2ccc(F)cc2)C1]

### Step 1. Synthesis of trans-3-(((tert-butoxycarbonyl)amino)methyl)cyclobutyl (S)-1-(4-fluorophenyl)-3,4-dihydroisoquinoline-2(1H)-carboxylate, 19

Starting from tert-butyl ((trans-3-hydroxycyclobutyl)methyl)carbamate (50 mg, 0.248 mmol) and (S)-1-(4-fluorophenyl)-1,2,3,4-tetrahydroisoquinoline, 1 (56.5 mg, 0.248 mmol, trans-3-(((tert-butoxycarbonyl)amino)methyl)cyclobutyl (S)-1-(4-fluorophenyl)-3,4-dihydroisoquinoline-2(1H)-carboxylate, 19 (76 mg, 50% (w/w)) as a mixture of about 1:1 with 4-nitrophenyl (S)-1-(4-fluorophenyl)-3,4-dihydroisoquinoline-2(1H)-carboxylate was prepared according to the procedure described for 3-(((benzyloxy)carbonyl)amino)bicyclo[1.1.1]pentan-1-yl (S)-1-(4-fluorophenyl)-3,4-dihydroisoquinoline-2(1H)-carboxylate (see Scheme 1) and purified by acidic preparative MPLC (Linear Gradient: t=0 min 5% A, t=1 min 5% A; t=2 min 20%, t=17 min 60% A; t=18 min 100%; t=26 min 100% A; detection: 210/220 nm). White solid. LCMS: RT=2.266 min., (M+Na)⁺=477 (method A).

### Step 2. Synthesis of (trans)-3-(aminomethyl)cyclobutyl (S)-1-(4-fluorophenyl)-3,4-dihydroisoquinoline-2(1H)-carboxylate, Compound 4031

Starting from a mixture of about 1:1 of (S)-1-(4-fluorophenyl)-3,4-dihydroisoquinoline-2(1H)-carboxylate, 19 (76 mg, 50% (w/w), 0.084 mmol) and 4-nitrophenyl (S)-1-(4-fluorophenyl)-3,4-dihydroisoquinoline-2(1H)-carboxylate, (trans)-3-(aminomethyl)cyclobutyl (S)-1-(4-fluorophenyl)-3,4-dihydroisoquinoline-2(1H)-carboxylate, compound 4031 (8 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by basic preparative MPLC (Linear Gradient: t=0 min 5% A, t=1 min 5% A; t=2 min 20%, t=17 min 60% A; t=18 min 100%; t=23 min 100% A; detection: 210/220/285 nm) after basic workup (saturated aqueous NaHCO₃/dichloromethane extraction). Sticky solid. LCMS: 98%, RT=2.810 min., (M+H)⁺=355 (Method C). ¹H NMR (400 MHz, chloroform-d) mixture of rotamers δ 7.24-7.12 (m, 5H), 7.02 (d, J=7.4 Hz, 1H), 6.96 (t, J=8.5 Hz, 2H), 6.49-6.15 (m, 1H), 5.15-5.01 (m, 1H), 4.22-3.90 (m, 1H), 3.29-3.10 (m, 1H), 3.06-2.91 (m, 1H), 2.86-2.67 (m, 3H), 2.38-2.09 (m, 5H).

### Example 30. (S)—N-(cis-3-(aminomethyl)cyclobutyl)-1-(4-fluorophenyl)-3,4-dihydroisoquinoline-2(1H)-carboxamide, Compound 4032

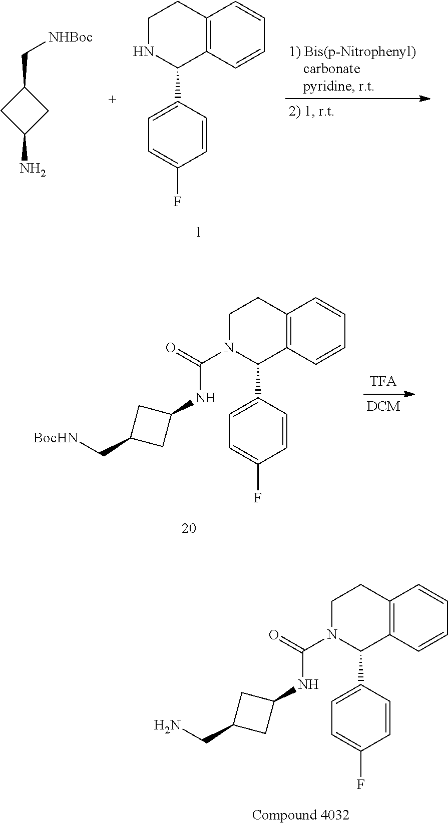 [SMILES: CC(C)(C)OC(=O)NC[C@H]1C[C@@H](N)C1 ; Fc1ccc([C@@H]2NCCc3ccccc32)cc1 ; CC(C)(C)OC(=O)NC[C@H]1C[C@@H](NC(=O)N2CCc3ccccc3[C@@H]2c2ccc(F)cc2)C1 ; NC[C@H]1C[C@@H](NC(=O)N2CCc3ccccc3[C@@H]2c2ccc(F)cc2)C1]

### Step 1. Synthesis of tert-butyl ((cis-3-((S)-1-(4-fluorophenyl)-1,2,3,4-tetrahydroisoquinoline-2-carboxamido)cyclobutyl)methyl)carbamate, 20

Starting from tert-butyl ((cis-3-aminocyclobutyl)methyl)carbamate (50 mg, 0.250 mmol), ((cis-3-((S)-1-(4-fluorophenyl)-1,2,3,4-tetrahydroisoquinoline-2-carboxamido)cyclobutyl)methyl)carbamate, 20 (82 mg) was prepared according to the procedure described for 3-(((benzyloxy)carbonyl)amino)bicyclo[1.1.1]pentan-1-yl (S)-1-(4-fluorophenyl)-3,4-dihydroisoquinoline-2(1H)-carboxylate (see Scheme 1) and purified by acidic preparative MPLC (Linear Gradient: t=0 min 5% A, t=1 min 5% A; t=2 min 30%, t=17 min 70% A; t=18 min 100%; t=23 min 100% A; detection: 210/220 nm). White powder. LCMS: 100%, RT=2.115 min., (M+Na)⁺=476 (Method A). ¹H NMR (400 MHz, chloroform-d) δ 7.27-7.13 (m, 6H), 6.97 (m, 2H), 6.43 (s, 1H), 4.73-4.58 (m, 1H), 4.58-4.44 (m, 1H), 4.31-4.16 (m, 1H), 3.64-3.45 (m, 2H), 3.27-3.03 (m, 2H), 2.99-2.87 (m, 1H), 2.85-2.72 (m, 1H), 2.61-2.41 (m, 2H), 2.25-2.02 (m, 1H), 1.63-1.49 (m, 2H), 1.46 (s, 9H).

### Step 2. Synthesis of (S)—N-(cis-3-(aminomethyl)cyclobutyl)-1-(4-fluorophenyl)-3,4-dihydroisoquinoline-2(1H)-carboxamide, Compound 4032

Starting from tert-butyl ((cis-3-((S)-1-(4-fluorophenyl)-1,2,3,4-tetrahydroisoquinoline-2-carboxamido)cyclobutyl)methyl)carbamate, 20 (82 mg, 0.181 mmol) (S)—N-(cis-3-(aminomethyl)cyclobutyl)-1-(4-fluorophenyl)-3,4-dihydroisoquinoline-2(1H)-carboxamide, compound 4032 (37 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by SCX-2 ion exchange chromatography and acidic preparative MPLC (Linear Gradient: t=0 min 5% A, t=1 min 5% A; t=2 min 10%, t=17 min 50% A; t=18 min 100%; t=23 min 100% A; detection: 210/220/270 nm), after SCX-2 ion exchange chromatography. Yellow powder. LCMS: 100%, RT=2.710 min., (M+H)⁺=354 (Method C). ¹H NMR (400 MHz, chloroform-d) δ 7.25-7.08 (m, 6H), 6.99-6.88 (m, 2H), 6.40 (s, 1H), 4.67 (br s, 1H), 4.30-4.16 (m, 1H), 3.59-3.44 (m, 2H), 2.96-2.84 (m, 1H), 2.83-2.71 (m, 1H), 2.68 (d, J=6.6 Hz, 2H), 2.56-2.42 (m, 2H), 2.09-1.96 (m, 1H), 1.55-1.35 (m, 2H).

### Example 31. (1-Aminocyclopropyl)methyl (S)-1-(4-fluorophenyl)-3,4-dihydroisoquinoline-2(1H)-carboxylate hydrochloride, Compound 4033

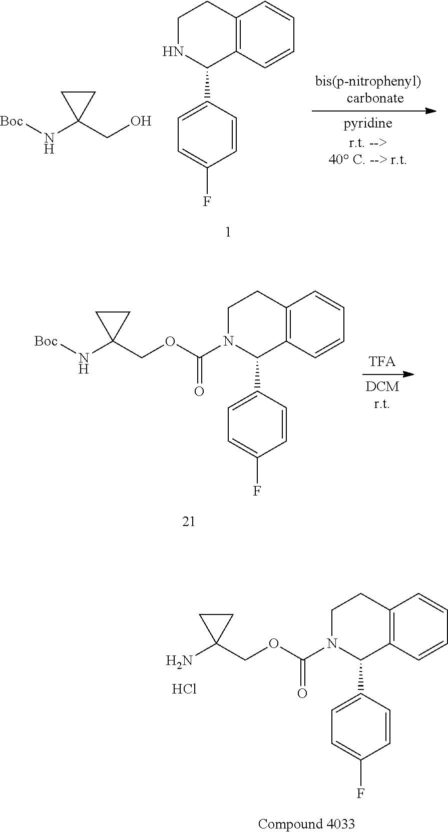

### Step 1. Synthesis of (1-((tert-butoxycarbonyl)amino)cyclopropyl)methyl (S)-1-(4-fluorophenyl)-3,4-dihydroisoquinoline-2(1H)-carboxylate, 21

Starting from tert-butyl (1-(hydroxymethyl)cyclopropyl)carbamate (50 mg, 0.267 mmol) and (S)-1-(4-fluorophenyl)-1,2,3,4-tetrahydroisoquinoline (66.8 mg, 0.294 mmol), (1-((tert-butoxycarbonyl)amino)cyclopropyl)methyl (S)-1-(4-fluorophenyl)-3,4-dihydroisoquinoline-2(1H)-carboxylate (55 mg) was prepared according to the procedure described for 3-(((benzyloxy)carbonyl)amino)bicyclo[1.1.1]pentan-1-yl (S)-1-(4-fluorophenyl)-3,4-dihydroisoquinoline-2(1H)-carboxylate (see Scheme 1) and purified by flash column chromatography (silica, eluting with 0 to 75% ethyl acetate in heptane) after basic workup (aqueous NaOH (0.25 M)/dichloromethane extraction). Colorless oil. LCMS: 94.7%, RT=2.280 min., (M-Boc+H)⁺=341 (Method B). ¹H NMR (400 MHz, chloroform-d) mixture of rotamers δ 7.25-7.12 (m, 5H), 7.07-6.91 (m, 3H), 6.51-6.19 (m, 1H), 5.06-4.65 (m, 1H), 4.24-3.98 (m, 3H), 3.26 (ddd, J=13.4, 10.7, 4.3 Hz, 1H), 3.09-2.94 (m, 1H), 2.87-2.71 (m, 1H), 1.43 (s, 9H), 0.93-0.77 (m, 4H).

### Step 2. Synthesis of (1-aminocyclopropyl)methyl (S)-1-(4-fluorophenyl)-3,4-dihydroisoquinoline-2(1H)-carboxylate hydrochloride, Compound 4033

Starting from (1-((tert-butoxycarbonyl)amino)cyclopropyl)methyl (S)-1-(4-fluorophenyl)-3,4-dihydroisoquinoline-2(1H)-carboxylate, 21 (55 mg, 0.125 mmol), (1-aminocyclopropyl)methyl (S)-1-(4-fluorophenyl)-3,4-dihydroisoquinoline-2(1H)-carboxylate hydrochloride, compound 4033 (22.1 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by basic preparative MPLC (Linear Gradient: t=0 min 20% A, t=1 min 20% A; t=16 min 60% A; t=17 min 100% A; t=22 min 100% A; detection: 220 nm). The residue was lyophilized from a mixture of acetonitrile and water (1:2, 3 mL), and aqueous HCl (1.00 M, 100 μL, 0.100 mmol). White solid. LCMS: 99.1a %, RT=1.656 min., (M+H)⁺=341 (Method C). ¹H NMR (400 MHz, DMSO-d₆) mixture of rotamers δ 8.55 (br s, 3H), 7.36-6.96 (m, 8H), 6.66-6.21 (m, 1H), 4.46-3.82 (m, 3H), 3.28-3.12 (m, 1H), 3.03-2.70 (m, 2H), 1.17-0.97 (m, 2H), 0.95-0.79 (m, 2H).

### Example 32. (S)—N-(cis-3-aminocyclobutyl)-1-(4-fluorophenyl)-3,4-dihydroisoquinoline-2(1H)-carboxamide, Compound 4034

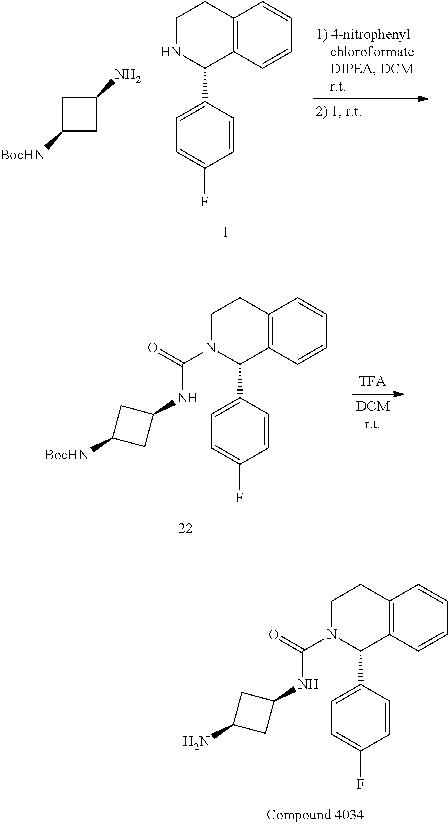 [SMILES: CC(C)(C)OC(=O)N[C@H]1C[C@@H](N)C1 ; Fc1ccc([C@@H]2NCCc3ccccc32)cc1 ; CC(C)(C)OC(=O)N[C@H]1C[C@@H](NC(=O)N2CCc3ccccc3[C@@H]2c2ccc(F)cc2)C1 ; N[C@H]1C[C@@H](NC(=O)N2CCc3ccccc3[C@@H]2c2ccc(F)cc2)C1]

### Step 1. Synthesis of tert-butyl (cis-3-((S)-1-(4-fluorophenyl)-1,2,3,4-tetrahydroisoquinoline-2-carboxamido)cyclobutyl)carbamate, 22

4-Nitrophenyl chloroformate (108 mg, 0.537 mmol) was added to a solution of tert-butyl (cis-3-aminocyclobutyl)carbamate (100 mg, 0.537 mmol) and pyridine (0.130 mL, 1.61 mmol) in dichloromethane (2.5 mL). After the reaction mixture was stirred for 1 hour at room temperature, N,N-diisopropylethylamine (0.122 mL, 0.698 mmol) was added. After another 30 minutes, (S)-1-(4-fluorophenyl)-1,2,3,4-tetrahydroisoquinoline (146 mg, 0.644 mmol) was added and the reaction mixture was stirred for 2 days. The reaction mixture was diluted with dichloromethane (10 mL), washed with aqueous HCl (0.5 M, 3×10 mL), a mixture of water and saturated aqueous K₂CO₃ (1:1, 10 mL), and brine (10 mL). The organic fraction was dried on Na₂SO₄ and evaporated under reduced pressure to give tert-butyl (cis-3-((S)-1-(4-fluorophenyl)-1,2,3,4-tetrahydroisoquinoline-2-carboxamido)cyclobutyl)carbamate, 22 (202 mg) as a yellow solid. LCMS: 98%, RT=2.079 min., (M+H)⁺=440 (Method A). ¹H NMR (400 MHz, chloroform-d) δ 7.25-7.11 (m, 6H), 6.99-6.89 (m, 2H), 6.38 (s, 1H), 4.78-4.56 (m, 2H), 4.07-3.91 (m, 1H), 3.86-3.66 (m, 1H), 3.59-3.45 (m, 2H), 2.96-2.86 (m, 1H), 2.86-2.72 (m, 3H), 1.91-1.67 (m, 2H), 1.43 (s, 9H).

### Step 2. Synthesis of (S)—N-(cis-3-aminocyclobutyl)-1-(4-fluorophenyl)-3,4-dihydroisoquinoline-2(1H)-carboxamide, Compound 4034

Starting from tert-butyl (cis-3-((S)-1-(4-fluorophenyl)-1,2,3,4-tetrahydroisoquinoline-2-carboxamido)cyclobutyl)carbamate, 22 (0.20 g, 0.46 mmol), (S)—N-(cis-3-aminocyclobutyl)-1-(4-fluorophenyl)-3,4-dihydroisoquinoline-2(1H)-carboxamide, compound 4034 (94 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by basic preparative MPLC (Linear Gradient: t=0 min 5% A, t=1 min 5% A; t=2 min 20% A; t=17 min 60% A; t=18 min 100% A; t=23 min 100% A; detection 210/256 nm) after basic workup (saturated aqueous NaHCO₃/dichloromethane extraction). Pale brown solid. LCMS: 99%, RT=2.524 min., (M+H)⁺=340 (Method C). ¹H NMR (400 MHz, chloroform-d) δ 7.26-7.12 (m, 6H), 7.00-6.90 (m, 2H), 6.39 (s, 1H), 4.60 (d, J=7.3 Hz, 1H), 4.02-3.89 (m, 1H), 3.58-3.45 (m, 2H), 3.23-3.12 (m, 1H), 2.96-2.85 (m, 1H), 2.83-2.69 (m, 3H), 1.59-1.39 (m, 2H).

### Example 33. (S)—N-(trans-3-aminocyclobutyl)-1-(4-fluorophenyl)-3,4-dihydroisoquinoline-2(1H)-carboxamide hydrochloride, Compound 4035

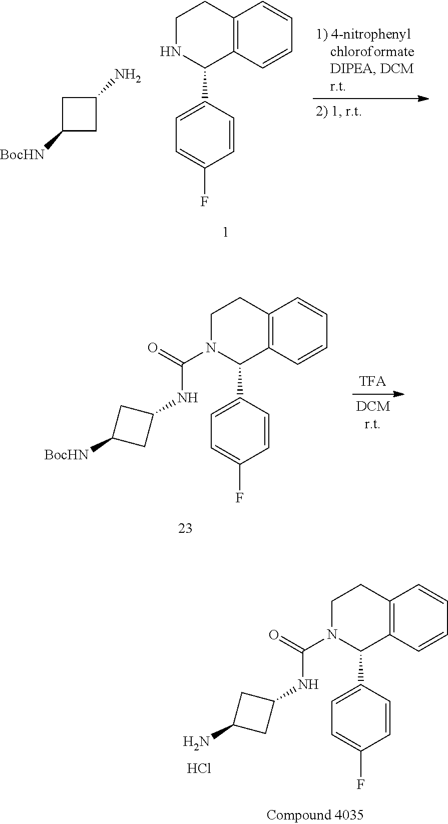 [SMILES: CC(C)(C)OC(=O)N[C@H]1C[C@H](N)C1 ; Fc1ccc([C@@H]2NCCc3ccccc32)cc1 ; CC(C)(C)OC(=O)N[C@H]1C[C@H](NC(=O)N2CCc3ccccc3[C@@H]2c2ccc(F)cc2)C1 ; Cl ; N[C@H]1C[C@H](NC(=O)N2CCc3ccccc3[C@@H]2c2ccc(F)cc2)C1]

### Step 1. Synthesis of tert-butyl (trans-3-((S)-1-(4-fluorophenyl)-1,2,3,4-tetrahydroisoquinoline-2-carboxamido)cyclobutyl)carbamate, 23

Starting from tert-butyl (trans-3-aminocyclobutyl)carbamate (100 mg, 0.537 mmol), tert-butyl (trans-3-((S)-1-(4-fluorophenyl)-1,2,3,4-tetrahydroisoquinoline-2-carboxamido)cyclobutyl)carbamate (144 mg) was prepared according to the procedure described for tert-butyl (cis-3-((S)-1-(4-fluorophenyl)-1,2,3,4-tetrahydroisoquinoline-2-carboxamido)cyclobutyl)carbamate (see Scheme 32) and purified by acidic preparative MPLC (Linear Gradient: t=0 min 5% A, t=1 min 5% A; t=2 min 20% A; t=17 min 60% A; t=18 min 100% A; t=26 min 100% A; detection 220/270 nm). White solid. LCMS: 99%, RT=4.024 min., (M+H)⁺=440 (Method C). ¹H NMR (400 MHz, chloroform-d) δ 7.25-7.11 (m, 6H), 6.99-6.90 (m, 2H), 6.38 (s, 1H), 4.84-4.74 (m, 1H), 4.73-4.63 (m, 1H), 4.39-4.27 (m, 1H), 4.25-4.12 (m, 1H), 3.61-3.46 (m, 2H), 2.91 (dt, J=15.8, 6.5 Hz, 1H), 2.78 (dt, J=15.8, 5.9 Hz, 1H), 2.35-2.13 (m, 4H), 1.44 (s, 9H).

### Step 2. Synthesis of (S)—N-(trans-3-aminocyclobutyl)-1-(4-fluorophenyl)-3,4-dihydroisoquinoline-2(1H)-carboxamide hydrochloride, Compound 4035

Starting from tert-butyl (trans-3-((S)-1-(4-fluorophenyl)-1,2,3,4-tetrahydroisoquinoline-2-carboxamido)cyclobutyl)carbamate, 23 (0.14 g, 0.32 mmol), (S)—N-(trans-3-aminocyclobutyl)-1-(4-fluorophenyl)-3,4-dihydroisoquinoline-2(1H)-carboxamide hydrochloride, compound 4035 (28 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, compound 4005 (see Scheme 5) and purified by basic preparative MPLC (Linear Gradient: t=0 min 5% A, t=1 min 5% A; t=2 min 20% A; t=17 min 60% A; t=18 min 100% A; t=23 min 100% A; detection 220/280 nm). The residue was lyophilized from a mixture of acetonitrile (1 mL) and aqueous HCl (0.5 M, 0.3 mL). Yellow solid. LCMS: 99%, RT=2.593 min., (M+H)⁺=340 (method C). ¹H NMR (400 MHz, methanol-d₄) δ 7.30-7.09 (m, 6H), 7.05-6.92 (m, 2H), 6.44 (s, 1H), 4.56-4.41 (m, 1H), 3.92-3.78 (m, 1H), 3.78-3.63 (m, 1H), 3.41 (ddd, J=13.4, 8.7, 5.0 Hz, 1H), 2.94 (ddd, J=14.9, 8.8, 5.7 Hz, 1H), 2.75 (dt, J=16.0, 5.3 Hz, 1H), 2.58-2.41 (m, 4H).

### Example 34. Trans-3-aminocyclobutyl (S)-1-(4-fluorophenyl)-3,4-dihydroisoquinoline-2(1H)-carboxylate hydrochloride, Compound 4036

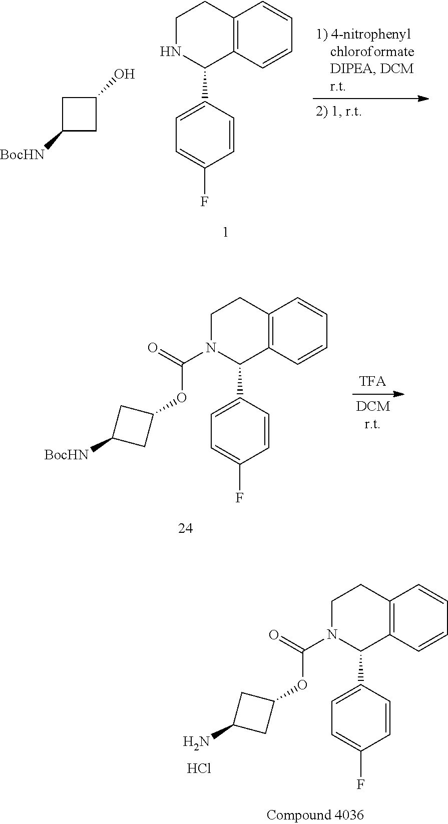 [SMILES: CC(C)(C)OC(=O)N[C@H]1C[C@H](O)C1 ; Fc1ccc([C@@H]2NCCc3ccccc32)cc1 ; CC(C)(C)OC(=O)N[C@H]1C[C@H](OC(=O)N2CCc3ccccc3[C@@H]2c2ccc(F)cc2)C1 ; Cl ; N[C@H]1C[C@H](OC(=O)N2CCc3ccccc3[C@@H]2c2ccc(F)cc2)C1]

### Step 1. Synthesis of trans-3-((tert-butoxycarbonyl)amino)cyclobutyl (S)-1-(4-fluorophenyl)-3,4-dihydroisoquinoline-2(1H)-carboxylate, 24

Starting from tert-butyl (trans-3-hydroxycyclobutyl)carbamate (100 mg, 0.534 mmol) and (S)-1-(4-fluorophenyl)-1,2,3,4-tetrahydroisoquinoline, 1 (146 mg, 0.641 mmol), trans-3-((tert-butoxycarbonyl)amino)cyclobutyl (S)-1-(4-fluorophenyl)-3,4-dihydroisoquinoline-2(1H)-carboxylate, 24 (130 mg) was prepared according to the procedure described for tert-butyl (cis-3-((S)-1-(4-fluorophenyl)-1,2,3,4-tetrahydroisoquinoline-2-carboxamido)cyclobutyl)carbamate (see Scheme 32) and purified by acidic preparative MPLC (Linear Gradient: t=0 min 5% A, t=1 min 5% A; t=2 min 30% A; t=25 min 70% A; t=26 min 100% A; t=31 min 100% A; detection 220/270 nm). White solid. LCMS: 99%, RT=4.547 min., (M+Na)⁺=463 (Method C). ¹H NMR (400 MHz, chloroform-d) mixture of rotamers δ 7.26-7.10 (m, 5H), 7.08-6.85 (m, 3H), 6.51-6.09 (m, 1H), 5.24-5.02 (m, 1H), 5.00-4.74 (m, 1H), 4.43-3.87 (m, 2H), 3.31-3.12 (m, 1H), 3.07-2.90 (m, 1H), 2.77 (dt, J=16.2, 3.9 Hz, 1H), 2.48 (s, 2H), 2.39-2.25 (m, 2H), 1.44 (s, 9H).

### Step 2. Synthesis of trans-3-aminocyclobutyl (S)-1-(4-fluorophenyl)-3,4-dihydroisoquinoline-2(1H)-carboxylate hydrochloride, Compound 4036

Starting from (S)-1-(4-fluorophenyl)-3,4-dihydroisoquinoline-2(1H)-carboxylate, 24 (0.12 g, 0.28 mmol), trans-3-aminocyclobutyl (S)-1-(4-fluorophenyl)-3,4-dihydroisoquinoline-2(1H)-carboxylate hydrochloride, compound 4036 (57 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, compound 4005 (See Scheme 5), purified by acidic preparative MPLC (Linear Gradient: t=0 min 5% A, t=1 min 5% A; t=16 min 40% A; t=17 min 100% A; t=23 min 100% A; detection 220/280 nm) and SCX-2 (1 g) ion exchange chromatography, and lyophilized from a mixture of acetonitrile (0.5 mL) and aqueous HCl (0.5 M, 0.5 mL). White solid. LCMS: 99%, RT=2.734 min., (M+H)⁺=341 (Method C). ¹H NMR (400 MHz, methanol-d₄) mixture of rotamers δ 7.31-7.14 (m, 5H), 7.10-6.95 (m, 3H), 6.31 (s, 1H), 5.24-5.14 (m, 1H), 4.01 (dt, J=13.3, 4.9 Hz, 1H), 3.97-3.85 (m, 1H), 3.40-3.33 (m, 1H), 2.96 (ddd, J=16.2, 10.1, 5.7 Hz, 1H), 2.89-2.78 (m, 1H), 2.68-2.51 (m, 4H).

### Example 35. (1R,2S)-2-aminocyclobutyl (S)-1-(4-fluorophenyl)-3,4-dihydroisoquinoline-2(1H)-carboxylate, Compound 4037, and (1S,2R)-2-aminocyclobutyl (S)-1-(4-fluorophenyl)-3,4-dihydroisoquinoline-2(1H)-carboxylate, Compound 4038

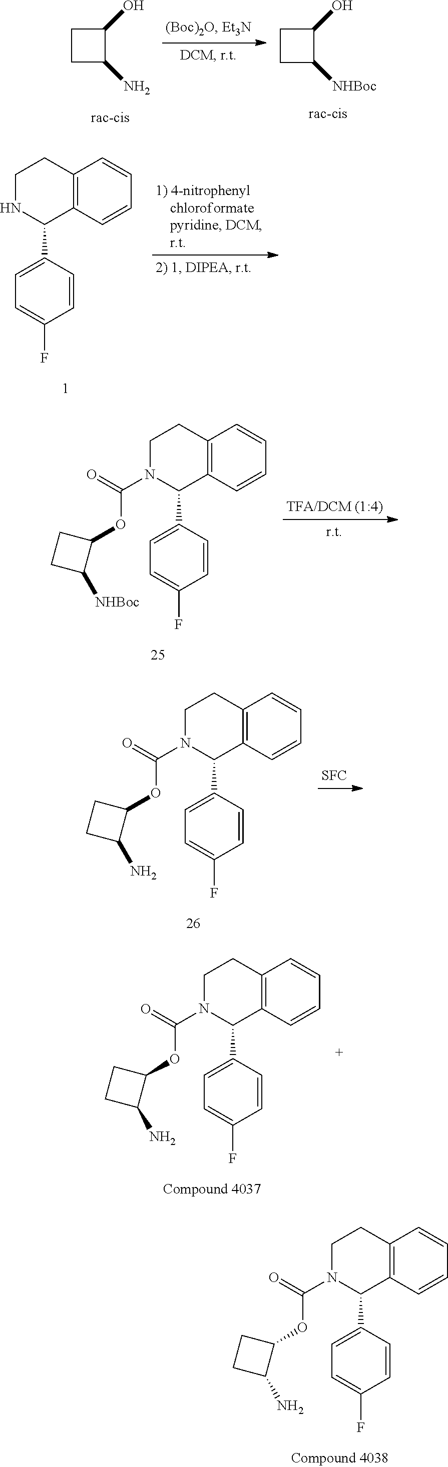

### Step 1. Synthesis of racemic tert-butyl (cis-2-hydroxycyclobutyl)carbamate

Di-tert-butyl dicarbonate (0.951 mL, 4.10 mmol) was added to a suspension of cis-2-aminocyclobutan-1-ol hydrochloride (250 mg, 2.02 mmol) and triethylamine (0.571 mL, 4.10 mmol) in dichloromethane (2.5 mL). The reaction mixture was stirred at room temperature. After 6 hours, a small amount of dichloromethane was added until the reaction mixture was mostly clear. The solution was then left to stir overnight. The next day, water was added and the organic layer was collected and evaporated under reduced pressure. The residue was purified by flash column chromatography (silica, eluting with 0 to 100% ethyl acetate in heptane) to give tert-butyl (cis-2-hydroxycyclobutyl)carbamate (259 mg) as a colorless solid. ¹H NMR (400 MHz, chloroform-d) δ 5.15 (s, 1H), 4.42 (s, 1H), 4.09 (s, 1H), 2.65-2.49 (m, 1H), 2.24-2.04 (m, 2H), 2.02-1.76 (m, 2H), 1.44 (s, 9H).

### Step 2. Synthesis of cis-2-((tert-butoxycarbonyl)amino)cyclobutyl (S)-1-(4-fluorophenyl)-3,4-dihydroisoquinoline-2(1H)-carboxylate, 25

Starting from racemic tert-butyl cis-2-hydroxycyclobutyl)carbamate (100 mg, 0.534 mmol) and (S)-1-(4-fluorophenyl)-1,2,3,4-tetrahydroisoquinoline (146 mg, 0.641 mmol), cis-2-((tert-butoxycarbonyl)amino)cyclobutyl (S)-1-(4-fluorophenyl)-3,4-dihydroisoquinoline-2(1H)-carboxylate, 25 (132 mg) was prepared according to the procedure described for tert-butyl (cis-3-((S)-1-(4-fluorophenyl)-1,2,3,4-tetrahydroisoquinoline-2-carboxamido)cyclobutyl)carbamate (see Scheme 32) and purified by acidic preparative MPLC (Linear Gradient: t=0 min 5% A, t=1 min 5% A; t=2 min 30% A; t=27 min 70% A; t=28 min 100% A; t=33 min 100% A; detection 210/277 nm). White solid. LCMS: 98%, RT=2.226 min., (M+Na)⁺=463 (Method A). ¹H NMR (400 MHz, chloroform-d) mixture of rotamers δ 7.30-7.11 (m, 5H), 7.09-6.88 (m, 3H), 6.50-6.06 (m, 1H), 5.30-5.07 (m, 1H), 4.99-4.43 (m, 1H), 4.42-3.88 (m, 2H), 3.45-3.20 (m, 1H), 3.12-2.91 (m, 1H), 2.89-2.70 (m, 1H), 2.30-2.12 (m, 2H), 2.08-1.73 (m, 2H), 1.40 (s, 9H).

### Step 3. Synthesis of (1R,2S)-2-aminocyclobutyl (S)-1-(4-fluorophenyl)-3,4-dihydroisoquinoline-2(1H)-carboxylate, compound 4037 and (1S,2R)-2-aminocyclobutyl (S)-1-(4-fluorophenyl)-3,4-dihydroisoquinoline-2(1H)-carboxylate, compound 4038

Starting from cis-2-((tert-butoxycarbonyl)amino)cyclobutyl (S)-1-(4-fluorophenyl)-3,4-dihydroisoquinoline-2(1H)-carboxylate, 25 (0.13 g, 0.30 mmol), (1R,2S)-2-aminocyclobutyl (S)-1-(4-fluorophenyl)-3,4-dihydroisoquinoline-2(1H)-carboxylate, compound 4037 (24 mg) and (1S,2R)-2-aminocyclobutyl (S)-1-(4-fluorophenyl)-3,4-dihydroisoquinoline-2(1H)-carboxylate, compound 4038 (26 mg) were prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate, compound 4005 after basic workup (saturated aqueous NaHCO₃/dichloromethane extraction). The diastereomers were separated by preparative SFC (Method Y).

Compound 4037: First eluting diastereomer. Chiral SFC: 95%, RT=3.285 min., (M+H)⁺=341 (Method X). LCMS: 98%, RT=2.630 min., (M+H)⁺=341 (Method C). ¹H NMR (400 MHz, chloroform-d) mixture of rotamers δ 7.27-7.13 (m, 5H), 7.04 (d, J=7.5 Hz, 1H), 6.96 (t, J=8.5 Hz, 2H), 6.49-6.22 (m, 1H), 5.01 (s, 1H), 4.21-3.98 (m, 1H), 3.82-3.61 (m, 1H), 3.36-3.18 (m, 1H), 3.01 (ddd, J=16.5, 10.7, 5.9 Hz, 1H), 2.80 (dt, J=16.3, 4.0 Hz, 1H), 2.27-2.10 (m, 2H), 2.10-1.95 (m, 1H), 1.82-1.65 (m, 1H).

Compound 4038: Second eluting diastereomer. Chiral SFC: 98%, RT=3.723 min., (M+H)⁺=341 (Method X). LCMS: 98%, RT=2.627 min., (M+H)⁺=341 (Method C). ¹H NMR (400 MHz, chloroform-d) mixture of rotamers δ 7.26-7.11 (m, 5H), 7.10-6.86 (m, 3H), 6.49-6.13 (m, 1H), 5.06-4.92 (m, 1H), 4.37-3.97 (m, 1H), 3.82-3.59 (m, 1H), 3.38-3.22 (m, 1H), 3.01 (ddd, J=16.3, 10.6, 5.8 Hz, 1H), 2.88-2.73 (m, 1H), 2.28-1.95 (m, 3H), 1.83-1.61 (m, 1H).

Absolute stereochemistry of cis-cyclobutyl motif was assigned arbitrarily and therefore may be switched between compounds 4037 and 4038.

### Example 36. (1S,2S)-2-aminocyclobutyl (S)-1-(4-fluorophenyl)-3,4-dihydroisoquinoline-2(1H)-carboxylate hydrochloride, Compound 4039, and (1R,2R)-2-aminocyclobutyl (S)-1-(4-fluorophenyl)-3,4-dihydroisoquinoline-2(1H)-carboxylate hydrochloride, Compound 4040

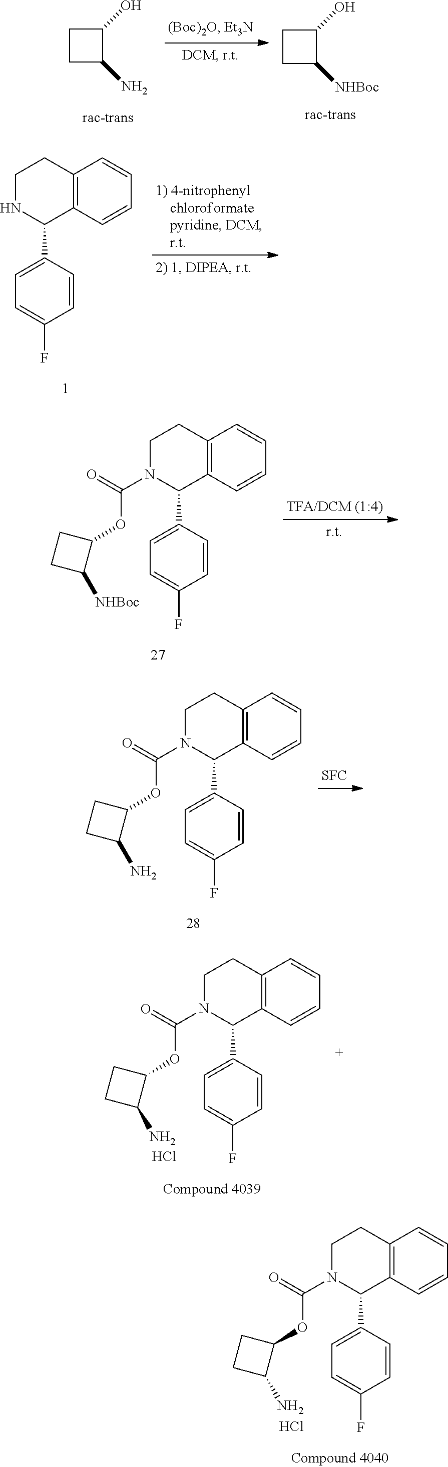

### Step 1. Synthesis of tert-butyl (trans-2-hydroxycyclobutyl)carbamate

Di-tert-butyl dicarbonate (0.951 mL, 4.10 mmol) was added to a suspension of trans-2-aminocyclobutan-1-ol hydrochloride (250 mg, 2.02 mmol) and triethylamine (0.571 mL, 4.10 mmol) in dichloromethane (5.0 mL). The reaction mixture was stirred overnight at room temperature. The next day the mixture was diluted with dichloromethane (15 mL) and water (10 mL). The organic layer was collected and evaporated under reduced pressure. The residue was purified by flash column chromatography (silica, eluting with 0 to 100% ethyl acetate in heptane) to give tert-butyl (trans-2-hydroxycyclobutyl)carbamate (280 mg) as a white solid. ¹H NMR (400 MHz, chloroform-d) δ 4.78 (s, 1H), 4.06-3.87 (m, 1H), 3.78-3.59 (m, 1H), 3.04 (s, 1H), 2.26-1.95 (m, 2H), 1.69-1.61 (m, 1H), 1.44 (s, 9H) 1.36-1.20 (m, 1H).

### Step 2. Synthesis of trans-2-((tert-butoxycarbonyl)amino)cyclobutyl (S)-1-(4-fluorophenyl)-3,4-dihydroisoquinoline-2(1H)-carboxylate, 27

Starting from tert-butyl (trans-2-hydroxycyclobutyl)carbamate (150 mg, 0.801 mmol) and (S)-1-(4-fluorophenyl)-1,2,3,4-tetrahydroisoquinoline, 1 (218 mg, 0.961 mmol), trans-2-((tert-butoxycarbonyl)amino)cyclobutyl (S)-1-(4-fluorophenyl)-3,4-dihydroisoquinoline-2(1H)-carboxylate, 27 (330 mg) was prepared according to the procedure described for tert-butyl (cis-3-((S)-1-(4-fluorophenyl)-1,2,3,4-tetrahydroisoquinoline-2-carboxamido)cyclobutyl)carbamate (see Scheme 32). Yellow oil. LCMS: 92%, RT=2.270 min., (M+Na)⁺=463 (Method A). ¹H NMR (400 MHz, chloroform-d) mixture of rotamers δ 7.34-7.12 (m, 5H), 7.09-6.91 (m, 3H), 6.52-6.18 (m, 1H), 5.38-4.95 (m, 1H), 4.81-4.65 (m, 1H), 4.30-3.79 (m, 2H), 3.31-2.65 (m, 3H), 2.37-1.93 (m, 2H), 1.84-1.54 (m, 2H), 1.52-1.35 (m, 9H).

### Step 3. Synthesis of (1S,2S)-2-aminocyclobutyl (S)-1-(4-fluorophenyl)-3,4-dihydroisoquinoline-2(1H)-carboxylate hydrochloride, Compound 4039, and (1R,2R)-2-aminocyclobutyl (S)-1-(4-fluorophenyl)-3,4-dihydroisoquinoline-2(1H)-carboxylate hydrochloride, Compound 4040

Starting from trans-2-((tert-butoxycarbonyl)amino)cyclobutyl (S)-1-(4-fluorophenyl)-3,4-dihydroisoquinoline-2(1H)-carboxylate, 27 (0.33 g, 0.74 mmol), (1S,2S)-2-aminocyclobutyl (S)-1-(4-fluorophenyl)-3,4-dihydroisoquinoline-2(1H)-carboxylate hydrochloride, Compound 4039 (23 mg) and (1R,2R)-2-aminocyclobutyl (S)-1-(4-fluorophenyl)-3,4-dihydroisoquinoline-2(1H)-carboxylate hydrochloride, Compound 4040 (20 mg) were prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by acidic preparative MPLC (Linear Gradient: t=0 min 5% A, t=1 min 5% A; t=2 min 10% A; t=18 min 41% A; t=20 min 100% A; t=24 min 100% A; detection 210/270 nm). The diastereomers were separated by preparative SFC (Method Z) and lyophilized from a mixture of acetonitrile (0.5 mL) and aqueous HCl (0.5 M, 0.5 mL).

Compound 4039: First eluting diastereomer. Chiral SFC: 98%, RT=2.959 min., (M+H)⁺=341 (Method W). LCMS: 99%, RT=2.831 min., (M+H)⁺=341 (Method C). ¹H NMR (400 MHz, methanol-d₄) mixture of rotamers δ 7.30-7.15 (m, 5H), 7.11-6.96 (m, 3H), 6.47-6.21 (m, 1H), 5.01-4.90 (m, 1H), 4.16-3.93 (m, 1H), 3.85-3.70 (m, 1H), 3.42-3.33 (m, 1H), 3.05-2.91 (m, 1H), 2.91-2.75 (m, 1H), 2.45-2.20 (m, 2H), 2.13-1.94 (m, 1H), 1.85-1.67 (m, 1H).

Compound 4040: Second eluting diastereomer. Chiral SFC: 97%, RT=3.374 min., (M+H)⁺=341 (Method W). LCMS: 99%, RT=2.852 min., (M+H)⁺=341 (Method C). ¹H NMR (400 MHz, methanol-d₄) mixture of rotamers δ 7.30-7.15 (m, 5H), 7.11-6.96 (m, 3H), 6.47-6.21 (m, 1H), 5.01-4.90 (m, 1H), 4.16-3.93 (m, 1H), 3.85-3.70 (m, 1H), 3.42-3.33 (m, 1H), 3.05-2.91 (m, 1H), 2.91-2.75 (m, 1H), 2.45-2.20 (m, 2H), 2.13-1.94 (m, 1H), 1.85-1.67 (m, 1H).

Absolute stereochemistry of trans-cyclobutyl motif was assigned arbitrarily and therefore may switched between compounds 4039 and 4040.

### Example 37. (S)—N-(cis-3-amino-3-methylcyclobutyl)-1-(4-fluorophenyl)-3,4-dihydroisoquinoline-2(1H)-carboxamide hydrochloride, Compound 4041

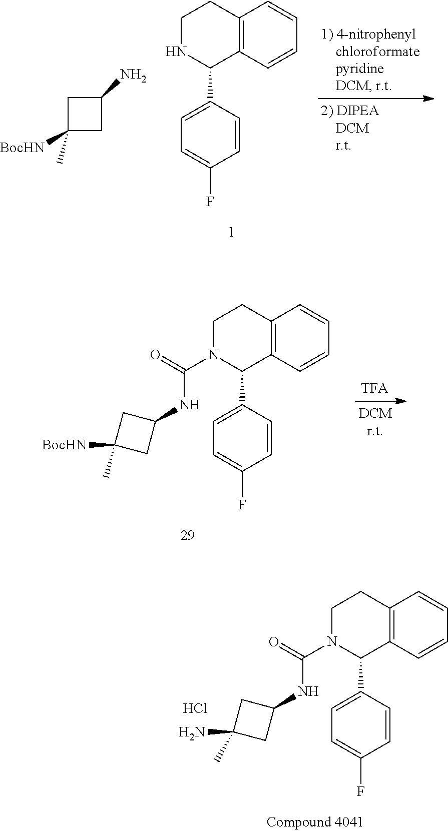 [SMILES: CC(C)(C)OC(=O)N[C@]1(C)C[C@H](N)C1 ; Fc1ccc([C@@H]2NCCc3ccccc32)cc1 ; CC(C)(C)OC(=O)N[C@]1(C)C[C@H](NC(=O)N2CCc3ccccc3[C@@H]2c2ccc(F)cc2)C1 ; Cl ; C[C@]1(N)C[C@@H](NC(=O)N2CCc3ccccc3[C@@H]2c2ccc(F)cc2)C1]

### Step 1. Synthesis of tert-butyl (cis-3-((S)-1-(4-fluorophenyl)-1,2,3,4-tetrahydroisoquinoline-2-carboxamido)-1-methylcyclobutyl)carbamate, 29

Starting from tert-butyl (cis-3-amino-1-methylcyclobutyl)carbamate (100 mg, 0.499 mmol) and (S)-1-(4-fluorophenyl)-1,2,3,4-tetrahydroisoquinoline, 1 (136 mg, 0.599 mmol), tert-butyl (cis-3-((S)-1-(4-fluorophenyl)-1,2,3,4-tetrahydroisoquinoline-2-carboxamido)-1-methylcyclobutyl)carbamate, 29 (0.144 g) was prepared according to the procedure described for tert-butyl (cis-3-((S)-1-(4-fluorophenyl)-1,2,3,4-tetrahydroisoquinoline-2-carboxamido)cyclobutyl)carbamate (see Scheme 32) and purified by flash column chromatography (silica, eluting with 0% to 50% ethyl acetate in heptane). LCMS: 92%, RT=2.14 min., (M+H)⁺=454 (Method B). ¹H NMR (400 MHz, chloroform-d) δ 7.25-7.09 (m, 6H), 7.03-6.87 (m, 2H), 6.42 (s, 1H), 5.54 (br s, 1H), 4.63 (br s, 1H), 4.38-4.08 (m, 1H), 3.70-3.55 (m, 1H), 3.62-3.34 (m, 1H), 3.05-2.87 (m, 1H), 2.84-2.70 (m, 1H), 2.60-2.33 (m, 4H), 1.44 (s, 9H), 1.34 (s, 3H).

### Step 2. Synthesis of (S)—N-(cis-3-amino-3-methylcyclobutyl)-1-(4-fluorophenyl)-3,4-dihydroisoquinoline-2(1H)-carboxamide hydrochloride, Compound 4041

Starting from tert-butyl (cis-3-((S)-1-(4-fluorophenyl)-1,2,3,4-tetrahydroisoquinoline-2-carboxamido)-1-methylcyclobutyl)carbamate, 29 (140 mg, 0.309 mmol), (S)—N-(cis-3-amino-3-methylcyclobutyl)-1-(4-fluorophenyl)-3,4-dihydroisoquinoline-2(1H)-carboxamide hydrochloride, compound 4041 (80 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5), purified by acidic preparative MPLC (Linear Gradient: t=0 min 5% A, t=1 min 5% A; t=16 min 50% A; t=17 min 100%; t=22 min 100% A; detection: 220/254 nm) and lyophilized from aqueous HCl (0.1 M, 2.5 mL), after SCX-2 (2 g) ion exchange chromatography. White solid. LCMS: 99%, RT=1.04 min., (M+H)⁺=354 (Method P). ¹H NMR (400 MHz, DMSO-d₆) δ 8.29 (s, 3H), 7.28-7.03 (m, 9H), 6.45 (s, 1H), 4.18-3.96 (m, 1H), 3.89-3.72 (m, 1H), 3.32-3.16 (m, 1H), 2.99-2.80 (m, 1H), 2.78-2.62 (m, 1H), 2.45-2.27 (m, 4H), 1.38 (s, 3H).

### Example 38. (S)—N-(trans-3-amino-3-methylcyclobutyl)-1-(4-fluorophenyl)-3,4-dihydroisoquinoline-2(1H)-carboxamide hydrochloride, Compound 4042

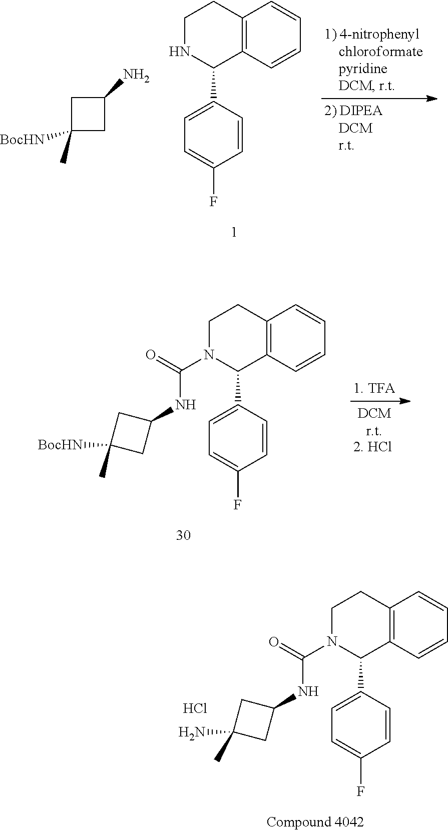 [SMILES: CC(C)(C)OC(=O)N[C@]1(C)C[C@@H](N)C1 ; Fc1ccc([C@@H]2NCCc3ccccc32)cc1 ; CC(C)(C)OC(=O)N[C@]1(C)C[C@@H](NC(=O)N2CCc3ccccc3[C@@H]2c2ccc(F)cc2)C1 ; Cl ; C[C@]1(N)C[C@H](NC(=O)N2CCc3ccccc3[C@@H]2c2ccc(F)cc2)C1]

### Step 1. Synthesis of tert-butyl (trans-3-((S)-1-(4-fluorophenyl)-1,2,3,4-tetrahydroisoquinoline-2-carboxamido)-1-methylcyclobutyl)carbamate, 30

Starting from tert-butyl (trans-3-amino-1-methylcyclobutyl)carbamate (100 mg, 0.499 mmol) and (S)-1-(4-fluorophenyl)-1,2,3,4-tetrahydroisoquinoline, 1 (136 mg, 0.599 mmol), tert-butyl ((trans-3-((S)-1-(4-fluorophenyl)-1,2,3,4-tetrahydroisoquinoline-2-carboxamido)-1-methylcyclobutyl)carbamate, 30 (113 mg) was prepared according to the procedure described for tert-butyl (cis-3-((S)-1-(4-fluorophenyl)-1,2,3,4-tetrahydroisoquinoline-2-carboxamido)cyclobutyl)carbamate (see Scheme 32) and purified by flash column chromatography (silica, eluting with 0% to 50% ethyl acetate in heptane), followed by basic preparative MPLC (Linear Gradient: t=0 min 5% A, t=1 min 5% A; t=16 min 70% A; t=17 min 100% A; t=22 min 100% A; detection: 220/254 nm). White solid. LCMS: 96.7%, RT=2.21 min., (M+H)⁺=454 (Method B). ¹H NMR (400 MHz, chloroform-d) δ 7.25-7.11 (m, 6H), 7.03-6.89 (m, 2H), 6.38 (s, 1H), 4.66 (s, 1H), 4.58 (d, J=6.8 Hz, 1H), 4.40 (q, J=7.8 Hz, 1H), 3.72-3.33 (m, 2H), 3.05-2.85 (m, 1H), 2.84-2.73 (m, 1H), 2.72-2.51 (m, 2H), 1.80 (m, 2H), 1.44 (s, 9H), 1.41 (s, 3H).

### Step 2. Synthesis of (S)—N-(trans-3-amino-3-methylcyclobutyl)-1-(4-fluorophenyl)-3,4-dihydroisoquinoline-2(1H)-carboxamide hydrochloride, Compound 4042

Starting from tert-butyl (trans-3-((S)-1-(4-fluorophenyl)-1,2,3,4-tetrahydroisoquinoline-2-carboxamido)-1-methylcyclobutyl)carbamate, 30 (113 mg, 0.249 mmol), (S)—N-(trans-3-amino-3-methylcyclobutyl)-1-(4-fluorophenyl)-3,4-dihydroisoquinoline-2(1H)-carboxamide hydrochloride, compound 4042 (95 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5), purified by SCX-2 (2 g) ion exchange chromatography, and lyophilized from aqueous HCl (0.1 M, 2.5 mL). White solid. LCMS: 98.8%, RT=1.07 min., (M+H)⁺=354 (Method P). ¹H NMR (400 MHz, DMSO-d6) δ 8.14 (s, 3H), 7.34-7.06 (m, 8H), 6.91 (d, J=7.3 Hz, 1H), 6.42 (s, 1H), 4.53-4.40 (m, 1H), 3.78-3.67 (m, 1H), 3.26-3.15 (m, 1H), 2.91-2.79 (m, 1H), 2.74-2.62 (m, 1H), 2.49-2.39 (m, 2H), 2.23-2.07 (m, 2H), 1.39 (s, 3H).

### Example 39. (S)-1-(4-fluorophenyl)-N-(trans-3-(methylamino)cyclobutyl)-3,4-dihydroisoquinoline-2(1H)-carboxamide hydrochloride, Compound 4043

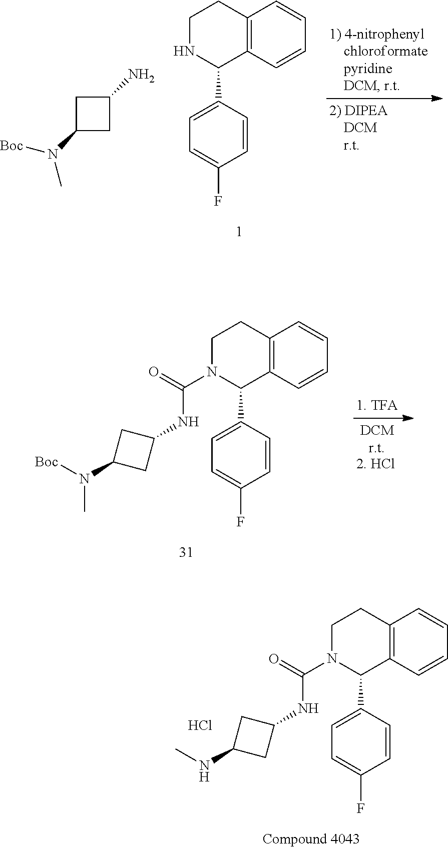 [SMILES: CN(C(=O)OC(C)(C)C)[C@H]1C[C@H](N)C1 ; Fc1ccc([C@@H]2NCCc3ccccc32)cc1 ; CN(C(=O)OC(C)(C)C)[C@H]1C[C@H](NC(=O)N2CCc3ccccc3[C@@H]2c2ccc(F)cc2)C1 ; CN[C@H]1C[C@H](NC(=O)N2CCc3ccccc3[C@@H]2c2ccc(F)cc2)C1 ; Cl]

### Step 1. Synthesis of tert-butyl (trans-3-((S)-1-(4-fluorophenyl)-1,2,3,4-tetrahydroisoquinoline-2-carboxamido)cyclobutyl)(methyl)carbamate, 31

Starting from tert-butyl (trans-3-aminocyclobutyl)(methyl)carbamate (100 mg, 0.499 mmol) and (S)-1-(4-fluorophenyl)-1,2,3,4-tetrahydroisoquinoline, 1 (136 mg, 0.599 mmol), tert-butyl (trans-3-((S)-1-(4-fluorophenyl)-1,2,3,4-tetrahydroisoquinoline-2-carboxamido)cyclobutyl)(methyl)carbamate, 31 (127 mg) was prepared according to the procedure described for tert-butyl (cis-3-((S)-1-(4-fluorophenyl)-1,2,3,4-tetrahydroisoquinoline-2-carboxamido)cyclobutyl)carbamate (see Scheme 32) and purified by basic preparative MPLC (Linear Gradient: t=0 min 5% A, t=1 min 5% A; t=16 min 70% A; t=17 min 100% A; t=22 min 100% A; detection: 220/254 nm). LCMS: 95%, RT=2.206 min., (M+H)⁺=454 (Method B). ¹H NMR (400 MHz, chloroform-d) δ 7.25-7.12 (m, 6H), 6.95 (t, J=8.7 Hz, 2H), 6.39 (s, 1H), 5.04-4.40 (m, 2H), 4.23-4.13 (m, 1H), 3.61-3.46 (m, 2H), 2.98-2.87 (m, 1H), 2.86-2.73 (m, 4H), 2.58-2.44 (m, 2H), 2.21-2.04 (m, 2H), 1.45 (s, 9H).

### Step 2. Synthesis of (S)-1-(4-fluorophenyl)-N-(trans-3-(methylamino)cyclobutyl)-3,4-dihydroisoquinoline-2(1H)-carboxamide hydrochloride, Compound 4043

Starting from tert-butyl (trans-3-((S)-1-(4-fluorophenyl)-1,2,3,4-tetrahydroisoquinoline-2-carboxamido)cyclobutyl)(methyl)carbamate, 31 (20 mg, 0.044 mmol), (S)-1-(4-fluorophenyl)-N-(trans-3-(methylamino)cyclobutyl)-3,4-dihydroisoquinoline-2(1H)-carboxamide hydrochloride, Compound 4043 (15 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5), purified by SCX-2 (2 g) ion exchange chromatography, and lyophilized from aqueous HCl (0.1 M, 0.40 mL). White solid. LCMS: 98.5%, RT=1.061 min., (M+H)⁺=354 (Method P). ¹H NMR (400 MHz, DMSO-d6) δ 8.87 (s, 2H), 7.29-7.07 (m, 8H), 6.97 (d, J=6.8 Hz, 1H), 6.42 (s, 1H), 4.45-4.32 (m, 1H), 3.79-3.68 (m, 1H), 3.65-3.55 (m, 1H), 3.26-3.15 (m, 1H), 2.93-2.80 (m, 1H), 2.74-2.63 (m, 1H), 2.47 (s, 3H), 2.42-2.22 (m, 4H).

### Example 40. (S)-1-(4-fluorophenyl)-N-methyl-N-((trans)-3-(methylamino)cyclobutyl)-3,4-dihydroisoquinoline-2(1H)-carboxamide hydrochloride, Compound 4044

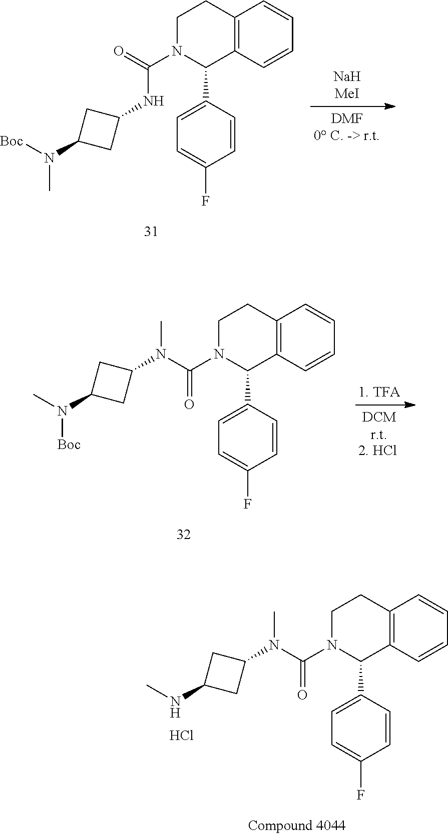 [SMILES: CN(C(=O)OC(C)(C)C)[C@H]1C[C@H](NC(=O)N2CCc3ccccc3[C@@H]2c2ccc(F)cc2)C1 ; CN(C(=O)OC(C)(C)C)[C@H]1C[C@H](N(C)C(=O)N2CCc3ccccc3[C@@H]2c2ccc(F)cc2)C1 ; CN[C@H]1C[C@H](N(C)C(=O)N2CCc3ccccc3[C@@H]2c2ccc(F)cc2)C1 ; Cl]

### Step 1. tert-butyl (trans-3-((S)-1-(4-fluorophenyl)-N-methyl-1,2,3,4-tetrahydroisoquinoline-2-carboxamido)cyclobutyl)(methyl)carbamate, 32

Sodium hydride (60% dispersion in mineral oil, 10.58 mg, 0.265 mmol) was added to a solution of tert-butyl ((trans)-3-((S)-1-(4-fluorophenyl)-1,2,3,4-tetrahydroisoquinoline-2-carboxamido)cyclobutyl)(methyl)carbamate, 31 (80 mg, 0.176 mmol) in N,N-dimethylformamide (dry, 2 mL) at 0° C. under argon atmosphere. After stirring for 15 minutes, iodomethane (33 μl, 0.529 mmol) was added dropwise. The reaction mixture was allowed to slowly reach room temperature and stirred for 5 hours. The reaction mixture was quenched with water and extracted with dichloromethane (3×15 mL). The combined organics were dried on Na₂SO₄ and evaporated under reduced pressure. The residue was purified by flash column chromatography (silica, eluting with 0 to 40% ethyl acetate in heptane) to give tert-butyl (trans-3-((S)-1-(4-fluorophenyl)-N-methyl-1,2,3,4-tetrahydroisoquinoline-2-carboxamido)cyclobutyl)(methyl)carbamate, 32 (27 mg): LCMS: 99.4%, RT=2.263 min., (M+H)⁺=468 (Method B). ¹H NMR (400 MHz, chloroform-d) δ 7.23-7.11 (m, 5H), 7.03-6.89 (m, 3H), 6.21 (s, 1H), 4.65 (br s, 1H), 4.04-3.94 (m, 1H), 3.80-3.71 (m, 1H), 3.31-3.19 (m, 1H), 3.07-2.94 (m, 1H), 2.86-2.77 (m, 1H), 2.84 (s, 3H), 2.75 (s, 3H), 2.43-2.22 (m, 4H), 1.45 (s, 9H).

### Step 2. Synthesis of (S)-1-(4-fluorophenyl)-N-methyl-N-(trans-3-(methylamino)cyclobutyl)-3,4-dihydroisoquinoline-2(1H)-carboxamide hydrochloride, Compound 4044

Starting from tert-butyl ((trans)-3-((S)-1-(4-fluorophenyl)-N-methyl-1,2,3,4-tetrahydroisoquinoline-2-carboxamido)cyclobutyl)(methyl)carbamate, 32 (27 mg, 0.058 mmol), (S)-1-(4-fluorophenyl)-N-methyl-N-((trans)-3-(methylamino)cyclobutyl)-3,4-dihydroisoquinoline-2(1H)-carboxamide hydrochloride (23 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5), purified by SCX-2 (1 g) ion exchange chromatography, and lyophilized from aqueous HCl (0.1 M, 0.6 mL). White solid LCMS: 97.8%, RT=1.09 min., (M+H)⁺=368 (Method P). ¹H NMR (400 MHz, chloroform-d) δ 9.78 (br s, 2H), 7.24-7.10 (m, 5H), 7.03-6.90 (m, 3H), 6.19 (s, 1H), 4.38-4.26 (m, 1H), 3.75-3.56 (m, 2H), 3.35-3.21 (m, 1H), 3.07-2.93 (m, 1H), 2.88-2.77 (m, 1H), 2.75-2.60 (m, 2H), 2.72 (s, 3H), 2.59-2.45 (m, 2H), 2.56 (s, 3H).

### Example 41. (S)-1-(4-fluorophenyl)-N-(cis-3-(methylamino)cyclobutyl)-3,4-dihydroisoquinoline-2(1H)-carboxamide hydrochloride, Compound 4045

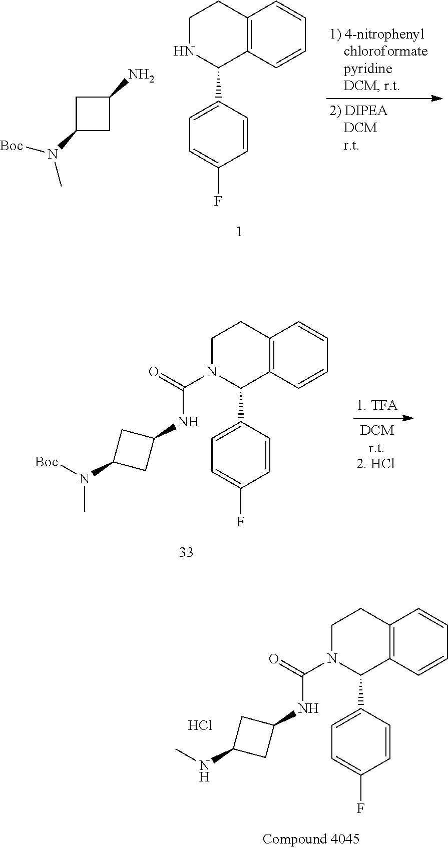 [SMILES: CN(C(=O)OC(C)(C)C)[C@H]1C[C@@H](N)C1 ; Fc1ccc([C@@H]2NCCc3ccccc32)cc1 ; CN(C(=O)OC(C)(C)C)[C@H]1C[C@@H](NC(=O)N2CCc3ccccc3[C@@H]2c2ccc(F)cc2)C1 ; CN[C@H]1C[C@@H](NC(=O)N2CCc3ccccc3[C@@H]2c2ccc(F)cc2)C1 ; Cl]

### Step 1. Synthesis of tert-butyl (cis-3-((S)-1-(4-fluorophenyl)-1,2,3,4-tetrahydroisoquinoline-2-carboxamido)cyclobutyl)(methyl)carbamate

Starting from tert-butyl (cis-3-aminocyclobutyl)(methyl)carbamate (100 mg, 0.499 mmol) and (S)-1-(4-fluorophenyl)-1,2,3,4-tetrahydroisoquinoline, 1 (136 mg, 0.599 mmol), tert-butyl (cis-3-((S)-1-(4-fluorophenyl)-1,2,3,4-tetrahydroisoquinoline-2-carboxamido)cyclobutyl)(methyl)carbamate, 33 (75 mg) was prepared according to the procedure described for tert-butyl (cis-3-((S)-1-(4-fluorophenyl)-1,2,3,4-tetrahydroisoquinoline-2-carboxamido)cyclobutyl)carbamate (see Scheme 32) and purified by basic preparative MPLC (Linear Gradient: t=0 min 5% A, t=1 min 30% A; t=16 min 70% A; t=17 min 100% A; t=22 min 100% A; detection: 220/254 nm). LCMS: 92.5%, RT=2.14 min., (M+H)⁺=454 (Method B). ¹H NMR (400 MHz, chloroform-d) δ 7.24-7.08 (m, 6H), 7.02-6.89 (m, 2H), 6.39 (s, 1H), 4.63 (br s, 1H), 4.05-3.92 (m, 2H), 3.58-3.44 (m, 2H), 2.97-2.85 (m, 1H), 2.83-2.71 (s, 4H), 2.69-2.54 (m, 2H), 2.02-1.79 (m, 2H), 1.45 (s, 9H).

### Step 2. Synthesis of (S)-1-(4-fluorophenyl)-N-(cis-3-(methylamino)cyclobutyl)-3,4-dihydroisoquinoline-2(1H)-carboxamide hydrochloride, Compound 4045

Starting from tert-butyl (cis-3-((S)-1-(4-fluorophenyl)-1,2,3,4-tetrahydroisoquinoline-2-carboxamido)cyclobutyl)(methyl)carbamate, 33 (20 mg, 0.044 mmol), (S)-1-(4-fluorophenyl)-N-(cis-3-(methylamino)cyclobutyl)-3,4-dihydroisoquinoline-2(1H)-carboxamide hydrochloride, compound 4045 (3.7 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5), purified by acidic preparative MPLC (Linear Gradient: t=0 min 5% A, t=1 min 5% A; t=16 min 50% A; t=17 min 100%; t=22 min 100% A; detection: 220 nm) and lyophilized from aqueous HCl (0.1 M, 0.10 mL), after SCX-2 (1 g) ion exchange chromatography. White solid. LCMS: 99.2%, RT=1.032 min., (M+H)⁺=354 (Method P). ¹H NMR (400 MHz, chloroform-d) δ 9.64 (s, 2H), 7.24-7.09 (m, 6H), 6.93 (t, J=8.7 Hz, 1H), 6.48 (s, 1H), 6.07 (d, J=8.8 Hz, 1H), 4.44-4.29 (m, 1H), 3.76-3.64 (m, 1H), 3.47-3.37 (m, 1H), 3.36-3.25 (m, 1H), 3.01-2.85 (m, 3H), 2.79-2.67 (m, 1H), 2.60-2.41 (m, 5H).

### Example 42. (Cis-3-aminocyclobutyl)methyl (S)-1-(4-fluorophenyl)-3,4-dihydroisoquinoline-2(1H)-carboxylate, Compound 4046

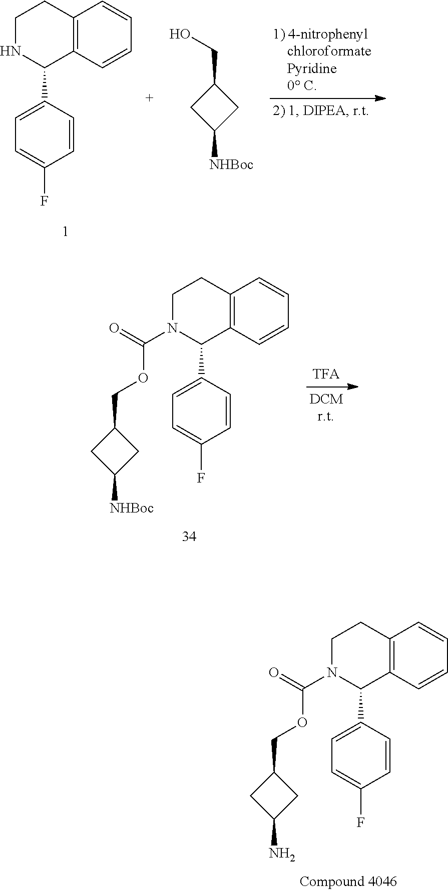 [SMILES: Fc1ccc([C@@H]2NCCc3ccccc32)cc1 ; CC(C)(C)OC(=O)N[C@H]1C[C@@H](CO)C1 ; CC(C)(C)OC(=O)N[C@H]1C[C@@H](COC(=O)N2CCc3ccccc3[C@@H]2c2ccc(F)cc2)C1 ; N[C@H]1C[C@@H](COC(=O)N2CCc3ccccc3[C@@H]2c2ccc(F)cc2)C1]

### Step 1. Synthesis of (cis-3-((tert-butoxycarbonyl)amino)cyclobutyl)methyl (S)-1-(4-fluorophenyl)-3,4-dihydroisoquinoline-2(1H)-carboxylate, 34

4-Nitrophenyl chloroformate (123 mg, 0.608 mmol) was added to a solution of tert-butyl (cis-3-(hydroxymethyl)cyclobutyl)carbamate (102 mg, 0.507 mmol) in pyridine (dry, 1 mL) at 0° C. The mixture was stirred for 2.5 hours at room temperature. Then (S)-1-(4-fluorophenyl)-1,2,3,4-tetrahydroisoquinoline, 1 (138 mg, 0.608 mmol) followed by N,N-diisopropylethylamine (180 μL, 1.01 mmol) were added and stirring was continued overnight. The mixture was diluted with ethyl acetate (20 mL) and washed with ice cold aqueous HCl (1 M, 10 mL). The aqueous layer was extracted with ethyl acetate (10 mL). The combined organic layers were washed with saturated aqueous NaHCO₃ (10 mL) and brine (2×10 mL), dried on Na₂SO₄ and evaporated under reduced pressure. The residue was dissolved in a minimal amount of heptane and dichloromethane, some triethylamine was added (2 layer system), then coated on hydromatrix and purified by flash column chromatography (silica, eluting with 0 to 100% ethyl acetate in heptane) to give (cis-3-((tert-butoxycarbonyl)amino)cyclobutyl)methyl (S)-1-(4-fluorophenyl)-3,4-dihydroisoquinoline-2(1H)-carboxylate, 34 (54 mg) as an oil. LCMS: 100%, RT=2.28 min., (M+Na)⁺=477 (Method A). ¹H NMR (400 MHz, chloroform-d) mixture of rotamers δ 7.25-7.10 (m, 5H), 7.03 (d, J=7.4 Hz, 1H), 7.00-6.92 (m, 2H), 6.60-6.04 (m, 1H), 4.77-4.28 (m, 1H), 4.25-3.83 (m, 4H), 3.24 (ddd, J=13.3, 10.6, 4.4 Hz, 1H), 3.08-2.88 (m, 1H), 2.84-2.71 (m, 1H), 2.52-2.37 (m, 2H), 2.37-2.18 (m, 1H), 1.67-1.57 (m, 2H), 1.44 (s, 9H).

### Step 2. Synthesis of (cis-3-aminocyclobutyl)methyl (S)-1-(4-fluorophenyl)-3,4-dihydroisoquinoline-2(1H)-carboxylate, Compound 4046

Starting from (cis-3-((tert-butoxycarbonyl)amino)cyclobutyl)methyl (S)-1-(4-fluorophenyl)-3,4-dihydroisoquinoline-2(1H)-carboxylate, 34 (54 mg, 0.119 mmol), (cis-3-aminocyclobutyl)methyl (S)-1-(4-fluorophenyl)-3,4-dihydroisoquinoline-2(1H)-carboxylate, compound 4046 (38 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by SCX-2 (1 g) ion exchange chromatography. Colorless oil. LCMS: 99%, RT=2.85 min., (M+H)⁺=355 (Method C). ¹H NMR (400 MHz, chloroform-d) mixture of rotamers δ 7.25-7.10 (m, 5H), 7.07-6.90 (m, 3H), 6.62-6.03 (m, 1H), 4.27-3.89 (m, 3H), 3.38-3.13 (m, 2H), 2.98 (s, 1H), 2.77 (dt, J=16.1, 3.9 Hz, 1H), 2.49-2.32 (m, 2H), 2.29-2.10 (m, 1H), 1.50-1.36 (m, 2H).

### Example 43. (Trans-3-aminocyclobutyl)methyl (S)-1-(4-fluorophenyl)-3,4-dihydroisoquinoline-2(1H)-carboxylate, Compound 4047

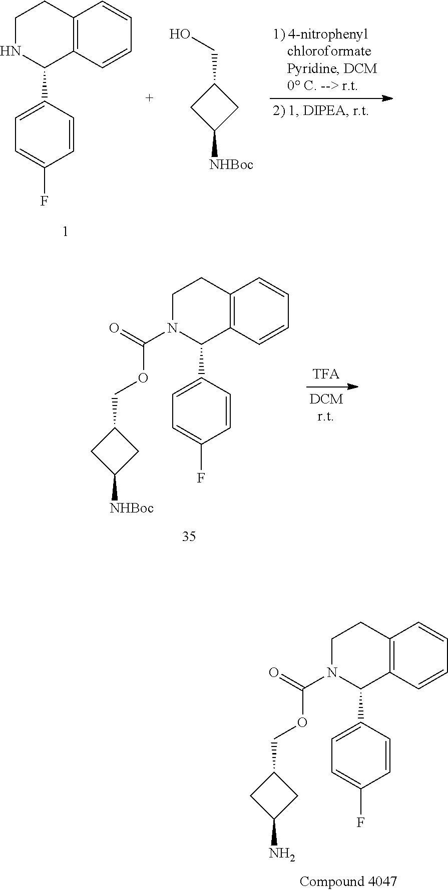 [SMILES: Fc1ccc([C@@H]2NCCc3ccccc32)cc1 ; CC(C)(C)OC(=O)N[C@H]1C[C@H](CO)C1 ; CC(C)(C)OC(=O)N[C@H]1C[C@H](COC(=O)N2CCc3ccccc3[C@@H]2c2ccc(F)cc2)C1 ; N[C@H]1C[C@H](COC(=O)N2CCc3ccccc3[C@@H]2c2ccc(F)cc2)C1]

### Step 1. Synthesis of (trans-3-((tert-butoxycarbonyl)amino)cyclobutyl)methyl (S)-1-(4-fluorophenyl)-3,4-dihydroisoquinoline-2(1H)-carboxylate, 35

4-Nitrophenyl chloroformate (110 mg, 0.547 mmol) and pyridine (dry, 80 μL, 0.994 mmol) were added to a solution of tert-butyl (trans-3-(hydroxymethyl)cyclobutyl)carbamate (100 mg, 0.497 mmol) in dichloromethane (2 mL) at 0° C. The mixture was stirred at room temperature for 5.5 hours. Then (S)-1-(4-fluorophenyl)-1,2,3,4-tetrahydroisoquinoline, 1 (136 mg, 0.596 mmol) followed by N,N-diisopropylethylamine (174 μL, 0.994 mmol) were added. The mixture was stirred overnight, diluted with ethyl acetate (10 mL) and washed with a mixture of water and saturated aqueous K₂CO₃ (1:1, 10 mL). The water layer was extracted with ethyl acetate (10 mL). The combined organic layers were washed with brine (10 mL), dried on Na₂SO₄ and evaporated under reduced pressure. The residue was purified by flash column chromatography (silica, eluting with 5 to 100% ethyl acetate in heptane) and basic preparative MPLC (Linear Gradient: t=0 min 40% A, t=2 min 5% A; t=15 min 80% A; t=1 min 100% A; t=5 min 100% A; detection: 270/215 nm). The product containing fractions were combined and concentrated under reduced pressure to remove acetonitrile. The residue was stirred with dichloromethane (10 mL) and brine (10 mL). The layers were separated using a phase separator and the organic filtrate was evaporated under reduced pressure to give (trans-3-((tert-butoxycarbonyl)amino)cyclobutyl)methyl (S)-1-(4-fluorophenyl)-3,4-dihydroisoquinoline-2(1H)-carboxylate, 35 (70.6 mg) as an oil. LCMS: 99%, RT=2.29 min., (M+Na)⁺=477 (Method A). ¹H NMR (400 MHz, chloroform-d) mixture of rotamers δ 7.26-7.08 (m, 5H), 7.08-6.89 (m, 3H), 6.64-5.93 (m, 1H), 4.89-4.37 (m, 1H), 4.32-3.79 (m, 4H), 3.23 (ddd, J=13.3, 10.8, 4.4 Hz, 1H), 3.09-2.86 (m, 1H), 2.77 (dt, J=16.4, 4.0 Hz, 1H), 2.64-2.43 (m, 1H), 2.34-2.11 (m, 2H), 2.11-1.90 (m, 2H), 1.43 (s, 9H).

### Step 2. Synthesis of (trans-3-aminocyclobutyl)methyl (S)-1-(4-fluorophenyl)-3,4-dihydroisoquinoline-2(1H)-carboxylate, Compound 4047

Starting from (trans-3-((tert-butoxycarbonyl)amino)cyclobutyl)methyl (S)-1-(4-fluorophenyl)-3,4-dihydroisoquinoline-2(1H)-carboxylate, 35 (70.6 mg, 0.155 mmol), (trans-3-aminocyclobutyl)methyl (S)-1-(4-fluorophenyl)-3,4-dihydroisoquinoline-2(1H)-carboxylate, compound 4047 (49 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by SCX-2 (1 g) ion exchange chromatography. Yellow oil. LCMS: 98%, RT=2.82 min., (M+H)⁺=355 (Method C). ¹H NMR (400 MHz, chloroform-d) mixture of rotamers δ 7.26-7.10 (m, 5H), 7.09-6.90 (m, 3H), 6.64-6.07 (m, 1H), 4.36-3.87 (m, 3H), 3.57 (p, J=7.4 Hz, 1H), 3.22 (ddd, J=13.1, 10.7, 4.3 Hz, 1H), 3.08-2.86 (m, 1H), 2.85-2.68 (m, 1H), 2.64-2.39 (m, 1H), 2.24-2.06 (m, 2H), 1.96-1.75 (m, 2H).

### Example 44. 2-(3-Aminobicyclo[1.1.1]pentan-1-yl)propan-2-yl (S)-1-(4-fluorophenyl)-3,4-dihydroisoquinoline-2(1H)-carboxylate, Compound 4048

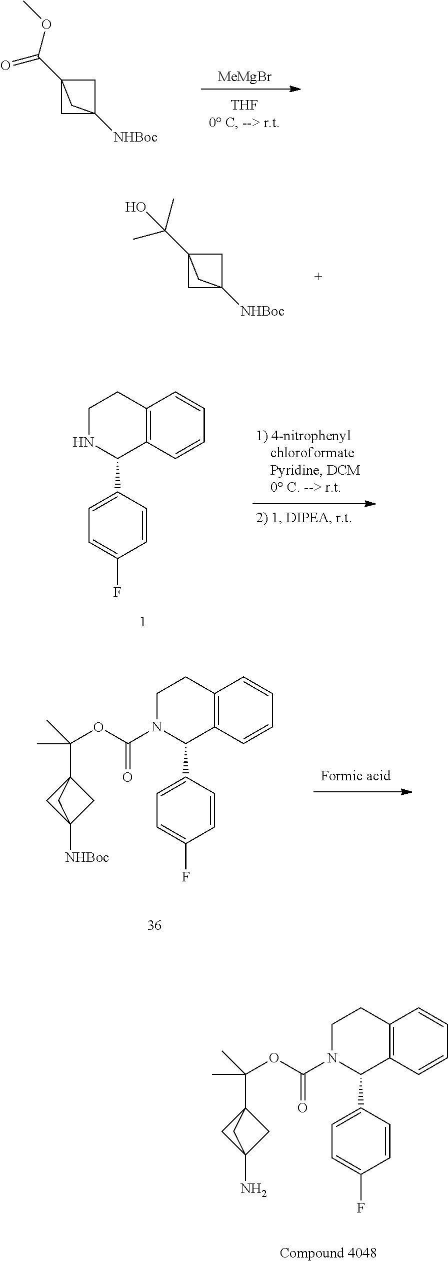 [SMILES: "MeMgBr" ; COC(=O)C12CC(NC(=O)OC(C)(C)C)(C1)C2 ; CC(C)(C)OC(=O)NC12CC(C(C)(C)O)(C1)C2 ; Fc1ccc([C@@H]2NCCc3ccccc32)cc1 ; CC(C)(C)OC(=O)NC12CC(C(C)(C)OC(=O)N3CCc4ccccc4[C@@H]3c3ccc(F)cc3)(C1)C2 ; CC(C)(OC(=O)N1CCc2ccccc2[C@@H]1c1ccc(F)cc1)C12CC(N)(C1)C2]

### Step 1. Synthesis of tert-butyl (3-(2-hydroxypropan-2-yl)bicyclo[1.1.1]pentan-1-yl)carbamate

Under an argon atmosphere, methylmagnesium bromide (3.0 M in diethyl ether, 1.84 mL, 5.52 mmol) was added to a solution of methyl 3-((tert-butoxycarbonyl)amino)bicyclo[1.1.1]pentane-1-carboxylate (444 mg, 1.840 mmol) in dry tetrahydrofuran (5 mL) at 0° C. The mixture was stirred at 0° C. for 2 hours, and at room temperature for 1 hour. Ethyl acetate (30 mL) was added dropwise to the reaction mixture. The resultant mixture was washed with a mixture of brine and water (1:1, 30 mL) containing citric acid (1 g), brine (20 mL), dried on Na₂SO₄ and evaporated under reduced pressure. The residue was evaporated once from a mixture of diethyl ether and pentane (1:1, 10 mL) and the residue (partly crystallized) was recrystallized from a mixture of diethyl ether and pentane to give tert-butyl (3-(2-hydroxypropan-2-yl)bicyclo[1.1.1]pentan-1-yl)carbamate (256 mg) as a colorless solid. Crystallization of the mother liquor gave another batch of tert-butyl (3-(2-hydroxypropan-2-yl)bicyclo[1.1.1]pentan-1-yl)carbamate (89 mg) as a colorless solid. ¹H NMR (400 MHz, chloroform-d) δ 4.92 (br s, 1H), 1.91 (s, 6H), 1.45 (s, 9H), 1.19 (s, 6H).

### Step 2. Synthesis of 2-(3-((tert-butoxycarbonyl)amino)bicyclo[1.1.1]pentan-1-yl)propan-2-yl (S)-1-(4-fluorophenyl)-3,4-dihydroisoquinoline-2(1H)-carboxylate, 36

Starting from tert-butyl (3-(2-hydroxypropan-2-yl)bicyclo[1.1.1]pentan-1-yl)carbamate (91 mg, 0.377 mmol) and (S)-1-(4-fluorophenyl)-1,2,3,4-tetrahydroisoquinoline, 1 (129 mg, 0.566 mmol), 2-(3-((tert-butoxycarbonyl)amino)bicyclo[1.1.1]pentan-1-yl)propan-2-yl (S)-1-(4-fluorophenyl)-3,4-dihydroisoquinoline-2(1H)-carboxylate, 36 (23 mg) was prepared according to the procedure described for (trans-3-((tert-butoxycarbonyl)amino)cyclobutyl)methyl (S)-1-(4-fluorophenyl)-3,4-dihydroisoquinoline-2(1H)-carboxylate (see Scheme 43). Fluffy solid. LCMS: 100%, RT=2.42 min., (M+Na)⁺=517 (Method B). ¹H NMR (400 MHz, chloroform-d) mixture of rotamers δ 7.25-7.08 (m, 5H), 7.08-7.01 (m, 1H), 6.99-6.90 (m, 2H), 6.50-6.09 (m, 1H), 5.16-4.66 (m, 1H), 4.15-3.75 (m, 1H), 3.29-3.05 (m, 1H), 3.05-2.82 (m, 1H), 2.82-2.62 (m, 1H), 1.95 (s, 6H), 1.49 (s, 6H), 1.44 (s, 9H).

### Step 3. Synthesis of 2-(3-aminobicyclo[1.1.1]pentan-1-yl)propan-2-yl (S)-1-(4-fluorophenyl)-3,4-dihydroisoquinoline-2(1H)-carboxylate, Compound 4048

2-(3-((Tert-butoxycarbonyl)amino)bicyclo[1.1.1]pentan-1-yl)propan-2-yl (S)-1-(4-fluorophenyl)-3,4-dihydroisoquinoline-2(1H)-carboxylate, 36 (23 mg, 47 μmol) was dissolved in formic acid (1 mL). After 20 minutes, the reaction mixture was dropped into saturated aqueous NaHCO₃ (30 mL). The mixture was extracted with dichloromethane (2×10 mL). The combined organic layers were dried on Na₂SO₄ and evaporated under reduced pressure. The residue was purified by basic preparative MPLC (Linear Gradient: t=0 min 30% A, t=2 min 30% A; t=15 min 70% A; t=1 min 100% A; t=5 min 100% A; detection: 210/264 nm) The product containing fractions were combined and evaporated under reduced pressure. The residue was lyophilized from a mixture of acetonitrile and water (1:1, 2 mL) to give 2-(3-aminobicyclo[1.1.1]pentan-1-yl)propan-2-yl (S)-1-(4-fluorophenyl)-3,4-dihydroisoquinoline-2(1H)-carboxylate, compound 4048 (10 mg) as a sticky solid. LCMS: 99%, RT=4.16 min., (M+H)⁺=395 (Method D). ¹H NMR (400 MHz, chloroform-d) mixture of rotamers δ 7.25-7.09 (m, 5H), 7.09-7.00 (m, 1H), 6.99-6.90 (m, 2H), 6.53-6.06 (m, 1H), 4.15-3.78 (m, 1H), 3.28-3.05 (m, 1H), 3.05-2.83 (m, 1H), 2.83-2.68 (m, 1H), 1.73 (s, 6H), 1.48 (s, 6H).

### Example 45. 3-Morpholinobicyclo[1.1.1]pentan-1-yl (S)-1-(4-fluorophenyl)-3,4-dihydroisoquinoline-2(1H)-carboxylate hydrochloride, Compound 4049

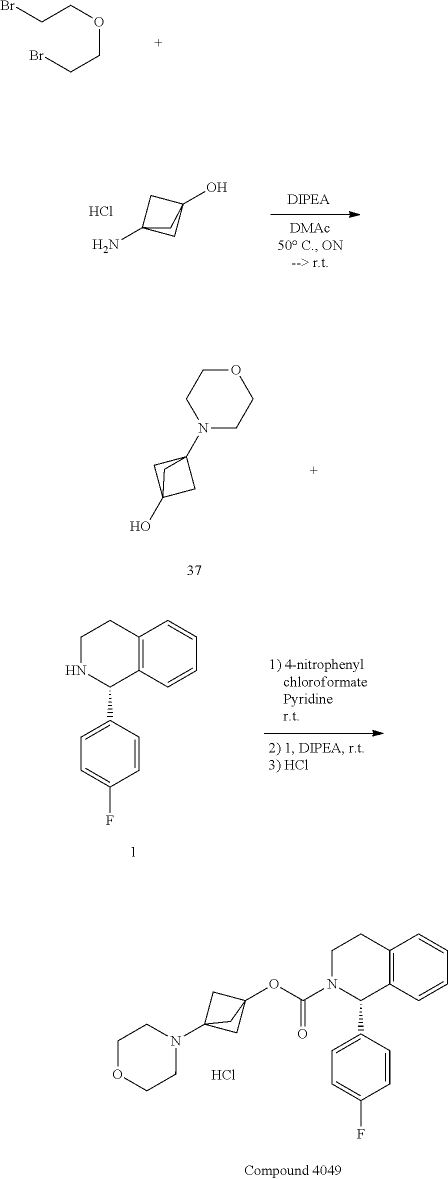

### Step 1. Synthesis of 3-morpholinobicyclo[1.1.1]pentan-1-ol, 37

N,N-diisopropylethylamine (141 μL, 0.807 mmol) and 1-bromo-2-(2-bromoethoxy)ethane (25 μL, 0.199 mmol) were added to a solution of 3-aminobicyclo[1.1.1]pentan-1-ol hydrochloride (20 mg, 0.148 mmol) in N,N-dimethylacetamide (dry, 0.5 mL). The mixture was stirred at 50° C. overnight and at room temperature for 1 day. Then, the mixture was diluted with diethyl ether (10 mL) and washed with a mixture of brine and a saturated aqueous NaHCO₃ (1:1, 10 mL), and brine (2×5 mL). The combined water layers were extracted with ethyl acetate (2×10 mL). The combined organic layers were dried on Na₂SO₄ and evaporated under reduced pressure. The residue was purified by flash column chromatography (silica, eluting with 0 to 10% (7M ammonia in methanol) in dichloromethane) to give 3-morpholinobicyclo[1.1.1]pentan-1-ol (8.5 mg) as a colorless liquid that solidified on standing. ¹H NMR (400 MHz, chloroform-d) b 3.86-3.64 (m, 4H), 2.64-2.25 (m, 5H), 1.94 (s, 6H).

### Step 2. Synthesis of 3-morpholinobicyclo[1.1.1]pentan-1-yl (S)-1-(4-fluorophenyl)-3,4-dihydroisoquinoline-2(1H)-carboxylate hydrochloride, Compound 4049

4-Nitrophenyl chloroformate (13.16 mg, 0.065 mmol) was added to a solution of 3-morpholinobicyclo[1.1.1]pentan-1-ol, 37 (8.5 mg, 0.050 mmol) in pyridine (dry, 0.2 ml) and the mixture was stirred at room temperature. After 2 hours, (S)-1-(4-fluorophenyl)-1,2,3,4-tetrahydroisoquinoline, 1 (14.84 mg, 0.065 mmol) and N,N-diisopropylethylamine (18 μL, 0.100 mmol) were added and stirring was continued overnight. The mixture was diluted with methanol (1 mL) and filtered through a Nylon filter (0.45 μm). The filtrate was purified by basic preparative MPLC (Linear Gradient: t=0 min 40% A, t=2 min 40% A; t=15 min 80% A; t=1 min 100% A; t=5 min 100% A; detection: 220/254/280 nm) and acidic preparative MPLC (Linear Gradient: t=0 min 20% A, t=2 min 20% A; t=15 min 60% A; t=1 min 100%; t=8 min 100% A; detection: 215/263 nm). The product containing fractions were combined, aqueous HCl (1 M, 0.065 mL) was added, and the mixture was lyophilized to give 3-morpholinobicyclo[1.1.1]pentan-1-yl (S)-1-(4-fluorophenyl)-3,4-dihydroisoquinoline-2(1H)-carboxylate hydrochloride, Compound 4049 (4.5 mg) as a colorless solid. LCMS: 95%, RT=3.43 min., (M+H)⁺=423 (Method C). ¹H NMR (400 MHz, DMSO-d₆-D₂O, 80° C.) δ 7.23-7.12 (m, 5H), 7.12-7.00 (m, 3H), 6.14 (s, 1H), 3.86-3.76 (m, 1H), 3.74-3.66 (m, 4H), 3.28 (ddd, J=13.6, 9.3, 5.0 Hz, 1H), 2.92-2.71 (m, 6H), 2.26 (s, 6H).

### Example 46. (S)—N-(2-(3-aminobicyclo[1.1.1]pentan-1-yl)propan-2-yl)-1-(4-fluorophenyl)-3,4-dihydroisoquinoline-2(1H)-carboxamide hydrochloride, Compound 4050

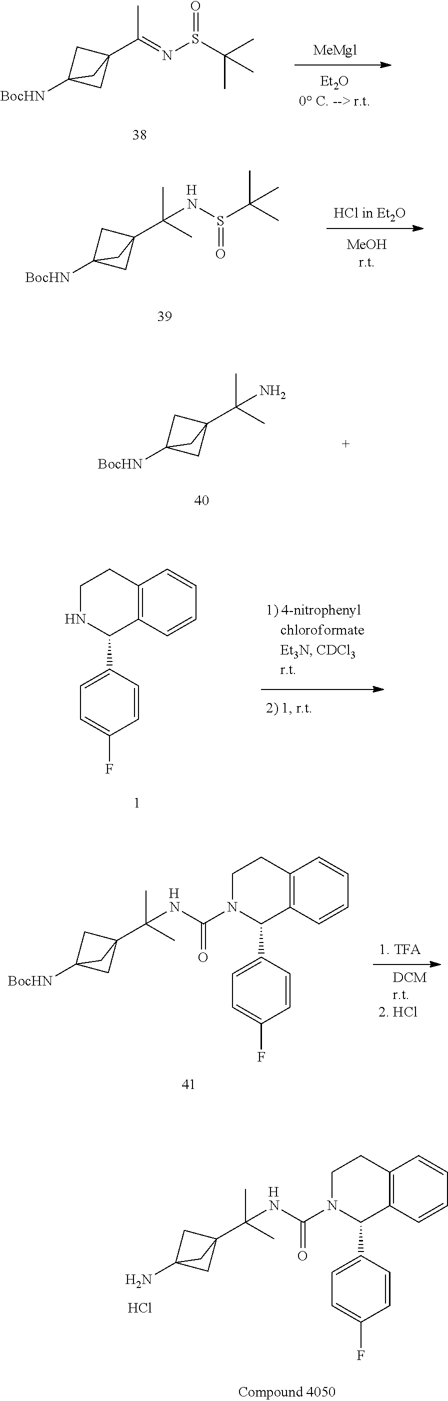

### Step 1. Synthesis of tert-butyl (3-(2-((tert-butylsulfinyl)amino)propan-2-yl)bicyclo[1. 1. 1]pentan-1-yl)carbamate, 39

Under nitrogen atmosphere, iodomethane (398 μL, 6.37 mmol) was added dropwise to a mixture of magnesium (119 mg, 4.90 mmol) in diethyl ether (dry, 11 mL) at such a rate that the mixture maintained a spontaneous reflux. Afterwards, reflux was maintained by warming the reaction mixture in a water bath of 40° C. until the magnesium had reacted. Under argon atmosphere, a portion of the prepared methylmagnesium iodide solution (2.4 mL) was added dropwise to a solution of tert-butyl (E)-(3-(1-((tert-butylsulfinyl)imino)ethyl)bicyclo[1.1.1]pentan-1-yl)carbamate, 38 (prepared according to the procedure described in patent WO2019/94552, 161 mg, 0.490 mmol) in dry diethyl ether (3 ml) at 0° C. The reaction mixture was stirred at room temperature for 3 hours after which another portion of the prepared methyl magnesium iodide solution (1.2 mL) was added dropwise. Stirring was continued for 4 hours and then the reaction mixture was poured into saturated aqueous NH₄Cl (20 mL), The mixture was extracted with a mixture of diethyl ether and ethyl acetate (1:1, 50 mL), and ethyl acetate (20 mL). The combined organic layers were dried on Na₂SO₄ and evaporated under reduced pressure. The residue was purified by flash column chromatography (silica, eluting with 50 to 100% ethyl acetate in heptane) to give tert-butyl (3-(2-((tert-butylsulfinyl)amino)propan-2-yl)bicyclo[1.1.1]pentan-1-yl)carbamate, 39 (64 mg) as a colorless oil. LCMS: 87%, RT=1.96 min., (M+H)⁺=345 (Method B). ¹H NMR (400 MHz, chloroform-d) δ 4.95 (br s, 1H), 2.99 (s, 1H), 1.92 (s, 6H), 1.44 (s, 9H), 1.27 (s, 3H), 1.23 (s, 3H), 1.18 (s, 9H).

### Step 2. Synthesis of tert-butyl (3-(2-aminopropan-2-yl)bicyclo[1.1.1]pentan-1-yl)carbamate, 40

HCl (1 M in diethyl ether, 0.241 mL, 0.241 mmol) was added to a solution of tert-butyl (3-(2-((tert-butylsulfinyl)amino)propan-2-yl)bicyclo[1.1.1]pentan-1-yl)carbamate, 39 (64 mg, 0.186 mmol) in methanol (0.5 mL). After stirring for 1 hour, diethyl ether (10 mL) and aqueous saturated NaHCO₃ (10 mL) were added followed by Na₂CO₃ (0.5 g). The mixture was extracted with diethyl ether (2×10 mL). The combined organic layers were dried on Na₂SO₄ and evaporated under reduced pressure to give tert-butyl (3-(2-aminopropan-2-yl)bicyclo[1.1.1]pentan-1-yl)carbamate, 40 (28.5 mg) as an oil. ¹H NMR (400 MHz, chloroform-d) δ 5.00 (br s, 1H), 1.85 (s, 6H), 1.45 (s, 9H), 1.06 (s, 6H).

### Step 3. Synthesis of tert-butyl (S)-(3-(2-(1-(4-fluorophenyl)-1,2,3,4-tetrahydroisoquinoline-2-carboxamido)propan-2-yl)bicyclo[1.1.1]pentan-1-yl)carbamate, 41

4-Nitrophenyl chloroformate (23.9 mg, 0.119 mmol) was added to a solution of tert-butyl (3-(2-aminopropan-2-yl)bicyclo[1.1.1]pentan-1-yl)carbamate, 40 (28.5 mg, 0.119 mmol) in chloroform-d (1.5 mL). Triethylamine (17 μL, 0.119 mmol) was added and the mixture was stirred at room temperature. After 3 hours, (S)-1-(4-fluorophenyl)-1,2,3,4-tetrahydroisoquinoline, 1 (27.0 mg, 0.119 mmol) was added. After stirring overnight, the reaction mixture was dropped in a mixture of saturated aqueous K₂CO₃ solution and water (1:3, 10 mL). Dichloromethane (10 mL) was added and the mixture was stirred. The layers were separated using a phase separator and the organic filtrate was evaporated under reduced pressure. The residue was purified by flash column chromatography (silica, eluting with 5 to 100% ethyl acetate in heptane) to give tert-butyl (S)-(3-(2-(1-(4-fluorophenyl)-1,2,3,4-tetrahydroisoquinoline-2-carboxamido)propan-2-yl)bicyclo[1.1.1]pentan-1-yl)carbamate, 41 (31 mg) as a colorless solid. LCMS: 100%, RT=2.24 min., (M+H)⁺=494 (Method B). ¹H NMR (400 MHz, chloroform-d) δ 7.24-7.11 (m, 6H), 7.02-6.90 (m, 2H), 6.29 (s, 1H), 4.94 (br s, 1H), 4.36 (s, 1H), 3.51 (dtd, J=19.3, 11.7, 5.6 Hz, 2H), 2.93-2.72 (m, 2H), 1.87 (s, 6H), 1.45 (s, 9H), 1.36 (s, 3H), 1.32 (s, 3H).

### Step 4. Synthesis of (S)—N-(2-(3-aminobicyclo[1.1.1]pentan-1-yl)propan-2-yl)-1-(4-fluorophenyl)-3,4-dihydroisoquinoline-2(1H)-carboxamide hydrochloride, Compound 4050

Starting from tert-butyl (S)-(3-(2-(1-(4-fluorophenyl)-1,2,3,4-tetrahydroisoquinoline-2-carboxamido)propan-2-yl)bicyclo[1.1.1]pentan-1-yl)carbamate, 41 (29.2 mg, 0.059 mmol), (S)—N-(2-(3-aminobicyclo[1.1.1]pentan-1-yl)propan-2-yl)-1-(4-fluorophenyl)-3,4-dihydroisoquinoline-2(1H)-carboxamide hydrochloride, compound 4050 (20.8 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) after basic workup (aqueous K₂CO₃ and water (1:5)/dichloromethane extraction) and lyophilization from acetonitrile (1 mL), aqueous HCl (1 M, 55 μL) and water (2 mL). Colorless solid. LCMS: 97%, RT=3.79 min., (M+H)⁺=394 (method D). ¹H NMR (400 MHz, DMSO-d₆) δ 8.60 (s, 3H), 7.26-7.08 (m, 8H), 6.40 (s, 1H), 5.62 (s, 1H), 3.73 (dt, J=11.9, 5.5 Hz, 1H), 3.27 (ddd, J=13.3, 8.8, 4.8 Hz, 1H), 2.84 (ddd, J=14.9, 8.8, 5.6 Hz, 1H), 2.69 (dt, J=16.0, 4.9 Hz, 1H), 1.82 (s, 6H), 1.25 (s, 3H), 1.24 (s, 3H).

### Example 47. (1-(Methylamino)cyclopropyl)methyl (S)-1-(4-fluorophenyl)-3,4-dihydroisoquinoline-2(1H)-carboxylate hydrochloride, Compound 4051

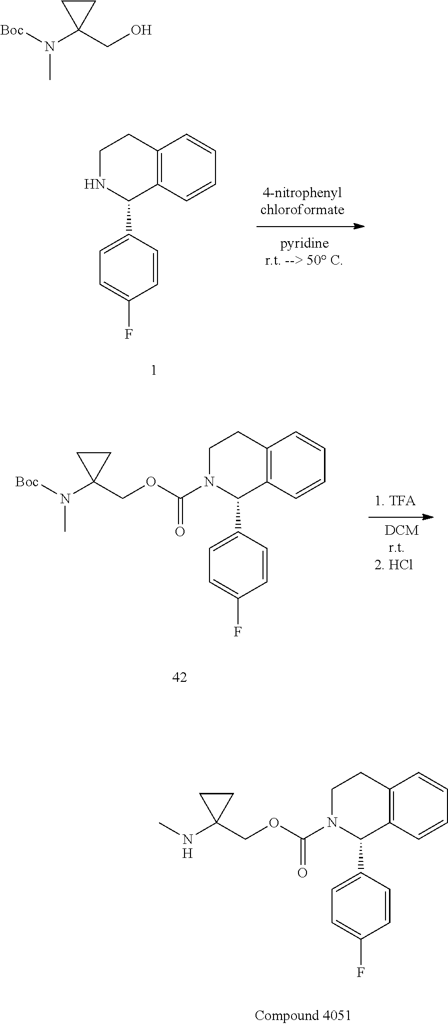

### Step 1. Synthesis of (1-((tert-butoxycarbonyl)(methyl)amino)cyclopropyl)methyl (S)-1-(4-fluorophenyl)-3,4-dihydroisoquinoline-2(1H)-carboxylate, 42

4-Nitrophenyl chloroformate (79 mg, 0.392 mmol) was added to a solution of tert-butyl (1-(hydroxymethyl)cyclopropyl)(methyl)carbamate (52.4 mg, 0.260 mmol) in pyridine (dry, 1.0 mL) at room temperature. After 2 hours and 20 minutes, (S)-1-(4-fluorophenyl)-1,2,3,4-tetrahydroisoquinoline, 1 (119 mg, 0.524 mmol) was added and the mixture was stirred overnight at room temperature. Then, the mixture was warmed to 50° C. and stirred for 8 hours. The reaction mixture was concentrated to dryness under reduced pressure. The residue was partitioned between dichloromethane (4 mL) and aqueous NaOH (0.25 M, 4 mL). The layers were separated using a phase-separator and the organic filtrate was evaporated under reduced pressure. The residue was purified by flash column chromatography (silica, eluting with 10 to 60% ethyl acetate in heptane) to give (1-((tert-butoxycarbonyl)(methyl)amino)cyclopropyl)methyl (S)-1-(4-fluorophenyl)-3,4-dihydroisoquinoline-2(1H)-carboxylate, 42 (70.7 mg) as a colorless oil. LCMS: 98.8%, RT=2.320 min., (M+Na)⁺=477 (Method A). ¹H NMR (400 MHz, chloroform-d) mixture of rotamers δ 7.26-7.09 (m, 5H), 7.03 (d, J=7.5 Hz, 1H), 6.96 (t, J=8.6 Hz, 2H), 6.49-6.19 (m, 1H), 4.36-3.91 (m, 3H), 3.33-3.15 (m, 1H), 3.07-2.91 (m, 1H), 2.88 (br s, 3H), 2.77 (dt, J=16.3, 4.1 Hz, 1H), 1.45 (s, 9H), 1.01-0.80 (m, 4H).

### Step 2. Synthesis of (1-(methylamino)cyclopropyl)methyl (S)-1-(4-fluorophenyl)-3,4-dihydroisoquinoline-2(1H)-carboxylate hydrochloride, Compound 4051

Starting from 1-((tert-butoxycarbonyl)(methyl)amino)cyclopropyl)methyl (S)-1-(4-fluorophenyl)-3,4-dihydroisoquinoline-2(1H)-carboxylate, 42 (70.7 mg, 0.156 mmol), (1-(methylamino)cyclopropyl)methyl (S)-1-(4-fluorophenyl)-3,4-dihydroisoquinoline-2(1H)-carboxylate hydrochloride, Compound 4051 (58.5 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5), purified by SCX-2 (2 g) ion exchange chromatography, and lyophilized from a mixture of acetonitrile and water (1:1, 4 mL), aqueous HCl (1.00 M, 159 μL, 0.159 mmol). White solid. LCMS: 99.2%, RT=2.715 min., (M+H)⁺=355 (Method C). ¹H NMR (400 MHz, DMSO-d₆) mixture of rotamers δ 9.23 (s, 2H), 7.35-7.03 (m, 8H), 6.56-6.19 (m, 1H), 4.54-3.85 (m, 3H), 3.39-3.23 (m, 1H), 3.03-2.71 (m, 2H), 2.62 (s, 3H), 1.27-1.05 (m, 2H), 1.02-0.85 (m, 2H).

### Example 48. (S)—N-(3-(3,3-difluoroazetidin-1-yl)bicyclo[1.1.1]pentan-1-yl)-1-(4-fluorophenyl)-3,4-dihydroisoquinoline-2(1H)-carboxamide, Compound 4052

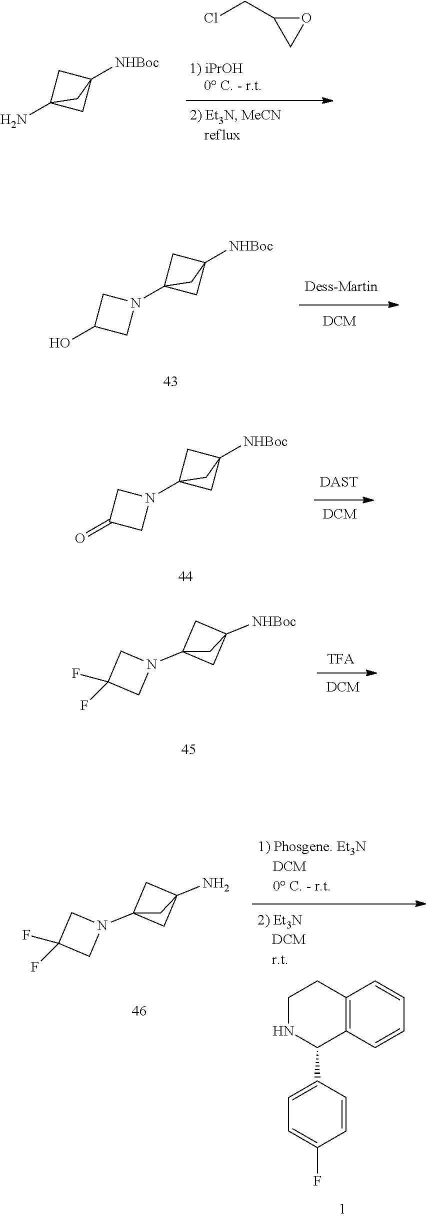

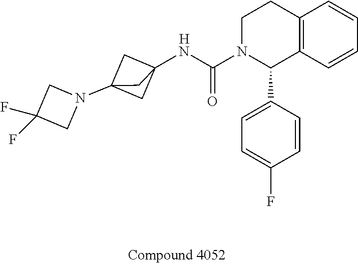

### Step 1. Synthesis of tert-butyl (3-(3-hydroxyazetidin-1-yl)bicyclo[1.1.1]pentan-1-yl)carbamate, 43

At 0° C., epichlorohydrin (617 μL, 7.87 mmol) was added dropwise to a stirred solution of tert-butyl (3-aminobicyclo[1.1.1]pentan-1-yl)carbamate (1.56 g, 7.87 mmol) in 2-propanol (15 mL). The cooling bath was removed and stirring was continued at room temperature overnight. The reaction mixture was concentrated under reduced pressure and co-evaporated twice from acetonitrile. The residue was dissolved in acetonitrile (15 mL), triethylamine (3.3 mL, 23.6 mmol) was added, and the mixture was heated at reflux overnight. The mixture was concentrated under reduced pressure and the residue was purified by flash column chromatography (silica, eluting with 0 to 7% methanol in dichloromethane). The product was stirred in diethyl ether for 15 minutes, filtered through a thin layer of Celite, and the filtrate was evaporated under reduced pressure to give tert-butyl (3-(3-hydroxyazetidin-1-yl)bicyclo[1.1.1]pentan-1-yl)carbamate, 43 (480 mg) as a white foam. ¹H NMR (300 MHz, chloroform-d) δ 4.91 (s, 1H), 4.44 (p, J=5.9 Hz, 1H), 3.62-3.41 (m, 2H), 3.01 (td, J=6.3, 1.3 Hz, 2H), 1.96 (s, 6H), 1.44 (s, 9H).

### Step 2. Synthesis of tert-butyl (3-(3-oxoazetidin-1-yl)bicyclo[1.1.1]pentan-1-yl)carbamate, 44

Dess-Martin periodinane (1.20 g, 2.83 mmol) was added in portions to a stirred solution of tert-butyl (3-(3-hydroxyazetidin-1-yl)bicyclo[1.1.1]pentan-1-yl)carbamate (480 mg, 1.89 mmol) in dichloromethane (10 mL). After stirring overnight at room temperature, a mixture of saturated aqueous NaHCO₃ and saturated aqueous Na₂S₂O₃ (1:1, 4 mL) was added. The resulting mixture was extracted with dichloromethane (3×10 mL) and the combined extracts were dried on Na₂SO₄ and evaporated under reduced pressure. The residue was purified by flash column chromatography (silica, eluting with 0 to 4% methanol in dichloromethane) to obtain tert-butyl (3-(3-oxoazetidin-1-yl)bicyclo[1.1.1]pentan-1-yl)carbamate, 44 (170 mg) as a white solid. ¹H NMR (300 MHz, chloroform-d) δ 5.14 (s, 1H), 4.12 (d, J=0.7 Hz, 4H), 2.04 (s, 6H), 1.42 (s, 9H).

### Step 3. Synthesis of tert-butyl (3-(3,3-difluoroazetidin-1-yl)bicyclo[1.1.1]pentan-1-yl)carbamate, 45

Under a nitrogen atmosphere, diethylaminosulfur trifluoride (178 μL, 1.35 mmol) was added to a stirred solution of tert-butyl (3-(3-oxoazetidin-1-yl)bicyclo[1.1.1]pentan-1-yl)carbamate, 44 (170 mg, 674 μmol) in dichloromethane (3 mL). After stirring overnight at room temperature, the reaction mixture was poured into cold water (10 mL) and extracted with dichloromethane (3×10 mL). The combined extracts were dried on Na₂SO₄ and evaporated under reduced pressure. The residue was purified by flash column chromatography (silica, eluting with 0 to 30% ethyl acetate in heptane) to obtain tert-butyl (3-(3,3-difluoroazetidin-1-yl)bicyclo[1.1.1]pentan-1-yl)carbamate, 45 (77 mg). ¹H NMR (300 MHz, chloroform-d) δ 4.98 (s, 1H), 3.61 (t, J=12.2 Hz, 4H), 2.03 (s, 6H), 1.43 (s, 9H).

### Step 4. Synthesis of 3-(3,3-difluoroazetidin-1-yl)bicyclo[1.1.1]pentan-1-amine bis(2,2,2-trifluoroacetate, 46

To a solution of tert-butyl (3-(3,3-difluoroazetidin-1-yl)bicyclo[1.1.1]pentan-1-yl)carbamate, 45 (77 mg, 0.28 mmol) in dichloromethane (1.6 mL), trifluoroacetic acid (0.4 mL) was added. After 1 hour, the reaction mixture was concentrated to dryness under reduced pressure and co-evaporated from toluene to give 3-(3,3-difluoroazetidin-1-yl)bicyclo[1.1.1]pentan-1-amine bis(2,2,2-trifluoroacetate), 46 (113 mg) as a solid. ¹H NMR (300 MHz, methanol-d₄) b 3.92 (t, J=12 Hz, 4H), 2.18 (s, 6H).

### Step 5. Synthesis of (S)—N-(3-(3,3-difluoroazetidin-1-yl)bicyclo[1.1.1]pentan-1-yl)-1-(4-fluorophenyl)-3,4-dihydroisoquinoline-2(1H)-carboxamide, Compound 4052

At 0° C., phosgene (20% in toluene, 112 μL, 0.211 mmol) was added to a stirred solution of (S)-1-(4-fluorophenyl)-1,2,3,4-tetrahydroisoquinoline (32.0 mg, 0.141 mmol) and triethylamine (29 μL, 0.211 mmol) in dichloromethane (1 mL). After stirring for 2 hours at room temperature, the reaction mixture was concentrated to dryness under reduced pressure. The residue was dissolved in dichloromethane (3 mL), triethylamine (49 μL, 0.352 mmol) was added followed by 3-(3,3-difluoroazetidin-1-yl)bicyclo[1.1.1]pentan-1-amine bis(2,2,2-trifluoroacetate), 46 (40.6 mg, 0.101 nmol). After stirring overnight, the reaction mixture was purified by flash column chromatography (silica, eluting with 0 to 10% methanol in dichloromethane). The product was dissolved in methanol (1 mL) and brought onto an SCX-2 column and eluted with methanol until neutral. Next, the column was eluted with ammonia in methanol (7 M). The basic fraction was concentrated to dryness under reduced pressure. The residue was purified acidic preparative MPLC (Linear Gradient: t=0 min 5% A, t=1 min 5% A, t=2 min 20% A, t=17 min 60% A; t=18 min 100% A; t=26 min 100%; detection: 220/265 nm). The product fractions were combined and lyophilized to afford (S)—N-(3-(3,3-difluoroazetidin-1-yl)bicyclo[1.1.1]pentan-1-yl)-1-(4-fluorophenyl)-3,4-dihydroisoquinoline-2(1H)-carboxamide, compound 4052 7.2 mg) as a white solid. LCMS: 98.5%, RT=3.796 min., (M+H)⁺=428 (Method C). ¹H NMR (400 MHz, chloroform-d) δ 7.25-7.21 (m, 2H), 7.21-7.12 (m, 4H), 6.99-6.89 (m, 2H), 6.38 (s, 1H), 4.95 (s, 1H), 3.62 (t, J=12.1 Hz, 4H), 3.53-3.47 (m, 2H), 2.96-2.85 (m, 1H), 2.83-2.70 (m, 1H), 2.06 (s, 6H).

### Example 49. (S)—N-((trans)-3-amino-1-methylcyclobutyl)-1-(4-fluorophenyl)-3,4-dihydroisoquinoline-2(1H)-carboxamide, Compound 4053

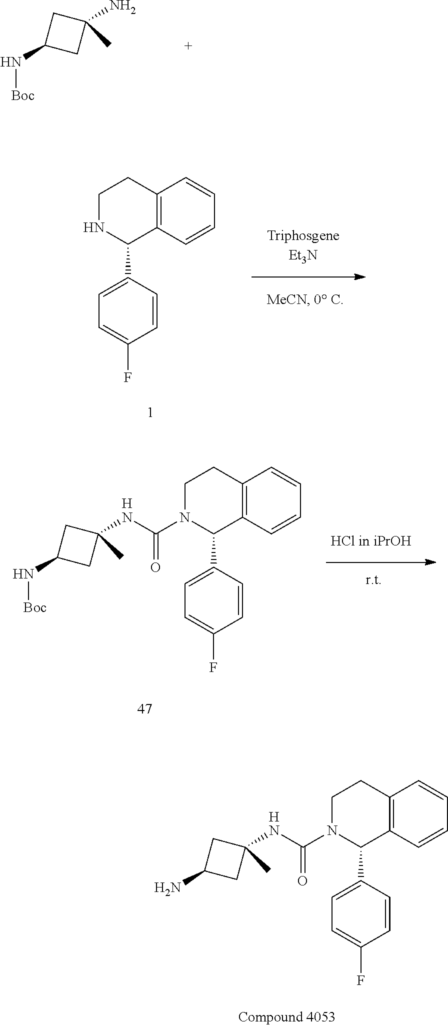

### Step 1. Synthesis of tert-butyl ((trans)-3-((S)-1-(4-fluorophenyl)-1,2,3,4-tetrahydroisoquinoline-2-carboxamido)-3-methylcyclobutyl)carbamate, 47

A solution of tert-butyl (trans-3-amino-3-methylcyclobutyl)carbamate (75 mg, 0.375 mmol) and triethylamine (0.107 mL, 0.768 mmol) in acetonitrile (dry, 1.0 mL) was added dropwise to a solution of triphosgene (36.67 mg, 0.123 mmol) in acetonitrile (dry, 1.0 mL) at 0° C. in 5 minutes. After 1 minute, a solution of (S)-1-(4-fluorophenyl)-1,2,3,4-tetrahydroisoquinoline, 1 (93.6 mg, 0.413 mmol) and triethylamine (0.107 mL, 0.768 mmol) in acetonitrile (dry, 1.0 mL) was added dropwise in 2 minutes. The reaction mixture was stirred at 0° C. for an additional 30 minutes and then evaporated to dryness under reduced pressure. The residue was partitioned between a mixture of water and saturated aqueous NaHCO₃ (1:1, 15 mL) and dichloromethane (15 mL). The layers were separated, and the aqueous phase was extracted with dichloromethane (10 mL). The combined organic layers were passed through a phase separator and evaporated under reduced pressure. The residue was purified by flash column chromatography (silica, 0 to 60% ethyl acetate in heptane). The product was dissolved in methanol (1.5 mL), brought onto an SCX-2 column (2 g), eluted with methanol, and concentrated to dryness under reduced pressure to give tert-butyl ((trans)-3-((S)-1-(4-fluorophenyl)-1,2,3,4-tetrahydroisoquinoline-2-carboxamido)-3-methylcyclobutyl)carbamate, 47 (105.5 mg) as a white solid. LCMS: 98%, RT=2.195 min., (M+H)⁺=454 (Method B). ¹H-NMR (400 MHz, chloroform-d₆) δ 7.25-7.11 (m, 6H), 6.99-6.91 (m, 2H), 6.36 (s, 1H), 4.76-4.62 (m, 1H), 4.51 (s, 1H), 4.24-4.08 (m, 1H), 3.51 (t, J=6.3 Hz, 2H), 2.91 (dt, J=15.8, 6.4 Hz, 1H), 2.78 (dt, J=15.8, 5.9 Hz, 1H), 2.73-2.63 (m, 1H), 2.63-2.51 (m, 1H), 1.87 (ddd, J=12.5, 7.7, 4.6 Hz, 2H), 1.46 (s, 3H), 1.43 (s, 9H).

### Step 2. Synthesis of (S)—N-((trans)-3-amino-1-methylcyclobutyl)-1-(4-fluorophenyl)-3,4-dihydroisoquinoline-2(1H)-carboxamide, Compound 4053

HCl (6 M in 2-propanol, 0.25 mL, 1.5 mmol) was added to a solution of tert-butyl ((trans)-3-((S)-1-(4-fluorophenyl)-1,2,3,4-tetrahydroisoquinoline-2-carboxamido)-3-methylcyclobutyl)carbamate, 47 (98 mg, 0.216 mmol) in 2-propanol (1 mL) at room temperature. After stirring the reaction mixture for 6 hours, additional HCl (6 M in 2-propanol, 0.25 mL, 1.5 mmol) was added stirring was continued for 72 hours. Then, the reaction mixture was diluted with aqueous K₂CO₃ (2 M, 10 mL) and extracted with dichloromethane (2×25 mL). The combined organic layer was dried on Na₂SO₄ and evaporated under reduced pressure. The residue was lyophilized from a mixture of acetonitrile and water (1:1, 3 mL) to give (S)—N-((trans)-3-amino-1-methylcyclobutyl)-1-(4-fluorophenyl)-3,4-dihydroisoquinoline-2(1H)-carboxamide, compound 4053 (68 mg) as a white solid. LCMS: 99.7%, RT=1.054 min., (M+H)⁺=354 (Method P). ¹H-NMR (400 MHz, chloroform-d₆) δ 7.25-7.12 (m, 6H), 6.95 (t, J=8.7 Hz, 2H), 6.34 (s, 1H), 4.49 (s, 1H), 3.60-3.42 (m, 3H), 2.91 (dt, J=15.9, 6.4 Hz, 1H), 2.79 (dt, J=15.7, 5.9 Hz, 1H), 2.63-2.52 (m, 2H), 1.78-1.67 (m, 2H), 1.47 (s, 3H).

### Example 50. (S)—N-((cis)-3-amino-1-methylcyclobutyl)-1-(4-fluorophenyl)-3,4-dihydroisoquinoline-2(1H)-carboxamide, Compound 4054

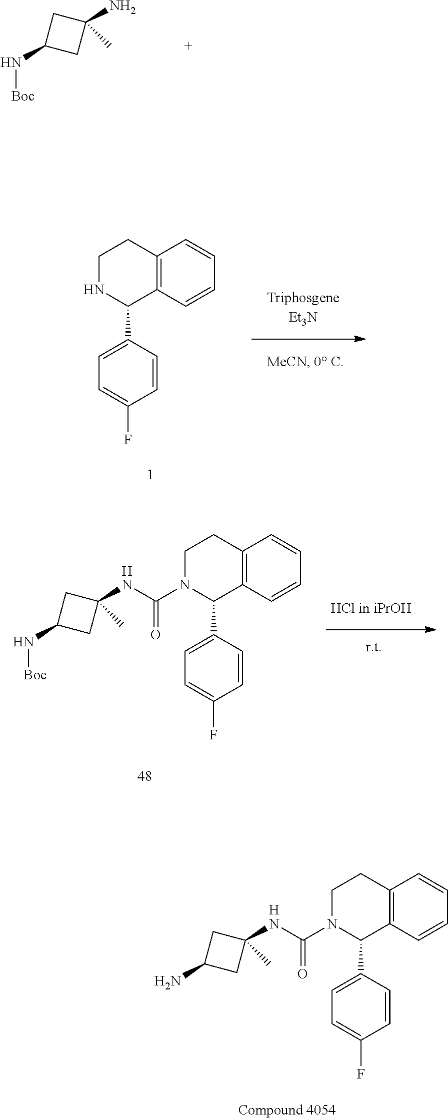

### Step 1. Synthesis of tert-butyl ((cis)-3-((S)-1-(4-fluorophenyl)-1,2,3,4-tetrahydroisoquinoline-2-carboxamido)-3-methylcyclobutyl)carbamate, 48

Starting from tert-butyl (cis-3-amino-3-methylcyclobutyl)carbamate (50 mg, 0.250 mmol) and (S)-1-(4-fluorophenyl)-1,2,3,4-tetrahydroisoquinoline, 1 (62.4 mg, 0.275 mmol), tert-butyl ((cis)-3-((S)-1-(4-fluorophenyl)-1,2,3,4-tetrahydroisoquinoline-2-carboxamido)-3-methylcyclobutyl)carbamate, 48 (64.8 mg) was prepared as described for tert-butyl ((trans)-3-((S)-1-(4-fluorophenyl)-1,2,3,4-tetrahydroisoquinoline-2-carboxamido)-3-methylcyclobutyl)carbamate (see Scheme 49). White solid. LCMS: 100%, RT=2.198 min., (M+H)⁺=454 (Method B). ¹H-NMR (400 MHz, chloroform-d₆) δ 7.25-7.13 (m, 6H), 6.99-6.91 (m, 2H), 6.37 (s, 1H), 5.08-4.96 (m, 1H), 4.54 (s, 1H), 4.07-3.93 (m, 1H), 3.50 (t, J=6.3 Hz, 2H), 2.89 (dt, J=15.8, 6.3 Hz, 1H), 2.77 (dt, J=15.7, 6.2 Hz, 1H), 2.54-2.37 (m, 4H), 1.42 (s, 9H), 1.39 (s, 3H).

### Step 2. Synthesis of (S)—N-((cis)-3-amino-1-methylcyclobutyl)-1-(4-fluorophenyl)-3,4-dihydroisoquinoline-2(1H)-carboxamide, Compound 4054

Starting from tert-butyl ((cis)-3-((S)-1-(4-fluorophenyl)-1,2,3,4-tetrahydroisoquinoline-2-carboxamido)-3-methylcyclobutyl)carbamate, 48 (64 mg, 0.141 mmol), (S)—N-((cis)-3-amino-1-methylcyclobutyl)-1-(4-fluorophenyl)-3,4-dihydroisoquinoline-2(1H)-carboxamide, compound 4054 (23 mg) was prepared as described for (S)—N-((trans)-3-amino-1-methylcyclobutyl)-1-(4-fluorophenyl)-3,4-dihydroisoquinoline-2(1H)-carboxamide (see Scheme 49). White solid. LCMS: 99.7%, RT=1.058 min., (M+H)⁺=354 (Method P). ¹H-NMR (400 MHz, chloroform-d₆) δ 7.25-7.13 (m, 6H), 6.95 (t, J=8.7 Hz, 2H), 6.37 (s, 1H), 4.59 (s, 1H), 3.50 (t, J=6.2 Hz, 2H), 3.29 (p, J=7.7 Hz, 1H), 2.90 (dt, J=15.9, 6.4 Hz, 1H), 2.77 (dt, J=15.7, 5.9 Hz, 1H), 2.51-2.41 (m, 2H), 2.10-1.98 (m, 2H), 1.40 (s, 3H).

### Scheme 51. (S)—N-((trans)-3-aminocyclobutyl)-1-(4-fluorophenyl)-N-methyl-3,4-dihydroisoquinoline-2(1H)-carboxamide, Compound 4055

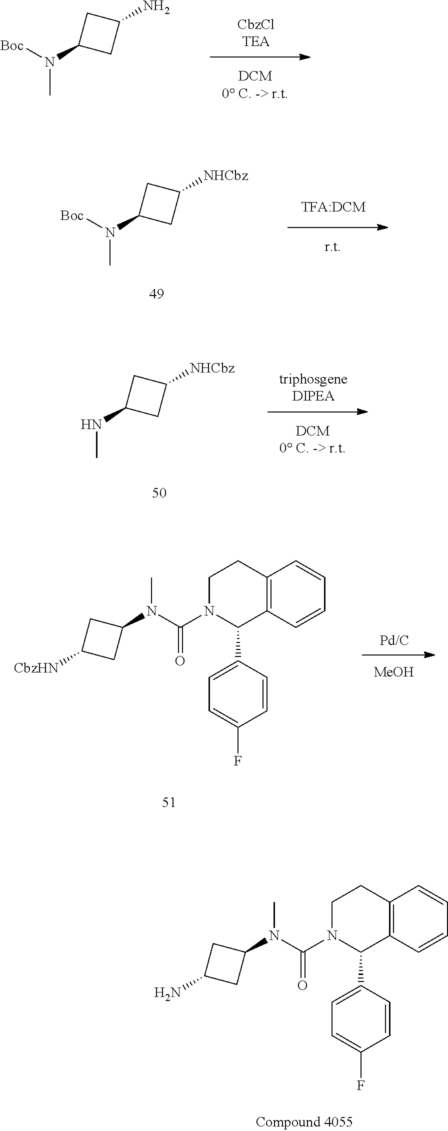

### Step 1. Synthesis of tert-butyl ((trans)-3-(((benzyloxy)carbonyl)amino)cyclobutyl)(methyl)carbamate, 49

Benzyl chloroformate (69 μL, 0.487 mmol) and triethylamine (62 μL, 0.449 mmol) were added to a solution of tert-butyl ((trans)-3-aminocyclobutyl)(methyl)carbamate (75 mg, 0.374 mmol) in dichloromethane (1 mL) at 0° C. The reaction mixture was allowed to gradually warm to room temperature and stirred overnight. The reaction mixture was diluted with dichloromethane (15 mL) and quenched with saturated aqueous NaHCO₃ (10 mL). The layers were separated and the aqueous phase was extracted with dichloromethane (2×10 mL). The combined organics were dried over Na₂SO₄ and evaporated under reduced pressure. The residue was purified by flash column chromatography (silica, eluting with 0 to 40% ethyl acetate in heptane) to give tert-butyl ((trans)-3-(((benzyloxy)carbonyl)amino)cyclobutyl)(methyl)carbamate, 49 (98 mg) as colorless oil. LCMS: 98%, RT=2.109 min., (M+Na)⁺=357 (Method B). ¹H NMR (400 MHz, chloroform-d) δ 7.51-7.28 (m, 5H), 5.09 (s, 2H), 5.00 (s, 1H), 4.70 (br s, 1H), 4.24-3.98 (m, 1H), 2.82 (s, 3H), 2.58-2.43 (m, 2H), 2.28-2.09 (m, 2H), 1.45 (s, 9H).

### Step 2. Synthesis of benzyl ((trans)-3-(methylamino)cyclobutyl)carbamate, 50

Trifluoroacetic acid (1 mL) was added to a solution of tert-butyl ((trans)-3-(((benzyloxy)carbonyl)amino)cyclobutyl)(methyl)carbamate, 49 (98 mg, 0.293 mmol) in dichloromethane (4 mL) at room temperature. After stirring for 45 minutes, the reaction mixture was diluted with dichloromethane (10 mL) and water (15 mL) and the layers were separated. The aqueous phase was basified with K₂CO₃ (solid) till pH-11 and extracted with dichloromethane (5×10 mL). The combined organics were dried on Na₂SO₄ and evaporated under reduced pressure. The residue was dissolved in methanol (1 mL) and brought onto an SCX-2 column (1 g) and eluted with methanol until neutral. Next, the column was eluted with ammonia in methanol (7 M). The basic fraction was concentrated to dryness under reduced pressure to give benzyl ((trans)-3-(methylamino)cyclobutyl)carbamate, 50 (48 mg) as colorless oil. ¹H NMR (400 MHz, chloroform-d) δ 7.41-7.29 (m, 5H), 5.09 (s, 2H), 4.94 (s, 1H), 4.32-4.16 (m, 1H), 3.36-3.20 (m, 1H), 2.34 (s, 3H), 2.25-1.99 (m, 4H).

### Step 3. Synthesis of benzyl ((trans)-3-((S)-1-(4-fluorophenyl)-N-methyl-1,2,3,4-tetrahydroisoquinoline-2-carboxamido)cyclobutyl)carbamate, 51

A solution of (S)-1-(4-fluorophenyl)-1,2,3,4-tetrahydroisoquinoline, 1 (45 mg, 0.198 mmol) and N,N-diisopropylethylamine (36 μL, 0.208 mmol) in dichloromethane (0.5 mL) was added dropwise to a solution of triphosgene (21 mg, 0.069 mmol) in dichloromethane (0.5 mL) under a argon atmosphere at 0° C. After stirring at room temperature overnight, the mixture was cooled to 0° C. and a solution of benzyl ((trans)-3-(methylamino)cyclobutyl)carbamate, 50 (50 mg, 0.198 mmol) and N,N-diisopropylethylamine (36 μL, 0.208 mmol) in dichloromethane (0.5 mL) was added dropwise. After stirring at room temperature for 4 hours, the reaction mixture was diluted with dichloromethane (15 mL). The mixture was washed with aqueous HCl (0.5 M, 10 mL) and saturated aqueous NaHCO₃ (10 mL), dried over Na₂SO₄, and evaporated under reduced pressure. The residue was purified by acidic preparative MPLC (Linear Gradient: t=0 min 5% A, t=1 min 5% A; t=16 min 70% A; t=17 min 100%; t=22 min 100% A; detection: 220/245 nm) to give benzyl ((trans)-3-((S)-1-(4-fluorophenyl)-N-methyl-1,2,3,4-tetrahydroisoquinoline-2-carboxamido)cyclobutyl)carbamate, 51 (37 mg) as white solid. LCMS: 85%, RT=2.184 min., (M+H)⁺=488 (Method A). ¹H NMR (400 MHz chloroform-d) δ 7.42-7.30 (m, 5H), 7.24-7.10 (m, 5H), 7.01-6.90 (m, 3H), 6.17 (s, 1H), 5.09 (s, 2H), 4.95 (s, 1H), 4.21-4.05 (m, 2H), 3.79-3.63 (m, 1H), 3.31-3.20 (m, 1H), 3.15-2.77 (m, 2H), 2.72 (s, 3H), 2.50-2.33 (m, 2H), 2.22-2.08 (m, 2H).

### Step 4. Synthesis of (S)—N-((trans)-3-aminocyclobutyl)-1-(4-fluorophenyl)-N-methyl-3,4-dihydroisoquinoline-2(1H)-carboxamide, Compound 4055

Under an argon atmosphere, palladium (10% on activated carbon, 50% wet, 16 mg, 7.59 μmol) was added to a solution of benzyl ((trans)-3-((S)-1-(4-fluorophenyl)-N-methyl-1,2,3,4-tetrahydroisoquinoline-2-carboxamido)cyclobutyl)carbamate, 51 (37 mg, 0.076 mmol) in methanol (2 mL). The resulting mixture was stirred under a hydrogen atmosphere for 5 hours. The reaction mixture was filtered through Celite and washed with methanol. The filtrate was evaporated under reduced pressure. The residue was purified by acidic preparative MPLC (Linear Gradient: t=0 min 5% A, t=1 min 5% A; t=16 min 50% A; t=17 min 100%; t=22 min 100% A; detection: 220/254 nm) to give (S)—N-((trans)-3-aminocyclobutyl)-1-(4-fluorophenyl)-N-methyl-3,4-dihydroisoquinoline-2(1H)-carboxamide, compound 4055 (11 mg) as white solid after lyophilization of the product containing fractions. LCMS: 98.7%, RT=1.069 min., (M+H)⁺=354 (Method P). ¹H NMR (400 MHz, CD₃OD) δ 7.33-7.09 (m, 5H), 7.06-6.91 (m, 3H), 6.15 (s, 1H), 4.40-4.16 (m, 1H), 3.85-3.64 (m, 2H), 3.31 (m, 1H), 3.06-2.93 (m, 1H), 2.91-2.82 (m, 1H), 2.80 (s, 3H), 2.68-2.46 (m, 2H), 2.42-2.26 (m, 2H).

### Example 52. (S)-2-(3-aminobicyclo[1.1.1]pentan-1-yl)-1-(1-(4-fluorophenyl)-3,4-dihydroisoquinolin-2(1H)-yl)ethan-1-one, Compound 4056

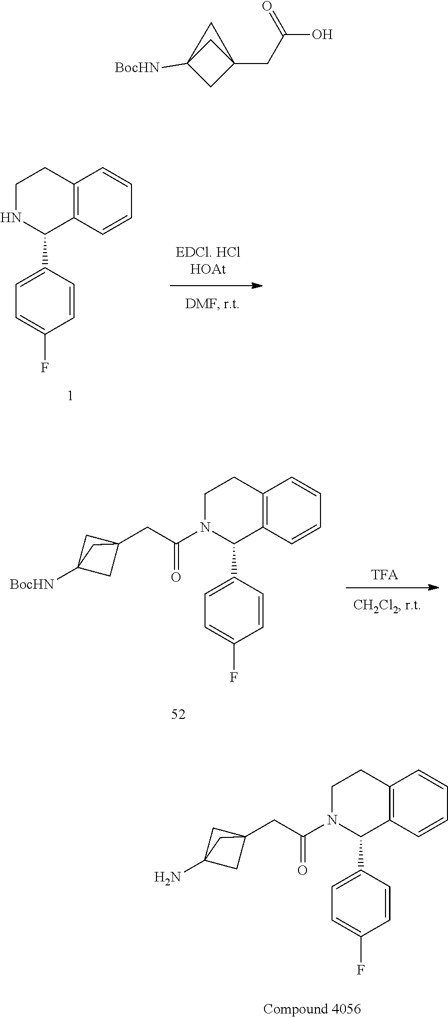 [SMILES: CC(C)(C)OC(=O)NC12CC(CC(=O)O)(C1)C2 ; Fc1ccc([C@@H]2NCCc3ccccc32)cc1 ; CC(C)(C)OC(=O)NC12CC(CC(=O)N3CCc4ccccc4[C@@H]3c3ccc(F)cc3)(C1)C2 ; NC12CC(CC(=O)N3CCc4ccccc4[C@@H]3c3ccc(F)cc3)(C1)C2]

### Step 1. Synthesis of tert-butyl (S)-(3-(2-(1-(4-fluorophenyl)-3,4-dihydroisoquinolin-2(1H)-yl)-2-oxoethyl)bicyclo[1.1.1]pentan-1-yl)carbamate, 52

2-(3-((Tert-butoxycarbonyl)amino)bicyclo[1.1.1]pentan-1-yl)acetic acid (79 mg, 0.33 mmol) and (S)-1-(4-fluorophenyl)-1,2,3,4-tetrahydroisoquinoline, 1 (74 mg, 0.33 mmol) were dissolved in N,N-dimethylformamide (2 mL). 1-ethyl-3-(3-dimethylaminopropyl)carbodiimide hydrochloride (63 mg, 0.33 mmol) was added, followed by 1-hydroxy-7-azabenzotriazole (4.5 mg, 0.033 mmol) and the resulting solution was stirred at room temperature for 5 hours. Water (10 mL) was added and the mixture was extracted with ethyl acetate (2×10 mL). The combined organic layers were washed with brine (2×5 mL), dried over Na₂SO₄ and concentrated under reduced pressure. The residue was purified by flash chromatography (silica, eluting with 10% to 60% ethyl acetate in heptane) to obtain (S)-(3-(2-(1-(4-fluorophenyl)-3,4-dihydroisoquinolin-2(1H)-yl)-2-oxoethyl)bicyclo[1.1.1]pentan-1-yl)carbamate, 52 (102 mg) as a white solid. LCMS: 100%, RT=2.14 min., (M+H)⁺=451 (Method A). ¹H NMR (400 MHz, chloroform-d) as a mixture of rotamers δ 7.24-6.87 (m, 8.8H), 5.91 (s, 0.2H), 4.88 (br s, 1H), 4.30-4.20 (m, 0.2H), 3.77-3.66 (m, 0.8H), 3.46-3.29 (m, 0.8H), 3.29-3.20 (m, 0.2H), 3.09-2.92 (m, 1H), 2.90-2.75 (m, 1.8H), 2.74-2.65 (m, 0.4H), 2.65-2.54 (m, 0.8H), 1.94 (s, 6H), 1.42 (s, 9H).

### Step 2. Synthesis of (S)-2-(3-aminobicyclo[1.1.1]pentan-1-yl)-1-(1-(4-fluorophenyl)-3,4-dihydroisoquinolin-2(1H)-yl)ethan-1-one, compound 4056

Starting from (S)-(3-(2-(1-(4-fluorophenyl)-3,4-dihydroisoquinolin-2(1H)-yl)-2-oxoethyl)bicyclo[1.1.1]pentan-1-yl)carbamate, 52 (100 mg, 0.209 mmol), (S)-2-(3-aminobicyclo[1.1.1]pentan-1-yl)-1-(1-(4-fluorophenyl)-3,4-dihydroisoquinolin-2(1H)-yl)ethan-1-one, compound 4056 (30 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by SCX-2 (1 g) ion exchange chromatography and acidic preparative MPLC (Linear Gradient: t=0 min 5% A, t=1 min 5% A; t=16 min 40% A; t=17 min 100%; t=22 min 100% A; detection: 220/260 nm) after basic workup (saturated aqueous NaHCO₃/dichloromethane extraction). White amorphous solid. LCMS: 96%, RT=2.51 min., (M-NH₂)⁺=334 (Method C). ¹H NMR (400 MHz, chloroform-d) mixture of rotamers δ 7.25-6.86 (m, 8,8H), 5.92 (s, 0.2H), 4.30-4.21 (m, 0.2H), 3.77-3.65 (m, 0.8H), 3.42-3.31 (m, 0.8H), 3.24-3.15 (m, 0.2H), 3.06-2.53 (m, 4H), 1.82-1.63 (m, 6H).

### Example 53. (S)-1-(1-(4-fluorophenyl)-3,4-dihydroisoquinolin-2(1H)-yl)-2-(3-(methylamino)bicyclo[1.1.1]pentan-1-yl)ethan-1-one, Compound 4057

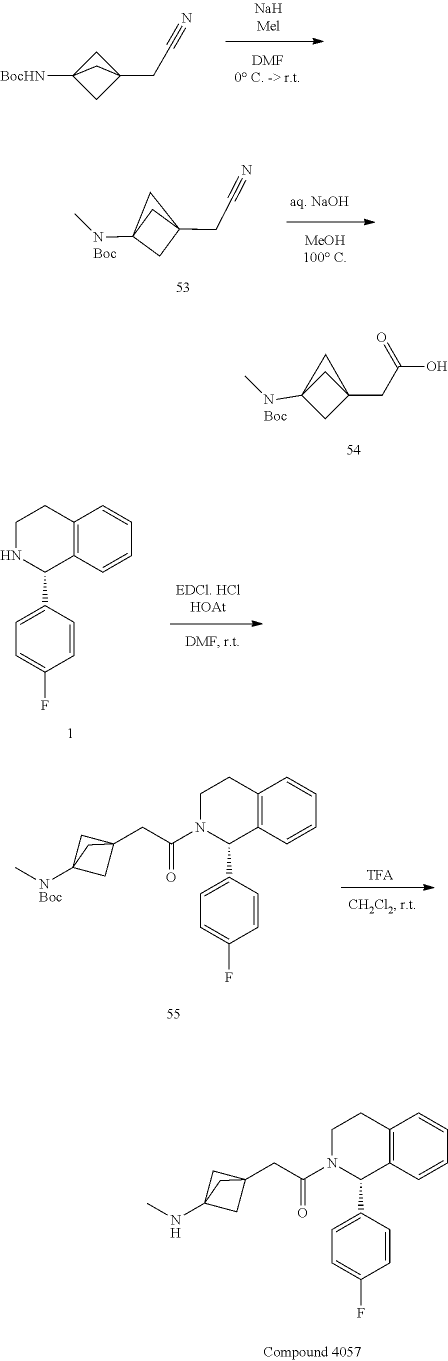

### Step 1. Synthesis of tert-butyl (3-(cyanomethyl)bicyclo[1.1.1]pentan-1-yl)(methyl)carbamate. 53

A solution of tert-butyl (3-(cyanomethyl)bicyclo[1.1.1]pentan-1-yl)carbamate (87 mg, 0.39 mmol) in N,N-dimethylformamide (1 mL) was cooled to 0° C. under an argon atmosphere. Sodium hydride (60% in mineral oil, 17 mg, 0.43 mmol) was added, followed by methyl iodide (0.027 ml, 0.43 mmol) after 5 mins. The mixture was stirred at room temperature for 2 hours. The mixture was diluted with water (10 mL) and extracted with ethyl acetate (2×10 mL). The combined organic layers were washed with brine (2×5 mL), dried over Na₂SO₄, and concentrated under reduced pressure. The residue was purified by flash column chromatography (silica, eluting with 5% to 40% ethyl acetate in heptane) to obtain tert-butyl (3-(cyanomethyl)bicyclo[1.1.1]pentan-1-yl)(methyl)carbamate, 53 (46 mg) as a colorless syrup. ¹H NMR (400 MHz, chloroform-d) δ 2.80 (s, 3H), 2.65 (s, 2H), 2.08 (s, 6H), 1.46 (s, 9H).

### Step 2. Synthesis of 2-(3-((tert-butoxycarbonyl)(methyl)amino)bicyclo[1.1.1]pentan-1-yl)acetic acid, 54

Tert-butyl (3-(cyanomethyl)bicyclo[1.1.1]pentan-1-yl)(methyl)carbamate, 53 (46 mg, 0.20 mmol) was dissolved in methanol (1 mL). Aqueous NaOH (2 M, 1.0 mL) was added and the mixture was heated at 100° C. for 7 hours. The mixture was acidified with aqueous HCl (1 M, 3 mL) and extracted with ethyl acetate (2×5 mL). The combined organic layers were dried over Na₂SO₄ and concentrated under reduced pressure to obtain 2-(3-((tert-butoxycarbonyl)(methyl)amino)bicyclo[1.1.1]pentan-1-yl)acetic acid, 54 (44 mg) as a solid. ¹H NMR (400 MHz, DMSO-d₆) δ 12.10 (s, 1H), 2.69 (s, 3H), 2.47 (s, 2H), 1.93 (s, 6H), 1.39 (s, 9H).

### Step 3. Synthesis of tert-butyl (S)-(3-(2-(1-(4-fluorophenyl)-3,4-dihydroisoquinolin-2(1H)-yl)-2-oxoethyl)bicyclo[1.1.1]pentan-1-yl)(methyl)carbamate, 55

Starting from 2-(3-((tert-butoxycarbonyl)(methyl)amino)bicyclo[1.1.1]pentan-1-yl)acetic acid, 54 (44 mg, 0.17 mmol) and (S)-1-(4-fluorophenyl)-1,2,3,4-tetrahydroisoquinoline, 1 (45 mg, 0.20 mmol), (tert-butyl (S)-(3-(2-(1-(4-fluorophenyl)-3,4-dihydroisoquinolin-2(1H)-yl)-2-oxoethyl)bicyclo[1.1.1]pentan-1-yl)(methyl)carbamate, 55 (61 mg) was prepared according to the procedure described for (S)-(3-(2-(1-(4-fluorophenyl)-3,4-dihydroisoquinolin-2(1H)-yl)-2-oxoethyl)bicyclo[1.1.1]pentan-1-yl)carbamate (see Scheme 52). White solid. LCMS: 97%, RT=2.25 min., (M+H)⁺=465 (Method A). ¹H NMR (400 MHz, chloroform-d) mixture of rotamers δ 7.25-6.88 (m, 8.8H), 5.92 (s, 0.2H), 4.32-4.22 (m, 0.2H), 3.78-3.65 (m, 0.8H), 3.46-3.32 (m, 0.8H), 3.28-3.16 (m, 0.2H), 3.13-2.88 (m, 1.2H), 2.87-2.57 (m, 5.8H), 2.02-1.84 (m, 6H), 1.50-1.36 (m, 9H).

### Step 4. Synthesis of (S)-1-(1-(4-fluorophenyl)-3,4-dihydroisoquinolin-2(1H)-yl)-2-(3-(methylamino)bicyclo[1.1.1]pentan-1-yl)ethan-1-one, Compound 4057

Starting from (tert-butyl (S)-(3-(2-(1-(4-fluorophenyl)-3,4-dihydroisoquinolin-2(1H)-yl)-2-oxoethyl)bicyclo[1.1.1]pentan-1-yl)(methyl)carbamate, 55 (60 mg, 0.13 mmol), (S)-1-(1-(4-fluorophenyl)-3,4-dihydroisoquinolin-2(1H)-yl)-2-(3-(methylamino)bicyclo[1.1.1]pentan-1-yl)ethan-1-one, Compound 4057 (24 mg) was prepared according to the procedure described for (S)-1-(4-fluorophenyl)-N-(3-(prop-2-yn-1-ylamino)bicyclo[1.1.1]pentan-1-yl)-3,4-dihydroisoquinoline-2(1H)-carboxamide 2,2,2-trifluoroacetate (see Scheme 5) and purified by basic preparative MPLC (Linear Gradient: t=0 min 5% A, t=1 min 20% A; t=16 min 60% A; t=17 min 100%; t=22 min 100% A; detection: 220/260 nm), acidic preparative MPLC (Linear Gradient: t=0 min 5% A, t=1 min 5% A; t=16 min 40% A; t=17 min 100%; t=22 min 100% A; detection: 220/260 nm), and SCX-2 (1 g) ion exchange chromatography. White amorphous solid. LCMS: 96%, RT=2.57 min., (M+H)⁺=365 (Method C). ¹H NMR (400 MHz, chloroform-d) mixture of rotamers δ 7.25-6.79 (m, 8.8H), 5.94 (s, 0.2H), 4.30-4.22 (m, 0.2H), 3.80-3.65 (m, 0.8H), 3.46-3.31 (m, 0.8H), 3.25-3.16 (m, 0.2H) 3.13-2.55 (m, 4H), 2.32 (s, 2.4H), 2.29 (s, 0.6H), 1.78-1.61 (m, 6H).

### Example 54. (S)-1-(1-(4-fluorophenyl)-3,4-dihydroisoquinolin-2(1H)-yl)-3-(3-(methylamino)bicyclo[1.1.1]pentan-1-yl)propan-1-one hydrochloride, Compound 4058

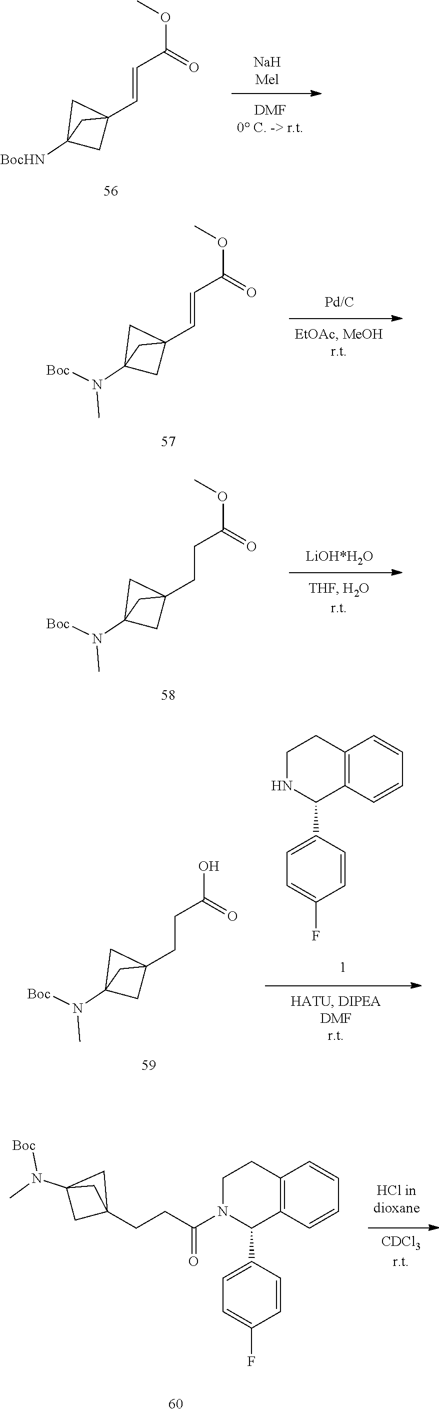 [SMILES: COC(=O)/C=C/C12CC(NC(=O)OC(C)(C)C)(C1)C2 ; COC(=O)/C=C/C12CC(N(C)C(=O)OC(C)(C)C)(C1)C2 ; COC(=O)CCC12CC(N(C)C(=O)OC(C)(C)C)(C1)C2 ; CN(C(=O)OC(C)(C)C)C12CC(CCC(=O)O)(C1)C2 ; Fc1ccc([C@@H]2NCCc3ccccc32)cc1 ; CN(C(=O)OC(C)(C)C)C12CC(CCC(=O)N3CCc4ccccc4[C@@H]3c3ccc(F)cc3)(C1)C2]

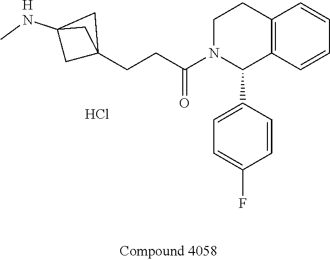

### Step 2. Synthesis of methyl 3-(3-((tert-butoxycarbonyl)(methyl)amino)bicyclo[1.1.1]pentan-1-yl)propanoate, 58

Palladium (10% on activated carbon, 50% wet, 37.8 mg, 0.018 mmol) was added to a solution of methyl (E)-3-(3-((tert-butoxycarbonyl)(methyl)amino)bicyclo[1.1.1]pentan-1-yl)acrylate, 57 (100 mg, 0.355 mmol) in ethyl acetate (5 mL) and methanol (5 mL) under an argon atmosphere. The resulting mixture was stirred under a hydrogen atmosphere overnight. The reaction mixture was filtered through Celite and washed with methanol. The filtrate was concentrated under reduced pressure to give methyl 3-(3-((tert-butoxycarbonyl)(methyl)amino)bicyclo[1.1.1]pentan-1-yl)propanoate 58 (100 mg). LCMS: RT=2.126 min., (M-t-Bu+H)⁺=228 (Method A). ¹H NMR (400 MHz, chloroform-d) b 3.67 (s, 3H), 2.77 (s, 3H), 2.29 (t, J=7.6 Hz, 2H), 1.92-1.82 (m, 8H), 1.45 (s, 9H).

### Step 3. Synthesis of 3-(3-((tert-butoxycarbonyl)(methyl)amino)bicyclo[1.1.1]pentan-1-yl)propanoic acid, 59

To a solution of methyl 3-(3-((tert-butoxycarbonyl)(methyl)amino)bicyclo[1.1.1]pentan-1-yl)propanoate, 58 (100 mg, 0.353 mmol) in tetrahydrofuran (1 mL) and water (1 mL), lithium hydroxide monohydrate (44.4 mg, 1.059 mmol) was added. After stirring for 4 hours, the reaction mixture was concentrated under reduced pressure. The residue was diluted with water and acidified with aqueous HCl (1 M). The aqueous layer was extracted with dichloromethane (3×15 mL). The combined organics were dried over Na₂SO₄ and evaporated under reduced pressure to give 3-(3-((tert-butoxycarbonyl)(methyl)amino)bicyclo[1.1.1]pentan-1-yl)propanoic acid, 59 (78 mg). LCMS: RT=1.968 min., (M−H)⁺=268 (Method A). ¹H NMR (400 MHz, chloroform-d) δ 2.78 (s, 3H), 2.34 (t, J=7.6 Hz, 2H), 1.93-1.82 (m, 8H), 1.45 (s, 9H).

### Step 4. Synthesis of tert-butyl (S)-(3-(3-(1-(4-fluorophenyl)-3,4-dihydroisoquinolin-2(1H)-yl)-3-oxopropyl)bicyclo[1.1.1]pentan-1-yl)(methyl)carbamate, 60

To a solution of 3-(3-((tert-butoxycarbonyl)(methyl)amino)bicyclo[1.1.1]pentan-1-yl)propanoic acid, 59 (78 mg, 0.290 mmol) in N,N-dimethylformamide (dry, 2.5 mL), 1-[bis(dimethylamino)methylene]-1H-1,2,3-triazolo[4,5-b]pyridinium 3-oxid hexafluorophosphate (92 mg, 0.242 mmol) followed by N,N-diisopropylethylamine (0.126 mL, 0.726 mmol) was added at room temperature under argon atmosphere. After stirring for 15 minutes, (S)-1-(4-fluorophenyl)-1,2,3,4-tetrahydroisoquinoline, 1 (55 mg, 0.242 mmol) was added. After stirring for 4 hours, the reaction mixture was diluted with chloroform and quenched with saturated aqueous NaHCO₃ (10 mL). The layers were separated and the aqueous phase was extracted with chloroform (3×15 mL). The combined organics were dried over Na₂SO₄ and evaporated under reduced pressure. The residue was purified by flash column chromatography (silica, eluting with 0 to 30% ethyl acetate in heptane) to give tert-butyl (S)-(3-(3-(1-(4-fluorophenyl)-3,4-dihydroisoquinolin-2(1H)-yl)-3-oxopropyl)bicyclo[1.1.1]pentan-1-yl)(methyl)carbamate, 60 (83 mg). LCMS: 98.1%, RT=2.36 min., (M-t-Bu+H)⁺=423 (Method B). ¹H NMR (400 MHz, chloroform-d) δ 7.25-7.11 (m, 5H), 7.05 (d, J=7.6 Hz, 1H), 6.98-6.86 (m, 3H), 3.82-3.70 (m, 1H), 3.51-3.33 (m, 1H), 3.06-2.93 (m, 1H), 2.85 (dt, J=15.8, 3.6 Hz, 1H), 2.76 (s, 3H), 2.51-2.24 (m, 2H), 1.98-1.88 (m, 2H), 1.86 (s, 6H), 1.44 (s, 9H).

### Step 5. Synthesis of (S)-1-(1-(4-fluorophenyl)-3,4-dihydroisoquinolin-2(1H)-yl)-3-(3-(methylamino)bicyclo[1.1.1]pentan-1-yl)propan-1-one hydrochloride, Compound 4058

To a solution of tert-butyl (S)-(3-(3-(1-(4-fluorophenyl)-3,4-dihydroisoquinolin-2(1H)-yl)-3-oxopropyl)bicyclo[1.1.1]pentan-1-yl)(methyl)carbamate, 60 (83 mg, 0.173 mmol) in chloroform-d (5 mL) a solution of hydrochloric acid in 1,4-dioxane (4 M, 0.434 mL, 1.734 mmol) was added at room temperature under argon atmosphere. After stirring overnight, the reaction mixture was concentrated under reduced pressure. The residue was dissolved in methanol (1 mL) and brought onto an SCX-2 column (1 g) and eluted with methanol until neutral. Next, the column was eluted with ammonia in methanol (7 M). The basic fraction was concentrated to dryness under reduced pressure. The crude was purified by basic preparative MPLC (Linear Gradient: t=0 min 5% A, t=1 min 5% A; t=16 min 60% A; t=17 min 100% A; t=22 min 100% A; detection: 220 nm) twice. The product containing fractions were concentrated and lyophilized from a mixture of acetonitrile and water (1:1, 4 mL). The residue was dissolved in aqueous HCl (0.1 M, 0.105 mL) and lyophilized to give (S)-1-(1-(4-fluorophenyl)-3,4-dihydroisoquinolin-2(1H)-yl)-3-(3-(methylamino)bicyclo[1.1.1]pentan-1-yl)propan-1-one hydrochloride, compound 4058 (5.2 mg). LCMS: 92.7%, RT=3.99 min., (M+H)⁺=379 (Method D). ¹H NMR (400 MHz, chloroform-d) mixture of rotamers (˜5:1); major isomer δ 10.19-9.91 (m, 2H), 7.25-7.10 (m, 5H), 7.05 (d, J=7.5 Hz, 1H), 6.94 (t, J=8.7 Hz, 2H), 6.87 (s, 1H), 3.78-3.68 (m, 1H), 3.48-3.36 (m, 1H), 2.98 (ddd, J=16.6, 11.0, 5.7 Hz, 1H), 2.86 (dt, J=16.3, 4.0 Hz, 1H), 2.63-2.51 (m, 3H), 2.43-2.23 (m, 2H), 2.12-1.90 (m, 8H).

### Example 55: ((1S,2R)-2-aminocyclobutyl)methyl (S)-1-(4-fluorophenyl)-3,4-dihydroisoquinoline-2(1H)-carboxylate (Compound 4132)

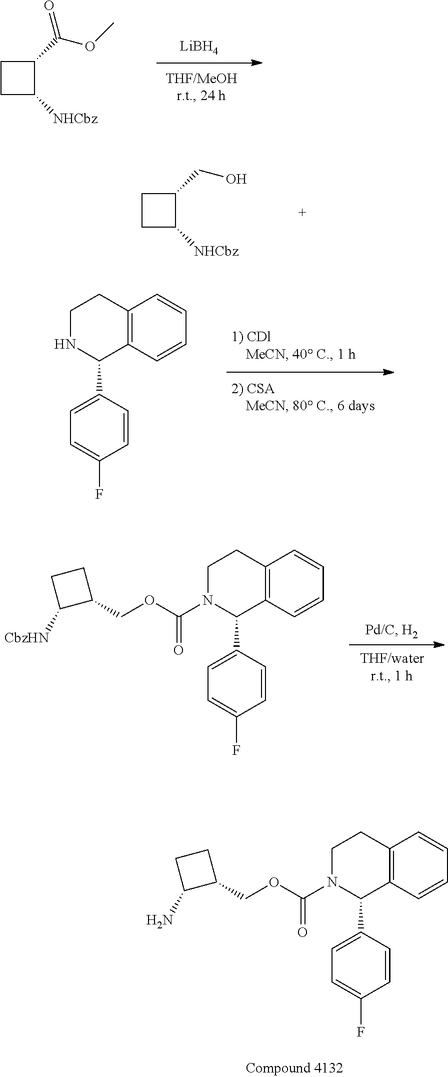 [SMILES: COC(=O)[C@H]1CC[C@H]1NC(=O)OCc1ccccc1 ; "LiBH4" ; O=C(N[C@@H]1CC[C@@H]1CO)OCc1ccccc1 ; Fc1ccc([C@@H]2NCCc3ccccc32)cc1 ; O=C(N[C@@H]1CC[C@@H]1COC(=O)N1CCc2ccccc2[C@@H]1c1ccc(F)cc1)OCc1ccccc1 ; N[C@@H]1CC[C@@H]1COC(=O)N1CCc2ccccc2[C@@H]1c1ccc(F)cc1]

### Synthesis of benzyl ((1R,2S)-2-(hydroxymethyl)cyclobutyl)carbamate

Lithium borohydride (2 M in tetrahydrofuran, 0.174 mL, 0.348 mmol) was added to a solution of methyl (1S,2R)-2-(((benzyloxy)carbonyl)amino)cyclobutane-1-carboxylate (52 mg, 0.174 mmol) in methanol (2 mL). After 1 hour, another portion of lithium borohydride (2 M in tetrahydrofuran, 0.348 mL, 0.695 mmol) was added and stirring was continued for 1 day. The reaction mixture was quenched with water (10 mL) and extracted with ethyl acetate (2×10 mL). The combined organic phase was washed with brine, dried over Na₂SO₄, and concentrated under reduced pressure. The residue was purified by flash column chromatography (silica, 25 to 75% ethyl acetate in heptane) to give benzyl ((1R,2S)-2-(hydroxymethyl)cyclobutyl)carbamate.

LCMS: 97%, RT=1.72 min., (M+H)⁺=236 (method A). ¹H NMR (400 MHz, chloroform-d) δ 7.42-7.28 (m, 5H), 5.56-5.27 (m, 1H), 5.10 (s, 2H), 4.38-4.24 (m, 1H), 3.90-3.74 (m, 1H), 3.74-3.60 (m, 1H), 2.82-2.64 (m, 1H), 2.47-2.29 (m, 1H), 2.16-2.05 (m, 1H), 2.02-1.81 (m, 2H), 1.74-1.60 (m, 1H).

### Synthesis of ((1S,2R)-2-(((benzyloxy)carbonyl)amino)cyclobutyl)methyl (S)-1-(4-fluorophenyl)-3,4-dihydroisoquinoline-2(1H)-carboxylate

A solution of benzyl ((1R,2S)-2-(hydroxymethyl)cyclobutyl)carbamate (26 mg, 0.111 mmol) and 1,1′-carbonyldiimidazole (20 mg, 0.122 mmol) in acetonitrile (1 mL) was heated at 40° C. for 1 hour. (S)-1-(4-fluorophenyl)-1,2,3,4-tetrahydroisoquinoline (27.6 mg, 0.122 mmol) and DL-10-camphorsulfonic acid (20.5 mg, 0.088 mmol) were added and the mixture was heated at 80° C. for 6 days. The mixture was concentrated under reduced pressure and the residue was purified by flash column chromatography (silica, 5 to 50% ethyl acetate in heptane) to give ((1S,2R)-2-(((benzyloxy)carbonyl)amino)cyclobutyl)methyl (S)-1-(4-fluorophenyl)-3,4-dihydroisoquinoline-2(1H)-carboxylate.

LCMS: 97%, RT=2.31 min., (M+H)⁺=489 (method B). ¹H NMR (400 MHz, chloroform-d) mixture of rotamers δ 7.38-7.28 (m, 3H), 7.26-7.05 (m, 7H), 7.04-6.89 (m, 3H), 6.51-6.10 (m, 1H), 5.41-5.19 (m, 1H), 5.08-5.03 (m, 1H), 4.98-4.70 (m, 1H), 4.61-4.17 (m, 3H), 4.08-3.86 (m, 1H), 3.28-3.12 (m, 1H), 3.02-2.78 (m, 2H), 2.78-2.62 (m, 1H), 2.42-2.30 (m, 1H), 2.00-1.87 (m, 2H), 1.56-1.46 (m, 1H).

### Synthesis of ((1S,2R)-2-aminocyclobutyl)methyl (S)-1-(4-fluorophenyl)-3,4-dihydroisoquinoline-2(1H)-carboxylate (Compound 4132)

Starting from a solution of ((1S,2R)-2-(((benzyloxy)carbonyl)amino)cyclobutyl)methyl (S)-1-(4-fluorophenyl)-3,4-dihydroisoquinoline-2(1H)-carboxylate (32 mg, 0.059 mmol) in tetrahydrofuran (2 mL) and water (1 mL), ((1S,2R)-2-aminocyclobutyl)methyl (S)-1-(4-fluorophenyl)-3,4-dihydroisoquinoline-2(1H)-carboxylate (Compound 4132) was prepared as described for ((1R,2R)-2-aminocyclobutyl)methyl (S)-1-(4-fluorophenyl)-3,4-dihydroisoquinoline-2(1H)-carboxylate (Compound 4062) and additionally purified by SCX-2 (1 g) ion exchange chromatography.

LCMS: 99%, RT=1.11 min., (M+H)⁺=355 (method P). ¹H NMR (400 MHz, chloroform-d) mixture of rotamers δ 7.25-7.12 (m, 5H), 7.08-7.01 (m, 1H), 7.01-6.90 (m, 2H), 6.58-6.01 (m, 1H), 4.63-4.43 (m, 1H), 4.35-3.85 (m, 2H), 3.70-3.60 (m, 1H), 3.31-3.21 (m, 10.6, 4.4 Hz, 1H), 3.09-2.88 (m, 1H), 2.86-2.74 (m, 1H), 2.74-2.57 (m, 1H), 2.37-2.24 (m, 1H), 1.97-1.69 (m, 2H), 1.66-1.57 (m, 1H).

### Example 56: ((1R,2S)-2-aminocyclobutyl)methyl (S)-1-(4-fluorophenyl)-3,4-dihydroisoquinoline-2(1H)-carboxylate (Compound 4133)

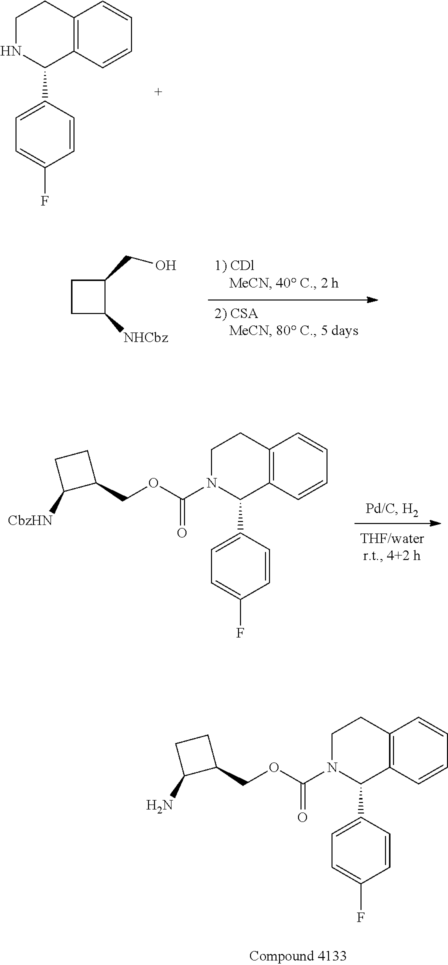 [SMILES: Fc1ccc([C@@H]2NCCc3ccccc32)cc1 ; O=C(N[C@H]1CC[C@H]1CO)OCc1ccccc1 ; O=C(N[C@H]1CC[C@H]1COC(=O)N1CCc2ccccc2[C@@H]1c1ccc(F)cc1)OCc1ccccc1 ; N[C@H]1CC[C@H]1COC(=O)N1CCc2ccccc2[C@@H]1c1ccc(F)cc1]

### Synthesis of ((1R,2S)-2-(((benzyloxy)carbonyl)amino)cyclobutyl)methyl (S)-1-(4-fluorophenyl)-3,4-dihydroisoquinoline-2(1H)-carboxylate

Starting from benzyl ((1S,2R)-2-(hydroxymethyl)cyclobutyl)carbamate (37 mg, 0.157 mmol) and (S)-1-(4-fluorophenyl)-1,2,3,4-tetrahydroisoquinoline (39 mg, 0.173 mmol), ((1R,2S)-2-(((benzyloxy)carbonyl)amino)cyclobutyl)methyl (S)-1-(4-fluorophenyl)-3,4-dihydroisoquinoline-2(1H)-carboxylate was prepared as described for ((1S,2R)-2-(((benzyloxy)carbonyl)amino)cyclobutyl)methyl (S)-1-(4-fluorophenyl)-3,4-dihydroisoquinoline-2(1H)-carboxylate (see Compound 4132).

LCMS: 99%, RT=2.28 min., (M+H)⁺=489 (method A). ¹H NMR (400 MHz, chloroform-d) mixture of rotamers δ 7.38-7.28 (m, 3H), 7.26-7.05 (m, 7H), 7.04-6.89 (m, 3H), 6.51-6.10 (m, 1H), 5.41-4.61 (m, 3H), 4.55-4.21 (m, 3H), 4.14-3.80 (m, 1H), 3.28-3.12 (m, 1H), 2.97-2.65 (m, 3H), 2.43-2.32 (m, 1H), 2.00-1.87 (m, 2H), 1.56-1.46 (m, 1H).

### Synthesis of ((1R,2S)-2-aminocyclobutyl)methyl (S)-1-(4-fluorophenyl)-3,4-dihydroisoquinoline-2(1H)-carboxylate (Compound 4133)

Starting from a solution of ((1R,2S)-2-(((benzyloxy)carbonyl)amino)cyclobutyl)methyl (S)-1-(4-fluorophenyl)-3,4-dihydroisoquinoline-2(1H)-carboxylate (53 mg, 0.108 mmol) in tetrahydrofuran (4 mL) and water (2 mL), ((1R,2S)-2-aminocyclobutyl)methyl (S)-1-(4-fluorophenyl)-3,4-dihydroisoquinoline-2(1H)-carboxylate (Compound 4133) was prepared as described for ((1R,2R)-2-aminocyclobutyl)methyl (S)-1-(4-fluorophenyl)-3,4-dihydroisoquinoline-2(1H)-carboxylate (Compound 4062) and additionally purified by preparative chiral SFC (Method AW). LCMS: 97%, RT=1.13 min., (M+H)⁺=355 (method P). ¹H NMR (400 MHz, chloroform-d) mixture of rotamers δ 7.24-7.09 (m, 5H), 7.08-7.00 (m, 1H), 7.00-6.80 (m, 2H), 6.60-6.01 (m, 1H), 4.51-4.38 (m, 1H), 4.35-4.21 (m, 1H), 4.19-3.82 (m, 1H), 3.72-3.55 (m, 1H), 3.42-3.10 (m, 1H), 3.07-2.86 (m, 1H), 2.83-2.72 (m, 1H), 2.72-2.60 (m, 1H), 2.36-2.17 (m, 1H), 1.94-1.69 (m, 2H), 1.68-1.50 (m, 1H).

### Example 57: ((1S,2S)-2-aminocyclobutyl)methyl (S)-1-(4-fluorophenyl)-3,4-dihydroisoquinoline-2(1H)-carboxylate (Compound 4134)

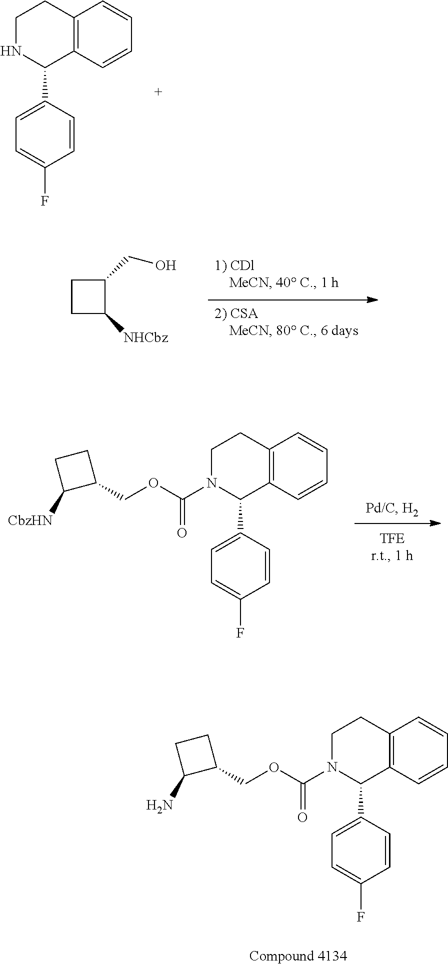 [SMILES: Fc1ccc([C@@H]2NCCc3ccccc32)cc1 ; O=C(N[C@H]1CC[C@@H]1CO)OCc1ccccc1 ; O=C(N[C@H]1CC[C@@H]1COC(=O)N1CCc2ccccc2[C@@H]1c1ccc(F)cc1)OCc1ccccc1 ; N[C@H]1CC[C@@H]1COC(=O)N1CCc2ccccc2[C@@H]1c1ccc(F)cc1]

### Synthesis of ((1S,2S)-2-(((benzyloxy)carbonyl)amino)cyclobutyl)methyl (S)-1-(4-fluorophenyl)-3,4-dihydroisoquinoline-2(1H)-carboxylate

Starting from benzyl ((1S,2S)-2-(hydroxymethyl)cyclobutyl)carbamate (36 mg, 0.138 mmol) and (S)-1-(4-fluorophenyl)-1,2,3,4-tetrahydroisoquinoline (34 mg, 0.151 mmol), ((1S,2S)-2-(((benzyloxy)carbonyl)amino)cyclobutyl)methyl (S)-1-(4-fluorophenyl)-3,4-dihydroisoquinoline-2(1H)-carboxylate was prepared as described for ((1S,2R)-2-(((benzyloxy)carbonyl)amino)cyclobutyl)methyl (S)-1-(4-fluorophenyl)-3,4-dihydroisoquinoline-2(1H)-carboxylate (see Compound 4132). LCMS: 98%, RT=2.31 min., (M+H)⁺=489 (method B). ¹H NMR (400 MHz, chloroform-d) mixture of rotamers δ 7.41-7.28 (m, 5H), 7.24-7.09 (m, 5H), 7.07-6.87 (m, 3H), 6.56-5.97 (m, 1H), 5.22-4.78 (m, 3H), 4.35-3.82 (m, 4H), 3.29-3.12 (m, 1H), 3.08-2.84 (m, 1H), 2.84-2.61 (m, 1H), 2.59-2.35 (m, 1H), 2.35-2.20 (m, 1H), 1.88-1.68 (m, 2H), 1.52-1.36 (m, 1H).

### Synthesis of ((1S,2S)-2-aminocyclobutyl)methyl (S)-1-(4-fluorophenyl)-3,4-dihydroisoquinoline-2(1H)-carboxylate (Compound 4134)

Starting from a solution of ((1S,2S)-2-(((benzyloxy)carbonyl)amino)cyclobutyl)methyl (S)-1-(4-fluorophenyl)-3,4-dihydroisoquinoline-2(1H)-carboxylate (44 mg, 0.090 mmol) in 2,2,2-trifluoroethanol (2.5 mL), ((1S,2S)-2-aminocyclobutyl)methyl (S)-1-(4-fluorophenyl)-3,4-dihydroisoquinoline-2(1H)-carboxylate (Compound 4134) was prepared as described for ((1R,2R)-2-aminocyclobutyl)methyl (S)-1-(4-fluorophenyl)-3,4-dihydroisoquinoline-2(1H)-carboxylate (Compound 4062).

LCMS: 99%, RT=1.12 min., (M+H)⁺=355 (method P). ¹H NMR (400 MHz, chloroform-d) mixture of rotamers δ 7.24-7.12 (m, 5H), 7.08-7.01 (m, 1H), 7.01-6.90 (m, 2H), 6.55-6.06 (m, 1H), 4.37-3.90 (m, 3H), 3.29-3.19 (m, 1H), 3.19-3.07 (m, 1H), 3.07-2.91 (m, 1H), 2.83-2.74 (m, 1H), 2.30-2.13 (m, 2H), 1.84-1.70 (m, 1H), 1.67-1.50 (m, 1H), 1.44-1.31 (m, 1H).

### Example 58: (S)—N-(3-aminobicyclo[1.1.1]pentan-1-yl)-1-phenyl-3,4-dihydroisoquinoline-2(1H)-carboxamide (Compound 4135)

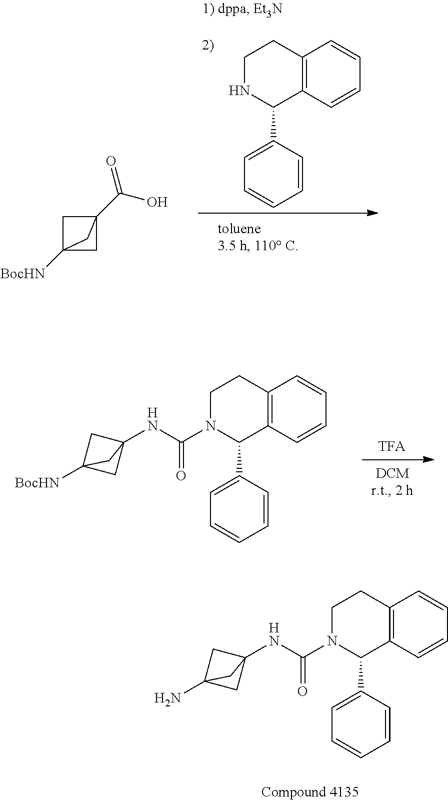

### Synthesis of tert-butyl (S)-(3-(1-phenyl-1,2,3,4-tetrahydroisoquinoline-2-carboxamido)bicyclo[1.1.1]pentan-1-yl)carbamate

Under nitrogen atmosphere, 3-((tert-butoxycarbonyl)amino)bicyclo[1.1.1]pentane-1-carboxylic acid (107.2 mg, 0.472 mmol) was dissolved in a mixture of toluene (dry, 5 ml) and triethylamine (99 μL, 0.710 mmol). At room temperature, diphenylphosphoryl azide (112 μL, 0.517 mmol) was added after which the reaction mixture was stirred at 110° C. for 2 hours. The mixture was cooled to room temperature, (S)-1-phenyl-1,2,3,4-tetrahydroisoquinoline (99.7 mg, 0.476 mmol) was added, and the mixture was warmed again to 110° C. After 1.5 hour, the reaction mixture was cooled to room temperature and concentrated to dryness under reduced pressure. The residue was taken up in dichloromethane (10 mL) and washed with aqueous HCl (1 M, 5 mL) and aqueous NaOH (1 M, 5 mL). The organic layer was passed through a phase-separator and the filtrate was evaporated under reduced pressure. The residue was purified by flash column chromatography (silica, 25 to 75% ethyl acetate in heptane) to give tert-butyl (S)-(3-(1-phenyl-1,2,3,4-tetrahydroisoquinoline-2-carboxamido)bicyclo[1.1.1]pentan-1-yl)carbamate.

LCMS: 99%, RT=2.19 min., (M+H)⁺=434 (method A). ¹H NMR (400 MHz, chloroform-d) δ 7.32-7.13 (m, 9H), 6.32 (s, 1H), 5.12-4.66 (m, 2H), 3.63-3.50 (m, 2H), 2.90 (dt, J=15.8, 6.4 Hz, 1H), 2.79 (dt, J=15.7, 5.9 Hz, 1H), 2.28 (s, 6H), 1.44 (s, 9H).

### Synthesis of (S)—N-(3-aminobicyclo[1.1.1]pentan-1-yl)-1-phenyl-3,4-dihydroisoquinoline-2(1H)-carboxamide (Compound 4135)

Trifluoroacetic acid (1 mL, 12.98 mmol) was added to a solution of tert-butyl (S)-(3-(1-phenyl-1,2,3,4-tetrahydroisoquinoline-2-carboxamido)bicyclo[1.1.1]pentan-1-yl)carbamate (158 mg, 0.364 mmol) in dichloromethane (4 mL). After 2 hours, the reaction mixture was concentrated to dryness under reduced pressure. The residue was dissolved in methanol (1 mL) and brought onto an SCX-2 column (2 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give (S)—N-(3-aminobicyclo[1.1.1]pentan-1-yl)-1-phenyl-3,4-dihydroisoquinoline-2(1H)-carboxamide (Compound 4135).

LCMS: 99%, RT=1.00 min., (M+H)⁺=334 (method P). ¹H NMR (400 MHz, chloroform-d) δ 7.32-7.12 (m, 9H), 6.32 (s, 1H), 4.93 (s, 1H), 3.62-3.49 (m, 2H), 2.89 (dt, J=15.9, 6.4 Hz, 1H), 2.78 (dt, J=15.9, 6.0 Hz, 1H), 2.08 (s, 6H).

### Example 59: 3-aminobicyclo[1.1.1]pentan-1-yl (S)-1-phenyl-3,4-dihydroisoquinoline-2(1H)-carboxylate (Compound 4136)

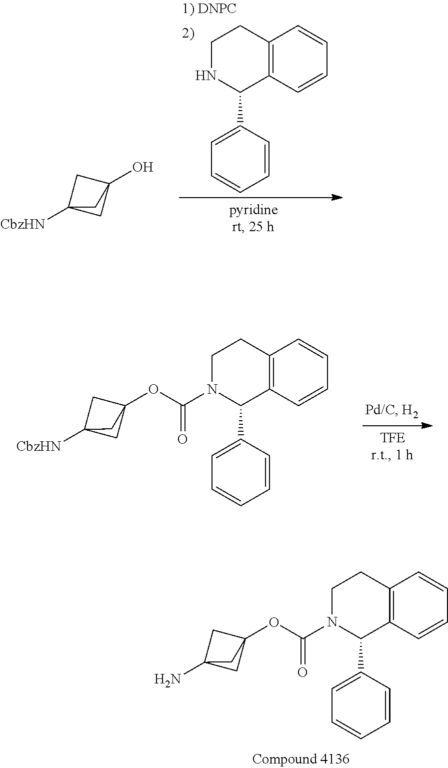 [SMILES: O=C(NC12CC(O)(C1)C2)OCc1ccccc1 ; c1ccc([C@@H]2NCCc3ccccc32)cc1 ; O=C(NC12CC(OC(=O)N3CCc4ccccc4[C@@H]3c3ccccc3)(C1)C2)OCc1ccccc1 ; NC12CC(OC(=O)N3CCc4ccccc4[C@@H]3c3ccccc3)(C1)C2]

### Synthesis of 3-(((benzyloxy)carbonyl)amino)bicyclo[1.1.1]pentan-1-yl (S)-1-phenyl-3,4-dihydroisoquinoline-2(1H)-carboxylate

Bis(p-nitrophenyl) carbonate (312 mg, 1.025 mmol) was added to a solution of benzyl (3-hydroxybicyclo[1.1.1]pentan-1-yl)carbamate (See Compound 4001, 239 mg, 1.025 mmol) in pyridine (dry, 10 mL). The mixture was stirred under argon atmosphere at room temperature for 19 hours before (S)-1-phenyl-1,2,3,4-tetrahydroisoquinoline (214 mg, 1.022 mmol) was added. Stirring was continued for another 6 hours after which the reaction mixture was concentrated to dryness under reduced pressure. The residue was taken up in dichloromethane (50 ml), washed with saturated aqueous NaHCO₃ (2×30 ml), dried over Na₂SO₄, and evaporated under reduced pressure. The residue was purified by flash column chromatography (silica, 0 to 40% ethyl acetate in heptane). The product was taken up in dichloromethane (10 mL) and washed with aqueous NaOH (0.5 M, 10 mL). The aqueous layer was extracted with dichloromethane (5 mL) and the combined organics were dried on Na₂SO₄ and evaporated under reduced pressure to give 3-(((benzyloxy)carbonyl)amino)bicyclo[1.1.1]pentan-1-yl (S)-1-phenyl-3,4-dihydroisoquinoline-2(1H)-carboxylate.

LCMS: 98%, RT=2.35 min., (M+H)⁺=469 (method B). ¹H NMR (400 MHz, chloroform-d) mixture of rotamers δ 7.40-7.10 (m, 9H), 7.09-6.96 (m, 1H), 6.48-6.12 (m, 1H), 5.25-5.00 (m, 3H), 4.17-3.83 (m, 1H), 3.33-3.11 (m, 1H), 3.06-2.88 (m, 1H), 2.82-2.68 (m, 1H), 2.54-2.32 (m, 6H).

### Synthesis of 3-aminobicyclo[1.1.1]pentan-1-yl (S)-1-phenyl-3,4-dihydroisoquinoline-2(1H)-carboxylate (Compound 4136)

3-(((benzyloxy)carbonyl)amino)bicyclo[1.1.1]pentan-1-yl (S)-1-phenyl-3,4-dihydroisoquinoline-2(1H)-carboxylate (65 mg, containing 11 wt % dichloromethane, 0.123 mmol) was coevaporated from 2,2,2-trifluoroethanol (3×0.5 mL). The residue was dissolved in 2,2,2-trifluoroethanol (2.5 mL) and the solution was hydrogenated in the presence of palladium (10 wt % on carbon, containing 50% water, 13.1 mg, 0.012 mmol) at atmospheric hydrogen pressure. After 1 hour, the reaction mixture was filtered through a nylon 0.45 μm filter and the filtrate was evaporated under reduced pressure. The residue was purified by flash column chromatography (silica, 0 to 50% (7M ammonia in methanol) in dichloromethane) to give 3-aminobicyclo[1.1.1]pentan-1-yl (S)-1-phenyl-3,4-dihydroisoquinoline-2(1H)-carboxylate (Compound 4136) after lyophilization from a mixture of acetonitrile and water (4:3, 3.5 mL).

LCMS: 97%, RT=1.12 min., (M+H)⁺=335 (method P). ¹H NMR (400 MHz, DMSO-d₆) mixture of rotamers δ 7.36-7.07 (m, 9H), 6.29-60.4 (m, 1H), 3.95-3.63 (m, 1H), 3.39-3.14 (m, 1H), 2.92-2.70 (m, 2H), 2.21 (br s, 2H), 2.02 (s, 6H).

### Example 60: Synthesis of Compound 4137 and Compound 4138

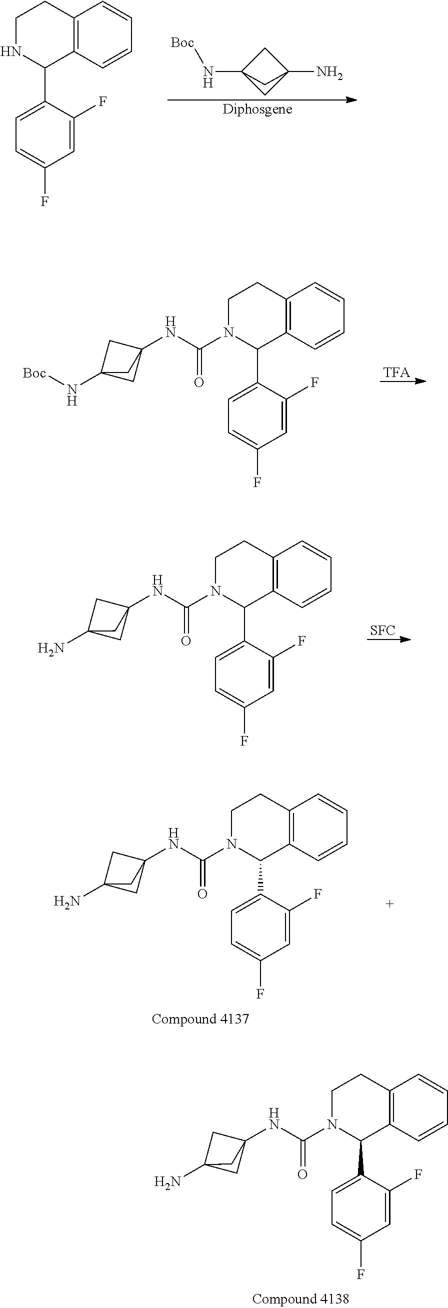

Step 1: To a solution of 1-(2,4-difluorophenyl)-1,2,3,4-tetrahydroisoquinoline (300 mg, 0.41 mmol) in dry acetonitrile (10 mL) was added diphosgene (0.22 mL, 1.83 mmol) at 0° C. After addition, the reaction mixture was stirred at room temperature for 1.5 h, then concentrated and the residue dissolved in dry DMF (8 mL). Tert-butyl (3-aminobicyclo[1.1.1]pentan-1-yl)carbamate (242 mg, 1.22 mmol) was added to the reaction mixture, followed by TEA (0.51 mL, 3.66 mmol). The reaction mixture was stirred at room temperature for 16 h before being poured into water (20 mL) and extracted with DCM (3×10 mL). The combined organic layers were dried over anhydrous Na₂SO₄, filtered and concentrated. The crude product was purified by prep-HPLC to give tert-butyl (3-(1-(2,4-difluorophenyl)-1,2,3,4-tetrahydroisoquinoline-2-carboxamido)bicyclo[1.1.1]pentan-1-yl)carbamate.

LCMS: (M+H)⁺=470.2; Retention time=1.81 min. LCMS CP Method 1

Step 2: To a solution of tert-butyl (R)-(3-(1-(2,4-difluorophenyl)-1,2,3,4-tetrahydroisoquinoline-2-carboxamido)bicyclo[1.1.1]pentan-1-yl)carbamate (400 mg, 0.85 mmol) in DCM (5 mL) was added TFA (1 mL) at 0° C. The mixture was stirred at rt for 2 h then concentrated and the residue dissolved in water. The mixture was adjusted to alkaline pH with 1 N NaOH and extracted with DCM (3×20 mL). The combined organic layers were washed with brine, dried over Na₂SO₄, filtered and concentrated. The crude residue was purified by prep-HPLC to give N-(3-aminobicyclo[1.1.1]pentan-1-yl)-1-(2,4-difluorophenyl)-3,4-dihydroisoquinoline-2(1H)-carboxamide.

LCMS: (M+H)⁺=369.6; Retention time=1.45 min. LCMS CP Method C1

The diastereomers (166 mg) were separated by chiral SFC eluting with CO₂/MeOH containing 0.2% methanolic ammonia=70:30 over a Daicel® IG column (20×250 mm, 10 μm) to give Compound 4137 and Compound 4138. Configuration at the 1 position of tetrahydroisoquinoline is assigned arbitrarily based on chromatographic elution order as compared to related analogues of known configuration.

Compound 4137: LCMS: (M+H)⁺=370.0; Retention time=1.68 min. LCMS CP Method C

¹H NMR (400 Hz, CDCl3): 7.22-7.12 (m, 3H), 7.00-6.95 (m, 2H), 6.85-6.74 (m, 2H), 6.25 (s, 1H), 5.22 (d, J=4 Hz, 1H), 3.99-3.94 (m, 1H), 3.34-3.27 (m, 1H), 3.05-2.97 (m, 1H), 2.85-2.79 (m, 1H), 2.06 (s, 6H).

Chiral SFC: CO₂/MeOH (75:25) containing 0.2% ammonia over CHIRALPAK® IG column (4.6×100 mm, 5 μm), retention time=1.584 min), 99.16% ee.

Compound 4138: LCMS: (M+H)⁺=370.0; Retention time=1.68 min. LCMS CP Method C

¹H NMR (400 Hz, CDCl3): 7.23-7.13 (m, 3H), 7.02-6.96 (m, 2H), 6.86-6.74 (m, 2H), 6.25 (s, 1H), 5.20 (d, J=3.6 Hz, 1H), 4.00-3.95 (m, 1H), 3.34-3.29 (m, 1H), 3.05-2.98 (m, 1H), 2.86-2.80 (m, 1H), 2.07 (s, 6H).

Chiral SFC: CO₂/MeOH (75:25) containing 0.2% ammonia over CHIRALPAK® IG column (4.6×100 mm, 5 μm), retention time=1.273 min), 100% ee.

### Example 61: (S)—N-((1S,5R)-5-amino-3,3-difluorocyclohexyl)-1-(4-fluorophenyl)-3,4-dihydroisoquinoline-2(1H)-carboxamide (Compound 4141) and (S)—N-((1R,5S)-5-amino-3,3-difluorocyclohexyl)-1-(4-fluorophenyl)-3,4-dihydroisoquinoline-2(1H)-carboxamide (Compound 4142)

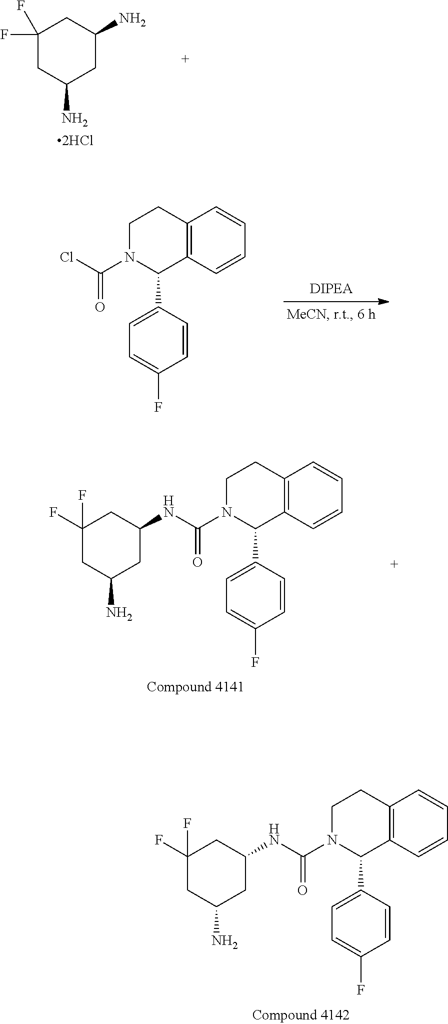 [SMILES: N[C@@H]1C[C@H](N)CC(F)(F)C1 ; "+" ; O=C(Cl)N1CCc2ccccc2[C@@H]1c1ccc(F)cc1 ; N[C@@H]1C[C@H](NC(=O)N2CCc3ccccc3[C@@H]2c2ccc(F)cc2)CC(F)(F)C1 ; "+" ; N[C@H]1C[C@@H](NC(=O)N2CCc3ccccc3[C@@H]2c2ccc(F)cc2)CC(F)(F)C1]

### Synthesis of (S)—N-((1S,5R)-5-amino-3,3-difluorocyclohexyl)-1-(4-fluorophenyl)-3,4-dihydroisoquinoline-2(1H)-carboxamide (Compound 4141); (S)—N-((1R,5S)-5-amino-3,3-difluorocyclohexyl)-1-(4-fluorophenyl)-3,4-dihydroisoquinoline-2(1H)-carboxamide (Compound 4142)

A solution of (S)-1-(4-fluorophenyl)-3,4-dihydroisoquinoline-2(1H)-carbonyl chloride (279 mg, 0.962 mmol) in acetonitrile (10 mL) was added to a solution of cis-5,5-difluorocyclohexane-1,3-diamine dihydrochloride (429 mg, 1.923 mmol) and N,N-diisopropylethylamine (0.840 mL, 4.81 mmol) in acetonitrile (10 mL). After stirring at room temperature for 6 hours, the reaction mixture was diluted with methanol (10 mL) and evaporated under reduced pressure. The residue was purified by flash column chromatography (0 to 10% (7M ammonia in methanol) in dichloromethane) and preparative SFC (Method AN). to give (S)—N-((1S,5R)-5-amino-3,3-difluorocyclohexyl)-1-(4-fluorophenyl)-3,4-dihydroisoquinoline-2(1H)-carboxamide (Compound 4141) as the first eluting SFC isomer and (S)—N-((1R,5S)-5-amino-3,3-difluorocyclohexyl)-1-(4-fluorophenyl)-3,4-dihydroisoquinoline-2(1H)-carboxamide (Compound 4142) as the second eluting SFC isomer after lyophilization from a mixture of acetonitrile and water (1:1, 3 mL).

Compound 4141: LCMS: 98%, RT=1.12 min., (M+H)⁺=404 (Method P). SFC: RT=2.49 min., (M+H)⁺=404 (Method AD). ¹H NMR (400 MHz, DMSO-d₆) δ 7.30-7.06 (m, 8H), 6.70 (d, J=8.0 Hz, 1H), 6.43 (s, 1H), 3.85-3.67 (m, 2H), 3.21 (ddd, J=13.6, 9.3, 4.8 Hz, 1H), 3.04-2.92 (m, 1H), 2.86 (ddd, J=15.4, 9.3, 5.7 Hz, 1H), 2.69 (dt, J=16.3, 4.8 Hz, 1H), 2.35-2.14 (m, 2H), 2.07-1.97 (m, 1H), 1.92-1.56 (m, 2H), 1.34 (q, J=12.0 Hz, 1H).

Compound 4142: LCMS: 99%, RT=1.11 min., (M+H)⁺=404 (Method P). SFC: RT=2.723 min., (M+H)⁺=404 (Method AD). ¹H NMR (400 MHz, DMSO-d₆) δ 7.29-7.06 (m, 8H), 6.65 (d, J=7.8 Hz, 1H), 6.43 (s, 1H), 3.83-3.67 (m, 2H), 3.21 (ddd, J=13.5, 9.3, 4.9 Hz, 1H), 2.98-2.80 (m, 2H), 2.68 (dt, J=16.4, 4.8 Hz, 1H), 2.31-2.11 (m, 2H), 2.01 (d, J=12.2 Hz, 1H), 1.85-1.49 (m, 2H), 1.28 (q, J=12.0 Hz, 1H).

### Example 62: Synthesis of Compound 4143

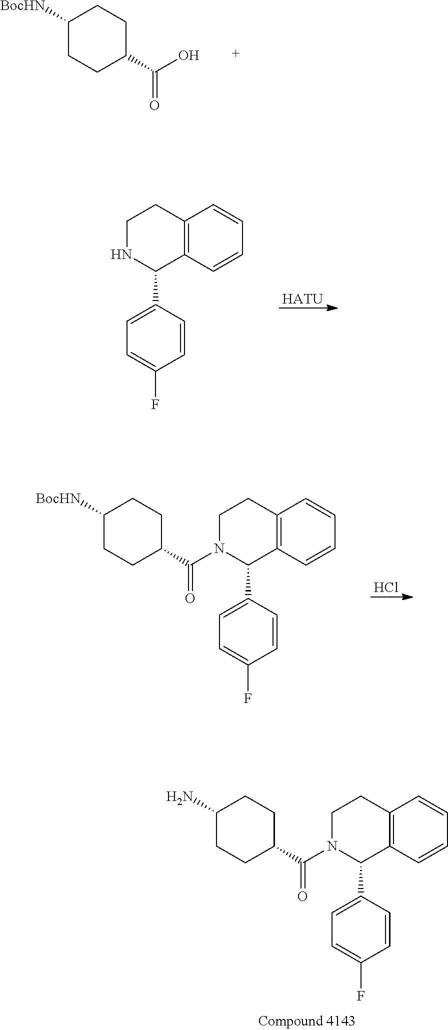

Step 1: To a solution of Cis-4-(tert-butoxycarbonylamino)cyclohexanecarboxylic acid (243 mg, 1 mmol) in DCM (5 mL) were added (S)-1-(4-fluorophenyl)-1,2,3,4-tetrahydroisoquinoline (227 mg, 1 mmol), Et₃N (202 g, 2 mmol) and HATU (456 mg, 1.2 mmol) at 0° C. and the reaction mixture was stirred at room temperature for 2 h. The reaction mixture was diluted with DCM (20 mL) and washed with saturated aqueous NH₄Cl solution (20 mL) and brine. The organic phase was dried over Na₂SO₄, filtered and concentrated to give (S)-tert-butyl 4-(1-(4-fluorophenyl)-1,2,3,4-tetrahydroisoquinoline-2-carbonyl)cyclohexylcarbamate which was used directly in the next step reaction without further purification.

LCMS: (M+H)⁺=453.1; Retention time=2.15 min. LCMS CP Method A

Step 2: To a solution of cis-(S)-tert-butyl 4-(1-(4-fluorophenyl)-1,2,3,4-tetrahydroisoquinoline-2-carbonyl)cyclohexylcarbamate (452 mg, 1 mmol) in DCM (5 mL) was added HCl solution (1 mL, 4M in dioxane) and the reaction mixture was stirred at room temperature overnight The reaction mixture was concentrated to give a residue which was purified by Prep-HPLC to give cis-(S)-(4-aminocyclohexyl)(1-(4-fluorophenyl)-3,4-dihydroisoquinolin-2(1H)-yl)methanone.

Compound 4143: LCMS: (M+H)⁺=353.1; Retention time=1.39 min. LCMS CP Method B

¹H NMR (400 Hz, MeOD): 8.54 (s, 1H), 7.27-7.17 (m, 6H), 7.09-7.00 (m, 3H), 6.84 (s, 1H), 3.97-3.93 (m, 1H), 3.52-3.48 (m, 1H), 3.104-2.89 (m, 3H), 2.04-2.00 (m, 1H), 1.97-1.74 (m, 8H).

Chiral SFC: CO₂/MeOH containing 0.2% ammonia=65:35 over CHIRALPAK® IG column (4.6×100 mm, 3 μm), retention time=3.651 min), 100% ee.

### Example 63: Synthesis of Compound 4144

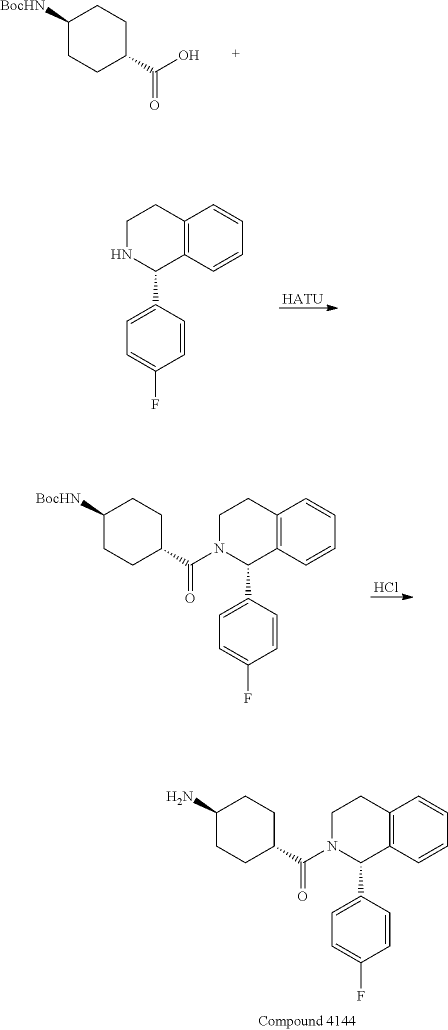

Compound 4144 was prepared following a similar synthesis as for Compound 4143.

Compound 4144: LCMS: (M+H)⁺=353.2; Retention time=1.37 min. LCMS CP Method B

¹H NMR (400 Hz, MeOD): 8.48 (s, 1H), 7.26-7.15 (m, 6H), 7.06 (d, J=7.6 Hz, 1H), 6.96 (t, J=8.8 Hz, 2H), 6.80 (s, 1H), 3.97-3.93 (m, 1H), 3.52-3.46 (m, 1H), 3.11-2.90 (m, 2H), 2.88-2.73 (m, 2H), 2.11-2.07 (m, 2H), 1.90-1.85 (m, 2H), 1.70-1.49 (m, 4H).

Chiral SFC: CO₂/MeOH containing 0.2% ammonia=65:35 over CHIRALPAK® IG column (4.6×100 mm, 5 μm), retention time=3.390 min), 100% ee.

### Example 64: cis-3-aminocyclobutyl (S)-1-(4-fluorophenyl)-3,4-dihydroisoquinoline-2(1H)-carboxylate (Compound 4155)

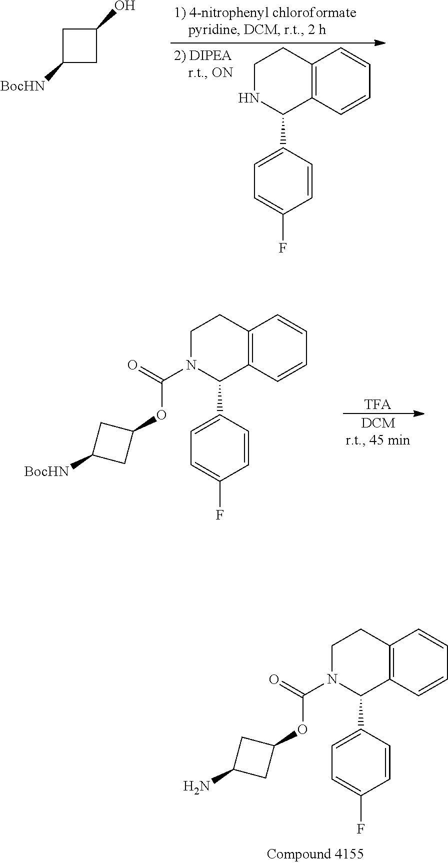 [SMILES: CC(C)(C)OC(=O)N[C@H]1C[C@@H](O)C1 ; Fc1ccc([C@@H]2NCCc3ccccc32)cc1 ; CC(C)(C)OC(=O)N[C@H]1C[C@@H](OC(=O)N2CCc3ccccc3[C@@H]2c2ccc(F)cc2)C1 ; "DCMr.t., 45 min" ; "TFA" ; N[C@H]1C[C@@H](OC(=O)N2CCc3ccccc3[C@@H]2c2ccc(F)cc2)C1]

### Synthesis of cis-3-((tert-butoxycarbonyl)amino)cyclobutyl (S)-1-(4-fluorophenyl)-3,4-dihydroisoquinoline-2(1H)-carboxylate

Under nitrogen atmosphere, 4-nitrophenyl chloroformate (108 mg, 0.534 mmol) was added to a solution of tert-butyl (cis-3-hydroxycyclobutyl)carbamate (100 mg, 0.534 mmol) and pyridine (0.130 mL, 1.61 mmol) in dichloromethane (2.5 mL). After 2 hours, N,N-diisopropylethylamine (0.121 mL, 0.694 mmol) and (S)-1-(4-fluorophenyl)-1,2,3,4-tetrahydroisoquinoline (146 mg, 0.641 mmol) were added and the mixture was stirred overnight. The reaction mixture was diluted with dichloromethane (10 mL), washed with aqueous HCl (0.5 M, 3×10 mL), a mixture of water and saturated aqueous K₂CO₃ (1:1, 10 mL), and brine (10 mL). The organic phase was dried over Na₂SO₄ and evaporated under reduced pressure to give cis-3-((tert-butoxycarbonyl)amino)cyclobutyl (S)-1-(4-fluorophenyl)-3,4-dihydroisoquinoline-2(1H)-carboxylate. The material was used as such.

LCMS: 100%, RT=4.45 min., (M+Na)⁺=463 (Method AK). ¹H NMR (400 MHz, chloroform-d) mixture of rotamers δ 7.24-7.10 (m, 5H), 7.07-6.90 (m, 3H), 6.47-6.14 (m, 1H), 4.83-4.54 (m, 2H), 4.20-3.90 (m, 1H), 3.81 (s, 1H), 3.28-3.11 (m, 1H), 3.07-2.69 (m, 4H), 1.94 (s, 2H), 1.44 (s, 9H).

### Synthesis of cis-3-aminocyclobutyl (S)-1-(4-fluorophenyl)-3,4-dihydroisoquinoline-2(1H)-carboxylate (Compound 4155)

Trifluoroacetic acid (1.0 mL) was added to a solution of cis-3-((tert-butoxycarbonyl)amino)cyclobutyl (S)-1-(4-fluorophenyl)-3,4-dihydroisoquinoline-2(1H)-carboxylate (123 mg, 0.279 mmol) in dichloromethane (4.0 mL). After stirring at room temperature for 45 minutes, the reaction mixture was diluted with dichloromethane (10 mL) and neutralised with saturated aqueous NaHCO₃. The organic layer was collected, washed with brine (10 mL), dried over Na₂SO₄, and evaporated under reduced pressure. The residue was purified by basic preparative MPLC (Linear Gradient: t=0 min 5% A, t=1 min 5% A; t=2 min 20% A; t=17 min 60% A; t=18 min 100% A; t=23 min 100% A; detection 210/256 nm). The compound-containing fractions were lyophilized from a mixture of acetonitrile and water (1:1, 30 mL) to give cis-3-aminocyclobutyl (S)-1-(4-fluorophenyl)-3,4-dihydroisoquinoline-2(1H)-carboxylate (Compound 4155).

LCMS: 98%, RT=2.68 min., (M+H)⁺=341 (Method AK). ¹H NMR (400 MHz, chloroform-d) mixture of rotamers δ 7.28-7.09 (m, 5H), 7.05-6.87 (m, 3H), 6.51-6.10 (m, 1H), 4.81-4.52 (m, 1H), 4.27-3.81 (m, 1H), 3.33-3.05 (m, 2H), 3.05-2.88 (m, 1H), 2.88-2.66 (m, 3H), 2.01-1.53 (m, 4H).

### Example 65: cis-3-((2-hydroxyethyl)amino)cyclobutyl (S)-1-(4-fluorophenyl)-3,4-dihydroisoquinoline-2(1H)-carboxylate (Compound 4145)

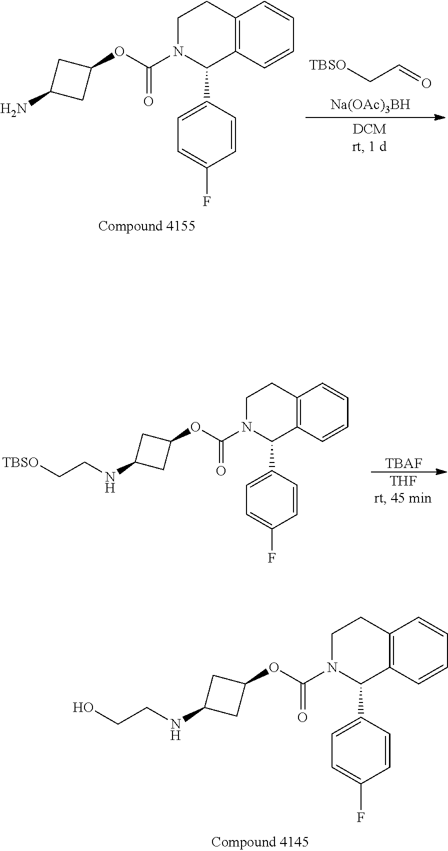

### Synthesis of cis-3-((2-((tert-butyldimethylsilyl)oxy)ethyl)amino)cyclobutyl (S)-1-(4-fluorophenyl)-3,4-dihydroisoquinoline-2(1H)-carboxylate

A solution of (tert-butyldimethylsilyloxy)acetaldehyde (308 mg, 1.767 mmol) in dichloromethane (dry, 5 mL) was added to a solution of cis-3-aminocyclobutyl (S)-1-(4-fluorophenyl)-3,4-dihydroisoquinoline-2(1H)-carboxylate (Compound 4155, 424 mg, 1.121 mmol) in dichloromethane (dry, 5 mL). After 15 minutes, sodium triacetoxyborohydride (335 mg, 1.581 mmol) was added and stirring was continued for 1 day. The mixture was diluted with dichloromethane (20 mL) and saturated aqueous NaHCO₃ (10 mL) and stirred vigorously for 0.5 hour. The layers were separated. The organic phase was washed with saturated aqueous NaHCO₃ (10 mL), dried over Na₂SO₄, and evaporated under reduced pressure The residue was purified by flash column chromatography twice (silica, 5 to 100% ethyl acetate in heptane and 50 to 100% ethyl acetate in heptane) to give cis-3-((2-((tert-butyldimethylsilyl)oxy)ethyl)amino)cyclobutyl (S)-1-(4-fluorophenyl)-3,4-dihydroisoquinoline-2(1H)-carboxylate.

LCMS: 91%, RT=1.73 min., (M+H)⁺=499 (method A). ¹H NMR (400 MHz, chloroform-d) δ 7.25-7.11 (m, 5H), 7.01 (d, J=7.5 Hz, 1H), 6.95 (t, J=8.6 Hz, 2H), 6.49-6.11 (m, 1H), 4.86-4.70 (m, 1H), 4.21-3.90 (m, 1H), 3.70 (t, J=5.3 Hz, 2H), 3.31-3.10 (m, 1H), 3.10-2.88 (m, 2H), 2.87-2.70 (m, 3H), 2.66 (t, J=5.2 Hz, 2H), 1.93-1.73 (m, 2H), 0.90 (s, 9H), 0.07 (s, 6H).

### Synthesis of cis-3-((2-hydroxyethyl)amino)cyclobutyl (S)-1-(4-fluorophenyl)-3,4-dihydroisoquinoline-2(1H)-carboxylate (Compound 4145)

Trifluoroacetic acid (0.4 mL, 5.19 mmol) was added to a solution of cis-3-((2-((tert-butyldimethylsilyl)oxy)ethyl)amino)cyclobutyl (S)-1-(4-fluorophenyl)-3,4-dihydroisoquinoline-2(1H)-carboxylate (93 mg, 0.186 mmol) in dichloromethane (2 mL). After 45 minutes, the reaction mixture was concentrated to dryness under reduced pressure at room temperature. The residue was dissolved in methanol (1 mL) and brought onto a SCX-2 column (1 g) and eluted with methanol until neutral. Next, the column was eluted with ammonia in methanol (1 M). The basic fraction was concentrated to dryness under reduced pressure. The residue was combined with another batch of the crude product (0.197 mmol) and purified by acidic preparative MPLC (Linear Gradient: t=0 min 5% A; t=1 min 5% A; t=2 min 10% A; t=17 min 50% A; t=17 min 100%; t=23 min 100% A; detection: 220 nm). The product containing fractions were pooled, diluted with saturated aqueous NaHCO₃ (3 mL), and the volatiles were removed under reduced pressure. The aqueous phase was partitioned with dichloromethane (10 mL) and the organic layer was passed through a phase separator and evaporated under reduced pressure to give cis-3-((2-hydroxyethyl)amino)cyclobutyl (S)-1-(4-fluorophenyl)-3,4-dihydroisoquinoline-2(1H)-carboxylate (Compound 4145) after lyophilization from a mixture of acetonitrile and water (1:1, 4 mL).

LCMS: 99%, RT=1.07 min., (M+H)⁺=385 (method P). SFC: 96%, RT=3.07 min., (M+H)⁺=385 (method W). ¹H NMR (400 MHz, chloroform-d) mixture of rotamers δ 7.25-7.11 (m, 5H), 7.02 (d, J=7.4 Hz, 1H), 6.96 (t, J=8.5 Hz, 2H), 6.47-6.15 (m, 1H), 4.84-4.71 (m, 1H), 4.20-3.88 (m, 1H), 3.64 (t, J=5.1 Hz, 2H), 3.26-3.12 (m, 1H), 3.06-2.91 (m, 2H), 2.84-2.69 (m, 5H), 1.94-1.58 (m, 4H).

### Example 66: trans-3-((2-hydroxyethyl)amino)cyclobutyl (S)-1-(4-fluorophenyl)-3,4-dihydroisoquinoline-2(1H)-carboxylate (Compound 4146)

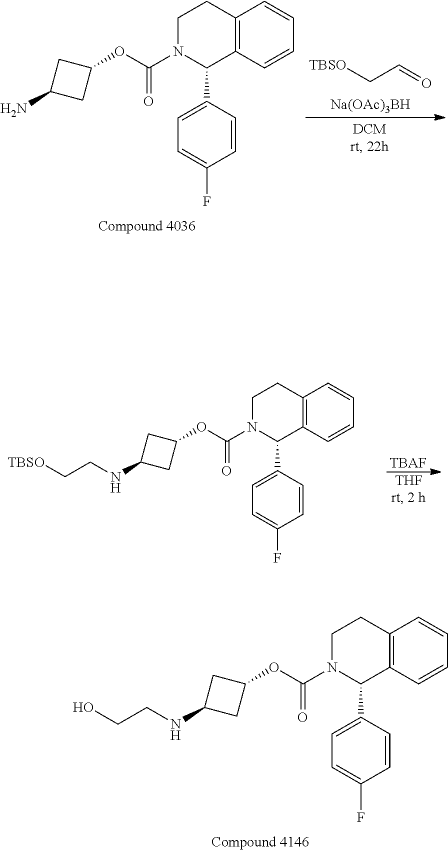

### Synthesis of trans-3-((2-((tert-butyldimethylsilyl)oxy)ethyl)amino)cyclobutyl (S)-1-(4-fluorophenyl)-3,4-dihydroisoquinoline-2(1H)-carboxylate

Starting from trans-3-aminocyclobutyl (S)-1-(4-fluorophenyl)-3,4-dihydroisoquinoline-2(1H)-carboxylate (see Compound 4036, free base, 429.5 mg, 1.173 mmol), trans-3-((2-((tert-butyldimethylsilyl)oxy)ethyl)amino)cyclobutyl (S)-1-(4-fluorophenyl)-3,4-dihydroisoquinoline-2(1H)-carboxylate was prepared as described for cis-3-((2-((tert-butyldimethylsilyl)oxy)ethyl)amino)cyclobutyl (S)-1-(4-fluorophenyl)-3,4-dihydroisoquinoline-2(1H)-carboxylate (see Compound 4145) and purified by acidic preparative MPLC (Linear Gradient: t=0 min 5% A; t=1 min 5% A; t=2 min 20% A; t=17 min 60% A; t=18 min 100%; t=23 min 100% A; detection: 220/254 nm).

LCMS: 97%, RT=1.95 min., (M+H)⁺=499 (method A). ¹H NMR (400 MHz, chloroform-d) δ 7.26-7.13 (m, 5H), 7.02 (d, J=7.4 Hz, 1H), 6.95 (t, J=8.5 Hz, 2H), 6.50-6.14 (m, 1H), 5.21-5.09 (m, 1H), 4.23-3.92 (m, 1H), 3.71 (t, J=5.3 Hz, 2H), 3.56-3.44 (m, 1H), 3.28-3.14 (m, 1H), 3.06-2.92 (m, 1H), 2.83-2.72 (m, 1H), 2.63 (t, J=5.3 Hz, 2H), 2.44-2.27 (m, 2H), 2.27-2.12 (m, 2H), 0.90 (s, 9H), 0.07 (s, 6H).

### Synthesis of trans-3-((2-hydroxyethyl)amino)cyclobutyl (S)-1-(4-fluorophenyl)-3,4-dihydroisoquinoline-2(1H)-carboxylate (Compound 4146)

Under a nitrogen atmosphere, tetrabutylammonium fluoride (1.0 M in tetrahydrofuran, 0.463 mL, 0.463 mmol) was added to a solution of trans-3-((2-((tert-butyldimethylsilyl)oxy)ethyl)amino)cyclobutyl (S)-1-(4-fluorophenyl)-3,4-dihydroisoquinoline-2(1H)-carboxylate (192.4 mg, 0.386 mmol) in tetrahydrofuran (2 mL). After 2 hours, the reaction mixture was concentrated to dryness under reduced pressure (at room temperature). The residue was taken up in methanol (1 mL) and brought onto an SCX-2 column (2 g) and eluted with methanol until neutral. Next, the column was eluted with ammonia in methanol (1 M). The basic fraction was concentrated to dryness under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220 nm). The product containing fractions were combined, diluted with saturated aqueous NaHCO₃ (5 mL), and acetonitrile was removed under reduced pressure. The aqueous residue was extracted with dichloromethane. The layers were separated using a phase-separator and the organic filtrate was evaporated under reduced pressure. The residue was lyophilized from a mixture of acetonitrile and water (1:1, 4 mL) to give trans-3-((2-hydroxyethyl)amino)cyclobutyl (S)-1-(4-fluorophenyl)-3,4-dihydroisoquinoline-2(1H)-carboxylate (Compound 4146).

LCMS: 99%, RT=1.09 min., (M+H)⁺=385 (method P). ¹H NMR (400 MHz, chloroform-d) mixture of rotamers δ 7.25-7.12 (m, 5H), 7.02 (d, J=7.3 Hz, 1H), 6.96 (t, J=8.5 Hz, 2H), 6.50-6.13 (m, 1H), 5.22-5.09 (m, 1H), 4.23-3.91 (m, 1H), 3.64 (t, J=5.1 Hz, 2H), 3.50 (ddd, J=12.5, 7.5, 5.2 Hz, 1H), 3.28-3.13 (m, 1H), 3.07-2.92 (m, 1H), 2.83-2.72 (m, 1H), 2.71 (t, J=5.1 Hz, 2H), 2.43-2.27 (m, 2H), 2.27-2.15 (m, 2H).

### Example 67: (trans-3-aminocyclobutyl)((S)-1-(4-fluorophenyl)-3,4-dihydroisoquinolin-2(1H)-yl)methanone (Compound 4147)

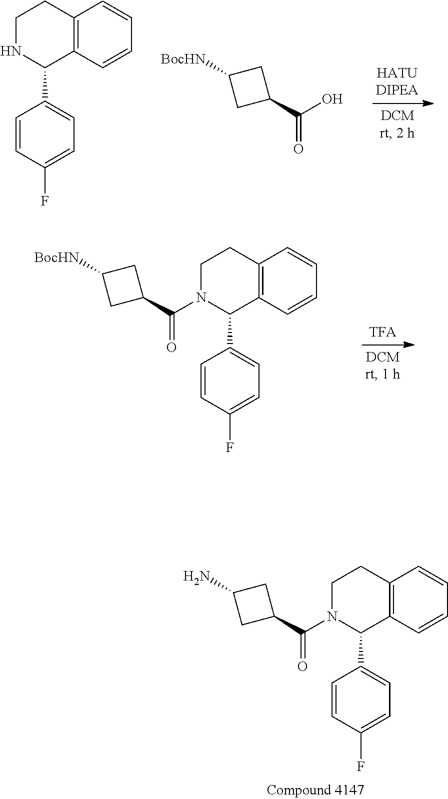 [SMILES: Fc1ccc([C@@H]2NCCc3ccccc32)cc1 ; CC(C)(C)OC(=O)N[C@H]1C[C@H](C(=O)O)C1 ; CC(C)(C)OC(=O)N[C@H]1C[C@H](C(=O)N2CCc3ccccc3[C@@H]2c2ccc(F)cc2)C1 ; N[C@H]1C[C@H](C(=O)N2CCc3ccccc3[C@@H]2c2ccc(F)cc2)C1]

### Synthesis of tert-butyl (trans-3-((S)-1-(4-fluorophenyl)-1,2,3,4-tetrahydroisoquinoline-2-carbonyl)cyclobutyl)carbamate

N,N-diisopropylethylamine (0.092 mL, 0.528 mmol) and 1-[bis(dimethylamino)methylene]-1H-1,2,3-triazolo[4,5-b]pyridinium 3-oxide hexafluorophosphate (HATU, 167 mg, 0.440 mmol) were added to a solution of trans-3-((tert-butoxycarbonyl)amino)cyclobutane-1-carboxylic acid (95 mg, 0.440 mmol) in dichloromethane (2.5 mL). After 10 minutes, (S)-1-(4-fluorophenyl)-1,2,3,4-tetrahydroisoquinoline (100 mg, 0.440 mmol) was added and the mixture was stirred for another 1 hour and 50 minutes. The mixture was diluted with dichloromethane (2.5 mL) and washed with saturated aqueous NaHCO₃ (2×2.5 mL) and aqueous HCl (1 M, 2×2.5 mL).

The organic phase was passed through a phase separator and evaporated under reduced pressure. The residue was purified by flash column chromatography (silica, 10 to 80% ethyl acetate in heptane) to give tert-butyl (trans-3-((S)-1-(4-fluorophenyl)-1,2,3,4-tetrahydroisoquinoline-2-carbonyl)cyclobutyl)carbamate.

LCMS: 99%, RT=2.16 min., (M+H)⁺=425 (method A).

### Synthesis of (trans-3-aminocyclobutyl)((S)-1-(4-fluorophenyl)-3,4-dihydroisoquinolin-2(1H)-yl)methanone (Compound 4147)

Trifluoroacetic acid (1.0 mL, 13 mmol) was added to a solution of tert-butyl (trans-3-((S)-1-(4-fluorophenyl)-1,2,3,4-tetrahydroisoquinoline-2-carbonyl)cyclobutyl)carbamate (157 mg, 0.370 mmol) in dichloromethane (5 mL). After 1 hour, the mixture was diluted with dichloromethane (20 mL) and a mixture of water (5 mL) and saturated aqueous K₂CO₃ (5 mL) and stirred for 30 minutes. The layers were separated over a phase separation filter and the organic layer was concentrated under reduced pressure. The residue was dissolved in methanol (1 mL) and brought onto an SCX-2 column (1 g) and eluted with methanol until neutral. Next, the column was eluted with ammonia in methanol (1 M). The basic fraction was concentrated to dryness under reduced pressure lyophilized from a mixture of acetonitrile and water to give (trans-3-aminocyclobutyl)((S)-1-(4-fluorophenyl)-3,4-dihydroisoquinolin-2(1H)-yl)methanone (Compound 4147).

LCMS: 99%, RT=2.52 min., (M+H)⁺=325 (method AK). ¹H NMR (400 MHz, chloroform-d) mixture of rotamers δ 7.30-7.10 (m, 5H), 7.08-6.90 (m, 3H), 6.89 (s, 0.85H), 5.81 (s, 0.15H), 4.23 (dd, J=13.1, 6.4 Hz, 0.15H) 3.70-3.56 (m, 1.85H), 3.53-3.40 (m, 0.15H), 3.38-3.21 (m, 1.85H), 2.95 (ddd, J=17.0, 11.6, 5.9 Hz, 1H), 2.80 (dt, J=16.2, 3.6 Hz, 1H), 2.71-2.60 (m, 1H), 2.60-2.50 (m, 0.85H), 2.43-2.31 (m, 0.15H), 2.07-1.81 (m, 2H).

### Example 68: cis-3-aminocyclobutyl)((S)-1-(4-fluorophenyl)-3,4-dihydroisoquinolin-2(1H)-yl)methanone (Compound 4148)

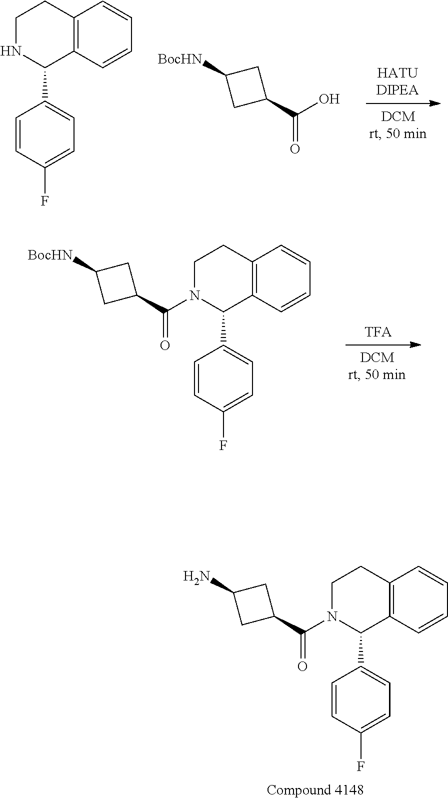 [SMILES: Fc1ccc([C@@H]2NCCc3ccccc32)cc1 ; CC(C)(C)OC(=O)N[C@H]1C[C@@H](C(=O)O)C1 ; CC(C)(C)OC(=O)N[C@H]1C[C@@H](C(=O)N2CCc3ccccc3[C@@H]2c2ccc(F)cc2)C1 ; N[C@H]1C[C@@H](C(=O)N2CCc3ccccc3[C@@H]2c2ccc(F)cc2)C1]

### Synthesis of tert-butyl (cis-3-((S)-1-(4-fluorophenyl)-1,2,3,4-tetrahydroisoquinoline-2-carbonyl)cyclobutyl)carbamate

Starting from cis-3-((tert-butoxycarbonyl)amino)cyclobutane-1-carboxylic acid (95 mg, 0.440 mmol) and (S)-1-(4-fluorophenyl)-1,2,3,4-tetrahydroisoquinoline (100 mg, 0.440 mmol), tert-butyl (cis-3-((S)-1-(4-fluorophenyl)-1,2,3,4-tetrahydroisoquinoline-2-carbonyl)cyclobutyl)carbamate was prepared as described for tert-butyl (trans-3-((S)-1-(4-fluorophenyl)-1,2,3,4-tetrahydroisoquinoline-2-carbonyl)cyclobutyl)carbamate (see Compound 4147.

LCMS: 99%, RT=2.16 min., (M+H)⁺=425 (method A). ¹H NMR (400 MHz, chloroform-d) mixture of rotamers δ 7.29-7.12 (m, 5H), 7.11-6.89 (m, 3H), 6.85 (s, 0.85H), 5.88 (s, 0.15H), 4.90-4.61 (m, 0.85H), 4.60-4.33 (m, 0.15H) 4.26-3.80 (m, 1.15H), 3.71-3.62 (m, 0.85H), 3.41-3.22 (m, 1H), 3.18-3.02 (m, 0.15H), 3.01-2.87 (m, 1.85H), 2.87-2.82 (dt, J=16.0, 3.9 Hz, 0.85H), 2.73-2.38 (m, 2.15H), 2.38-2.24 (m, 0.15H), 2.22-2.11 (m, 1.7H), 2.09-1.97 (m, 0.15H), 1.42 (2×s, 9H).

### Synthesis of (cis-3-aminocyclobutyl)((S)-1-(4-fluorophenyl)-3,4-dihydroisoquinolin-2(1H)-yl)methanone (Compound 4148)

Starting from tert-butyl (cis-3-((S)-1-(4-fluorophenyl)-1,2,3,4-tetrahydroisoquinoline-2-carbonyl)cyclobutyl)carbamate (154 mg, 0.363 mmol), (cis-3-aminocyclobutyl)((S)-1-(4-fluorophenyl)-3,4-dihydroisoquinolin-2(1H)-yl)methanone (Compound 4148) was prepared as described for (trans-3-aminocyclobutyl)((S)-1-(4-fluorophenyl)-3,4-dihydroisoquinolin-2(1H)-yl)methanone (Compound 4147).

LCMS: 99%, RT=2.53 min., (M+H)⁺=325 (method AK). ¹H NMR (400 MHz, chloroform-d) mixture of rotamers δ 7.31-7.07 (m, 5H), 7.07-6.89 (m, 3H), 6.86 (s, 0.85H), 5.90 (s, 0.15H), 4.24-4.14 (m, 0.15H), 3.74-3.65 (m, 0.85H), 3.42-3.22 (m, 2H), 3.05-2.87 (m, 1.15H), 2.87-2.77 (m, 1.7H), 2.72-2.61 (m, 0.15H), 2.59-2.40 (m, 2H), 2.25-2.14 (m, 0.15H), 2.09-1.97 (m, 1.7H), 1.95-1.85 (m, 0.15H).

### Example 69: (trans-3-(aminomethyl)cyclobutyl)((S)-1-(4-fluorophenyl)-3,4-dihydroisoquinolin-2(1H)-yl)methanone (Compound 4149)

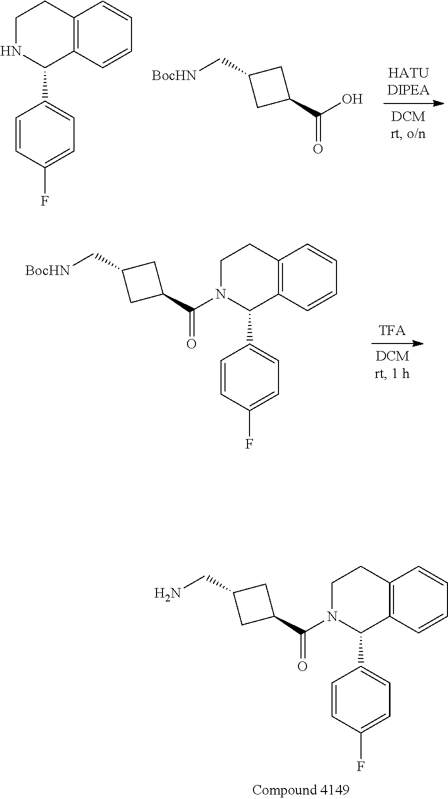 [SMILES: Fc1ccc([C@@H]2NCCc3ccccc32)cc1 ; CC(C)(C)OC(=O)NC[C@H]1C[C@H](C(=O)O)C1 ; CC(C)(C)OC(=O)NC[C@H]1C[C@H](C(=O)N2CCc3ccccc3[C@@H]2c2ccc(F)cc2)C1 ; NC[C@H]1C[C@H](C(=O)N2CCc3ccccc3[C@@H]2c2ccc(F)cc2)C1]

### Synthesis of tert-butyl ((trans-3-((S)-1-(4-fluorophenyl)-1,2,3,4-tetrahydroisoquinoline-2-carbonyl)cyclobutyl)methyl)carbamate

Starting from trans-3-((tert-butoxycarbonyl)amino)cyclobutane-1-carboxylic acid (100 mg, 0.436 mmol) and (S)-1-(4-fluorophenyl)-1,2,3,4-tetrahydroisoquinoline (99 mg, 0.436 mmol), tert-butyl ((trans-3-((S)-1-(4-fluorophenyl)-1,2,3,4-tetrahydroisoquinoline-2-carbonyl)cyclobutyl)methyl)carbamate was prepared as described for tert-butyl (trans-3-((S)-1-(4-fluorophenyl)-1,2,3,4-tetrahydroisoquinoline-2-carbonyl)cyclobutyl)carbamate (see Compound 4147).

LCMS: 99%, RT=2.19 min., (M+H)⁺=439 (method A). ¹H NMR (400 MHz, chloroform-d) mixture of rotamers δ 7.26-7.02 (m, 6H), 7.01-6.89 (m, 2H), 6.87 (s, 0.85H), 5.76 (s, 0.15H), 4.61-4.39 (m, 0.85H), 4.30-4.19 (m, 0.15H), 3.62-3.42 (m, 1H), 3.38-3.08 (m, 4H), 2.93 (ddd, J=17.3, 11.5, 5.8 Hz, 1H), 2.80 (dt, J=16.3, 3.6 Hz, 0.85H), 2.71-2.60 (m, 0.3H), 2.55-2.32 m, 2.7H), 2.30-2.18 (m, 0.15H), 2.05-1.80 (m, 2H), 1.44 (s, 9H).

### Synthesis of (trans-3-(aminomethyl)cyclobutyl)((S)-1-(4-fluorophenyl)-3,4-dihydroisoquinolin-2(1H)-yl)methanone (Compound 4149)

Starting from tert-butyl ((trans-3-((S)-1-(4-fluorophenyl)-1,2,3,4-tetrahydroisoquinoline-2-carbonyl)cyclobutyl)methyl)carbamate (167 mg, 0.381 mmol), (trans-3-(aminomethyl)cyclobutyl)((S)-1-(4-fluorophenyl)-3,4-dihydroisoquinolin-2(1H)-yl)methanone (Compound 4149) was prepared as described for (trans-3-aminocyclobutyl)((S)-1-(4-fluorophenyl)-3,4-dihydroisoquinolin-2(1H)-yl)methanone (Compound 4147).

LCMS: 99%, RT=1.05 min., (M+H)⁺=339 (method P). ¹H NMR (400 MHz, chloroform-d) mixture of rotamers δ 7.29-7.01 (m, 6H), 7.01-6.88 (m, 2H), 6.86 (s, 0.85H), 5.88 (s, 0.15H), 4.25-4.16 (m, 0.15H), 3.67 (dd, J=14.0, 3.5 Hz, 0.85H), 3.41-3.09 (m, 2.15H), 3.01-2.88 (m, 1H), 2.86-2.76 (m, 0.85H), 2.74-2.60 (m, 2H), 2.37-2.16 (m, 3H), 2.09-1.96 (m, 1.7H), 1.95-1.83 (m, 0.3H).

### Example 70: (cis-3-(aminomethyl)cyclobutyl)((S)-1-(4-fluorophenyl)-3,4-dihydroisoquinolin-2(1H)-yl)methanone (Compound 4150)

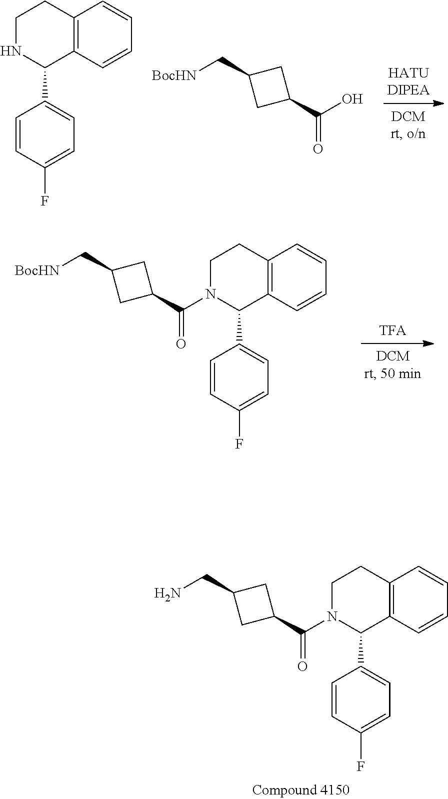 [SMILES: Fc1ccc([C@@H]2NCCc3ccccc32)cc1 ; CC(C)(C)OC(=O)NC[C@H]1C[C@@H](C(=O)O)C1 ; CC(C)(C)OC(=O)NC[C@H]1C[C@@H](C(=O)N2CCc3ccccc3[C@@H]2c2ccc(F)cc2)C1 ; NC[C@H]1C[C@@H](C(=O)N2CCc3ccccc3[C@@H]2c2ccc(F)cc2)C1]

### Synthesis of tert-butyl ((cis-3-((S)-1-(4-fluorophenyl)-1,2,3,4-tetrahydroisoquinoline-2-carbonyl)cyclobutyl)methyl)carbamate

Starting from cis-3-((tert-butoxycarbonyl)amino)cyclobutane-1-carboxylic acid (100 mg, 0.436 mmol) and (S)-1-(4-fluorophenyl)-1,2,3,4-tetrahydroisoquinoline (99 mg, 0.436 mmol), tert-butyl ((cis-3-((S)-1-(4-fluorophenyl)-1,2,3,4-tetrahydroisoquinoline-2-carbonyl)cyclobutyl)methyl)carbamate was prepared as described for tert-butyl (trans-3-((S)-1-(4-fluorophenyl)-1,2,3,4-tetrahydroisoquinoline-2-carbonyl)cyclobutyl)carbamate (see Compound 4147).

LCMS: 99%, RT=2.18 min., (M+H)⁺=439 (method A). ¹H NMR (400 MHz, chloroform-d) mixture of rotamers δ 7.28-7.01 (m, 6H), 7.01-6.88 (m, 2H), 6.85 (s, 0.85H), 5.84 (s, 0.15H), 4.74-4.54 (m, 0.85H), 4.35-4.14 (m, 0.3H), 3.69-3.60 (m, 0.85H), 3.32 (ddd, J=13.8, 11.4, 4.5 Hz, 1H), 3.28-3.20 (m, 0.15H), 3.20-3.02 (m, 2.85H), 2.94 (ddd, J=16.8, 11.1, 5.8 Hz, 1H), 2.81 (dt, J=16.3, 3.6 Hz, 0.85H), 2.71-2.59 (m, 0.15H), 2.53-2.36 (m, 1H), 2.35-2.14 (m, 2.15H), 2.10-1.98 (m, 1.7H), 1.95-1.83 (m, 0.15H), 1.42 (s, 9H).

### Synthesis of (cis-3-(aminomethyl)cyclobutyl)((S)-1-(4-fluorophenyl)-3,4-dihydroisoquinolin-2(1H)-yl)methanone (Compound 4150)

Starting from tert-butyl ((cis-3-((S)-1-(4-fluorophenyl)-1,2,3,4-tetrahydroisoquinoline-2-carbonyl)cyclobutyl)methyl)carbamate (161 mg, 0.367 mmol), (cis-3-(aminomethyl)cyclobutyl)((S)-1-(4-fluorophenyl)-3,4-dihydroisoquinolin-2(1H)-yl)methanone (Compound 4150) was prepared as described for (trans-3-aminocyclobutyl)((S)-1-(4-fluorophenyl)-3,4-dihydroisoquinolin-2(1H)-yl)methanone (Compound 4147).

LCMS: 97%, RT=1.04 min., (M+H)⁺=339 (method P). ¹H NMR (400 MHz, chloroform-d) mixture of rotamers δ 7.30-7.01 (m, 6H), 7.01-6.90 (m, 2H), 6.88 (s, 0.85H), 5.77 (s, 0.15H), 4.24 (dt, J=12.7, 6.0 Hz, 0.15H) 3.59 (dt, J=13.5, 4.1 Hz, 0.85H), 3.51-3.40 (m, 0.15H), 3.37-3.20 (m, 1.85H), 2.95 (ddd, J=16.7, 10.9, 5.8 Hz, 1H), 2.85-2.74 (m, 2.7H), 2.74-2.58 (m, 0.45H), 2.57-2.38 (m, 1.7H), 2.38-2.20 (m, 1.15H), 2.05-1.79 (m, 2H).

### Example 71: (cis-3-amino-3-(hydroxymethyl)cyclobutyl)((S)-1-(4-fluorophenyl)-3,4-dihydroisoquinolin-2(1H)-yl)methanone (Compound 4151)

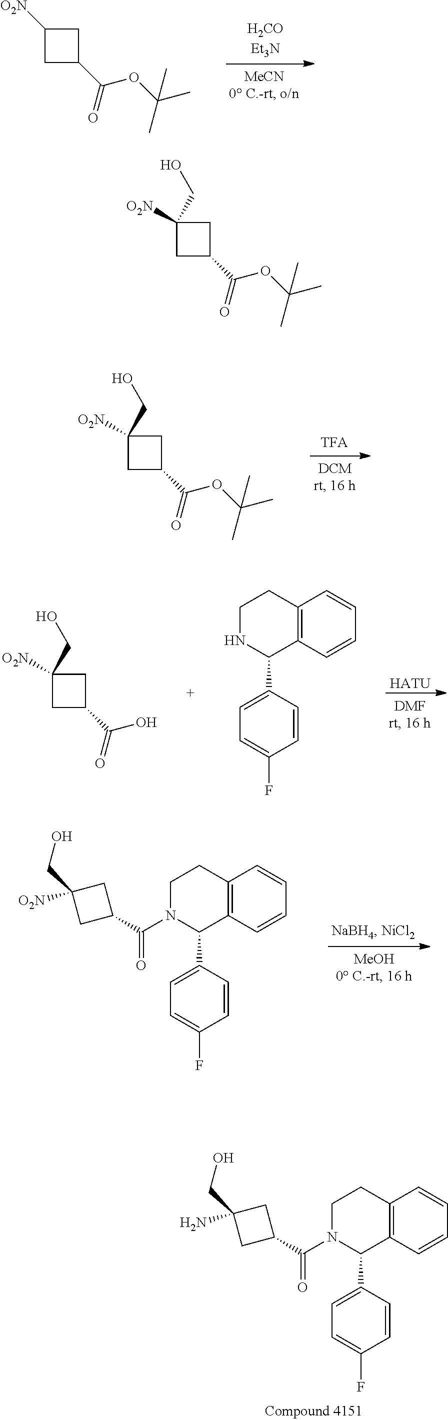

### Synthesis of tert-butyl trans-3-(hydroxymethyl)-3-nitrocyclobutane-1-carboxylate and tert-butyl cis-3-(hydroxymethyl)-3-nitrocyclobutane-1-carboxylate

At 0° C., formaldehyde (37 wt % solution in water stabilized with 10-15% methanol, 0.187 mL, 2.49 mmol) and triethylamine (0.259 mL, 1.864 mmol) were added to a solution of tert-butyl 3-nitrocyclobutane-1-carboxylate (250 mg, 1.242 mmol) in acetonitrile (5 mL). The mixture was allowed to warm to room temperature and stirred overnight. The mixture was concentrated to dryness under reduced pressure. The residue was purified by flash column chromatography (silica, 0 to 40% ethyl acetate in heptane) to give tert-butyl trans-3-(hydroxymethyl)-3-nitrocyclobutane-1-carboxylate as the first eluting isomer and tert-butyl cis-3-(hydroxymethyl)-3-nitrocyclobutane-1-carboxylate as the second eluting isomer.

First eluting isomer: ¹H NMR (400 MHz, chloroform-d) δ 4.09 (d, J=6.3 Hz, 2H), 3.26-3.13 (m, 1H), 3.03-2.91 (m, 2H), 2.67-2.58 (m, 2H), 2.55 (t, J=6.6 Hz, 1H), 1.47 (s, 9H).

Second eluting isomer: ¹H NMR (400 MHz, chloroform-d) δ 4.06-3.99 (m, 2H), 3.00-2.91 (m, 2H), 2.89-2.77 (m, 1H), 2.65-2.54 (m, 2H), 2.42 (br s, 1H), 1.45 (s, 9H).

### Synthesis of cis-3-(hydroxymethyl)-3-nitrocyclobutane-1-carboxylic acid

Trifluoroacetic acid (1 mL, 13.06 mmol) was added to a solution of tert-butyl cis-3-(hydroxymethyl)-3-nitrocyclobutane-1-carboxylate (75 mg, 0.324 mmol) in dichloromethane (4 mL) and stirred for 16 hours. The mixture was concentrated under reduced pressure to give cis-3-(hydroxymethyl)-3-nitrocyclobutane-1-carboxylic acid which was used as such.

¹H NMR (400 MHz, chloroform-d) δ 4.86 (s, 1H), 4.08 (s, 1H), 3.25-3.18 (m, 1H), 3.15-2.97 (m, 2H), 2.78-2.64 (m, 2H).

### Synthesis of ((S)-1-(4-fluorophenyl)-3,4-dihydroisoquinolin-2(1H)-yl)(cis-3-(hydroxymethyl)-3-nitrocyclobutyl)methanone

1-[Bis(dimethylamino)methylene]-1H-1,2,3-triazolo[4,5-b]pyridinium 3-oxide hexafluorophosphate (HATU, 156 mg, 0.411 mmol) was added to a solution of cis-3-(hydroxymethyl)-3-nitrocyclobutane-1-carboxylic acid (0.324 mmol) in N,N-dimethylformamide (4 mL). After 15 minutes, (S)-1-(4-fluorophenyl)-1,2,3,4-tetrahydroisoquinoline (86 mg, 0.377 mmol) was added and the reaction mixture was stirred for 16 hours. The reaction mixture was diluted with water and extracted with dichloromethane (3×5 mL). The combined organic phase was dried over Na₂SO₄ and evaporated under reduced pressure. The residue was purified by flash column chromatography (silica, 0 to 80% ethyl acetate in heptane) to give ((S)-1-(4-fluorophenyl)-3,4-dihydroisoquinolin-2(1H)-yl)(cis-3-(hydroxymethyl)-3-nitrocyclobutyl)methanone.

LCMS: 98%, RT=2.01 min., (M+H)⁺=385 (method A). SFC: RT=3.65 min., (M+H)⁺=385 (method W). ¹H NMR (400 MHz, chloroform-d) mixture of rotamers δ 7.32-7.02 (m, 6H), 7.02-6.88 (m, 2H), 6.84 (s, 0.85H), 5.79 (s, 0.15H), 4.20-3.98 (m, 2.15H), 3.60 (ddd, J=13.7, 6.0, 2.9 Hz, 0.85H), 3.45-3.29 (m, 1H), 3.28-3.16 (m, 0.3H), 3.14-2.89 (m, 3.7H), 2.89-2.81 (m, 0.85H), 2.81-2.42 (m, 3.15H).

### Synthesis of (cis-3-amino-3-(hydroxymethyl)cyclobutyl)((S)-1-(4-fluorophenyl)-3,4-dihydroisoquinolin-2(1H)-yl)methanone (Compound 4151)

Nickel(II) chloride hexahydrate (43.9 mg, 0.185 mmol) was added to a solution of ((S)-1-(4-fluorophenyl)-3,4-dihydroisoquinolin-2(1H)-yl)(cis-3-(hydroxymethyl)-3-nitrocyclobutyl)methanone (71 mg, 0.185 mmol) in methanol (4 mL) and the solution was cooled in an ice/water bath. Sodium borohydride (55.9 mg, 1.478 mmol) was added portionwise. After the addition, the reaction mixture was allowed to warm to room temperature and stirred for 16 hours. The reaction mixture was filtered and the filtrate evaporated under reduced pressure. The residue was dissolved in methanol (1 mL) and brought onto a SCX-2 column (1 g) and eluted with methanol until neutral. Next, the column was eluted with ammonia in methanol (3 M). The basic fraction was concentrated to dryness under reduced pressure to give (cis-3-amino-3-(hydroxymethyl)cyclobutyl)((S)-1-(4-fluorophenyl)-3,4-dihydroisoquinolin-2(1H)-yl)methanone (Compound 4151) after lyophilization from a mixture of acetonitrile and water (1:1, 4 mL).

LCMS: 98%, RT=1.01 min., (M+H)⁺=355 (method P). SFC: RT=4.34 min., (M+H)⁺=355 (method F). ¹H NMR (400 MHz, chloroform-d) mixture of rotamers δ 7.26-7.11 (m, 4.85H), 7.11-7.01 (m, 1.15H), 7.01-6.89 (m, 2H), 6.87 (s, 0.85H), 5.85 (s, 0.15H), 4.26-4.14 (m, 0.15H), 3.66 (ddd, J=13.7, 6.0, 2.9 Hz, 0.85H), 3.60-3.47 (m, 2H), 3.42-3.24 (m, 1H), 3.20-3.07 (m, 0.15H), 3.02-2.86 (m, 1.85H), 2.82 (dt, J=16.2, 3.8 Hz, 0.85H), 2.72-2.62 (m, 0.15H), 2.44-2.25 (m, 2H), 2.25-2.13 (m, 1.85H), 2.05-1.96 (m, 0.15H).

### Example 72: (trans-3-amino-3-(hydroxymethyl)cyclobutyl)((S)-1-(4-fluorophenyl)-3,4-dihydroisoquinolin-2(1H)-yl)methanone (Compound 4152)

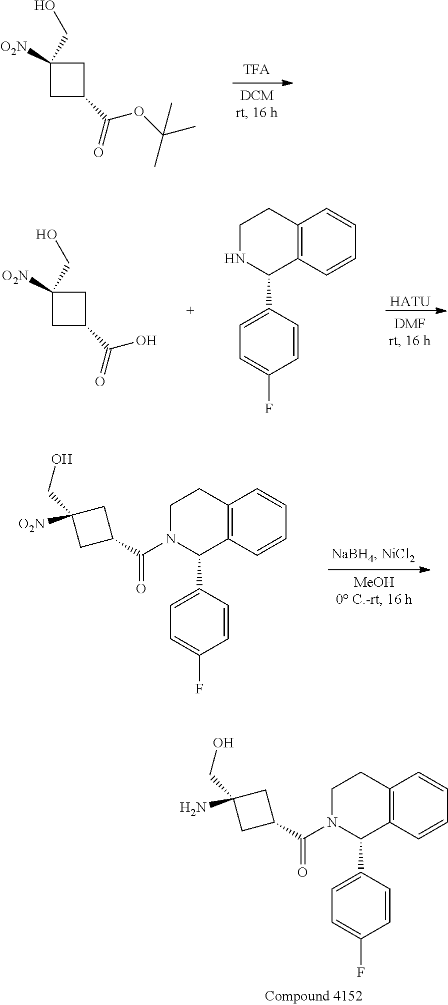

### Synthesis of trans-3-(hydroxymethyl)-3-nitrocyclobutane-1-carboxylic acid

Trifluoroacetic acid (1 mL, 13.06 mmol) was added to a solution of tert-butyl trans-3-(hydroxymethyl)-3-nitrocyclobutane-1-carboxylate (see Compound 4151, 38 mg, 0.164 mmol) in dichloromethane (4 mL) and stirred for 16 hours. The mixture was concentrated under reduced pressure to give trans-3-(hydroxymethyl)-3-nitrocyclobutane-1-carboxylic acid which was used as such.

### Synthesis of ((S)-1-(4-fluorophenyl)-3,4-dihydroisoquinolin-2(1H)-yl)(trans-3-(hydroxymethyl)-3-nitrocyclobutyl)methanone

Starting from trans-3-(hydroxymethyl)-3-nitrocyclobutane-1-carboxylic acid (0.148 mmol) and (S)-1-(4-fluorophenyl)-1,2,3,4-tetrahydroisoquinoline (37.1 mg, 0.163 mmol), ((S)-1-(4-fluorophenyl)-3,4-dihydroisoquinolin-2(1H)-yl)(trans-3-(hydroxymethyl)-3-nitrocyclobutyl)methanone was prepared as described for ((S)-1-(4-fluorophenyl)-3,4-dihydroisoquinolin-2(1H)-yl)(cis-3-(hydroxymethyl)-3-nitrocyclobutyl)methanone (see Compound 4151).

LCMS: 96%, RT=2.03 min., (M+H)⁺=385 (method A). SFC: RT=3.68 min., (M+H)⁺=385 (method W).

### Synthesis of (trans-3-amino-3-(hydroxymethyl)cyclobutyl)((S)-1-(4-fluorophenyl)-3,4-dihydroisoquinolin-2(1H)-yl)methanone (Compound 4152)

Starting from (S)-(1-(4-fluorophenyl)-3,4-dihydroisoquinolin-2(1H)-yl)(trans-3-hydroxy-3-(nitromethyl)cyclobutyl)methanone (32 mg, 0.083 mmol), (trans-3-amino-3-(hydroxymethyl)cyclobutyl)((S)-1-(4-fluorophenyl)-3,4-dihydroisoquinolin-2(1H)-yl)methanone (Compound 4152) was prepared as described for (cis-3-amino-3-(hydroxymethyl)cyclobutyl)((S)-1-(4-fluorophenyl)-3,4-dihydroisoquinolin-2(1H)-yl)methanone (Compound 4151) and additionally purified by preparative LCMS (method BJ).

LCMS: 99%, RT=0.99 min., (M+H)⁺=355 (method P). SFC: RT=4.12 min., (M+H)⁺=355 (method F). ¹H NMR (400 MHz, chloroform-d, D₂O) mixture of rotamers δ 7.29-7.00 (m, 6H), 7.00-6.88 (m, 2H), 6.85 (s, 0.85H), 5.83 (s, 0.15H), 4.25-4.14 (m, 0.15H), 3.68-3.56 (m, 1H), 3.47 (s, 2H), 3.47-3.23 (m, 1.85H), 3.00-2.86 (m, 1H), 2.80 (dt, J=16.2, 3.7 Hz, 0.85H), 2.72-2.58 (m, 0.3H), 2.44 (ddd, J=18.7, 12.1, 6.9 Hz, 1.85H), 2.25-2.19 (m, 0.15H), 2.19-2.04 (m, 1.7H), 2.04-1.95 (m, 0.15H).

### Example 73: (trans-3-(aminomethyl)-3-hydroxycyclobutyl)((S)-1-(4-fluorophenyl)-3,4-dihydroisoquinolin-2(1H)-yl)methanone (Compound 4154) and (cis-3-(aminomethyl)-3-hydroxycyclobutyl)((S)-1-(4-fluorophenyl)-3,4-dihydroisoquinolin-2(1/h)-yl)methanone (Compound 4153)

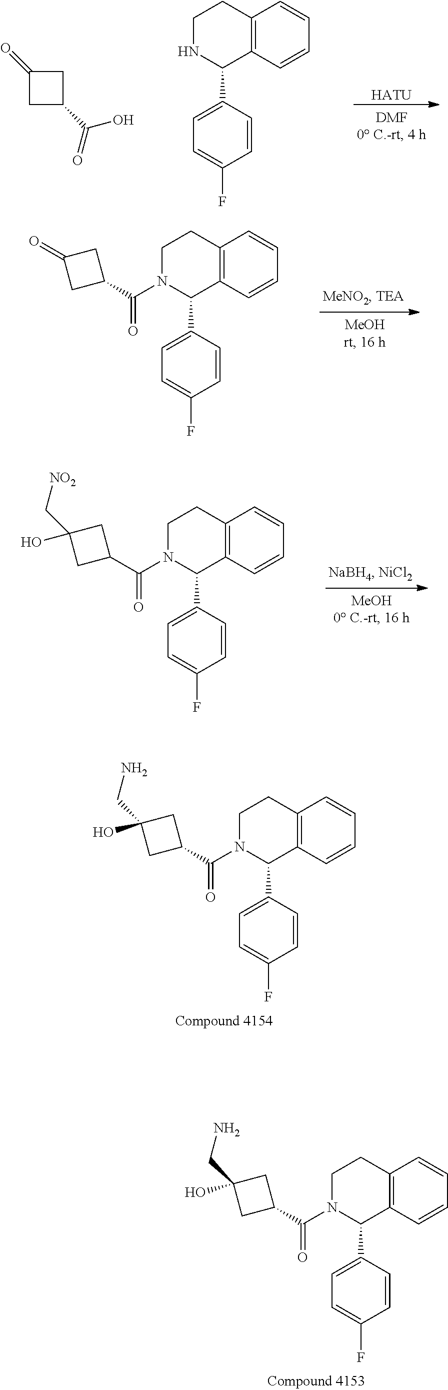

### Synthesis of (S)-3-(1-(4-fluorophenyl)-1,2,3,4-tetrahydroisoquinoline-2-carbonyl)cyclobutan-1-one

Starting from 3-oxocyclobutane-1-carboxylic acid (3 g, 26.3 mmol) and (S)-1-(4-fluorophenyl)-1,2,3,4-tetrahydroisoquinoline (6.57 g, 28.9 mmol), (S)-3-(1-(4-fluorophenyl)-1,2,3,4-tetrahydroisoquinoline-2-carbonyl)cyclobutan-1-one was prepared as described for ((S)-1-(4-fluorophenyl)-3,4-dihydroisoquinolin-2(1H)-yl)(cis-3-(hydroxymethyl)-3-nitrocyclobutyl)methanone (see Compound 4151).

LCMS: 99%, RT=2.01 min., (M+H)⁺=324 (method A). ¹H NMR (400 MHz, chloroform-d) mixture of rotamers δ 7.33-7.04 (m, 6H), 7.04-6.93 (m, 2H), 6.92 (s, 0.85H), 6.00 (s, 0.15H), 4.31-4.22 (m, 0.15H), 3.79 (ddd, J=13.6, 6.7, 3.1 Hz, 0.85H), 3.75-3.29 (m, 6H), 3.29-3.17 (m, 1.85H), 3.17-3.05 (m, 0.15H), 2.99 (ddd, J=16.8, 11.1, 5.8 Hz, 1H), 2.93-2.84 (m, 0.85H), 2.79-2.69 (m, 0.15H).

### Synthesis of (S)-(1-(4-fluorophenyl)-3,4-dihydroisoquinolin-2(1H)-yl)(3-hydroxy-3-(nitromethyl)cyclobutyl)methanone

Nitromethane (1.038 mL, 19.05 mmol) and triethylamine (1.324 mL, 9.52 mmol) were added to a solution of (S)-3-(1-(4-fluorophenyl)-1,2,3,4-tetrahydroisoquinoline-2-carbonyl)cyclobutan-1-one (770 mg, 2.381 mmol) in methanol (10 mL) and the reaction was stirred for 16 hours. The mixture was evaporated under reduced pressure and the residue purified by flash column chromatography (silica, 0 to 50% ethyl acetate in heptane) to give (S)-(1-(4-fluorophenyl)-3,4-dihydroisoquinolin-2(1H)-yl)(3-hydroxy-3-(nitromethyl)cyclobutyl)methanone.

LCMS: 97%, RT=1.99 min., (M+H)⁺=385 (method A). SFC: 30%, RT=3.86 min., (M+H)⁺=385; 70%, RT=4.41 min., (M+H)⁺=385 (method W). ¹H NMR (400 MHz, chloroform-d) mixture of rotamers (˜6:1) and diastereoisomers (˜3:1) δ 7.28-7.03 (m, 6H), 7.03-6.91 (m, 2H), 6.90+6.86 (2×s, 0.85H), 5.87+5.82 (2×s, 0.15H), 4.73-4.49 (m, 2H), 4.28-4.18 (m, 1H), 3.70-3.04 (m, 3H), 3.00-2.31 (m, 4H).

### Synthesis of (trans-3-(aminomethyl)-3-hydroxycyclobutyl)((S)-1-(4-fluorophenyl)-3,4-dihydroisoquinolin-2(1H)-yl)methanone (Compound 4154) and (cis-3-(aminomethyl)-3-hydroxycyclobutyl)((S)-1-(4-fluorophenyl)-3,4-dihydroisoquinolin-2(1H)-yl)methanone (Compound 4153)

Starting from (S)-(1-(4-fluorophenyl)-3,4-dihydroisoquinolin-2(1H)-yl)(3-hydroxy-3-(nitromethyl)cyclobutyl)methanone (270 mg, 0.702 mmol), (trans-3-(aminomethyl)-3-hydroxycyclobutyl)((S)-1-(4-fluorophenyl)-3,4-dihydroisoquinolin-2(1H)-yl)methanone (Compound 4154) as the first eluting SFC isomer after further purification by preparative LCMS (method BJ) and (cis-3-(aminomethyl)-3-hydroxycyclobutyl)((S)-1-(4-fluorophenyl)-3,4-dihydroisoquinolin-2(1H)-yl)methanone (Compound 4153) as the second eluting SFC isomer were prepared as described for (cis-3-amino-3-(hydroxymethyl)cyclobutyl)((S)-1-(4-fluorophenyl)-3,4-dihydroisoquinolin-2(1H)-yl)methanone (Compound 4151) and additionally purified by preparative chiral SFC (method Z). The stereochemistry of cyclobutyl moiety is arbitrarily assigned.

Compound 4153: LCMS: 96% RT=1.00 min., (M+H)⁺=355 (method P). SFC: RT=4.51 min., (M+H)⁺=355 (method W). ¹H NMR (400 MHz, chloroform-d) mixture of rotamers δ 7.31-7.00 (m, 6H), 7.00-6.89 (m, 2H), 6.89 (s, 0.85H), 5.88 (s, 0.15H), 4.28-4.16 (m, 0.15H), 3.74-3.60 (m, 0.85H), 3.42-3.25 (m, 1H), 3.13-2.61 (m, 5H), 2.54-2.15 (m, 4H).

Compound 4154: LCMS: 99% RT=1.01 min., (M+H)⁺=355 (method P). SFC: RT=3.61 min., (M+H)⁺=355 (method W). ¹H NMR (400 MHz, chloroform-d) mixture of rotamers δ 7.26-7.01 (m, 6H), 7.00-6.89 (m, 2H), 6.85 (s, 0.85H), 5.85 (s, 0.15H), 4.24-4.15 (m, 0.15H), 3.69-3.54 (m, 1H), 3.42-3.22 (m, 1.85H), 3.00-2.88 (m, 1H), 2.87-2.72 (m, 2.7H), 2.71-2.52 (m, 0.3H), 2.42-2.07 (m, 4H).

Cellular Assays: To measure the efficacy of compounds, a progranulin induction cellular assay in mouse primary microglia (pMG), primary cortical neurons, and BV-2 cell lines is used. BV-2 cells are split the day before plating into a 96 well plate format at approximately 80%. Cells should be plated the day before and allowed for 1 hour attachment period and for 16 hour incubation. Levels of progranulin secreted into the cell culture medium or retained in the cell lysate can be quantified using an ELISA-based readout and measurement of secreted mouse PGRN in the medium was assessed by the methodology published by Ghidoni et al. 2012. Standard ELISA kits to measure PGRN are available from vendors such as Adipogen, R&D, and Biovendor.

In vivo Assays: A mouse ELISA protocol to detect progranulin in brain, plasma, or cerebrospinal fluid (CSF) can be used, with GRN+/− mice or GRN+/+ mice (available from TACONIC). The mouse is administered a compound as disclosed herein and the amount of progranulin in the brain is assessed after a specific amount of time. Mice treated with a test compound or compounds are compared to control mice which are not treated with the compound. Treatment can be done with a single or multiple dosing of compounds. Control samples are assigned a relative value of 100%.

Other in vivo assays can be performed using a GRN+/− and GRN+/+ rats, non-human primates (e.g., monkey, dog) using a similar protocol.

Treatment with the test compound increases the progranulin secretion relative to the control is at least about 110%, at least about 130%, at least about 150%, at least about 180%, at least about 200%, at least about 250%, or at least about 300%.

Table B below presents the results of a PGRN assay as described above.

TABLE B

| Compound No. | EC₅₀ μM | Compound No. | EC₅₀ μM |
|---|---|---|---|
| 4002 | 0.266 | 4013 | >10.0 |
| 4001 | 0.44 | 4021 | >10.0 |
| 4005 | >10.0 | 4006 | >10.0 |
| 4007 | >10.0 | 4011 | 5.16 |
| 4009 | >10.0 | 4012 | 7.81 |
| 4010 | >10.0 | 4022 | 5.33 |
| 4008 | >10.0 | 4023 | >10.0 |
| 4017 | >10.0 | 4040 | 2.33 |
| 4016 | >10.0 | 4036 | 1.12 |
| 4024 | >10.0 | 4035 | 0.49 |
| 4052 | >10.0 | 4033 | 0.32 |
| 4014 | >10.0 | 4041 | 0.31 |
| 4004 | 0.225 | 4042 | 0.059 |
| 4015 | >10.0 | 4051 | 0.46 |
| 4018 | >10.0 | 4043 | 0.90 |
| 4026 | 0.85 | 4044 | 1.03 |
| 4025 | >10.0 | 4045 | 0.60 |
| 4049 | >10.0 | 4055 | 0.16 |
| 4046 | 0.92 | 4053 | 0.031 |
| 4056 | 1.44 | 4054 | 0.12 |
| 4057 | 1.75 | 4003 | 0.72 |
| 4019 | 2.37 | 4067 | 0.913 |
| 4020 | 3.29 | 4068 | 0.290 |
| 4028 | 2.32 | 4069 | 0.672 |
| 4029 | 3.4 | 4121 | 0.365 |
| 4047 | 1.64 | 4122 | 0.208 |
| 4030 | 9.64 | 4123 | >2.10 |
| 4031 | >10.0 | 4064 | 0.968 |
| 4032 | >10.0 | 4065 | 0.821 |
| 4048 | 1.09 | 4124 | 0.347 |
| 4058 | 0.90 | 4066 | 0.610 |
| 4037 | >10.0 | 4125 | 0.280 |
| 4038 | 1.69 | 4126 | 0.571 |
| 4034 | 0.37 | 4062 | 0.334 |
| 4050 | >10.0 | 4127 | 0.773 |
| 4027 | 1.06 | 4128 | 0.319 |
| 4039 | 1.21 | 4129 | 0.318 |
| 4130 | 0.740 | 4144 | 3.120 |
| 4132 | 1.010 | 4145 | 0.191 |
| 4133 | 0.864 | 4146 | 0.089 |
| 4134 | 1.290 | 4147 | 0.099 |
| 4131 | 0.359 | 4148 | 0.092 |
| 4135 | 1.610 | 4149 | 0.245 |
| 4136 | 0.604 | 4150 | 0.079 |
| 4137 | 0.091 | 4151 | 0.083 |
| 4138 | 5.590 | 4152 | 0.245 |
| 4141 | 0.201 | 4153 | 0.130 |
| 4142 | 0.810 | 4154 | 0.112 |
| 4143 | 0.084 | 4155 | 0.131 |

In view of the many possible embodiments to which the principles of the disclosure may be applied, it should be recognized that the illustrated embodiments are only examples and should not be taken as limiting the scope of the invention.

What is claimed:

1. A compound, or pharmaceutically acceptable salt thereof, having a structure of Formula (I): 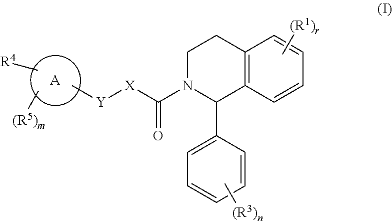
wherein
X is O, NR², or CR^{A}R^{B};
Y is C₀₋₆alkylene;
ring A is a 3- to 8-membered carbocycle;
R^{A} is hydrogen, OH, or O—C₁₋₃alkyl;
R^{B} is hydrogen; or
R^{A} and R^{B} together are oxo;
R¹ is hydrogen, C₁₋₆alkyl, C₀₋₃ haloalkyl, O—C₁₋₃ haloalkyl, C₀₋₃ alkylene-CN, C₀₋₃ alkylene-NR^{N}₂, C₀₋₆ alkylene-OR^{N}, C₀₋₆ alkylene-C(O)OR^{N}, C₀₋₆ alkylene-C(O)N(R^{N})₂, or C₀₋₆ alkylene-SOₚR^{N}; each R^{N} is independently hydrogen or C₁₋₆alkyl, and p is 0-2;
R² is hydrogen, C₁₋₆alkyl, C₃₋₆alkynyl, or C₁₋₆alkylene-SO₂—C₁₋₃alkyl;
each R³ is independently halo;
R⁴ is hydrogen, OH, halo, or C₀₋₆alkylene-NR⁶R⁷;
each R⁵ is independently hydrogen, C₁₋₃alkyl, C₀₋₃hydroxyalkyl, or halo;
R⁶ is hydrogen or C₁₋₃alkyl;
R⁷ is hydrogen, C₁₋₃alkyl, C₁₋₃haloalkyl, C₁₋₃hydroxyalkyl, C₂₋₆alkynyl, C₁₋₃alkylene-O—C₂₋₆alkynyl, C₀₋₃alkylene-SO₂—C₁₋₃alkyl, C₀₋₃alkylene-SO₂—C₁₋₃haloalkyl, C(O)C₁₋₆alkyl, or C(O)C₁₋₆haloalkyl; or
R⁶ and R⁷ together with the nitrogen to which they are attached form a 4- to 6-membered heterocycle having 0-1 additional ring heteroatoms selected from N, O, and S and optionally substituted with 1, 2, or 3 groups independently selected from halo and oxo;
m is 1 or 2;
n is 0-3; and
r is 1 or 2.

2. The compound or salt of claim 1, wherein X is O.

3. The compound or salt of claim 1, wherein X is CH₂.

4. The compound or salt of claim 1, wherein X is NR².

5. The compound or salt of claim 4, wherein R² is hydrogen.

6. The compound or salt of claim 4, wherein R² is CH₃.

7. The compound or salt of any one of claims 1 to 6, wherein Y is C₀alkylene.

8. The compound or salt of any one of claims 1 to 6, wherein Y is C₁₋₆alkylene.

9. The compound or salt of claim 8, wherein Y is C₀alkylene.

10. The compound or salt of claim 8, wherein Y is C₂alkylene.

11. The compound or salt of claim 8, wherein Y is C₀alkylene.

12. The compound or salt of any one of claims 1 to 11, wherein ring A is a 3- to 5-membered carbocycle.

13. The compound or salt of any one of claims 1 to 11, wherein ring A is a 6- to 8-membered carbocycle.

14. The compound or salt of any one of claims 1 to 11, wherein ring A is

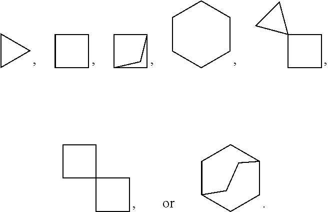

15. The compound or salt of claim 14, wherein ring A is [symbol] .

16. The compound or salt of any one of claims 1 to 15, wherein R⁴ is hydrogen.

17. The compound or salt of any one of claims 1 to 15, wherein R⁴ is OH.

18. The compound or salt of any one of claims 1 to 15, wherein R⁴ is halo.

19. The compound or salt of claim 18, wherein R⁴ is F.

20. The compound or salt of any one of claims 1 to 15, wherein R⁴ is C₀₋₆alkylene-NR⁶R⁷.

21. The compound or salt of claim 20, wherein R⁴ is —CH₂NH₂, —CH₂NHCH₃, —CH₂CH₂NH₂, —CH₂CH(CH₃)NH₂, —CH₂C(CH₃)₂NH₂, or —CH₂CH₂N(CH₃)₂.

22. The compound or salt of claim 20, wherein R⁴ is NR⁶R⁷.

23. The compound or salt of claim 20, wherein R⁴ is C₁alkylene-NR⁶R⁷.

24. The compound or salt of claim 22 or 23, wherein R⁶ is hydrogen.

25. The compound or salt of claim 22 or 23, wherein R⁶ is C₁₋₃alkyl.

26. The compound or salt of claim 25, wherein R⁶ is methyl.

27. The compound or salt of any one of claims 22 to 26, wherein R⁷ is hydrogen.

28. The compound or salt of any one of claims 22 to 26, wherein R⁷ is C₁₋₃alkyl.

29. The compound or salt of claim 28, wherein R⁷ is methyl.

30. The compound or salt of claim 28, wherein R⁷ is ethyl.

31. The compound or salt of any one of claims 22 to 26, wherein R⁷ is C₁₋₃haloalkyl.

32. The compound or salt of claim 31, wherein R⁷ is trifluoroethyl.

33. The compound or salt of any one of claims 22 to 26, wherein R⁷ is C₂₋₆alkynyl.

34. The compound or salt of claim 33 wherein R⁷ is propargyl.

35. The compound or salt of any one of claims 22 to 26, wherein R⁷ is C₀₋₃alkylene-SO₂—C₁₋₃alkyl or C₀₋₃alkylene-SO₂—C₁₋₃haloalkyl.

36. The compound or salt of claim 35, wherein R⁷ is SO₂-methyl or SO₂CF₃.

37. The compound or salt of claim 35, wherein R⁷ is C₂alkylene-SO₂-methyl.

38. The compound or salt of any one of claims 22 to 26, wherein R⁷ is C(O)C₁₋₆alkyl or C(O)C₁₋₆haloalkyl.

39. The compound or salt of claim 38, wherein R⁷ is C(O)CH₃ or C(O)CF₃.

40. The compound or salt of claim 23 or 24, wherein R⁶ and R⁷ together with the nitrogen to which they are attached form a 4- to 6-membered heterocycle.

41. The compound or salt of claim 40, wherein R⁶ and R⁷ together with the nitrogen to which they are attached form a heterocycle selected from 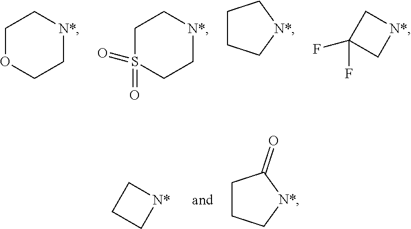
wherein N* indicates the nitrogen to which R⁶ and R⁷ are attached.

42. The compound or salt of any one of claims 1 to 15, wherein R⁴ is —NH₂, —NHCH₃, —N(CH₃)₂, —NHCH₂CH₃, —NHCH₂CF₃, —NH-propargyl, —NHC(O)CH₃, —NHC(O)CF₃, —NHSO₂CH₃, —NHSO₂CF₃, or —NHCH₂CH₂SO₂CH₃.

43. The compound or salt of claim 42, wherein R⁴ is —NH₂.

44. The compound or salt of any one of claims 1 to 43, wherein m is 1.

45. The compound or salt of any one of claims 1 to 43, wherein m is 2.

46. The compound or salt of any one of claims 1 to 45, wherein at least one R⁵ is H.

47. The compound or salt of claim 46, wherein each R⁵ is hydrogen.

48. The compound or salt of any one of claims 1 to 45, wherein at least one R⁵ is C₁₋₃alkyl.

49. The compound or salt of claim 48, wherein at least one R⁵ is methyl.

50. The compound or salt of claim 49, wherein each R⁵ is methyl.

51. The compound or salt of any one of claims 1 to 45, wherein at least one R⁵ is halo.

52. The compound or salt of claim 51, wherein at least one R⁵ is fluoro.

53. The compound or salt of claim 52, wherein each R⁵ is fluoro.

54. The compound or salt of any one of claims 1 to 53, wherein n is 1.

55. The compound or salt of any one of claims 1 to 54, wherein R³ is fluoro.

56. The compound or salt of any one of claims 1 to 55, wherein R^{N} is hydrogen.

57. The compound or salt of any one of claims 1 to 55, wherein R^{N} is C₁₋₆alkyl.

58. The compound or salt of any one of claims 1 to 57, wherein R¹ is F, Cl, OH, OMe, OiPr, OBn, O-cyclopropyl, CF₃, OCF₃, CN, SO₂Me, SO₂-cyclopropyl, SO₂iPr, oxo, imidazolyl, triazolyl, pyrrolidinyl, pyrrolidinonyl, thiadiazolyl, methyl-thiadiazolyl, trifluoromethyl-thiadiazolyl, oxadiazolyl, methyl-oxadiazolyl, trifluoromethyl-oxadiazolyl, or phenyl.

59. The compound or salt of claim 57, wherein R¹ is F or C₁.

60. The compound or salt of claim 59, wherein R¹ is F.

61. A compound, or pharmaceutically acceptable salt thereof, having a structure as shown in Table A.

62. The compound or salt of claim 61, selected from the group consisting of 4001, 4002, 4004, 40033, 4034, 4035, 4041, 4042, 4051, 4053, 4054, and 4055.

63. The compound or salt of claim 61, selected from the group consisting of 4137, 4150, 4151, 4146, 4036, 4155, 4141, and 4143.

64. The compound or salt of claim 61, having a structure of compound 4001:

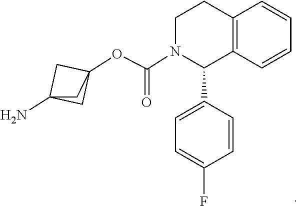 [SMILES: NC12CC(OC(=O)N3CCc4ccccc4[C@@H]3c3ccc(F)cc3)(C1)C2]

65. The compound or salt of claim 61, having a structure of having a structure of compound 4002:

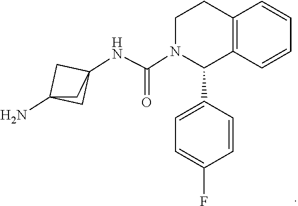 [SMILES: NC12CC(NC(=O)N3CCc4ccccc4[C@@H]3c3ccc(F)cc3)(C1)C2]

66. The compound or salt of claim 61, having a structure of having a structure of compound 4004:

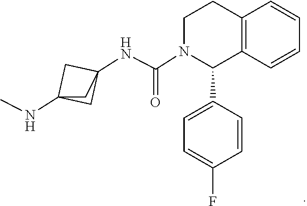 [SMILES: CNC12CC(NC(=O)N3CCc4ccccc4[C@@H]3c3ccc(F)cc3)(C1)C2]

67. The compound or salt of claim 61, having a structure of having a structure of compound 4033:

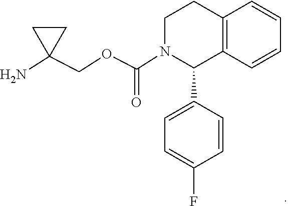 [SMILES: NC1(COC(=O)N2CCc3ccccc3[C@@H]2c2ccc(F)cc2)CC1]

68. The compound or salt of claim 61, having a structure of having a structure of compound 4034:

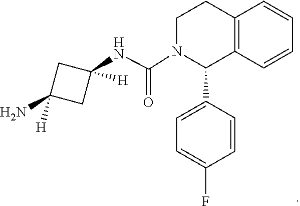 [SMILES: N[C@H]1C[C@@H](NC(=O)N2CCc3ccccc3[C@@H]2c2ccc(F)cc2)C1]

69. The compound or salt of claim 61, having a structure of having a structure of compound 4035:

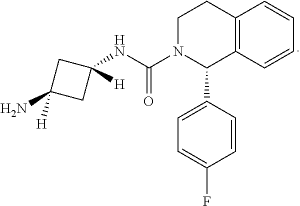 [SMILES: N[C@H]1C[C@H](NC(=O)N2CCc3ccccc3[C@@H]2c2ccc(F)cc2)C1]

70. The compound or salt of claim 61, having a structure of having a structure of compound 4041:

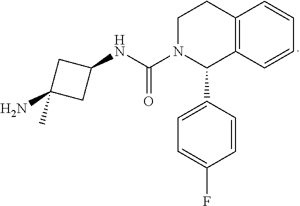 [SMILES: C[C@]1(N)C[C@@H](NC(=O)N2CCc3ccccc3[C@@H]2c2ccc(F)cc2)C1]

71. The compound or salt of claim 61, having a structure of having a structure of compound 4042:

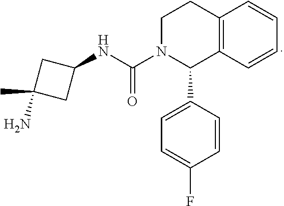 [SMILES: C[C@]1(N)C[C@H](NC(=O)N2CCc3ccccc3[C@@H]2c2ccc(F)cc2)C1]

72. The compound or salt of claim 61, having a structure of having a structure of compound 4051:

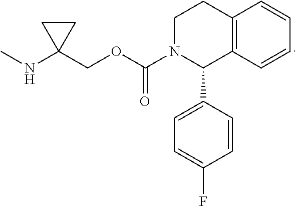 [SMILES: CNC1(COC(=O)N2CCc3ccccc3[C@@H]2c2ccc(F)cc2)CC1]

73. The compound or salt of claim 61, having a structure of having a structure of compound 4053:

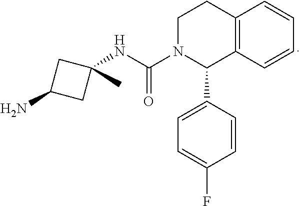 [SMILES: C[C@]1(NC(=O)N2CCc3ccccc3[C@@H]2c2ccc(F)cc2)C[C@H](N)C1]

74. The compound or salt of claim 61, having a structure of having a structure of compound 4054:

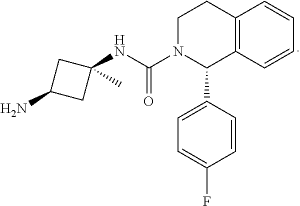 [SMILES: C[C@]1(NC(=O)N2CCc3ccccc3[C@@H]2c2ccc(F)cc2)C[C@@H](N)C1]

75. The compound or salt of claim 61, having a structure of having a structure of compound 4055:

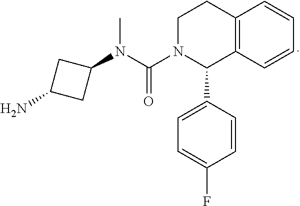 [SMILES: CN(C(=O)N1CCc2ccccc2[C@@H]1c1ccc(F)cc1)[C@H]1C[C@H](N)C1]

76. The compound or salt of claim 61, having a structure of having a structure of compound 4036:

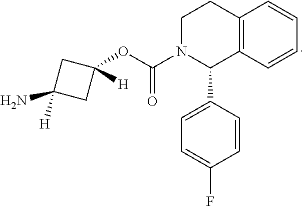 [SMILES: N[C@H]1C[C@H](OC(=O)N2CCc3ccccc3[C@@H]2c2ccc(F)cc2)C1]

77. The compound or salt of claim 61, having a structure of having a structure of compound 4137:

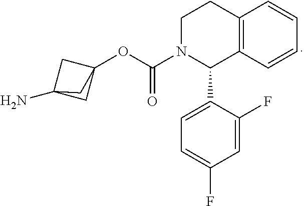 [SMILES: NC12CC(OC(=O)N3CCc4ccccc4[C@@H]3c3ccc(F)cc3F)(C1)C2]

78. The compound or salt of claim 61, having a structure of having a structure of compound 4150:

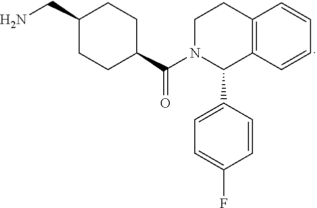 [SMILES: NC[C@H]1CC[C@@H](C(=O)N2CCc3ccccc3[C@@H]2c2ccc(F)cc2)CC1]

79. The compound or salt of claim 61, having a structure of having a structure of compound 4151:

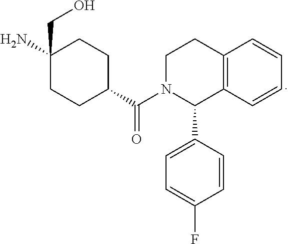 [SMILES: N[C@]1(CO)CC[C@H](C(=O)N2CCc3ccccc3[C@@H]2c2ccc(F)cc2)CC1]

80. The compound or salt of claim 61, having a structure of having a structure of compound 4146:

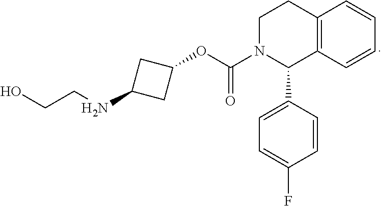 [SMILES: O=C(O[C@H]1C[C@H](NCCO)C1)N1CCc2ccccc2[C@@H]1c1ccc(F)cc1]

81. The compound or salt of claim 61, having a structure of having a structure of compound 4141:

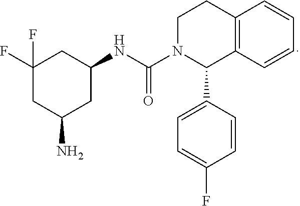 [SMILES: N[C@@H]1C[C@H](NC(=O)N2CCc3ccccc3[C@@H]2c2ccc(F)cc2)CC(F)(F)C1]

82. The compound or salt of claim 61, having a structure of having a structure of compound 4143:

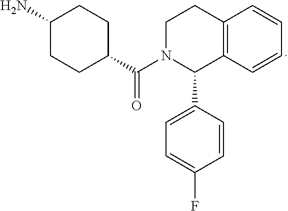 [SMILES: N[C@H]1CC[C@@H](C(=O)N2CCc3ccccc3[C@@H]2c2ccc(F)cc2)CC1]

83. The compound or salt of claim 61, having a structure of having a structure of compound 4155:

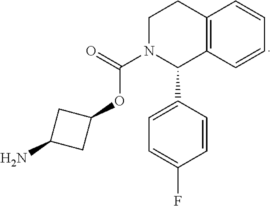 [SMILES: N[C@H]1C[C@@H](OC(=O)N2CCc3ccccc3[C@@H]2c2ccc(F)cc2)C1]

84. The compound or salt of any one of claims 1 to 83 in the form of a salt.

85. A method of synthesizing a compound or salt of any one of claims 1 to 84, comprising:
heating an acyl azide compound to undergo a Curtius rearrangement to form an isocyanate compound; and
admixing the isocyanate compound with an amine compound to form a urea derivative.

86. The method of claim 85, wherein the isocyanate compound is prepared in situ.

87. The method of claim 85 or 86, further comprising synthesizing the acyl azide by admixing a carboxylic acid and an azide compound to form the acyl azide.

88. The method of claim 87, wherein the azide compound is diphenyl phosphoryl azide.

89. A pharmaceutical composition comprising the compound of salt of any one of claims 1 to 84 and a pharmaceutically acceptable excipient.

90. Use of the compound or salt of any one of claims 1 to 84 as a medicament for the modulation of progranulin.

91. The use of claim 90, wherein progranulin secretion is increased.

92. A method of modulating progranulin in a subject in need thereof comprising administering to the subject the compound or salt of any one of claims 1 to 84 in an amount effective to increase the level of progranulin or granulin in the subject.

93. A method of treating a progranulin-associated disorder in a subject in need thereof comprising administering a therapeutically effective amount of the compound or salt of any one of claims 1 to 84 to the subject.

94. The method of claim 93, wherein the progranulin-associated disorder is Alzheimer's disease (AD), Parkinson's disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington's disease (HD), Spinocerebellar ataxia (SCA), Spinal muscular atrophy (SMA), a lysosomal storage disease, nephropathy, a disease associated with inclusions and/or misfunction of C₉orf72, TDP-43, FUS, UBQLN2, VCP, CHMP28, and/or MAPT, an acute neurological disorder, glioblastoma, or neuroblastoma.

95. The method of claim 94, wherein the lysosomal storage disease is Paget's disease, Gaucher's disease, Nieman's Pick disease, Tay-Sachs Disease, Fabry Disease, Pompes disease, or Naso-Hakula disease.

96. The method of claim 94, wherein the acute neurological disorder is stroke, cerebral hemorrhage, traumatic brain injury or head trauma.

97. The method of claim 96, wherein the progranulin-associated disorder is Frontotemporal dementia (FTD).
